SEQUENCE LISTING (394 sequences)

SEQ ID NO: 1 - protein, 21 aa, synthetic construct
  source 1..21: note = CD8alpha signal peptide
MALPVTALLLPLALLLHAARP

SEQ ID NO: 2 - protein, 20 aa, synthetic construct
  source 1..20: note = IgK signal peptide
METDTLLLWVLLLWVPGSTG

SEQ ID NO: 3 - protein, 22 aa, synthetic construct
  source 1..22: note = GM-CSF signal peptide
MLLLVTSLLLCELPHPAFLLIP

SEQ ID NO: 4 - protein, 274 aa, synthetic construct
  source 1..274: note = Pembrolizumab-derived anti-PD-1 scFv VH-linker-VL
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTA

SEQ ID NO: 5 - protein, 125 aa, synthetic construct
  source 1..125: note = Pembrolizumab-derived anti-PD-1 scFv VH
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKG

SEQ ID NO: 6 - protein, 9 aa, synthetic construct
  source 1..9: note = Pembrolizumab-derived anti-PD-1 scFv VH CDR1
YTFTNYYMY

SEQ ID NO: 7 - protein, 14 aa, synthetic construct
  source 1..14: note = Pembrolizumab-derived anti-PD-1 scFv VH CDR2
WMGGINPSNGGTNF

SEQ ID NO: 8 - protein, 12 aa, synthetic construct
  source 1..12: note = Pembrolizumab-derived anti-PD-1 scFv VH CDR3
RRDYRFDMGFDY

SEQ ID NO: 9 - protein, 15 aa, synthetic construct
  source 1..15: note = 3xGGGGS linker
GGGGSGGGGSGGGGS

SEQ ID NO: 10 - protein, 134 aa, synthetic construct
  source 1..134: note = Pembrolizumab-derived anti-PD-1 scFv VL
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTA

SEQ ID NO: 11 - protein, 12 aa, synthetic construct
  source 1..12: note = Pembrolizumab-derived anti-PD-1 scFv VL CDR1
KGVSTSGYSYLH

SEQ ID NO: 12 - protein, 11 aa, synthetic construct
  source 1..11: note = Pembrolizumab-derived anti-PD-1 scFv VL CDR2
LLIYLASYLES

SEQ ID NO: 13 - protein, 8 aa, synthetic construct
  source 1..8: note = Pembrolizumab-derived anti-PD-1 scFv VL CDR3
QHSRDLPL

SEQ ID NO: 14 - protein, 274 aa, synthetic construct
  source 1..274: note = Pembrolizumab-derived anti-PD-1 scFv VL-linker-VH
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKG

SEQ ID NO: 15 - protein, 18 aa, synthetic construct
  source 1..18: note = Whitlow linker
GSTSGSGKPGSGEGSTKG

SEQ ID NO: 16 - protein, 45 aa, synthetic construct
  source 1..45: note = CD8alpha hinge domain
TTTPAPRPPTPAPTIASQPLSLRPEACRPAAGGAVHTRGLDFACD

SEQ ID NO: 17 - protein, 39 aa, synthetic construct
  source 1..39: note = CD28 hinge domain
IEVMYPPPYLDNEKSNGTIIHVKGKHLCPSPLFPGPSKP

SEQ ID NO: 18 - protein, 12 aa, synthetic construct
  source 1..12: note = IgG4 hinge domain short
ESKYGPPCPPCP

SEQ ID NO: 19 - protein, 119 aa, synthetic construct
  source 1..119: note = IgG4 hinge domain medium
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 223 aa, synthetic construct
  source 1..223: note = IgG4 hinge domain long
ESKYGPPCPPCPGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 24 aa, synthetic construct
  source 1..24: note = CD8alpha transmembrane domain
IYIWAPLAGTCGVLLLSLVITLYC

SEQ ID NO: 22 - protein, 28 aa, synthetic construct
  source 1..28: note = CD28 transmembrane domain
MFWVLVVVGGVLACYSLLVTVAFIIFWV

SEQ ID NO: 23 - protein, 42 aa, synthetic construct
  source 1..42: note = 4-1BB costimulatory domain
KRGRKKLLYIFKQPFMRPVQTTQEEDGCSCRFPEEEEGGCEL

SEQ ID NO: 24 - protein, 41 aa, synthetic construct
  source 1..41: note = CD28 costimulatory domain
RSKRSRLLHSDYMNMTPRRPGPTRKHYQPYAPPRDFAAYRS

SEQ ID NO: 25 - protein, 112 aa, synthetic construct
  source 1..112: note = CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 112 aa, synthetic construct
  source 1..112: note = CD3zeta signaling domain_Q to K mutation
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 490 aa, synthetic construct
  source 1..490: note = Anti-PD-1 CAR with pembrolizumab-derived scFv (VH- linker-VL), short IgG4 hinge domain, CD28 transmembrane domain, 4-1BB costimulatory domain, and CD3zeta signaling domain
MLLLVTSLLLCELPHPAFLLIPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 597 aa, synthetic construct
  source 1..597: note = Anti-PD-1 CAR with pembrolizumab-derived scFv (VH- linker-VL), medium IgG4 hinge domain, CD28 transmembrane domain, 4-1BB costimulatory domain, and CD3zeta signaling domain
MLLLVTSLLLCELPHPAFLLIPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 701 aa, synthetic construct
  source 1..701: note = Anti-PD-1 CAR with pembrolizumab-derived scFv (VH- linker-VL), long IgG4 hinge domain, CD28 transmembrane domain, 4-1BB costimulatory domain, and CD3zeta signaling domain
MLLLVTSLLLCELPHPAFLLIPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAESKYGPPCPPCPGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 490 aa, synthetic construct
  source 1..490: note = Anti-PD-1 CAR with pembrolizumab-derived scFv (VL- linker-VH), short IgG4 hinge domain, CD28 transmembrane domain, 4-1BB costimulatory domain, and CD3zeta signaling domain
MLLLVTSLLLCELPHPAFLLIPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 597 aa, synthetic construct
  source 1..597: note = Anti-PD-1 CAR with pembrolizumab-derived scFv (VL- linker-VH), medium IgG4 hinge domain, CD28 transmembrane domain, 4-1BB costimulatory domain, and CD3zeta signaling domain
MLLLVTSLLLCELPHPAFLLIPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 701 aa, synthetic construct
  source 1..701: note = Anti-PD-1 CAR with pembrolizumab-derived scFv (VL- linker-VH), long IgG4 hinge domain, CD28 transmembrane domain, 4-1BB costimulatory domain, and CD3zeta signaling domain
MLLLVTSLLLCELPHPAFLLIPEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGESKYGPPCPPCPGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 1030 aa, synthetic construct
  source 1..1030: note = Anti-PD-1 CAR with pembrolizumab-derived scFv (VH- linker-VL), short IgG4 hinge domain, CD28 transmembrane domain, 4-1BB costimulatory domain, and CD3zeta signaling domain; T2A truncated EGFR (tEGFR), C46 fusion inhibitor
MLLLVTSLLLCELPHPAFLLIPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMLEGGGEGRGSLLTCGDVEENPGPRMGAGATGRAMDGPRLLLLLLLGVSLGGARSWMEWDREINNYTSLIHSLIEESQNQQEKNEQELLELDKWASLWNWFRSERKCCVECPPCPAPPVAGPLIALVTSGALLAVLGITGYFLMNRRSWSPTGERLELEP

SEQ ID NO: 34 - protein, 335 aa, synthetic construct
  source 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 35 - protein, 135 aa, synthetic construct [sequence omitted]

SEQ ID NO: 36 - DNA, 13 bp, synthetic construct
  source 1..13: mol_type = other DNA; note = Kozak Consensus Sequence
gccgccrccatgg

SEQ ID NO: 37 - DNA, 589 bp, synthetic construct
  source 1..589: mol_type = other DNA; note = WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 38 - protein, 21 aa, synthetic construct
  source 1..21: note = T2A
GSGEGRGSLLTCGDVEENPGP

SEQ ID NO: 39 - protein, 22 aa, synthetic construct
  source 1..22: note = P2A
GSGATNFSLLKQAGDVEENPGP

SEQ ID NO: 40 - protein, 23 aa, synthetic construct
  source 1..23: note = E2A
GSGQCTNYALLKLAGDVESNPGP

SEQ ID NO: 41 - protein, 25 aa, synthetic construct
  source 1..25: note = F2A
GSGVKQTLNFDLLKLAGDVESNPGP

SEQ ID NO: 42 - DNA, 16 bp, synthetic construct
  source 1..16: mol_type = other DNA; note = Primer For
tggaatcagcagaaag

SEQ ID NO: 43 - DNA, 15 bp, synthetic construct
  source 1..15: mol_type = other DNA; note = Primer Rev
atgttctgggtgctc

SEQ ID NO: 44 - protein, 490 aa, synthetic construct
  source 1..490: note = VH VL S anti-PD-1 CAR BD
MLLLVTSLLLCELPHPAFLLIPQVQLVQSGVEVKKPGASVKVSCKASGYTFANYFMYWVRQAPGQGLEWMGGIAPAAGGAAFNEKFKNRVTLTTDSSTTTAYMELKSLQFDDTAVYYCARADFAFDMGFDYWGQGTTVTVSSASTKGGGGGSGGGGSGGGGSEIVLTQSPATLSLSPGERATLSCRASKGVSTAGFSYLHWYQQKPGQAPRLLIFLASFLESGVPARFSGSGSGTDFTLTISSLEPEDFAVYYCQHARALPLTFGGGTKVEIKRTVAAPSVFIFPPSDEQLKSGTAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 378 aa, synthetic construct
  source 1..378: note = VH VL S anti-PD-1 CAR deltaCD3z
MLLLVTSLLLCELPHPAFLLIPQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTAESKYGPPCPPCPMFWVLVVVGGVLACYSLLVTVAFIIFWVKRGRKKLLYIFKQPFMRPVQTTQEEDGCSCRFPEEEEGGCEL

SEQ ID NO: 46 - protein, 378 aa, synthetic construct
  source 1..378: note = VH VL S anti-PD-1 CAR BD deltaCD3z
MLLLVTSLLLCELPHPAFLLIPQVQLVQSGVEVKKPGASVKVSCKASGYTFANYFMYWVRQAPGQGLEWMGGIAPAAGGAAFNEKFKNRVTLTTDSSTTTAYMELKSLQFDDTAVYYCARADFAFDMGFDYWGQGTTVTVSSASTKGGGGGSGGGGSGGGGSEIVLTQSPATLSLSPGERATLSCRASKGVSTAGFSYLHWYQQKPGQAPRLLIFLASFLESGVPARFSGSGSGTDFTLTISSLEPEDFAVYYCQHARALPLTFGGGTKVEIKRTVAAPSVFIFPPSDEQLKSGTAESKYGPPCPPCPMFWVLVVVGGVLACYSLLVTVAFIIFWVKRGRKKLLYIFKQPFMRPVQTTQEEDGCSCRFPEEEEGGCEL

SEQ ID NO: 47 - protein, 443 aa, synthetic construct
  source 1..443: note = camrelizumab Heavy Chain Amino Acid Sequence
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 444 aa, synthetic construct
  source 1..444: note = cemiplimab Heavy Chain Amino Acid Sequence
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 443 aa, synthetic construct
  source 1..443: note = dostarlimab Heavy Chain Amino Acid Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 440 aa, synthetic construct
  source 1..440: note = nivolumab Heavy Chain Amino Acid Sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 448 aa, synthetic construct
  source 1..448: note = penpulimab Heavy Chain Amino Acid Sequence
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 459 aa, synthetic construct
  source 1..459: note = prolgolimab Heavy Chain Amino Acid Sequence
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 443 aa, synthetic construct
  source 1..443: note = serplulimab Heavy Chain Amino Acid Sequence
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 447 aa, synthetic construct
  source 1..447: note = sintilimab Heavy Chain Amino Acid Sequence
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 445 aa, synthetic construct
  source 1..445: note = tislelizumab Heavy Chain Amino Acid Sequence
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 56 - protein, 452 aa, synthetic construct
  source 1..452: note = toripalimab Heavy Chain Amino Acid Sequence
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 450 aa, synthetic construct
  source 1..450: note = zimberelimab Heavy Chain Amino Acid Sequence
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 441 aa, synthetic construct
  source 1..441: note = acrixolimab Heavy Chain Amino Acid Sequence
QVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 450 aa, synthetic construct
  source 1..450: note = zeluvalimab Heavy Chain Amino Acid Sequence
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 448 aa, synthetic construct
  source 1..448: note = budigalimab Heavy Chain Amino Acid Sequence
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 446 aa, synthetic construct
  source 1..446: note = ezabenlimab Heavy Chain Amino Acid Sequence
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 446 aa, synthetic construct
  source 1..446: note = iparomlimab Heavy Chain Amino Acid Sequence
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 449 aa, synthetic construct
  source 1..449: note = peresolimab Heavy Chain Amino Acid Sequence
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 445 aa, synthetic construct
  source 1..445: note = pimivalimab Heavy Chain Amino Acid Sequence
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 451 aa, synthetic construct
  source 1..451: note = rosnilimab Heavy Chain Amino Acid Sequence
QVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 448 aa, synthetic construct
  source 1..448: note = rulonilimab Heavy Chain Amino Acid Sequence
EVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 67 - protein, 443 aa, synthetic construct
  source 1..443: note = spartalizumab Heavy Chain Amino Acid Sequence
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 68 - protein, 447 aa, synthetic construct
  source 1..447: note = sudubrilimab Heavy Chain Amino Acid Sequence
EVQLVESGGGLVQPGGSLRLSCAASGFTFSETWLHWVRQAPGKGLEWVAWVSPFGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 445 aa, synthetic construct
  source 1..445: note = finotonlimab Heavy Chain Amino Acid Sequence
EVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 450 aa, synthetic construct
  source 1..450: note = cetrelimab Heavy Chain Amino Acid Sequence
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 442 aa, synthetic construct
  source 1..442: note = nofazinlimab Heavy Chain Amino Acid Sequence
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 445 aa, synthetic construct
  source 1..445: note = pucotenlimab Heavy Chain Amino Acid Sequence
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMITRTPEVTCVVVDVSQEDPEVQFNWYVDGVEVHNAKTKPREEQFNSTYRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 73 - protein, 444 aa, synthetic construct
  source 1..444: note = sasanlimab Heavy Chain Amino Acid Sequence
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 440 aa, synthetic construct
  source 1..440: note = balstilimab Heavy Chain Amino Acid Sequence
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 440 aa, synthetic construct
  source 1..440: note = geptanolimab Heavy Chain Amino Acid Sequence
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 445 aa, synthetic construct
  source 1..445: note = retifanlimab Heavy Chain Amino Acid Sequence
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 77 - protein, 214 aa, synthetic construct
  source 1..214: note = camrelizumab Light Chain Amino Acid Sequence
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 214 aa, synthetic construct
  source 1..214: note = cemiplimab Light Chain Amino Acid Sequence
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 214 aa, synthetic construct
  source 1..214: note = dostarlimab Light Chain Amino Acid Sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 214 aa, synthetic construct
  source 1..214: note = nivolumab Light Chain Amino Acid Sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 214 aa, synthetic construct
  source 1..214: note = penpulimab Light Chain Amino Acid Sequence
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 214 aa, synthetic construct
  source 1..214: note = prolgolimab Light Chain Amino Acid Sequence
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 214 aa, synthetic construct
  source 1..214: note = serplulimab Light Chain Amino Acid Sequence
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 214 aa, synthetic construct
  source 1..214: note = sintilimab Light Chain Amino Acid Sequence
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 214 aa, synthetic construct
  source 1..214: note = tislelizumab Light Chain Amino Acid Sequence
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 219 aa, synthetic construct
  source 1..219: note = toripalimab Light Chain Amino Acid Sequence
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 216 aa, synthetic construct
  source 1..216: note = zimberelimab Light Chain Amino Acid Sequence
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 88 - protein, 220 aa, synthetic construct
  source 1..220: note = acrixolimab Light Chain Amino Acid Sequence
DIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 214 aa, synthetic construct
  source 1..214: note = zeluvalimab Light Chain Amino Acid Sequence
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 219 aa, synthetic construct
  source 1..219: note = budigalimab Light Chain Amino Acid Sequence
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 218 aa, synthetic construct
  source 1..218: note = ezabenlimab Light Chain Amino Acid Sequence
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 220 aa, synthetic construct
  source 1..220: note = iparomlimab Light Chain Amino Acid Sequence
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 217 aa, synthetic construct
  source 1..217: note = peresolimab Light Chain Amino Acid Sequence
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 214 aa, synthetic construct
  source 1..214: note = pimivalimab Light Chain Amino Acid Sequence
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 215 aa, synthetic construct
  source 1..215: note = rosnilimab Light Chain Amino Acid Sequence
EIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 218 aa, synthetic construct
  source 1..218: note = rulonilimab Light Chain Amino Acid Sequence
EIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 220 aa, synthetic construct
  source 1..220: note = spartalizumab Light Chain Amino Acid Sequence
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 214 aa, synthetic construct
  source 1..214: note = sudubrilimab Light Chain Amino Acid Sequence
DIQMTQSPSSLSASVGDRVTITCRASQDVSTAVAWYQQKPGKAPKLLIYSASFLYSGVPSRFSGSGSGTDFTLTISSLQPEDFATYYCQQFLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 218 aa, synthetic construct
  source 1..218: note = finotonlimab Light Chain Amino Acid Sequence
EIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 214 aa, synthetic construct
  source 1..214: note = cetrelimab Light Chain Amino Acid Sequence
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 219 aa, synthetic construct
  source 1..219: note = nofazinlimab Light Chain Amino Acid Sequence
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 218 aa, synthetic construct
  source 1..218: note = pucotenlimab Light Chain Amino Acid Sequence
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 220 aa, synthetic construct
  source 1..220: note = sasanlimab Light Chain Amino Acid Sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 214 aa, synthetic construct
  source 1..214: note = balstilimab Light Chain Amino Acid Sequence
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 217 aa, synthetic construct
  source 1..217: note = geptanolimab Light Chain Amino Acid Sequence
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 218 aa, synthetic construct
  source 1..218: note = retifanlimab Light Chain Amino Acid Sequence
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 116 aa, synthetic construct
  source 1..116: note = Camrelizumab Vh Amino Acid Sequence
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 108 - protein, 117 aa, synthetic construct [sequence omitted]

SEQ ID NO: 109 - protein, 116 aa, synthetic construct
  source 1..116: note = Dostarlimab Vh Amino Acid Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 110 - protein, 113 aa, synthetic construct [sequence omitted]

SEQ ID NO: 111 - protein, 118 aa, synthetic construct
  source 1..118: note = Penpulimab Vh Amino Acid Sequence
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 112 - protein, 129 aa, synthetic construct
  source 1..129: note = Prolgolimab Vh Amino Acid Sequence
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSS

SEQ ID NO: 113 - protein, 116 aa, synthetic construct
  source 1..116: note = Serplulimab Vh Amino Acid Sequence
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 114 - protein, 120 aa, synthetic construct
  source 1..120: note = Sintilimab Vh Amino Acid Sequence
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 115 - protein, 118 aa, synthetic construct
  source 1..118: note = Tislelizumab Vh Amino Acid Sequence
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 116 - protein, 125 aa, synthetic construct [sequence omitted]

SEQ ID NO: 117 - protein, 123 aa, synthetic construct
  source 1..123: note = Zimberelimab Vh Amino Acid Sequence
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 118 - protein, 114 aa, synthetic construct
  source 1..114: note = Acrixolimab Vh Amino Acid Sequence
QVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSS

SEQ ID NO: 119 - protein, 120 aa, synthetic construct
  source 1..120: note = Zeluvalimab Vh Amino Acid Sequence
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 120 - protein, 118 aa, synthetic construct
  source 1..118: note = Budigalimab Vh Amino Acid Sequence
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 121 - protein, 120 aa, synthetic construct
  source 1..120: note = Ezabenlimab Vh Amino Acid Sequence
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 122 - protein, 119 aa, synthetic construct
  source 1..119: note = Iparomlimab Vh Amino Acid Sequence
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 123 - protein, 119 aa, synthetic construct
  source 1..119: note = Peresolimab Vh Amino Acid Sequence
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSS

SEQ ID NO: 124 - protein, 118 aa, synthetic construct
  source 1..118: note = Pimivalimab Vh Amino Acid Sequence
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 125 - protein, 121 aa, synthetic construct
  source 1..121: note = Rosnilimab Vh Amino Acid Sequence
QVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSS

SEQ ID NO: 126 - protein, 118 aa, synthetic construct
  source 1..118: note = Rulonilimab Vh Amino Acid Sequence
EVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSS

SEQ ID NO: 127 - protein, 117 aa, synthetic construct
  source 1..117: note = Spartalizumab Vh Amino Acid Sequence
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 128 - protein, 118 aa, synthetic construct [sequence omitted]

SEQ ID NO: 129 - protein, 118 aa, synthetic construct
  source 1..118: note = Finotonlimab Vh Amino Acid Sequence
EVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSS

SEQ ID NO: 130 - protein, 123 aa, synthetic construct
  source 1..123: note = Cetrelimab Vh Amino Acid Sequence
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 131 - protein, 115 aa, synthetic construct
  source 1..115: note = Nofazinlimab Vh Amino Acid Sequence
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 132 - protein, 118 aa, synthetic construct
  source 1..118: note = Pucotenlimab Vh Amino Acid Sequence
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSS

SEQ ID NO: 133 - protein, 117 aa, synthetic construct
  source 1..117: note = Sasanlimab Vh Amino Acid Sequence
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 134 - protein, 113 aa, synthetic construct
  source 1..113: note = balstilimab Vh Amino Acid Sequence
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 135 - protein, 114 aa, synthetic construct
  source 1..114: note = geptanolimab Vh Amino Acid Sequence
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSS

SEQ ID NO: 136 - protein, 119 aa, synthetic construct
  source 1..119: note = retifanlimab Vh Amino Acid Sequence
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 137 - protein, 107 aa, synthetic construct
  source 1..107: note = Camrelizumab Vl Chain Amino Acid Sequence
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 138 - protein, 107 aa, synthetic construct [sequence omitted]

SEQ ID NO: 139 - protein, 107 aa, synthetic construct
  source 1..107: note = Dostarlimab Vl Chain Amino Acid Sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 140 - protein, 107 aa, synthetic construct [sequence omitted]

SEQ ID NO: 141 - protein, 107 aa, synthetic construct
  source 1..107: note = Penpulimab Vl Chain Amino Acid Sequence
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 142 - protein, 107 aa, synthetic construct
  source 1..107: note = Prolgolimab Vl Chain Amino Acid Sequence
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQ

SEQ ID NO: 143 - protein, 107 aa, synthetic construct
  source 1..107: note = Serplulimab Vl Chain Amino Acid Sequence
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 144 - protein, 107 aa, synthetic construct
  source 1..107: note = Sintilimab Vl Chain Amino Acid Sequence
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 145 - protein, 107 aa, synthetic construct
  source 1..107: note = Tislelizumab Vl Chain Amino Acid Sequence
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 146 - protein, 112 aa, synthetic construct [sequence omitted]

SEQ ID NO: 147 - protein, 110 aa, synthetic construct
  source 1..110: note = Zimberelimab Vl Chain Amino Acid Sequence
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 148 - protein, 113 aa, synthetic construct
  source 1..113: note = Acrixolimab Vl Chain Amino Acid Sequence
DIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIK

SEQ ID NO: 149 - protein, 107 aa, synthetic construct
  source 1..107: note = Zeluvalimab Vl Chain Amino Acid Sequence
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 150 - protein, 112 aa, synthetic construct
  source 1..112: note = Budigalimab Vl Chain Amino Acid Sequence
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 151 - protein, 111 aa, synthetic construct
  source 1..111: note = Ezabenlimab Vl Chain Amino Acid Sequence
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIK

SEQ ID NO: 152 - protein, 113 aa, synthetic construct
  source 1..113: note = Iparomlimab Vl Chain Amino Acid Sequence
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 153 - protein, 110 aa, synthetic construct
  source 1..110: note = Peresolimab Vl Chain Amino Acid Sequence
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIK

SEQ ID NO: 154 - protein, 107 aa, synthetic construct
  source 1..107: note = Pimivalimab Vl Chain Amino Acid Sequence
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 155 - protein, 108 aa, synthetic construct
  source 1..108: note = Rosnilimab Vl Chain Amino Acid Sequence
EIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIK

SEQ ID NO: 156 - protein, 111 aa, synthetic construct
  source 1..111: note = Rulonilimab Vl Chain Amino Acid Sequence
EIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIK

SEQ ID NO: 157 - protein, 113 aa, synthetic construct
  source 1..113: note = Spartalizumab Vl Chain Amino Acid Sequence
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 158 - protein, 107 aa, synthetic construct [sequence omitted]

SEQ ID NO: 159 - protein, 111 aa, synthetic construct
  source 1..111: note = Finotonlimab Vl Chain Amino Acid Sequence
EIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIK

SEQ ID NO: 160 - protein, 107 aa, synthetic construct
  source 1..107: note = Cetrelimab Vl Chain Amino Acid Sequence
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 161 - protein, 112 aa, synthetic construct
  source 1..112: note = Nofazinlimab Vl Chain Amino Acid Sequence
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 162 - protein, 111 aa, synthetic construct
  source 1..111: note = Pucotenlimab Vl Chain Amino Acid Sequence
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIK

SEQ ID NO: 163 - protein, 113 aa, synthetic construct
  source 1..113: note = Sasanlimab Vl Chain Amino Acid Sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 164 - protein, 107 aa, synthetic construct
  source 1..107: note = Balstilimab Vl Chain Amino Acid Sequence
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 165 - protein, 110 aa, synthetic construct
  source 1..110: note = Geptanolimab Vl Chain Amino Acid Sequence
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIK

SEQ ID NO: 166 - protein, 111 aa, synthetic construct
  source 1..111: note = Retifanlimab Vl Chain Amino Acid Sequence
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 167 - protein, 238 aa, synthetic construct
  source 1..238: note = Camrelizumab Vh Vl scFv
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSGGGGSGGGGSGGGGSDIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 168 - protein, 239 aa, synthetic construct
  source 1..239: note = cemiplimab Vh Vl scFv
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSGGGGSGGGGSGGGGSDIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFR

SEQ ID NO: 169 - protein, 238 aa, synthetic construct
  source 1..238: note = dostarlimab Vh Vl scFv
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSGGGGSGGGGSGGGGSDIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 170 - protein, 235 aa, synthetic construct
  source 1..235: note = nivolumab Vh Vl scFv
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

SEQ ID NO: 171 - protein, 240 aa, synthetic construct
  source 1..240: note = penpulimab Vh Vl scFv
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 172 - protein, 251 aa, synthetic construct
  source 1..251: note = prolgolimab Vh Vl scFv
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSGGGGSGGGGSGGGGSQPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQ

SEQ ID NO: 173 - protein, 238 aa, synthetic construct
  source 1..238: note = serplulimab Vh Vl scFv
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSSGGGGSGGGGSGGGGSDIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 174 - protein, 242 aa, synthetic construct
  source 1..242: note = sintilimab Vh Vl scFv
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSGGGGSGGGGSGGGGSDIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 175 - protein, 240 aa, synthetic construct
  source 1..240: note = tislelizumab Vh Vl scFv
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSGGGGSGGGGSGGGGSDIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 176 - protein, 252 aa, synthetic construct
  source 1..252: note = toripalimab Vh Vl scFv
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSGGGGSGGGGSGGGGSDVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIK

SEQ ID NO: 177 - protein, 248 aa, synthetic construct
  source 1..248: note = zimberelimab Vh Vl scFv
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSSGGGGSGGGGSGGGGSQSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 178 - protein, 242 aa, synthetic construct
  source 1..242: note = acrixolimab Vh Vl scFv
QVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSSGGGGSGGGGSGGGGSDIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIK

SEQ ID NO: 179 - protein, 242 aa, synthetic construct
  source 1..242: note = zeluvalimab Vh Vl scFv
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSGGGGSGGGGSGGGGSDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 180 - protein, 245 aa, synthetic construct
  source 1..245: note = budigalimab Vh Vl scFv
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSGGGGSGGGGSGGGGSDVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 181 - protein, 246 aa, synthetic construct
  source 1..246: note = ezabenlimab Vh Vl scFv
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSGGGGSGGGGSGGGGSEIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIK

SEQ ID NO: 182 - protein, 247 aa, synthetic construct
  source 1..247: note = iparomlimab Vh Vl scFv
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSSGGGGSGGGGSGGGGSDIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 183 - protein, 244 aa, synthetic construct
  source 1..244: note = peresolimab Vh Vl scFv
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSSGGGGSGGGGSGGGGSDIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIK

SEQ ID NO: 184 - protein, 240 aa, synthetic construct
  source 1..240: note = pimivalimab Vh Vl scFv
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 185 - protein, 244 aa, synthetic construct
  source 1..244: note = rosnilimab Vh Vl scFv
QVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSSGGGGSGGGGSGGGGSEIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIK

SEQ ID NO: 186 - protein, 244 aa, synthetic construct
  source 1..244: note = rulonilimab Vh Vl scFv
EVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSSGGGGSGGGGSGGGGSEIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIK

SEQ ID NO: 187 - protein, 245 aa, synthetic construct
  source 1..245: note = spartalizumab Vh Vl scFv
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSGGGGSGGGGSGGGGSEIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 188 - protein, 240 aa, synthetic construct
  source 1..240: note = sudubrilimab Vh Vl scFv
EVQLVESGGGLVQPGGSLRLSCAASGFTFSETWLHWVRQAPGKGLEWVAWVSPFGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQFLYHPATFGQGTKVEIK

SEQ ID NO: 189 - protein, 244 aa, synthetic construct
  source 1..244: note = finotonlimab Vh Vl scFv
EVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSSGGGGSGGGGSGGGGSEIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIK

SEQ ID NO: 190 - protein, 245 aa, synthetic construct
  source 1..245: note = cetrelimab Vh Vl scFv
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 191 - protein, 242 aa, synthetic construct
  source 1..242: note = nofazinlimab Vh Vl scFv
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSGGGGSGGGGSGGGGSDVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 192 - protein, 244 aa, synthetic construct
  source 1..244: note = pucotenlimab Vh Vl scFv
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSSGGGGSGGGGSGGGGSDIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIK

SEQ ID NO: 193 - protein, 245 aa, synthetic construct
  source 1..245: note = sasanlimab Vh Vl scFv
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSGGGGSGGGGSGGGGSDIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 194 - protein, 235 aa, synthetic construct
  source 1..235: note = balstilimab Vh Vl scFv
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 195 - protein, 239 aa, synthetic construct
  source 1..239: note = geptanolimab Vh Vl scFv
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSSGGGGSGGGGSGGGGSDIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIK

SEQ ID NO: 196 - protein, 245 aa, synthetic construct
  source 1..245: note = retifanlimab Vh Vl scFv
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGGGSGGGGSGGGGSEIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 197 - protein, 238 aa, synthetic construct
  source 1..238: note = Camrelizumab Vl Vh scFv
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKGGGGSGGGGSGGGGSEVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 198 - protein, 239 aa, synthetic construct
  source 1..239: note = cemiplimab Vl Vh scFv
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRGGGGSGGGGSGGGGSEVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSS

SEQ ID NO: 199 - protein, 238 aa, synthetic construct
  source 1..238: note = dostarlimab Vl Vh scFv
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKGGGGSGGGGSGGGGSEVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 200 - protein, 235 aa, synthetic construct
  source 1..235: note = nivolumab Vl Vh scFv
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 201 - protein, 240 aa, synthetic construct
  source 1..240: note = penpulimab Vl Vh scFv
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 202 - protein, 251 aa, synthetic construct
  source 1..251: note = prolgolimab Vl Vh scFv
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQGGGGSGGGGSGGGGSQVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSS

SEQ ID NO: 203 - protein, 238 aa, synthetic construct
  source 1..238: note = serplulimab Vl Vh scFv
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKGGGGSGGGGSGGGGSQVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 204 - protein, 242 aa, synthetic construct
  source 1..242: note = sintilimab Vl Vh scFv
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKGGGGSGGGGSGGGGSQVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 205 - protein, 240 aa, synthetic construct
  source 1..240: note = tislelizumab Vl Vh scFv
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKGGGGSGGGGSGGGGSQVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 206 - protein, 252 aa, synthetic construct
  source 1..252: note = toripalimab Vl Vh scFv
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKGGGGSGGGGSGGGGSQGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSS

SEQ ID NO: 207 - protein, 248 aa, synthetic construct
  source 1..248: note = zimberelimab Vl Vh scFv
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVLGGGGSGGGGSGGGGSQLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 208 - protein, 242 aa, synthetic construct
  source 1..242: note = acrixolimab Vl Vh scFv
DIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIKGGGGSGGGGSGGGGSQVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSS

SEQ ID NO: 209 - protein, 242 aa, synthetic construct
  source 1..242: note = zeluvalimab Vl Vh scFv
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKGGGGSGGGGSGGGGSEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 210 - protein, 245 aa, synthetic construct
  source 1..245: note = budigalimab Vl Vh scFv
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKGGGGSGGGGSGGGGSEIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 211 - protein, 246 aa, synthetic construct
  source 1..246: note = ezabenlimab Vl Vh scFv
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKGGGGSGGGGSGGGGSEVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 212 - protein, 247 aa, synthetic construct
  source 1..247: note = iparomlimab Vl Vh scFv
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIKGGGGSGGGGSGGGGSQVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 213 - protein, 244 aa, synthetic construct
  source 1..244: note = peresolimab Vl Vh scFv
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIKGGGGSGGGGSGGGGSQVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSS

SEQ ID NO: 214 - protein, 240 aa, synthetic construct
  source 1..240: note = pimivalimab Vl Vh scFv
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKGGGGSGGGGSGGGGSQVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 215 - protein, 244 aa, synthetic construct
  source 1..244: note = rosnilimab Vl Vh scFv
EIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIKGGGGSGGGGSGGGGSQVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSS

SEQ ID NO: 216 - protein, 244 aa, synthetic construct
  source 1..244: note = rulonilimab Vl Vh scFv
EIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIKGGGGSGGGGSGGGGSEVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSS

SEQ ID NO: 217 - protein, 245 aa, synthetic construct
  source 1..245: note = spartalizumab Vl Vh scFv
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKGGGGSGGGGSGGGGSEVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 218 - protein, 240 aa, synthetic construct
  source 1..240: note = sudubrilimab Vl Vh scFv
DIQMTQSPSSLSASVGDRVTITCRASQDVSTAVAWYQQKPGKAPKLLIYSASFLYSGVPSRFSGSGSGTDFTLTISSLQPEDFATYYCQQFLYHPATFGQGTKVEIKGGGGSGGGGSGGGGSEVQLVESGGGLVQPGGSLRLSCAASGFTFSETWLHWVRQAPGKGLEWVAWVSPFGGSTYYADSVKGRFTISADTSKNTAYLQMNSLRAEDTAVYYCARRHWPGGFDYWGQGTLVTVSS

SEQ ID NO: 219 - protein, 244 aa, synthetic construct
  source 1..244: note = finotonlimab Vl Vh scFv
EIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIKGGGGSGGGGSGGGGSEVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSS

SEQ ID NO: 220 - protein, 245 aa, synthetic construct
  source 1..245: note = cetrelimab Vl Vh scFv
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKGGGGSGGGGSGGGGSQVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 221 - protein, 242 aa, synthetic construct
  source 1..242: note = nofazinlimab Vl Vh scFv
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKGGGGSGGGGSGGGGSQVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 222 - protein, 244 aa, synthetic construct
  source 1..244: note = pucotenlimab Vl Vh scFv
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIKGGGGSGGGGSGGGGSEVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSS

SEQ ID NO: 223 - protein, 245 aa, synthetic construct
  source 1..245: note = sasanlimab Vl Vh scFv
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKGGGGSGGGGSGGGGSQVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 224 - protein, 235 aa, synthetic construct
  source 1..235: note = balstilimab Vl Vh scFv
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 225 - protein, 239 aa, synthetic construct
  source 1..239: note = geptanolimab Vl Vh scFv
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIKGGGGSGGGGSGGGGSQIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSS

SEQ ID NO: 226 - protein, 245 aa, synthetic construct
  source 1..245: note = retifanlimab Vl Vh scFv
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGGSGGGGSGGGGSQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 227 - protein, 9 aa, synthetic construct
  source 1..9: note = Camrelizumab Vh CDR1
FTFSSYMMS

SEQ ID NO: 228 - protein, 9 aa, synthetic construct
  source 1..9: note = cemiplimab Vh CDR1
FTFSNFGMT

SEQ ID NO: 229 - protein, 9 aa, synthetic construct
  source 1..9: note = dostarlimab and zeluvalimab Vh CDR1
FTFSSYDMS

SEQ ID NO: 230 - protein, 9 aa, synthetic construct
  source 1..9: note = nivolumab Vh CDR1
ITFSNSGMH

SEQ ID NO: 231 - protein, 9 aa, synthetic construct
  source 1..9: note = penpulimab Vh CDR1
FAFSSYDMS

SEQ ID NO: 232 - protein, 9 aa, synthetic construct
  source 1..9: note = prolgolimab Vh CDR1
FTFSSYWMY

SEQ ID NO: 233 - protein, 9 aa, synthetic construct
  source 1..9: note = serplulimab Vh CDR1
FTFSNYGMS

SEQ ID NO: 234 - protein, 9 aa, synthetic construct
  source 1..9: note = sintilimab and cetrelimab Vh CDR1
GTFSSYAIS

SEQ ID NO: 235 - protein, 9 aa, synthetic construct
  source 1..9: note = tislelizumab Vh CDR1
FSLTSYGVH

SEQ ID NO: 236 - protein, 9 aa, synthetic construct
  source 1..9: note = toripalimab Vh CDR1
YTFTDYEMH

SEQ ID NO: 237 - protein, 12 aa, synthetic construct
  source 1..12: note = zimberelimab Vh CDR1
ADSISSTTYYWV

SEQ ID NO: 238 - protein, 9 aa, synthetic construct
  source 1..9: note = acrixolimab Vh CDR1
FTFLRYAMH

SEQ ID NO: 239 - protein, 10 aa, synthetic construct
  source 1..10: note = budigalimab Vh CDR1
YTFTHYGMNW

SEQ ID NO: 240 - protein, 9 aa, synthetic construct
  source 1..9: note = ezabenlimab Vh CDR1
FTFSKSAMS

SEQ ID NO: 241 - protein, 9 aa, synthetic construct
  source 1..9: note = iparomlimab Vh CDR1
YTFTNYWIH

SEQ ID NO: 242 - protein, 9 aa, synthetic construct
  source 1..9: note = peresolimab Vh CDR1
YSLSKYDMS

SEQ ID NO: 243 - protein, 9 aa, synthetic construct
  source 1..9: note = pimivalimab Vh CDR1
YTFPSYYMH

SEQ ID NO: 244 - protein, 10 aa, synthetic construct
  source 1..10: note = rosnilimab Vh CDR1
NYTFTDYSMH

SEQ ID NO: 245 - protein, 9 aa, synthetic construct
  source 1..9: note = rulonilimab, finotonlimab and pucotenlimab Vh CDR1
FTFSSYGMS

SEQ ID NO: 246 - protein, 9 aa, synthetic construct
  source 1..9: note = spartalizumab Vh CDR1
YTFTTYWMH

SEQ ID NO: 247 - protein, 9 aa, synthetic construct
  source 1..9: note = sudubrilimab Vh CDR1
FTFSETWLH

SEQ ID NO: 248 - protein, 9 aa, synthetic construct
  source 1..9: note = nofazinlimab Vh CDR1
FTFTTYYIS

SEQ ID NO: 249 - protein, 9 aa, synthetic construct
  source 1..9: note = sasanlimab Vh CDR1
YTFTSYWIN

SEQ ID NO: 250 - protein, 9 aa, synthetic construct
  source 1..9: note = balstilimab Vh CDR1
FTFSSYGMH

SEQ ID NO: 251 - protein, 10 aa, synthetic construct
  source 1..10: note = geptanolimab Vh CDR1
YTFTNFGMNW

SEQ ID NO: 252 - protein, 9 aa, synthetic construct
  source 1..9: note = retifanlimab Vh CDR1
YSFTSYWMN

SEQ ID NO: 253 - protein, 15 aa, synthetic construct
  source 1..15: note = Camrelizumab Vh CDR2
WVATISGGGANTYYP

SEQ ID NO: 254 - protein, 15 aa, synthetic construct
  source 1..15: note = cemiplimab Vh CDR2
WVSGISGGGRDTYFA

SEQ ID NO: 255 - protein, 15 aa, synthetic construct
  source 1..15: note = dostarlimab Vh CDR2
WVSTISGGGSYTYYQ

SEQ ID NO: 256 - protein, 14 aa, synthetic construct
  source 1..14: note = nivolumab Vh CDR2
WVAVIWYDGSKRYY

SEQ ID NO: 257 - protein, 15 aa, synthetic construct
  source 1..15: note = penpulimab Vh CDR2
WVATISGGGRYTYYP

SEQ ID NO: 258 - protein, 15 aa, synthetic construct
  source 1..15: note = prolgolimab Vh CDR2
WVSAIDTGGGRTYYA

SEQ ID NO: 259 - protein, 14 aa, synthetic construct
  source 1..14: note = serplulimab Vh CDR2
WVSTISGGGSNIYY

SEQ ID NO: 260 - protein, 14 aa, synthetic construct
  source 1..14: note = sintilimab Vh CDR2
WMGLIIPMFDTAGY

SEQ ID NO: 261 - protein, 13 aa, synthetic construct
  source 1..13: note = tislelizumab Vh CDR2
WIGVIYADGSTNY

SEQ ID NO: 262 - protein, 14 aa, synthetic construct
  source 1..14: note = toripalimab Vh CDR2
WIGVIESETGGTAY

SEQ ID NO: 263 - protein, 13 aa, synthetic construct
  source 1..13: note = zimberelimab Vh CDR2
WIGSISYSGSTYY

SEQ ID NO: 264 - protein, 14 aa, synthetic construct
  source 1..14: note = acrixolimab Vh CDR2
WVAVISYDGRYKYY

SEQ ID NO: 265 - protein, 15 aa, synthetic construct
  source 1..15: note = zeluvalimab Vh CDR2
WVSLISGGGSQTYYA

SEQ ID NO: 266 - protein, 14 aa, synthetic construct
  source 1..14: note = budigalimab Vh CDR2
WVGWVNTYTGEPTY

SEQ ID NO: 267 - protein, 14 aa, synthetic construct
  source 1..14: note = ezabenlimab Vh CDR2
WVAYISGGGGDTYY

SEQ ID NO: 268 - protein, 14 aa, synthetic construct
  source 1..14: note = iparomlimab Vh CDR2
WMGEIDPYDSYTNY

SEQ ID NO: 269 - protein, 13 aa, synthetic construct
  source 1..13: note = peresolimab Vh CDR2
WMGIIYTSGYTDY

SEQ ID NO: 270 - protein, 14 aa, synthetic construct
  source 1..14: note = pimivalimab Vh CDR2
WMGIINPEGGSTAY

SEQ ID NO: 271 - protein, 14 aa, synthetic construct
  source 1..14: note = rosnilimab Vh CDR2
WMGWINIETYYPTY

SEQ ID NO: 272 - protein, 14 aa, synthetic construct
  source 1..14: note = rulonilimab, finotonlimab and pucotenlimab Vh CDR2
WVATISGGGRDTYY

SEQ ID NO: 273 - protein, 15 aa, synthetic construct
  source 1..15: note = spartalizumab Vh CDR2
WMGNIYPGTGGSNFD

SEQ ID NO: 274 - protein, 14 aa, synthetic construct
  source 1..14: note = sudubrilimab Vh CDR2
WVAWVSPFGGSTYY

SEQ ID NO: 275 - protein, 14 aa, synthetic construct
  source 1..14: note = cetrelimab Vh CDR2
WMGGIIPIFDTANY

SEQ ID NO: 276 - protein, 15 aa, synthetic construct
  source 1..15: note = nofazinlimab Vh CDR2
YLGYINMGSGGTNYN

SEQ ID NO: 277 - protein, 15 aa, synthetic construct
  source 1..15: note = sasanlimab Vh CDR2
WMGNIYPGSSLTNYN

SEQ ID NO: 278 - protein, 14 aa, synthetic construct
  source 1..14: note = balstilimab Vh CDR2
WVAVIWYDGSNKYY

SEQ ID NO: 279 - protein, 14 aa, synthetic construct
  source 1..14: note = geptanolimab Vh CDR2
WMGWISGYTREPTY

SEQ ID NO: 280 - protein, 15 aa, synthetic construct
  source 1..15: note = retifanlimab Vh CDR2
WIGVIHPSDSETWLD

SEQ ID NO: 281 - protein, 9 aa, synthetic construct
  source 1..9: note = Camrelizumab Vh CDR3
ARQLYYFDY

SEQ ID NO: 282 - protein, 10 aa, synthetic construct
  source 1..10: note = cemiplimab Vh CDR3
VKWGNIYFDY

SEQ ID NO: 283 - protein, 8 aa, synthetic construct
  source 1..8: note = dostarlimab Vh CDR3
SPYYAMDY

SEQ ID NO: 284 - protein, 5 aa, synthetic construct
  source 1..5: note = nivolumab Vh CDR3
TNDDY

SEQ ID NO: 285 - protein, 11 aa, synthetic construct
  source 1..11: note = penpulimab Vh CDR3
ANRYGEAWFAY

SEQ ID NO: 286 - protein, 22 aa, synthetic construct
  source 1..22: note = prolgolimab Vh CDR3
ARDEGGGTGWGVLKDWPYGLDA

SEQ ID NO: 287 - protein, 9 aa, synthetic construct
  source 1..9: note = serplulimab Vh CDR3
VSYYYGIDF

SEQ ID NO: 288 - protein, 12 aa, synthetic construct
  source 1..12: note = sintilimab Vh CDR3
RAEHSSTGTFDY

SEQ ID NO: 289 - protein, 11 aa, synthetic construct
  source 1..11: note = tislelizumab Vh CDR3
RAYGNYWYIDV

SEQ ID NO: 290 - protein, 18 aa, synthetic construct
  source 1..18: note = toripalimab Vh CDR3
AREGITTVATTYYWYFDV

SEQ ID NO: 291 - protein, 14 aa, synthetic construct
  source 1..14: note = zimberelimab Vh CDR3
RHLGYNGRYLPFDY

SEQ ID NO: 292 - protein, 7 aa, synthetic construct
  source 1..7: note = acrixolimab Vh CDR3
TTTTFDS

SEQ ID NO: 293 - protein, 13 aa, synthetic construct
  source 1..13: note = zeluvalimab Vh CDR3
ASPSGHYFYAMDV

SEQ ID NO: 294 - protein, 10 aa, synthetic construct
  source 1..10: note = budigalimab Vh CDR3
REGEGLGFGD

SEQ ID NO: 295 - protein, 12 aa, synthetic construct
  source 1..12: note = ezabenlimab Vh CDR3
RHSNVNYYAMDY

SEQ ID NO: 296 - protein, 11 aa, synthetic construct
  source 1..11: note = iparomlimab Vh CDR3
RPGFTYGGMDF

SEQ ID NO: 297 - protein, 12 aa, synthetic construct
  source 1..12: note = peresolimab Vh CDR3
TGNPYYTNGFNS

SEQ ID NO: 298 - protein, 10 aa, synthetic construct
  source 1..10: note = pimivalimab Vh CDR3
RGGTYYDYTY

SEQ ID NO: 299 - protein, 13 aa, synthetic construct
  source 1..13: note = rosnilimab Vh CDR3
RDYYGRFYYAMDY

SEQ ID NO: 300 - protein, 10 aa, synthetic construct
  source 1..10: note = rulonilimab Vh CDR3
RQKDTSWFVH

SEQ ID NO: 301 - protein, 10 aa, synthetic construct
  source 1..10: note = spartalizumab Vh CDR3
TRWTTGTGAY

SEQ ID NO: 302 - protein, 10 aa, synthetic construct
  source 1..10: note = sudubrilimab Vh CDR3
RRHWPGGFDY

SEQ ID NO: 303 - protein, 10 aa, synthetic construct
  source 1..10: note = finotonlimab Vh CDR3
RQYGTVWFFN

SEQ ID NO: 304 - protein, 15 aa, synthetic construct
  source 1..15: note = cetrelimab Vh CDR3
RPGLAAAYDTGSLDY

SEQ ID NO: 305 - protein, 7 aa, synthetic construct
  source 1..7: note = nofazinlimab Vh CDR3
IIGYFDY

SEQ ID NO: 306 - protein, 11 aa, synthetic construct
  source 1..11: note = pucotenlimab Vh CDR3
ARQKGEAWFAY

SEQ ID NO: 307 - protein, 9 aa, synthetic construct
  source 1..9: note = sasanlimab Vh CDR3
RLSTGTFAY

SEQ ID NO: 308 - protein, 5 aa, synthetic construct
  source 1..5: note = balstilimab Vh CDR3
SNGDH

SEQ ID NO: 309 - protein, 8 aa, synthetic construct
  source 1..8: note = geptanolimab Vh CDR3
CARDVFDY

SEQ ID NO: 310 - protein, 11 aa, synthetic construct
  source 1..11: note = retifanlimab Vh CDR3
REHYGTSPFAY

SEQ ID NO: 311 - protein, 8 aa, synthetic construct
  source 1..8: note = Camrelizumab Vl CDR1
QTIGTWLT

SEQ ID NO: 312 - protein, 8 aa, synthetic construct
  source 1..8: note = cemiplimab Vl CDR1
LSINTFLN

SEQ ID NO: 313 - protein, 8 aa, synthetic construct
  source 1..8: note = dostarlimab Vl CDR1
QDVGTAVA

SEQ ID NO: 314 - protein, 8 aa, synthetic construct
  source 1..8: note = nivolumab Vl CDR1
QSVSSYLA

SEQ ID NO: 315 - protein, 8 aa, synthetic construct
  source 1..8: note = penpulimab Vl CDR1
QDINTYLS

SEQ ID NO: 316 - protein, 8 aa, synthetic construct
  source 1..8: note = prolgolimab Vl CDR1
NIGSKNVH

SEQ ID NO: 317 - protein, 8 aa, synthetic construct
  source 1..8: note = serplulimab Vl CDR1
QDVTTAVA

SEQ ID NO: 318 - protein, 8 aa, synthetic construct
  source 1..8: note = sintilimab Vl CDR1
QGISSWLA

SEQ ID NO: 319 - protein, 8 aa, synthetic construct
  source 1..8: note = tislelizumab Vl CDR1
ESVSNDVA

SEQ ID NO: 320 - protein, 13 aa, synthetic construct
  source 1..13: note = toripalimab Vl CDR1
QSIVHSNGNTYLE

SEQ ID NO: 321 - protein, 11 aa, synthetic construct
  source 1..11: note = zimberelimab Vl CDR1
SSDVGFYNYVS

SEQ ID NO: 322 - protein, 14 aa, synthetic construct
  source 1..14: note = acrixolimab Vl CDR1
QSLLDSEDGNTYLD

SEQ ID NO: 323 - protein, 8 aa, synthetic construct
  source 1..8: note = zeluvalimab Vl CDR1
QGISNWLA

SEQ ID NO: 324 - protein, 13 aa, synthetic construct
  source 1..13: note = budigalimab Vl CDR1
QSIVHSHGDTYLE

SEQ ID NO: 325 - protein, 12 aa, synthetic construct
  source 1..12: note = ezabenlimab Vl CDR1
ENIDVSGISFMN

SEQ ID NO: 326 - protein, 14 aa, synthetic construct
  source 1..14: note = iparomlimab Vl CDR1
QSLFNSGNQKNYLA

SEQ ID NO: 327 - protein, 8 aa, synthetic construct
  source 1..8: note = peresolimab Vl CDR1
QSPNNLLA

SEQ ID NO: 328 - protein, 8 aa, synthetic construct
  source 1..8: note = pimivalimab Vl CDR1
QSISSWLA

SEQ ID NO: 329 - protein, 9 aa, synthetic construct
  source 1..9: note = rosnilimab Vl CDR1
SSVSSSYFH

SEQ ID NO: 330 - protein, 12 aa, synthetic construct
  source 1..12: note = rulonilimab Vl CDR1
ESVDDYGISFMN

SEQ ID NO: 331 - protein, 14 aa, synthetic construct
  source 1..14: note = spartalizumab Vl CDR1
QSLLDSGNQKNFLT

SEQ ID NO: 332 - protein, 8 aa, synthetic construct
  source 1..8: note = sudubrilimab Vl CDR1
QDVSTAVA

SEQ ID NO: 333 - protein, 12 aa, synthetic construct
  source 1..12: note = finotonlimab Vl CDR1
ESVDSYGNSFMH

SEQ ID NO: 334 - protein, 8 aa, synthetic construct
  source 1..8: note = cetrelimab Vl CDR1
QSVRSYLA

SEQ ID NO: 335 - protein, 13 aa, synthetic construct
  source 1..13: note = nofazinlimab Vl CDR1
QSLLDSDGGTYLY

SEQ ID NO: 336 - protein, 12 aa, synthetic construct
  source 1..12: note = pucotenlimab Vl CDR1
ESVDNYGISFMN

SEQ ID NO: 337 - protein, 14 aa, synthetic construct
  source 1..14: note = sasanlimab Vl CDR1
QSLWDSGNQKNFLT

SEQ ID NO: 338 - protein, 8 aa, synthetic construct
  source 1..8: note = balstilimab Vl CDR1
QSVSSNLA

SEQ ID NO: 339 - protein, 12 aa, synthetic construct
  source 1..12: note = geptanolimab Vl CDR1
ESVDNYGYSFMN

SEQ ID NO: 340 - protein, 12 aa, synthetic construct
  source 1..12: note = retifanlimab Vl CDR1
ESVDNYGMSFMN

SEQ ID NO: 341 - protein, 11 aa, synthetic construct
  source 1..11: note = Camrelizumab Vl CDR2
LLIYTATSLAD

SEQ ID NO: 342 - protein, 12 aa, synthetic construct
  source 1..12: note = cemiplimab Vl CDR2
NLLIYAASSLHG

SEQ ID NO: 343 - protein, 11 aa, synthetic construct
  source 1..11: note = dostarlimab Vl CDR2
LLIYWASTLHT

SEQ ID NO: 344 - protein, 11 aa, synthetic construct
  source 1..11: note = nivolumab and cetrelimab Vl CDR2
LLIYDASNRAT

SEQ ID NO: 345 - protein, 11 aa, synthetic construct
  source 1..11: note = penpulimab Vl CDR2
TLIYRANRLVS

SEQ ID NO: 346 - protein, 11 aa, synthetic construct
  source 1..11: note = prolgolimab Vl CDR2
LVIYRDSNRPS

SEQ ID NO: 347 - protein, 11 aa, synthetic construct
  source 1..11: note = serplulimab Vl CDR2
LLIYWASTRHT

SEQ ID NO: 348 - protein, 11 aa, synthetic construct
  source 1..11: note = sintilimab Vl CDR2
LLISAASSLQS

SEQ ID NO: 349 - protein, 11 aa, synthetic construct
  source 1..11: note = tislelizumab Vl CDR2
LLINYAFHRFT

SEQ ID NO: 350 - protein, 11 aa, synthetic construct
  source 1..11: note = toripalimab and budigalimab Vl CDR2
LLIYKVSNRFS

SEQ ID NO: 351 - protein, 11 aa, synthetic construct
  source 1..11: note = zimberelimab Vl CDR2
LMIYDVSNRPS

SEQ ID NO: 352 - protein, 11 aa, synthetic construct
  source 1..11: note = acrixolimab Vl CDR2
LLIYTLSHRAS

SEQ ID NO: 353 - protein, 11 aa, synthetic construct
  source 1..11: note = zeluvalimab Vl CDR2
LLIFAASSLQS

SEQ ID NO: 354 - protein, 11 aa, synthetic construct
  source 1..11: note = ezabenlimab and rulonilimab Vl CDR2
LLIYVASNQGS

SEQ ID NO: 355 - protein, 11 aa, synthetic construct
  source 1..11: note = iparomlimab Vl CDR2
LLIYGASTRDS

SEQ ID NO: 356 - protein, 11 aa, synthetic construct
  source 1..11: note = peresolimab Vl CDR2
LLIYGASDLPS

SEQ ID NO: 357 - protein, 11 aa, synthetic construct
  source 1..11: note = pimivalimab Vl CDR2
LLIYEASSLES

SEQ ID NO: 358 - protein, 11 aa, synthetic construct
  source 1..11: note = rosnilimab Vl CDR2
LLIYSTSNLAS

SEQ ID NO: 359 - protein, 11 aa, synthetic construct
  source 1..11: note = spartalizumab Vl CDR2
LLIYWASTRES

SEQ ID NO: 360 - protein, 11 aa, synthetic construct
  source 1..11: note = sudubrilimab Vl CDR2
LLIYSASFLYS

SEQ ID NO: 361 - protein, 11 aa, synthetic construct
  source 1..11: note = finotonlimab Vl CDR2
LLIYAASNQGS

SEQ ID NO: 362 - protein, 11 aa, synthetic construct
  source 1..11: note = nofazinlimab Vl CDR2
RLIYLVSTLGS

SEQ ID NO: 363 - protein, 11 aa, synthetic construct
  source 1..11: note = pucotenlimab Vl CDR2
LLIYAASNKGT

SEQ ID NO: 364 - protein, 11 aa, synthetic construct
  source 1..11: note = sasanlimab Vl CDR2
LLIYWTSYRES

SEQ ID NO: 365 - protein, 11 aa, synthetic construct
  source 1..11: note = balstilimab Vl CDR2
LLIYGASTRAT

SEQ ID NO: 366 - protein, 11 aa, synthetic construct
  source 1..11: note = geptanolimab Vl CDR2
LLIYRASNLES

SEQ ID NO: 367 - protein, 11 aa, synthetic construct
  source 1..11: note = retifanlimab Vl CDR2
LLIHAASNQGS

SEQ ID NO: 368 - protein, 8 aa, synthetic construct
  source 1..8: note = Camrelizumab Vl CDR3
QQVYSIPW

SEQ ID NO: 369 - protein, 8 aa, synthetic construct
  source 1..8: note = cemiplimab Vl CDR3
QQSSNTPF

SEQ ID NO: 370 - protein, 8 aa, synthetic construct
  source 1..8: note = dostarlimab Vl CDR3
QHYSSYPW

SEQ ID NO: 371 - protein, 8 aa, synthetic construct
  source 1..8: note = nivolumab Vl CDR3
QQSSNWPR

SEQ ID NO: 372 - protein, 8 aa, synthetic construct
  source 1..8: note = penpulimab Vl CDR3
LQYDEFPL

SEQ ID NO: 373 - protein, 8 aa, synthetic construct
  source 1..8: note = prolgolimab Vl CDR3
QVWDSSTA

SEQ ID NO: 374 - protein, 8 aa, synthetic construct
  source 1..8: note = serplulimab Vl CDR3
QQHYTIPW

SEQ ID NO: 375 - protein, 8 aa, synthetic construct
  source 1..8: note = sintilimab Vl CDR3
QQANHLPF

SEQ ID NO: 376 - protein, 8 aa, synthetic construct
  source 1..8: note = tislelizumab Vl CDR3
HQAYSSPY

SEQ ID NO: 377 - protein, 8 aa, synthetic construct
  source 1..8: note = toripalimab Vl CDR3
FQGSHVPL

SEQ ID NO: 378 - protein, 9 aa, synthetic construct
  source 1..9: note = zimberelimab Vl CDR3
SSYTSISTW

SEQ ID NO: 379 - protein, 8 aa, synthetic construct
  source 1..8: note = acrixolimab Vl CDR3
MQRRDFPF

SEQ ID NO: 380 - protein, 8 aa, synthetic construct
  source 1..8: note = zeluvalimab Vl CDR3
QQAESFPH

SEQ ID NO: 381 - protein, 8 aa, synthetic construct
  source 1..8: note = budigalimab Vl CDR3
FQGSHIPV

SEQ ID NO: 382 - protein, 8 aa, synthetic construct
  source 1..8: note = ezabenlimab, rulonilimab, finotonlimab and pucotenlimab Vl CDR3
QQSKEVPW

SEQ ID NO: 383 - protein, 8 aa, synthetic construct
  source 1..8: note = iparomlimab Vl CDR3
QNDHYYPY

SEQ ID NO: 384 - protein, 12 aa, synthetic construct
  source 1..12: note = peresolimab Vl CDR3
QNNYYVGPVSYA

SEQ ID NO: 385 - protein, 8 aa, synthetic construct
  source 1..8: note = pimivalimab Vl CDR3
QQYNSFPP

SEQ ID NO: 386 - protein, 8 aa, synthetic construct
  source 1..8: note = rosnilimab Vl CDR3
HQYHRSPL

SEQ ID NO: 387 - protein, 8 aa, synthetic construct
  source 1..8: note = spartalizumab Vl CDR3
QNDYSYPY

SEQ ID NO: 388 - protein, 8 aa, synthetic construct
  source 1..8: note = sudubrilimab Vl CDR3
QQFLYHPA

SEQ ID NO: 389 - protein, 8 aa, synthetic construct
  source 1..8: note = cetrelimab Vl CDR3
QQRNYWPL

SEQ ID NO: 390 - protein, 8 aa, synthetic construct
  source 1..8: note = nofazinlimab Vl CDR3
MQLTHWPY

SEQ ID NO: 391 - protein, 8 aa, synthetic construct
  source 1..8: note = sasanlimab Vl CDR3
QNDYFYPH

SEQ ID NO: 392 - protein, 8 aa, synthetic construct
  source 1..8: note = balstilimab Vl CDR3
QQYNNWPR

SEQ ID NO: 393 - protein, 8 aa, synthetic construct
  source 1..8: note = geptanolimab Vl CDR3
QQSNADPT

SEQ ID NO: 394 - protein, 8 aa, synthetic construct
  source 1..8: note = retifanlimab Vl CDR3
QQSKEVPY

(19) United States
(12) Patent Application Publication
(10) Pub. No.: US 2026/0240908 A1
(43) Pub. Date: Aug. 20, 2026

(54) ANTI-PD-1 CHIMERIC ANTIGEN RECEPTOR T CELLS AND THERAPEUTIC USES THEREOF

(71) Applicant: FRED HUTCHINSON CANCER CENTER, Seattle, WA (US)

(72) Inventor: Karsten EICHHOLZ, Seattle, WA (US)

(73) Assignee: FRED HUTCHINSON CANCER CENTER, Seattle, WA (US)

(21) Appl. No.: 19/161,473

(22) PCT Filed: Mar. 1, 2024
(86) PCT No.: PCT/US2024/018181

§ 371 Date: Sep. 2, 2025

Related U.S. Application Data
(60) Provisional application No. 63/488,113, filed on Mar. 2, 2023.

(51) Int. Cl. A61K 35/17; A61K 40/11; A61K 40/31; A61K 40/42; A61K 45/06; A61P 35/02; C07K 14/71; C07K 16/28; C12N 15/86

(52) U.S. Cl. CPC A61K 35/17; A61K 40/11; A61K 40/31; A61K 40/421; A61K 45/06; A61P 35/02; C07K 14/71; C07K 16/2818; C12N 15/86; A61K 2239/13; A61K 2239/17; A61K 2239/21; A61K 2239/22; A61K 2239/25; A61K 2239/48; C07K 2317/565; C07K 2317/622; C07K 2319/50; C12N 2740/15043; C12N 2800/22

(57) ABSTRACT

The present technology provides chimeric antigen receptors (CARs) targeting programmed death protein 1 (PD-1), host cells (e.g., T cells) that contain and/or express the anti-PD-1 CARs, and methods of using the CAR T cells for treating a variety of diseases including HIV, HIV-associated conditions, cancer, and autoimmune diseases.

### CROSS-REFERENCE TO RELATED APPLICATIONS

This application claims the benefit of U.S. Provisional Patent Application No. 63/488,113 filed Mar. 2, 2023. The contents this provisional application are incorporated by reference in their entirety.

### STATEMENT OF GOVERNMENT LICENSING RIGHTS

This invention was made with government support under AI126623 awarded by the National Institutes of Health. The government has certain rights in the invention.

### SEQUENCE LISTING INCORPORATED BY REFERENCE

This application contains an ST.26 compliant Sequence Listing, which is submitted concurrently in .xml format via Patent Center and is hereby incorporated by reference in its entirety. The .xml copy, created on Feb. 27, 2024, is named 0703548007WO00.xml and is 467,574 bytes in size.

### BACKGROUND

Human immunodeficiency virus (HIV) is a virus that attacks the body's immune system, such as T cells (especially CD4+ T cells), dendritic cells, and macrophages, and if untreated, it may lead to acquired immunodeficiency syndrome (AIDS). Despite the availability of antiretroviral drugs to manage HIV/AIDS and lower detectable viral load, there is currently no effective cure. HIV latency, and the consequent viral reservoir in CD4+ T cells and other immune cells, is the main barrier to eradication of the virus. A need thus exists for identification of novel therapeutic targets and development of new treatment for HIV/AIDS.

With the advent of antiretroviral therapy (ART), HIV infection has turned into a lifelong controllable disease. During ART, HIV-infected cells mainly reside in lymphoid tissues, including immune privileged sites such as lymph node B cell follicles and gut-associated lymphoid tissues. PD-1+ CD4+ T cells and Follicular helper T cells (TFH cells), which are characterized by high PD-1 and CXCR5 expression, are major sites for HIV/SIV infection and persistence during Moreover, PD-1 expressing central, transitional, and effector memory CD4+ T cells harbor more HIV provirus and produce more viral particles upon reactivation than their PD-1-counterparts, and ex vivo and in vitro data indicate that HIV preferentially replicates in TFH CD4+ T cells. Similarly, CD4+ T cells in blood with a TFH-like PD-1+/CXCR5+ phenotype show the highest HIV gag RNA and protein expression levels in viremic individuals ex vivo.

An emerging cell therapy approach called adoptive cell transfer (ACT) involves collecting cells from a patient (autologous) or healthy donors (allogeneic), adapting these cells, and transferring the cells into the patient to fight diseases. The ACT that has gained the most clinical success is chimeric antigen receptor (CAR) T cell therapy, where specially altered T cells specific to a disease-associated antigen (e.g., tumor antigen, viral antigen) are used to kill cells that harbor the antigen on their surface. However, new therapies to treat HIV and cancer are needed.

### SUMMARY

In some embodiments, the present technology comprises a chimeric antigen receptor (CAR) comprising a signal peptide, an extracellular binding domain specific to programmed death protein 1 (PD-1), a hinge domain, a transmembrane domain, an intracellular costimulatory domain, and/or an intracellular signaling domain.

In some embodiments, the present technology comprises a nucleic acid comprising a nucleotide sequence encoding the CAR.

In some embodiments, the present technology comprises a vector comprising the nucleic acid.

In some embodiments, the present technology comprises a virus comprising the nucleic acid or the vector.

In some embodiments, the present technology comprises a composition comprising the vector or the virus.

In some embodiments, the present technology comprises a host cell expressing the CAR, comprising the nucleic acid, and/or comprising the vector.

In some embodiments, the extracellular binding domain specific to PD-1 comprises an scFv derived from a portion of pembrolizumab.

In some embodiments, the scFv comprises one or more complementarity determining regions (CDRs) having amino acid sequences at least about 50%, 55%, 60%, 65%, 70%, 75%, 80%, 85%, 90%, 95%, or 100% identical to SEQ ID NOs: 6-8 and 11-13.

In some embodiments, the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and/or a light chain variable region comprising an amino acid sequence that is at least about 50%, 55%, 60%, 65%, 70%, 75%, 80%, 85%, 90%, 95%, or 100% identical to SEQ ID NO: 10.

In some embodiments, the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and a light chain variable region comprising an amino acid sequence that is at least about 50%, 55%, 60%, 65%, 70%, 75%, 80%, 85%, 90%, 95%, or 100% identical to SEQ ID NO: 10.

In some embodiments, the scFV comprises an amino acid sequence that is at least about 50%, 55%, 60%, 65%, 70%, 75%, 80%, 85%, 90%, 95%, or 100% identical to SEQ ID NO: 4 or SEQ ID NO: 14.

In some embodiments, the scFV comprises an amino acid sequence that at least about 50%, 55%, 60%, 65%, 70%, 75%, 80%, 85%, 90%, 95%, or 100% identical to SEQ ID NO: 4.

In some embodiments, the scFV comprises an amino acid sequence that is at least about 50%, 55%, 60%, 65%, 70%, 75%, 80%, 85%, 90%, 95%, or 100% identical to SEQ ID NO: 14.

In some embodiments, the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

In some embodiments, the hinge domain comprises an IgG4 hinge domain having an amino acid sequence at least about 50%, 55%, 60%, 65%, 70%, 75%, 80%, 85%, 90%, 95%, or 100% identical to SEQ ID NOs: 18-20.

In some embodiments, the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

In some embodiments, the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

In some embodiments, the intracellular signaling domain comprises a CD34 signaling domain having an amino acid sequence at least about 50%, 55%, 60%, 65%, 70%, 75%, 80%, 85%, 90%, 95%, or 100% identical to SEQ ID NO: 25.

In some embodiments, the extracellular binding domain specific to PD-1 comprises or consists of an amino acid sequence set forth in any one of SEQ ID NOS: 27-32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in any one of SEQ ID NOs: 27-32.

In some embodiments, the extracellular binding domain specific to PD-1 comprises or consists of an amino acid sequence set forth in SEQ ID NO: 27, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 27.

In some embodiments, the extracellular binding domain specific to PD-1 comprises or consists of an amino acid sequence set forth in SEQ ID NO: 28, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 28.

In some embodiments, the extracellular binding domain specific to PD-1 comprises or consists of an amino acid sequence set forth in SEQ ID NO: 29, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 29.

In some embodiments, the extracellular binding domain specific to PD-1 comprises or consists of an amino acid sequence set forth in SEQ ID NO: 30, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 30.

In some embodiments, the extracellular binding domain specific to PD-1 comprises or consists of an amino acid sequence set forth in SEQ ID NO: 31, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 31.

In some embodiments, the extracellular binding domain specific to PD-1 comprises or consists of an amino acid sequence set forth in SEQ ID NO: 32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 32.

In some embodiments, the CAR comprises an inducible hepatitis C-derived NS3 protease domain.

In some embodiments, the nucleic acid comprises a nucleotide sequence encoding the CAR is codon-optimized.

In some embodiments, the nucleic acid further comprises a nucleotide sequence encoding a truncated epidermal growth factor receptor (tEGFR) having an amino acid sequence at least about 50%, 55%, 60%, 65%, 70%, 75%, 80%, 85%, 90%, 95%, or 100% identical to SEQ ID NO: 34.

In some embodiments, the nucleic acid further comprises a nucleotide sequence encoding a c46 fusion inhibitor peptide having an amino acid sequence at least about 50%, 55%, 60%, 65%, 70%, 75%, 80%, 85%, 90%, 95%, or 100% identical to SEQ ID NO: 35.

In some embodiments, the nucleic acid comprises a nucleotide sequence encoding an amino acid sequence that is at least about 50%, 55%, 60%, 65%, 70%, 75%, 80%, 85%, 90%, 95%, or 100% identical to SEQ ID NO: 33.

In some embodiments, the vector is a plasmid, an adenoviral vector, an adeno-associated viral vector, a retroviral vector, or a lentiviral vector.

In some embodiments, the virus is an adenovirus, an adeno-associated virus, a retrovirus, a lentivirus, or a phage.

In some embodiments, the host cell is a T cell.

In some embodiments, the T cell is a CD4+ T cell, a CD8+ T cell, or a mixture thereof.

In some embodiments, the host cell is modified to express a safety switch.

In some embodiments, the safety switch is an inducible hepatitis C-derived NS3 protease domain or a tEGFR.

In some embodiments, the host cell is modified to have reduced or eliminated expression of an endogenous TCR.

In some embodiments, the extracellular binding domain specific to PD-1 comprises an scFv derived from a portion of an anti-PD-1 antibody at least about 50%, 55%, 60%, 65%, 70%, 75%, 80%, 85%, 90%, 95%, or 100% identical to an antibody in Table 8.

In some embodiments, the scFv comprises one or more complementarity determining regions (CDRs) having amino acid sequences at least about 50%, 55%, 60%, 65%, 70%, 75%, 80%, 85%, 90%, 95%, or 100% identical to a sequence in Table 8.

In some embodiments, the scFV comprises a heavy chain variable region comprising an amino acid sequence that is at least about 50%, 55%, 60%, 65%, 70%, 75%, 80%, 85%, 90%, 95%, or 100% identical to any of the heavy chain variable regions set forth in Table 8 and/or a light chain variable region comprising an amino acid sequence that is at least about 80% identical to any of the light chain variable regions set forth in Table 8.

In some embodiments, the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

In some embodiments, the hinge domain comprises an IgG4 hinge domain having an amino acid sequence set forth at least about 50%, 55%, 60%, 65%, 70%, 75%, 80%, 85%, 90%, 95%, or 100% identical to one or more of SEQ ID NOs: 18-20.

In some embodiments, the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

In some embodiments, the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

In some embodiments, the intracellular signaling domain comprises a CD3ζ signaling domain having an amino acid sequence at least about 50%, 55%, 60%, 65%, 70%, 75%, 80%, 85%, 90%, 95%, or 100% identical to SEQ ID NO: 25.

In some embodiments, the anti-PD-1 CAR comprises or consists of an amino acid sequence set forth in Table 8, or an amino acid sequence that is at least 80% identical, at least 85% identical, at least 90% identical, at least 95% identical, at least 96% identical, at least 97% identical, at least 98% identical, at least 99% identical, or 100% identical to any one of the amino acid sequences set forth in Table 8.

In some embodiments, the present technology comprises a pharmaceutical composition comprising the host cell.

In some embodiments, the present technology comprises a method of treating and/or preventing HIV infection, symptoms associated with HIV infection, and/or AIDS in a subject in need thereof, comprising administering to the subject a therapeutically effective amount of the host cell or the pharmaceutical composition.

In some embodiments, the method further comprises administering to the subject an antiretroviral therapy (ART).

In some embodiments, the present technology comprises a method of treating and/or preventing a PD-1-positive hematologic cancer in a subject in need thereof, comprising administering to the subject a therapeutically effective amount of the host cell or the pharmaceutical composition.

In some embodiments, the hematologic cancer is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T cell lymphoma, and B-cell lymphoma.

In some embodiments, the T-cell lymphoma is selected from the group consisting of angioimmunoblastic T-cell lymphoma, peripheral T-cell lymphoma with a follicular helper T cell (TFH) phenotype, and T follicular helper lymphoma.

In some embodiments, the method further comprises administering to the subject one or more additional therapeutic agents selected from the group consisting of an immunotherapy agent, a chemotherapy agent, and a biologic agent.

In some embodiments, the present technology comprises a method of treating and/or preventing an autoimmune disease in a subject in need thereof, comprising administering to the subject a therapeutically effective amount of the host cell or the pharmaceutical composition.

In some embodiments, the autoimmune disease is selected from the group consisting of type 1 diabetes, lupus, systemic lupus erythematosus (SLE), rheumatoid arthritis, psoriasis, psoriatic arthritis, multiple sclerosis, inflammatory bowel disease, Crohn's disease, ulcerative colitis, Addison's disease, Graves' disease, Sjogren's syndrome, Hashimoto's thyroiditis, myasthenia gravis, autoimmune vasculitis, pernicious anemia, and celiac disease.

In some embodiments, the present technology comprises a use of the host cell or the pharmaceutical composition for treating and/or preventing HIV infection, symptoms associated with HIV infection, and/or AIDS in a subject in need thereof.

In some embodiments, the present technology comprises a use of the host cell or the pharmaceutical composition for treating and/or preventing a PD-1-positive hematologic cancer in a subject in need thereof.

In some embodiments, the present technology comprises a use of the host cell or the pharmaceutical composition for treating and/or preventing an autoimmune disease in a subject in need thereof.

### BRIEF DESCRIPTION OF THE DRAWINGS

FIGS. 1A-1D: Shows a diagram of six anti-PD-1 CARs, which differ in the V_{H} V_{L} orientation of the pembrolizumab-derived scFv and the linker length between the transmembrane domain and scFv. CAR and EGFRt were interspersed with a T2A self-cleaving peptide (FIG. 1A). The six anti-PD-1 CAR vary in their paratope accessibility assessed with PD-1 Fc in Jurkat cells expressing the anti-PD-1 CAR or a VRC26.25-based CAR (FIG. 1B). Jurkat cells expressing the six anti-PD-1 CAR constructs exhibit low tonic signaling at baseline comparable to a control VRC26.25 CAR and upregulate Nur77 expression after induction with an anti-CD3/CD28 reagent for 4 h (FIG. 1C). Jurkat cells expressing the six anti-PD-1 CAR constructs upregulate Nur77 expression when cocultured for 4 h with K562 PD-1 GFP cells but not K562 GFP control cells. (FIG. 1D).

FIGS. 2A-2D illustrate that primary anti-PD-1 CAR T cells do not express PD-1 and kill PD-1-expressing cells. RM CD8+ T cells were transduced with the six anti-PD-1 CAR and analyzed by flow cytometry express EGFRt as a surrogate marker for CAR (FIG. 2A). Gating on EGFRt+ and − cells in the culture revealed a loss of PD-1 expression on CAR T and bystander cells in CAR T cells in comparison to non-transduced T cells (FIG. 2B). PD-1 Cell surface expression was quantified with quantibrite beads and anti-PD-1 antibody EH12 2H7 (FIG. 2C). Curves of a live cell microscopy killing assay of primary anti-PD-1 CAR CD8+ T cells with K562 PD1 GFP with low, medium, and high PD-1 expression or K562 GFP control cells were generated. Representative micrographs of the GFP expressing cells are shown below the kill curves (FIG. 2D).

FIGS. 3A-3F illustrate that anti-PD-1 CAR T cells attenuate SIV infection in vitro. FIG. 3A shows a schematic of the production of the infection of CD8-depleted peripheral blood mononuclear cells (PBMC) with SIVmac239 NefIRESGFP. CD8-depleted PBMC were activated for 3 days and infected by spinoculation for 2 h and cultured for 4 days. PD-1 expression on infected and non-infected cells after 4 days of infection (n=3) is shown using fluorescence minus one (FMO) controls for PD-1 and full staining samples (FIG. 3B). Schematic of preparation of CAR T cells and target cells infected with SIVmac239 NefIRESGFP for four days prior to experiment and representative images of GFP+ SIV-infected CD4+ T cells at the end of the 96 h coculture with effector cells (anti-PD-1 CAR, binding-deficient PD-1 CAR BD and non-transduced CD8+ T cells) at the indicated E:T ratio (n=3) (FIG. 3C). Representative quantification of GFP+ cells in the images acquired over 96 h in the killing assay shown in FIG. 3C (one representative experiment out of three); 2 replicates shown (FIG. 3D). Schematic of preparation of CAR T cells and target cells infected with SIVmac239 NefIRESGFP directly before the experiment and representative images at the end of the 96 h coculture with effector cells (anti-PD-1 CAR, binding-deficient PD-1 CAR BD and non-transduced CD8+ T cells) at the indicated E:T ratio (n=3) (FIG. 3E). Representative quantification of GFP+ cells in the images acquired over 96 h in the killing assay shown in FIG. 3E; 2 replicates shown (FIG. 3F).

FIGS. 4A-4H illustrate that anti-PD-1 CAR T cells expand in vivo and deplete PD-1+ CD4+ and CD8+ T cells in a SIV-naive RM. Schematic of the in vivo animal study in SIV-naive RMs (n=2). Tissues surgery, lymphodepletion, and Tocilizumab treatment are detailed in FIG. 4A. EGFRt and PD-1 cell marking in total CD3+ T cells in vivo at day 8 and 14 indicates robust expansion of anti-PD1 CAR T cells in animal B (FIG. 4B). Longitudinal PBMC sampling in animal B and animal A for percentages of total CAR T cell in CD3+ T cells (FIG. 4C) and PD-1+ in CD4+ and CD8+ T cells (FIG. 4D). Flow cytometric analysis of EGFR+ CD4+ or CD8+ CAR T cells in peripheral lymph nodes (PLN) (FIG. 4E). Expansion of CAR T cells in Peri.LN of animal B (FIG. 4F). Depletion of PD-1 expressing T_{FH} cells in lymph node was assessed by flow cytometry (FIG. 4G). Combined IHC and RNA FISH for CD3, CD20, PD-1, CD8α RNA, and CAR RNA, on B cell follicles of mesenteric lymph nodes on days-10, 8 and 24 relative to infusion. Arrows indicate the location of anti-PD-1 CAR CD8+ T cells within the B cell follicles (FIG. 4H). Asterisks (“*”) indicate exemplary CD20 staining. Plus symbols (“+”) indicate exemplary CD3 staining. Minus symbols (“−”) indicate exemplary PD-1 staining. “X” symbols indicate exemplary CD8a staining. Exemplary regions of CAR staining are circled.

FIGS. 5A-5H illustrate that anti-PD-1 CAR T cells deplete PD-1+ T cells in SIV-infected RM on antiretroviral therapies (ART). Schematic of the in vivo animal study (n=4). Dates for tissues surgery, lymphodepletion, duration of ART and Tocilizumab treatment are noted (FIG. 5A). Absolute count of EGFRt expressing CD3+ T cells in peripheral blood for the duration of the study (FIG. 5B). Longitudinal EGFRt expressing CD3+ T cells in peripheral lymph nodes (Peri.LN) (FIG. 5C). Flow cytometric analysis of PD-1 and CXCR5 expression on CD4+ and CD8+ total memory T cells in Peri.LN from animal 4 (FIG. 5D). Frequency of follicular helper T cells (T_{FH}) in CD4+ total memory T cells (FIG. 5E). Combined immunofluorescence and RNA FISH staining on lymph node tissue section for CD20, CD3, PD-1, CD8α RNA, and CAR RNA (FIG. 5F). black arrows point to intrafollicular CD8+ CAR T cells, white arrows point to residual T_{FH} cells post-infusion. T_{FH} cells characterized by CD3 and PD-1 dual staining within the B cell follicle and appear in the lightest shade on the pre-infusion panel and the day 49 non-expanded panel (FIG. 5F). Absolute count of PD-1+ CD4+ and CD8+ memory T cells in peripheral blood (FIG. 5G). PD-1 expression on Peri.LN CD4+ memory T cells and CD8+ memory T cells (FIG. 5H).

FIGS. 6A-6D illustrate that anti-PD-1 CAR T cell-mediated depletion of T_{FH} cells and PD-1+ T cells do not prevent viral recrudescence after removal of ART. Plasma viral load in four animals during anti-PD-1 CAR T cell treatment and after removal of ART (FIG. 6A). Absolute count of CD4+ memory and CD8+ memory T cells in blood (FIG. 6B). Combined immunofluorescence and RNA FISH staining on lymph node tissue section for CD3, CD20, PD-1, SIV RNA (FIG. 6C). Asterisks (“*”) indicate exemplary CD20 staining. Plus symbols (“+”) indicate exemplary CD3 staining. BCF: B cell follicle. TCZ: T cell zone. White dotted lines indicate the border between the TCZ and the BCF. FIG. 6D shows that cell-associated RNA and DNA levels in PBMC and lymph node preinfusion and 14 days post-CAR T cell infusion. PBMC and Peri.LN cell associated SIV DNA and RNA at the pre-anti-PD1-CAR-T cell infusion and at the time of ART cessation, 14 days post infusion are shown.

FIG. 7 illustrate effects of anti-PD-1 CAR T cells in SIV-infected RM. Cell distribution is depicted for a healthy lymph node, a SIV-infected lymph node, and a SIV-infected lymph node after anti-PD-1 CAR T cell expansion.

FIGS. 8A-8C illustrate that V_{H} V_{L} orientation and linker lengths affects paratope accessibility. Flow cytometry plots showing expression of EGFRt and paratope accessibility measured with PE-conjugated PD-1 Fc chimera of non-transduced Jurkat cells, a VRC26.25 CAR and the six anti-PD-1 CAR (FIG. 8A). Comparison of paratope accessibility of the V_{H} V_{L} and V_{L} V_{H} SCFv of anti-PD-1 CAR having the same linker length (FIG. 8B). Comparison of paratope accessibility dependent on the linker length for the V_{H} V_{L} and V_{L} V_{H} scFv orientation, respectively (FIG. 8C).

FIG. 9 illustrates PD-1 expression on primary anti-PD-1 CAR T cells. RM CD8+ T cells transduced with the six anti-PD-1 CAR and analyzed by flow cytometry detect EGFRt expression as a surrogate marker for CAR transduction.

FIGS. 10A-10E illustrate interaction of the anti-PD-1 CAR scFv and PD-1 in cis. Expression of EGFRt, CAR paratope (FIG. 10A) and PD-1 (FIG. 10B) on Jurkat cells containing a T2A-NeonGreen downstream of the Nur77 gene, transduced with anti-PD-1 CAR (V_{H}V_{L}S), and binding- or signaling-deficient versions of the same CAR (anti-PD-1 CAR BD, anti-PD-1 CAR ΔCD3ζ, and anti-PD-1 CAR BD ΔCD3ζ). Induction of Nur77 T2A NeonGreen expression in anti-PD-1 CAR, anti-PD-1 CAR BD, anti-PD-1 CAR ΔCD3ζ, and anti-PD-1 CAR BD ΔCD3ζ Jurkat reporter cell lines in presence of PD-1-expressing Molt 4 cells is shown (PMA/ionomycin shown as a positive control) (FIG. 10C). Expression of EGFRt, CAR paratope (FIG. 10D) and PD-1 (FIG. 10E) on Molt4 cells, which were transduced with anti-PD-1 CAR (V_{H}V_{L}S), and binding- or signaling-deficient versions of the same CAR (anti-PD-1 CAR BD, anti-PD-1 CAR ΔCD3ζ, and anti-PD-1 CAR BD ΔCD3ζ).

FIGS. 11A and 11B illustrate co-expression of CXCR5 and EGFRt in the anti-PD-1 CAR EGFRt c46 CXCR5 construct. Schematic of the anti-PD-1 CAR-EGFRt-c46 construct that coexpresses (FIG. 11A). Cell surface expression of EGFRt and CXCR5 on CD8+ T cells transduced with the indicated constructs (FIG. 11B).

FIG. 12 illustrates conservation of amino acids contributing to binding of pembrolizumab between Rhesus macaque and human PD-1. Underlined residues are implicated to interact with anti-PD-1 antibody pembrolizumab. Asterisks (*) indicate identical amino acids and full stop (.) and colon (:) indicate amino acids with similar properties. Grey background denotes the transmembrane domain.

FIGS. 13A-13C show kill curves of anti-PD-1 CAR T cells infused in SIV-naive RMs. Infusate anti-PD-1 CAR T cells were cocultured with K562 GFP (KG) PD-1 cells at the indicated E:T ratio and GFP expression was followed in live cell microscopy killing assay. Experimental replicates of kill curves of anti-PD-1 CAR T cells infused in SIV-naïve RMs are shown in FIG. 13C.

FIGS. 14A and 14B illustrates killing assays for the infusion products of the four SIVmac239-infected RM. Infusate anti-PD-1 CAR T cells were cocultured with K562 GFP (KG) PD-1 cells at the indicated E:T ratio and GFP expression was followed in live cell microscopy killing assay. Experimental replicates 1 (FIG. 14A) and 2 (FIG. 14B) are shown.

FIGS. 15A and 15B illustrate anti-PD-1 CAR T cell tissue traffic/expansion in various tissues. Flow pictures showing EGFRt+ T cells in whole blood and Peripheral lymph node (Peri.LN) in animal 4 (FIG. 15A). Frequency of anti-PD-1 CAR of total T cells in the biopsy and necropsy tissues (FIG. 15B).

FIG. 16 illustrate tissue distribution of anti-PD-1 CAR T cell at necropsy and frequency of (EGFR+) anti-PD-1 CAR T cells in the indicated tissues at necropsy.

FIG. 17A-17C illustrate the lack of PD-1 expression on anti-PD-1 CAR T cell infusion products and in vivo-expanded CAR T cells. Flow cytometry data showing PD-1 expression on memory CD4+ and CD8+T anti-PD-1 CAR T cells in the infusion product and in vivo-expanded cells on day 10 (FIG. 17A). Frequency of PD-1+ EGFRt+ T cells in vivo in longitudinal PBMC of SIV-infected RM animal 4 and animal 9 and (FIG. 17B) SIV-naive RM animal B (FIG. 17C).

FIGS. 18A and 18B show CAR T cell-mediated depletion of lymph node PD-1+ T_{FH} cells. Frequency of T_{FH} (CXCR5+, PD-1 hi) in CD4+ total memory T cells (FIG. 18A). Combined immunofluorescence and RNA FISH staining on lymph node tissue section from one animal without CAR T cell expansion and one animal with CAR T cell expansion (FIG. 18B). Single color staining is shown for DAPI, CD3, CD20, PD-1, CD8α RNA, and CAR RNA. Merged images are shown without the addition of the DAPI channel. CD3+ PD-1+ cells in the follicles appear as the lightest shade in panels 1, 2, and 3 and are defined as T_{FH} cells.

FIGS. 19A and 19B show depletion of PD-1+ T cell depletion in various tissues. Frequency of PD-1+ cells in CD4+ total memory T cells (FIG. 19A) and in total CD8+ memory T cells (FIG. 19B).

FIG. 20 show depletion of PD-1+ T cells in lymph nodes. Combined immunofluorescence and RNA FISH staining on D49/50 lymph node tissue section from one animal without CAR T cell expansion and one animal with CAR T cell expansion for CD3, CD20, PD-1, SIV RNA. The overlay CD20 and SIV RNA staining shows the enrichment of SIV infection in the extrafollicular T cell zone after successful anti-PD-1 CAR T cell expansion. Arrows point to exemplary CD20 staining. White circle indicates SIV RNA staining.

FIGS. 21A-21D illustrate the impact of anti-PD-1 CAR T cells on the T cell and B cell compartments. Absolute counts of CD2+, CD20+, CD3+, CD4+ and CD8+ cells in the SIV-naive RMs (FIG. 21A). Absolute count of CD4+ memory and CD8+ memory T cells in blood in the SIV-naive RMs (FIG. 21B). CD20 and Ki67 immunohistochemistry staining of pre-infusion and necropsy lymph node tissue section of animal B, an animal with anti-PD-1 CAR T cells expansion (FIG. 21C). CD3, CD20, and Ki67 immunofluorescence staining of pre-infusion and day 49/50 lymph node tissue section of one animal without and one with CAR T cell expansion (FIG. 21D). Arrows indicate exemplary CD20 staining. Asterisks indicate exemplary CD3 staining. Plus symbols (“+”) indicate exemplary Ki67 staining. BCF: B cell follicle. TCZ: T cell zone. White dotted lines indicate the border between the TCZ and the BCF.

### DETAILED DESCRIPTION

The present technology comprises chimeric antigen receptors (CARs) targeting PD-1, host cells (e.g., T cells) that contain and/or express the anti-PD-1 CARs, and methods of using the CAR T cells for treating a variety of diseases including HIV, HIV-associated conditions, cancer, and autoimmune diseases.

While the present technology is capable of being embodied in various forms, the description below of several embodiments is made with the understanding that the present technology is to be considered as an exemplification of the technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range, as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

To the extent any materials incorporated by reference herein conflict with the present technology, the present technology controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

The term “antibody” refers to natural antibodies or genetically engineered or otherwise modified forms of immunoglobulins or portions thereof, including chimeric antibodies, humanized antibodies, or synthetic antibodies. The antibodies may be monoclonal or polyclonal antibodies. In those embodiments wherein an antibody comprises an antigen-binding portion of an immunoglobulin molecule, the antibody may include, but is not limited to, a single-chain variable fragment antibody (scFv), a disulfide linked Fv, a single domain antibody (sdAb), a V_{H}H antibody, an antigen-binding fragment (Fab), a Fab′ fragment, a Fab₂ fragment, and a diabody. Specifically, an scFv antibody may be derived from a natural antibody by linking the variable regions of the heavy (V_{H}) and light (V_{L}) chains of the immunoglobulin with a short linker peptide. Similarly, a disulfide linked Fv antibody may be generated by linking the V_{H} and V_{L} using an interdomain disulfide bond. On the other hand, sdAbs consist of only the variable region from either the heavy or light chain and usually are the smallest antigen-binding fragments of antibodies. A V_{H}H antibody is the antigen-binding fragment of heavy chain only. A diabody is a dimer of scFv fragment that consists of the V_{H} and V_{L} regions noncovalently connected by a small peptide linker or covalently linked to each other. The antibodies of the present technology, including those that comprise an immunogenically active portion of an immunoglobulin molecule, retain the ability to bind a specific antigen.

The term “antigen” refers to a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term “antigen” broadly includes any type of molecule which is recognized by a host immune system as being foreign.

The term “binding domain” refers to an antibody or a portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, V_{H}H antibodies, Fab fragments, Fab′ fragments, Fab₂ fragments, diabodies, or other synthetic polypeptides selected for their specific ability to bind to a biological molecule, a molecular complex, or other target of interest.

The term “chimeric antigen receptors (CARs),” also known as chimeric T cell receptors or artificial T cell receptors, refers to artificially engineered receptors that combine both antigen-binding and T cell activating functions. CARs may include an extracellular portion comprising a binding domain, such as one obtained or derived from an antibody (e.g., an scFv). The extracellular portion may be linked through a transmembrane domain to one or more intracellular signaling and/or intracellular costimulatory domains. See, e.g., Sadelain et al., Cancer Discov. (2013) 3(4):388-398; see also Harris & Kranz, Trends Pharmacol. Sci. (2016) 37(3):220-230; Stone et al., Cancer Immunol. Immunother. (2014) 63(11):1163-1176, the entire disclosures of which are incorporated by reference herein. CARs may be introduced to be expressed on the surface of a T cell, so that the T cell may target and kill target cells that express the antigen the CAR is designed to bind.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods are known in the art, and include, for example, methods disclosed in at least U.S. Pat. Nos. 5,786,464 and 6,114,148, the entire disclosures of which are incorporated by reference herein.

The term “complementarity determining regions (CDRs)” is known in the art to refer to sequences of amino acids within antibody variable regions, which, in general, confer antigen specificity and/or binding affinity and are separated from one another in primary structure by framework sequence. In some cases, framework amino acids may also contribute to binding. In general, there are three CDRs in each variable region. Variable domain sequences may be aligned to a numbering scheme (e.g., Kabat, EU, international ImMunoGeneTics information System® (IMGT®), and Aho), which may allow equivalent residue positions to be annotated and for different molecules to be compared using the Antibody Numbering and Antigen Receptor Classification (ANARCI) software tool (2016, Bioinformatics 15:298-300).

The term “conservative substitution,” when referring to amino acid sequences, is recognized in the art as a substitution of one amino acid for another amino acid that has similar properties. A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine); and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively.

The term “construct” refers to any polynucleotide that contains a recombinant nucleic acid molecule. A construct may be present in a vector (e.g., a bacterial vector, a viral vector) or may be integrated into a genome.

The term “epitope” includes any molecule, structure, amino acid sequence, or protein determinant that is recognized and specifically bound by a cognate binding molecule, such as an antibody, or other binding molecule, domain, or protein.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “host cell” as used herein refers to a cell or microorganism targeted for genetic modification by introduction of a construct or vector carrying a nucleotide sequence for expression of a protein or polypeptide of interest. In some embodiments, when the protein to be expressed includes a CAR, the host cell is usually a T cell.

The term “hypoimmunogenicity,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and MHC II genes, and thus, to generate a hypoimmunogenic cell. Hypoimmunogenicity of a cell may be determined by evaluating the cell's ability to elicit adaptive and innate immune responses. Such immune response may be measured using assays recognized by those skilled in the art, for example, by measuring the effect of a hypoimmunogenic cell on T cell proliferation, T cell activation, T cell killing, natural killer (NK) cell proliferation, NK cell activation, and macrophage activity. Hypoimmunogenic cells may undergo decreased killing by T cells and/or NK cells upon administration to a subject or show decreased macrophage engulfment compared to an unmodified or wild-type cell. In some cases, a hypoimmunogenic cell elicits a reduced or diminished immune response in a recipient subject compared to a corresponding unmodified wild-type cell. In some cases, a hypoimmunogenic cell is nonimmunogenic or fails to elicit an immune response in a recipient subject.

An “intracellular signaling domain” is an intracellular portion or domain of a CAR or receptor that may directly or indirectly promote a biological or physiological response in a cell when receiving an appropriate signal. In some embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “operably linked” refers to the association of two or more nucleic acid molecules on a single nucleic acid fragment so that the function of one is affected by the other.

The term “prevention” or “preventing” in relation to a given disease or disorder includes preventing the onset of disease development if none had occurred; preventing the disease or disorder from occurring in a subject that may be predisposed to the disorder or disease but has not yet been diagnosed as having the disorder or disease; and/or preventing further disease/disorder development if already present.

The term “safety switch” used herein refers to a system for initiating the clearance or death of a host cell (e.g., through recognition by the host's immune system) or termination of the expression of a transgene in the host cell in a controllable manner. A safety switch may be designed to be triggered by an exogenous molecule in case of an adverse clinical event. A safety switch may be engineered by regulating the expression on the DNA, RNA and protein levels. A safety switch includes a protein or molecule that allows for the control of cellular activity in response to an adverse event. In some embodiments, the safety switch is a “kill switch” that is expressed in an inactive state and is fatal to a cell expressing the safety switch upon activation of the switch by a selective, externally provided agent.

The term “subject” refers to a mammalian subject, preferably a human. A “subject in need thereof” may refer to a subject who has been diagnosed with a disease, or is at an elevated risk of developing a disease. The phrases “subject” and “patient” are used interchangeably herein.

A “therapeutically effective amount” as used herein is an amount that produces a desired effect in a subject for treating a disease. In some embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or a pharmaceutical composition containing the same, and adjusting the dosage accordingly. For additional guidance, see, e.g., Remington, The Science and Practice of Pharmacy, 22^{nd} Edition, Pharmaceutical Press, London, 2012, and Goodman & Gilman, The Pharmacological Basis of Therapeutics, 12^{th} Edition, McGraw-Hill, New York, NY, 2011, the entire disclosures of which are incorporated by reference herein.

A “transmembrane domain” is a portion of a transmembrane protein that may insert into or span a cell membrane.

The term “treatment” or “treating” in relation to a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

The term “variable region” or “variable domain” refers to a portion of an antibody heavy or light chain that is involved in antigen binding. Variable domains of antibody heavy (V_{H}) and light (V_{L}) chains each generally comprise four generally conserved framework regions (FRs) and three CDRs. Framework regions separate CDRs, such that CDRs are situated between framework regions.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself.

### CARs, Nucleic Acids, Vectors, and Compositions Thereof

In some embodiments, the present technology comprises CARs that specifically recognize and/or target an antigen of interest, such as PD-1. CARs (also known as chimeric immunoreceptors, chimeric T cell receptors, or artificial T cell receptors) are receptor proteins that have been engineered to give host cells (e.g., T cells) the new ability to target a specific protein, especially cell surface proteins, in an MHC independent manner. The receptors are chimeric because they combine both an antigen-binding domain from an antibody or other non-TCR binding protein and T cell activating functions from TCR and/or other receptors into a single receptor. When a host cell, usually a T cell, is modified to express a CAR, it may specifically target the surface protein the CAR is designed to recognize and kill the cells bearing the surface protein.

In some embodiments, the CAR may comprise an extracellular binding domain (also referred to as a binder) that specifically binds a target antigen (e.g., PD-1), a transmembrane domain, and an intracellular signaling domain. In some embodiments, the CAR may further comprise one or more additional elements, including one or more signal peptides, one or more hinge domains, and/or one or more intracellular costimulatory domains. Domains may be directly adjacent to one another, or there may be one or more amino acids linking the domains. The signal peptide may be derived from an antibody, a T cell receptor (TCR), CD8, or other type 1 membrane proteins, preferably a protein expressed in a T or other immune cell. The transmembrane domain may be one associated with any of the potential intracellular domains or from another type 1 membrane protein, such as TCR alpha or beta chain, CD3 epsilon or zeta chain, CD4, CD8, or CD28, among other possibilities known in the art. The CAR may further comprise a hinge domain (also referred to as a spacer) of various lengths located between the extracellular binding domain and the hydrophobic membrane-spanning region of the transmembrane domain. The intracellular signaling domain may be derived from the CD3 zeta chain, CD27, CD28, 4-1BB, DAP12, FcγRIII, FcsRI, or an immunoreceptor tyrosine-based activation motif (ITAM) bearing cytoplasmic domain, among other possibilities known in the art.

The amino acid and/or nucleotide sequence of a CAR may be derived from a mammalian species, for example, rats, mice, primates, human, or combinations thereof. In the cases where the amino acid and/or nucleotide sequence of a CAR is nonhuman, the sequence of the CAR may be humanized. For example, the nucleotide sequence may also be codon-optimized for expression in a mammalian cell, for example, a human cell. In any of these embodiments, the amino acid and/or nucleotide sequence encoding a CAR may be at least 80% identical (e.g., at least 80%, at least 85%, at least 90%, at least 95%, at least 96%, at least 97%, at least 98%, at least 99%, or 100% identical) to any of the amino acid and/or nucleotide sequences of the present technology. The sequence variations may be due to codon-optimalization, humanization, restriction enzyme-based cloning scars, conservative substitution, and/or additional amino acid residues linking the functional domains.

In some embodiments, the CAR may comprise a signal peptide at the N-terminus. Non-limiting examples of signal peptides include CD8α signal peptide, IgK signal peptide, granulocyte-macrophage colony-stimulating factor (GM-CSF) signal peptide, and variants thereof, the amino acid sequences of which are provided in Table 1 below.

TABLE 1
Exemplary sequences of signal peptides

| SEQ | | |
|---|---|---|
| ID | | |
| NO: | Sequence | Description |
| 1 | MALPVTALLLPLALLLHAARP | CD8α signal peptide |
| 2 | METDTLLLWVLLLWVPGSTG | IgK signal peptide |
| 3 | MLLLVTSLLLCELPHPAFLLIP | GM-CSF signal peptide |

In some embodiments, the CAR may comprise a signal peptide having an amino acid sequence about 50%, about 55%, about 60%, about 65%, about 70%, about 75%, about 80%, about 85%, about 90%, about 95%, about 100% identical one of SEQ ID NOs: 1-3 at the N-terminus.

In some embodiments, the CAR may comprise a signal peptide having an amino acid sequence at least 50%, at least 55%, at least 60%, at least 65%, at least 70%, at least 75%, at least 80%, at least 85%, at least 90%, at least 95%, at least 100% identical one of SEQ ID NOs: 1-3 at the N-terminus.

In some embodiments, the CAR may comprise a signal peptide having an amino acid sequence at least about 50%, at least about 55%, at least about 60%, at least about 65%, at least about 70%, at least about 75%, at least about 80%, at least about 85%, at least about 90%, at least about 95%, at least about 100% identical one of SEQ ID NOs: 1-3 at the N-terminus.

In some embodiments, the CAR may comprise an extracellular binding domain, also referred to as a binder, that specifically binds a target antigen (e.g., PD-1). In some embodiments, the CAR is an anti-PD-1 CAR, i.e., the extracellular binding domain of the CAR is specific to PD-1, for example, human PD-1. The extracellular binding domain of the anti-PD-1 CAR may be codon-optimized for expression in a host cell or have variant sequences to increase functions of the extracellular binding domain. In some embodiments, the extracellular binding domain comprises an antibody or an antigen-binding portion of an immunoglobulin molecule, for example, an scFv. The scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, nxGGGGS (n may be a positive integer, e.g., 1, 2, 3, 4, 5, 6, etc.) linker, and variants thereof.

In some embodiments, the extracellular binding domain of the anti-PD-1 CAR comprises an scFv derived from the anti-PD-1 antibody pembrolizumab, which comprises the heavy chain variable region (V_{H}) and the light chain variable region (V_{L}) of pembrolizumab connected by a linker. Exemplary amino acid sequences of the pembrolizumab-derived scFv and its components are provided in Table 2 below. In some embodiments, the extracellular binding domain of the anti-PD-1 CAR comprises an scFv derived from any of the anti-PD-1 antibodies set forth in Table 8 which comprises the heavy chain variable region (V_{H}) and the light chain variable region (V_{L}) of these anti-PD-1 antibodies connected by a linker.

In some embodiments, the anti-PD-1 CAR comprises or consists of an amino acid sequence set forth in Table 8.

In some embodiments, the anti-PD-1 CAR comprises or consists of an amino acid sequence that is about 50% identical, about 55% identical, about 60% identical, about 65% identical, about 70% identical, about 75% identical, about 80% identical, about 85% identical, about 90% identical, about 95% identical, about 96% identical, about 97% identical, about 98% identical, about 99% identical, or 100% identical to any one of the amino acid sequences set forth in Table 8.

In some embodiments, the anti-PD-1 CAR comprises or consists of an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to any one of the amino acid sequences set forth in Table 8.

In some embodiments, the anti-PD-1 CAR comprises or consists of an amino acid sequence that is at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, or 100% identical to any one of the amino acid sequences set forth in Table 8.

Table 8 comprises sequences for certain human monoclonal IgG anti-PD-1 antibodies designated by their INN names, including heavy chains, light chains, heavy chain variable regions, light chain variable regions, heavy chain CDRs, light chain CDRs, and corresponding scFVs. The beginnings of the constant regions comprise the following sequences for each of the indicated chains: Gamma1=ASTK; Lambda1=GQPKANPT; Lambda2=GQPKAAPS; Kappa=RTVAAPS.

TABLE 8
Antibody Sequences

| | Heavy | Light | | Vl | | | | | | | | | |
|---|---|---|---|---|---|---|---|---|---|---|---|---|---|
| | Chain | Chain | Vh | Chain | | | | | | | | | |
| | Amino | Amino | Amino | Acid | | | | | | | | | |
| | Acid | Acid | Acid | Se- | Vh Vl | Vl Vh | Vh: | Vh: | Vh: | Vl: | Vl: | Vl: | INN |
| INN name | Sequence | Sequence | Sequence | quence | scFv | scFv | CDR1 | CDR2 | CDR3 | CDR1 | CDR2 | CDR3 | name |
| camrelizumab | EVQLVE | DIQMTQ | EVQLVE | DIQMTQ | EVQLVE | DIQMTQ | FTFS | WVA | ARQL | QTIG | LLIYT | QQV | cam- |
| | SGGGLV | SPSSLSA | SGGGLV | SPSSLSA | SGGGLV | SPSSLSA | SYM | TISG | YYFD | TWLT | ATSL | YSIP | relizumab |
| | QPGGSL | SVGDRV | QPGGSL | SVGDRV | QPGGSL | SVGDRV | MS | GGA | Y (97- | (27- | AD | W | |
| | RLSCAA | TITCLAS | RLSCAA | TITCLAS | RLSCAA | TITCLAS | (27- | NTYY | 105) | 34) | (46- | (89- | |
| | SGFTFS | QTIGTWL | SGFTFS | QTIGTWL | SGFTFS | QTIGTWL | 35) | P (47- | (SEQ | (SEQ | 56) | 96) | |
| | SYMMS | TWYQQK | SYMMS | TWYQQK | SYMMS | TWYQQK | (SEQ | 61) | ID | ID | (SEQ | (SEQ | |
| | WVRQAP | PGKAPK | WVRQAP | PGKAPK | WVRQAP | PGKAPK | ID | (SEQ | NO: | NO: | ID | ID | |
| | GKGLEW | LLIYTAT | GKGLEW | LLIYTAT | GKGLEW | LLIYTAT | NO: | ID | 281) | 311) | NO: | NO: | |
| | VATISGG | SLADGV | VATISGG | SLADGV | VATISGG | SLADGV | 227) | NO: | | | 341) | 368) | |
| | GANTYY | PSRFSG | GANTYY | PSRFSG | GANTYY | PSRFSG | | 253) | | | | | |
| | PDSVKG | SGSGTD | PDSVKG | SGSGTD | PDSVKG | SGSGTD | | | | | | | |
| | RFTISRD | FTLTISSL | RFTISRD | FTLTISS | RFTISRD | FTLTISSL | | | | | | | |
| | NAKNSL | QPEDFA | NAKNSL | LQPEDFA | NAKNSL | QPEDFA | | | | | | | |
| | YLQMNS | TYYCQQ | YLQMNS | TYYCQQ | YLQMNS | TYYCQQ | | | | | | | |
| | LRAEDT | VYSIPWT | LRAEDT | VYSIPWT | LRAEDT | VYSIPWT | | | | | | | |
| | AVYYCA | FGGGTK | AVYYCA | FGGGTK | AVYYCA | FGGGTK | | | | | | | |
| | RQLYYF | VEIKRTV | RQLYYF | VEIK | RQLYYF | VEIKGG | | | | | | | |
| | DYWGQ | AAPSVFI | DYWGQ | (SEQ ID | DYWGQ | GGSGGG | | | | | | | |
| | GTTVTV | FPPSDE | GTTVTV | NO: 137) | GTTVTV | GSGGGG | | | | | | | |
| | SSASTK | QLKSGT | SS (SEQ | | SSGGGG | SEVQLV | | | | | | | |
| | GPSVFP | ASVVCLL | ID NO: | | SGGGGS | ESGGGL | | | | | | | |
| | LAPCSR | NNFYPR | 107) | | GGGGSD | VQPGGS | | | | | | | |
| | STSESTA | EAKVQW | | | IQMTQS | LRLSCAA | | | | | | | |
| | ALGCLV | KVDNAL | | | PSSLSAS | SGFTFS | | | | | | | |
| | KDYFPE | QSGNSQ | | | VGDRVTI | SYMMS | | | | | | | |
| | PVTVSW | ESVTEQ | | | TCLASQ | WVRQAP | | | | | | | |
| | NSGALT | DSKDST | | | TIGTWLT | GKGLEW | | | | | | | |
| | SGVHTF | YSLSSTL | | | WYQQKP | VATISGG | | | | | | | |
| | PAVLQS | TLSKADY | | | GKAPKLL | GANTYY | | | | | | | |
| | SGLYSLS | EKHKVY | | | IYTATSL | PDSVKG | | | | | | | |
| | SVVTVP | ACEVTH | | | ADGVPS | RFTISRD | | | | | | | |
| | SSSLGT | QGLSSP | | | RFSGSG | NAKNSL | | | | | | | |
| | KTYTCN | VTKSFN | | | SGTDFTL | YLQMNS | | | | | | | |
| | VDHKPS | RGEC | | | TISSLQP | LRAEDT | | | | | | | |
| | NTKVDK | (SEQ ID | | | EDFATY | AVYYCA | | | | | | | |
| | RVESKY | NO: 77) | | | YCQQVY | RQLYYF | | | | | | | |
| | GPPCPP | | | | SIPWTFG | DYWGQ | | | | | | | |
| | CPAPEFL | | | | GGTKVEI | GTTVTV | | | | | | | |
| | GGPSVF | | | | K (SEQ | SS (SEQ | | | | | | | |
| | LFPPKPK | | | | ID NO: | ID NO: | | | | | | | |
| | DTLMISR | | | | 167) | 197) | | | | | | | |
| | TPEVTC | | | | | | | | | | | | |
| | VVVDVS | | | | | | | | | | | | |
| | QEDPEV | | | | | | | | | | | | |
| | QFNWYV | | | | | | | | | | | | |
| | DGVEVH | | | | | | | | | | | | |
| | NAKTKP | | | | | | | | | | | | |
| | REEQFN | | | | | | | | | | | | |
| | STYRVV | | | | | | | | | | | | |
| | SVLTVLH | | | | | | | | | | | | |
| | QDWLNG | | | | | | | | | | | | |
| | KEYKCK | | | | | | | | | | | | |
| | VSNKGL | | | | | | | | | | | | |
| | PSSIEKTI | | | | | | | | | | | | |
| | SKAKGQ | | | | | | | | | | | | |
| | PREPQV | | | | | | | | | | | | |
| | YTLPPS | | | | | | | | | | | | |
| | QEEMTK | | | | | | | | | | | | |
| | NQVSLT | | | | | | | | | | | | |
| | CLVKGF | | | | | | | | | | | | |
| | YPSDIAV | | | | | | | | | | | | |
| | EWESNG | | | | | | | | | | | | |
| | QPENNY | | | | | | | | | | | | |
| | KTTPPVL | | | | | | | | | | | | |
| | DSDGSF | | | | | | | | | | | | |
| | FLYSRLT | | | | | | | | | | | | |
| | VDKSRW | | | | | | | | | | | | |
| | QEGNVF | | | | | | | | | | | | |
| | SCSVMH | | | | | | | | | | | | |
| | EALHNH | | | | | | | | | | | | |
| | YTQKSL | | | | | | | | | | | | |
| | SLSLGK | | | | | | | | | | | | |
| | (SEQ ID | | | | | | | | | | | | |
| | NO: 47) | | | | | | | | | | | | |
| cemiplimab | EVQLLES | DIQMTQ | EVQLLES | DIQMTQ | EVQLLES | DIQMTQ | FTFS | WVS | VKW | LSINT | NLLIY | QQS | cemi- |
| | GGVLVQ | SPSSLSA | GGVLVQ | SPSSLSA | GGVLVQ | SPSSLSA | NFG | GISG | GNIY | FLN | AASS | SNTP | plimab |
| | PGGSLR | SVGDSIT | PGGSLR | SVGDSIT | PGGSLR | SVGDSIT | MT | GGR | FDY | (27- | LHG | F (89- | |
| | LSCAAS | ITCRASL | LSCAAS | ITCRASL | LSCAAS | ITCRASL | (27- | DTYF | (97- | 34) | (45- | 96) | |
| | GFTFSN | SINTFLN | GFTFSN | SINTFLN | GFTFSN | SINTFLN | 35) | A (47- | 106) | (SEQ | 56) | (SEQ | |
| | FGMTWV | WYQQKP | FGMTWV | WYQQKP | FGMTWV | WYQQKP | (SEQ | 61) | (SEQ | ID | (SEQ | ID | |
| | RQAPGK | GKAPNL | RQAPGK | GKAPNL | RQAPGK | GKAPNL | ID | (SEQ | ID | NO: | ID | NO: | |
| | GLEWVS | LIYAASS | GLEWVS | LIYAASS | GLEWVS | LIYAASS | NO: | ID | NO: | 312) | NO: | 369) | |
| | GISGGG | LHGGVP | GISGGG | LHGGVP | GISGGG | LHGGVP | 228) | NO: | 282) | | 342) | | |
| | RDTYFA | SRFSGS | RDTYFA | SRFSGS | RDTYFA | SRFSGS | | 254) | | | | | |
| | DSVKGR | GSGTDF | DSVKGR | GSGTDF | DSVKGR | GSGTDF | | | | | | | |
| | FTISRDN | TLTIRTL | FTISRDN | TLTIRTL | FTISRDN | TLTIRTL | | | | | | | |
| | SKNTLYL | QPEDFA | SKNTLYL | QPEDFA | SKNTLYL | QPEDFA | | | | | | | |
| | QMNSLK | TYYCQQ | QMNSLK | TYYCQQ | QMNSLK | TYYCQQ | | | | | | | |
| | GEDTAV | SSNTPFT | GEDTAV | SSNTPFT | GEDTAV | SSNTPFT | | | | | | | |
| | YYCVKW | FGPGTV | YYCVKW | FGPGTV | YYCVKW | FGPGTV | | | | | | | |
| | GNIYFDY | VDFRRT | GNIYFDY | VDFR | GNIYFDY | VDFRGG | | | | | | | |
| | WGQGTL | VAAPSV | WGQGTL | (SEQ ID | WGQGTL | GGSGGG | | | | | | | |
| | VTVSSA | FIFPPSD | VTVSS | NO: 138) | VTVSSG | GSGGGG | | | | | | | |
| | STKGPS | EQLKSG | (SEQ ID | | GGGSGG | SEVQLLE | | | | | | | |
| | VFPLAPC | TASVVCL | NO: 108) | | GGSGGG | SGGVLV | | | | | | | |
| | SRSTSE | LNNFYP | | | GSDIQM | QPGGSL | | | | | | | |
| | STAALG | REAKVQ | | | TQSPSS | RLSCAA | | | | | | | |
| | CLVKDY | WKVDNA | | | LSASVG | SGFTFS | | | | | | | |
| | FPEPVTV | LQSGNS | | | DSITITC | NFGMTW | | | | | | | |
| | SWNSGA | QESVTE | | | RASLSIN | VRQAPG | | | | | | | |
| | LTSGVH | QDSKDS | | | TFLNWY | KGLEWV | | | | | | | |
| | TFPAVLQ | TYSLSST | | | QQKPGK | SGISGG | | | | | | | |
| | SSGLYSL | LTLSKAD | | | APNLLIY | GRDTYF | | | | | | | |
| | SSVVTV | YEKHKV | | | AASSLH | ADSVKG | | | | | | | |
| | PSSSLG | YACEVT | | | GGVPSR | RFTISRD | | | | | | | |
| | TKTYTC | HQGLSS | | | FSGSGS | NSKNTL | | | | | | | |
| | NVDHKP | PVTKSF | | | GTDFTLT | YLQMNS | | | | | | | |
| | SNTKVD | NRGEC | | | IRTLQPE | LKGEDT | | | | | | | |
| | KRVESK | (SEQ ID | | | DFATYY | AVYYCV | | | | | | | |
| | YGPPCP | NO: 78) | | | CQQSSN | KWGNIY | | | | | | | |
| | PCPAPE | | | | TPFTFGP | FDYWGQ | | | | | | | |
| | FLGGPS | | | | GTVVDF | GTLVTVS | | | | | | | |
| | VFLFPPK | | | | R (SEQ | S (SEQ | | | | | | | |
| | PKDTLMI | | | | ID NO: | ID NO: | | | | | | | |
| | SRTPEV | | | | 168) | 198) | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 48) | | | | | | | | | | | | |
| dostarlimab | EVQLLES | DIQLTQS | EVQLLES | DIQLTQS | EVQLLES | DIQLTQS | FTFS | WVS | SPYY | QDV | LLIY | QHYS | dostar |
| | GGGLVQ | PSFLSAY | GGGLVQ | PSFLSAY | GGGLVQ | PSFLSAY | SYD | TISG | AMD | GTAV | WAS | SYP | limab |
| | PGGSLR | VGDRVTI | PGGSLR | VGDRVTI | PGGSLR | VGDRVTI | MS | GGS | Y (98- | A (27- | TLHT | W | |
| | LSCAAS | TCKASQ | LSCAAS | TCKASQ | LSCAAS | TCKASQ | (27- | YTYY | 105) | 34) | (46- | (89- | |
| | GFTFSS | DVGTAV | GFTFSS | DVGTAV | GFTFSS | DVGTAV | 35) | Q (47- | (SEQ | (SEQ | 56) | 96) | |
| | YDMSWV | AWYQQK | YDMSWV | AWYQQK | YDMSWV | AWYQQK | (SEQ | 61) | ID | ID | (SEQ | (SEQ | |
| | RQAPGK | PGKAPK | RQAPGK | PGKAPK | RQAPGK | PGKAPK | ID | (SEQ | NO: | NO: | ID | ID | |
| | GLEWVS | LLIYWAS | GLEWVS | LLIYWAS | GLEWVS | LLIYWAS | NO: | ID | 283) | 313) | NO: | NO: | |
| | TISGGGS | TLHTGV | TISGGGS | TLHTGV | TISGGGS | TLHTGV | 229) | NO: | | | 343) | 370) | |
| | YTYYQD | PSRFSG | YTYYQD | PSRFSG | YTYYQD | PSRFSG | | 255) | | | | | |
| | SVKGRF | SGSGTEF | SVKGRF | SGSGTEF | SVKGRF | SGSGTE | | | | | | | |
| | TISRDNS | TLTISSL | TISRDNS | TLTISSL | TISRDNS | FTLTISSL | | | | | | | |
| | KNTLYLQ | QPEDFA | KNTLYLQ | QPEDFA | KNTLYLQ | QPEDFA | | | | | | | |
| | MNSLRA | TYYCQH | MNSLRA | TYYCQH | MNSLRA | TYYCQH | | | | | | | |
| | EDTAVY | YSSYPW | EDTAVY | YSSYPW | EDTAVY | YSSYPW | | | | | | | |
| | YCASPY | TFGQGT | YCASPY | TFGQGT | YCASPY | TFGQGT | | | | | | | |
| | YAMDYW | KLEIKRT | YAMDYW | KLEIK | YAMDYW | KLEIKGG | | | | | | | |
| | GQGTTV | VAAPSV | GQGTTV | (SEQ ID | GQGTTV | GGSGGG | | | | | | | |
| | TVSSAST | FIFPPSD | TVSS | NO: 139) | TVSSGG | GSGGGG | | | | | | | |
| | KGPSVF | EQLKSG | (SEQ ID | | GGSGGG | SEVQLLE | | | | | | | |
| | PLAPCS | TASVVCL | NO: 109) | | GSGGGG | SGGGLV | | | | | | | |
| | RSTSES | LNNFYP | | | SDIQLTQ | QPGGSL | | | | | | | |
| | TAALGCL | REAKVQ | | | SPSFLSA | RLSCAA | | | | | | | |
| | VKDYFP | WKVDNA | | | YVGDRV | SGFTFS | | | | | | | |
| | EPVTVS | LQSGNS | | | TITCKAS | SYDMSW | | | | | | | |
| | WNSGAL | QESVTE | | | QDVGTA | VRQAPG | | | | | | | |
| | TSGVHT | QDSKDS | | | VAWYQQ | KGLEWV | | | | | | | |
| | FPAVLQ | TYSLSST | | | KPGKAP | STISGGG | | | | | | | |
| | SSGLYSL | LTLSKAD | | | KLLIYWA | SYTYYQ | | | | | | | |
| | SSVVTV | YEKHKV | | | STLHTG | DSVKGR | | | | | | | |
| | PSSSLG | YACEVT | | | VPSRFS | FTISRDN | | | | | | | |
| | TKTYTC | HQGLSS | | | GSGSGT | SKNTLYL | | | | | | | |
| | NVDHKP | PVTKSF | | | EFTLTIS | QMNSLR | | | | | | | |
| | SNTKVD | NRGEC | | | SLQPED | AEDTAV | | | | | | | |
| | KRVESK | (SEQ ID | | | FATYYC | YYCASP | | | | | | | |
| | YGPPCP | NO: 79) | | | QHYSSY | YYAMDY | | | | | | | |
| | PCPAPE | | | | PWTFGQ | WGQGTT | | | | | | | |
| | FLGGPS | | | | GTKLEIK | VTVSS | | | | | | | |
| | VFLFPPK | | | | (SEQ ID | (SEQ ID | | | | | | | |
| | PKDTLMI | | | | NO: 169) | NO: 199) | | | | | | | |
| | SRTPEV | | | | | | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 49) | | | | | | | | | | | | |
| niVQLumab | QVQLVE | EIVLTQS | QVQLVE | EIVLTQS | QVQLVE | EIVLTQS | ITFS | WVA | TNDD | QSVS | LLIYD | QQS | nivolu |
| | SGGGVV | PATLSLS | SGGGVV | PATLSLS | SGGGVV | PATLSLS | NSG | VIWY | Y (98- | SYLA | ASNR | SNW | mab |
| | QPGRSL | PGERAT | QPGRSL | PGERAT | QPGRSL | PGERAT | MH | DGSK | 102) | (27- | AT | PR | |
| | RLDCKA | LSCRAS | RLDCKA | LSCRAS | RLDCKA | LSCRAS | (27- | RYY | (SEQ | 34) | (46- | (89- | |
| | SGITFSN | QSVSSY | SGITFSN | QSVSSY | SGITFSN | QSVSSY | 35) | (47- | ID | (SEQ | 56) | 96) | |
| | SGMHW | LAWYQQ | SGMHW | LAWYQQ | SGMHW | LAWYQQ | (SEQ | 60) | NO: | ID | (SEQ | (SEQ | |
| | VRQAPG | KPGQAP | VRQAPG | KPGQAP | VRQAPG | KPGQAP | ID | (SEQ | 284) | NO: | ID | ID | |
| | KGLEWV | RLLIYDA | KGLEWV | RLLIYDA | KGLEWV | RLLIYDA | NO: | ID | | 314) | NO: | NO: | |
| | AVIWYD | SNRATGI | AVIWYD | SNRATGI | AVIWYD | SNRATGI | 230) | NO: | | | 344) | 371) | |
| | GSKRYY | PARFSG | GSKRYY | PARFSG | GSKRYY | PARFSG | | 256) | | | | | |
| | ADSVKG | SGSGTDF | ADSVKG | SGSGTD | ADSVKG | SGSGTD | | | | | | | |
| | RFTISRD | TLTISSL | RFTISRD | FTLTISS | RFTISRD | FTLTISSL | | | | | | | |
| | NSKNTLF | EPEDFA | NSKNTLF | LEPEDFA | NSKNTLF | EPEDFA | | | | | | | |
| | LQMNSL | VYYCQQ | LQMNSL | VYYCQQ | LQMNSL | VYYCQQ | | | | | | | |
| | RAEDTA | SSNWPR | RAEDTA | SSNWPR | RAEDTA | SSNWPR | | | | | | | |
| | VYYCAT | TFGQGT | VYYCAT | TFGQGT | VYYCAT | TFGQGT | | | | | | | |
| | NDDYWG | KVEIKRT | NDDYWG | KVEIK | NDDYWG | KVEIKGG | | | | | | | |
| | QGTLVT | VAAPSV | QGTLVT | (SEQ ID | QGTLVT | GGSGGG | | | | | | | |
| | VSSAST | FIFPPSD | VSS | NO: 140) | VSSGGG | GSGGGG | | | | | | | |
| | KGPSVF | EQLKSG | (SEQ ID | | GSGGGG | SQVQLV | | | | | | | |
| | PLAPCS | TASVVCL | NO: 110) | | SGGGGS | ESGGGV | | | | | | | |
| | RSTSES | LNNFYP | | | EIVLTQS | VQPGRS | | | | | | | |
| | TAALGCL | REAKVQ | | | PATLSLS | LRLDCK | | | | | | | |
| | VKDYFP | WKVDNA | | | PGERAT | ASGITFS | | | | | | | |
| | EPVTVS | LQSGNS | | | LSCRAS | NSGMH | | | | | | | |
| | WNSGAL | QESVTE | | | QSVSSY | WVRQAP | | | | | | | |
| | TSGVHT | QDSKDS | | | LAWYQQ | GKGLEW | | | | | | | |
| | FPAVLQ | TYSLSST | | | KPGQAP | VAVIWY | | | | | | | |
| | SSGLYSL | LTLSKAD | | | RLLIYDA | DGSKRY | | | | | | | |
| | SSVVTV | YEKHKV | | | SNRATGI | YADSVK | | | | | | | |
| | PSSSLG | YACEVT | | | PARFSG | GRFTISR | | | | | | | |
| | TKTYTC | HQGLSS | | | SGSGTD | DNSKNT | | | | | | | |
| | NVDHKP | PVTKSF | | | FTLTISSL | LFLQMN | | | | | | | |
| | SNTKVD | NRGEC | | | EPEDFA | SLRAED | | | | | | | |
| | KRVESK | (SEQ ID | | | VYYCQQ | TAVYYC | | | | | | | |
| | YGPPCP | NO: 80) | | | SSNWPR | ATNDDY | | | | | | | |
| | PCPAPE | | | | TFGQGT | WGQGTL | | | | | | | |
| | FLGGPS | | | | KVEIK | VTVSS | | | | | | | |
| | VFLFPPK | | | | (SEQ ID | (SEQ ID | | | | | | | |
| | PKDTLMI | | | | NO: 170) | NO: 200) | | | | | | | |
| | SRTPEV | | | | | | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 50) | | | | | | | | | | | | |
| penpulimab | EVQLVE | DIQMTQ | EVQLVE | DIQMTQ | EVQLVE | DIQMTQ | FAFS | WVA | ANRY | QDIN | TLIYR | LQYD | pen- |
| | SGGGLV | SPSSMS | SGGGLV | SPSSMS | SGGGLV | SPSSMS | SYD | TISG | GEA | TYLS | ANRL | EFPL | pulimab |
| | QPGGSL | ASVGDR | QPGGSL | ASVGDR | QPGGSL | ASVGDR | MS | GGR | WFA | (27- | VS | (89- | |
| | RLSCAA | VTFTCR | RLSCAA | VTFTCR | RLSCAA | VTFTCR | (27- | YTYY | Y (97- | 34) | (46- | 96) | |
| | SGFAFS | ASQDINT | SGFAFS | ASQDINT | SGFAFS | ASQDINT | 35) | P (47- | 107) | (SEQ | 56) | (SEQ | |
| | SYDMSW | YLSWFQ | SYDMSW | YLSWFQ | SYDMSW | YLSWFQ | (SEQ | 61) | (SEQ | ID | (SEQ | ID | |
| | VRQAPG | QKPGKS | VRQAPG | QKPGKS | VRQAPG | QKPGKS | ID | (SEQ | ID | NO: | ID | NO: | |
| | KGLDWV | PKTLIYR | KGLDWV | PKTLIYR | KGLDWV | PKTLIYR | NO: | ID | NO: | 315) | NO: | 372) | |
| | ATISGGG | ANRLVS | ATISGGG | ANRLVS | ATISGGG | ANRLVS | 231) | NO: | 285) | | 345) | | |
| | RYTYYP | GVPSRF | RYTYYP | GVPSRF | RYTYYP | GVPSRF | | 257) | | | | | |
| | DSVKGR | SGSGSG | DSVKGR | SGSGSG | DSVKGR | SGSGSG | | | | | | | |
| | FTISRDN | QDYTLTI | FTISRDN | QDYTLTI | FTISRDN | QDYTLTI | | | | | | | |
| | SKNNLYL | SSLQPE | SKNNLYL | SSLQPE | SKNNLYL | SSLQPE | | | | | | | |
| | QMNSLR | DMATYY | QMNSLR | DMATYY | QMNSLR | DMATYY | | | | | | | |
| | AEDTALY | CLQYDE | AEDTALY | CLQYDE | AEDTALY | CLQYDE | | | | | | | |
| | YCANRY | FPLTFGA | YCANRY | FPLTFGA | YCANRY | FPLTFGA | | | | | | | |
| | GEAWFA | GTKLELK | GEAWFA | GTKLELK | GEAWFA | GTKLELK | | | | | | | |
| | YWGQGT | RTVAAP | YWGQGT | (SEQ ID | YWGQGT | GGGGSG | | | | | | | |
| | LVTVSSA | SVFIFPP | LVTVSS | NO: 141) | LVTVSS | GGGSGG | | | | | | | |
| | STKGPS | SDEQLK | (SEQ ID | | GGGGSG | GGSEVQ | | | | | | | |
| | VFPLAPS | SGTASV | NO: 111) | | GGGSGG | LVESGG | | | | | | | |
| | SKSTSG | VCLLNNF | | | GGSDIQ | GLVQPG | | | | | | | |
| | GTAALG | YPREAK | | | MTQSPS | GSLRLS | | | | | | | |
| | CLVKDY | VQWKVD | | | SMSASV | CAASGF | | | | | | | |
| | FPEPVTV | NALQSG | | | GDRVTF | AFSSYD | | | | | | | |
| | SWNSGA | NSQESV | | | TCRASQ | MSWVR | | | | | | | |
| | LTSGVH | TEQDSK | | | DINTYLS | QAPGKG | | | | | | | |
| | TFPAVLQ | DSTYSLS | | | WFQQKP | LDWVATI | | | | | | | |
| | SSGLYSL | STLTLSK | | | GKSPKT | SGGGRY | | | | | | | |
| | SSVVTV | ADYEKH | | | LIYRANR | TYYPDS | | | | | | | |
| | PSSSLG | KVYACE | | | LVSGVP | VKGRFTI | | | | | | | |
| | TQTYICN | VTHQGL | | | SRFSGS | SRDNSK | | | | | | | |
| | VNHKPS | SSPVTK | | | GSGQDY | NNLYLQ | | | | | | | |
| | NTKVDK | SFNRGE | | | TLTISSL | MNSLRA | | | | | | | |
| | KVEPKS | C (SEQ | | | QPEDMA | EDTALYY | | | | | | | |
| | CDKTHT | ID NO: | | | TYYCLQ | CANRYG | | | | | | | |
| | CPPCPA | 81) | | | YDEFPLT | EAWFAY | | | | | | | |
| | PEAAGA | | | | FGAGTK | WGQGTL | | | | | | | |
| | PSVFLFP | | | | LELK | VTVSS | | | | | | | |
| | PKPKDTL | | | | (SEQ ID | (SEQ ID | | | | | | | |
| | MISRTPE | | | | NO: 171) | NO: 201) | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPREE | | | | | | | | | | | | |
| | QYNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KALPAPI | | | | | | | | | | | | |
| | EKTISKA | | | | | | | | | | | | |
| | KGQPRE | | | | | | | | | | | | |
| | PQVYTL | | | | | | | | | | | | |
| | PPSRDE | | | | | | | | | | | | |
| | LTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | KLTVDKS | | | | | | | | | | | | |
| | RWQQG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSP | | | | | | | | | | | | |
| | GK (SEQ | | | | | | | | | | | | |
| | ID NO: | | | | | | | | | | | | |
| | 51) | | | | | | | | | | | | |
| prolgolimab | QVQLVQ | QPVLTQ | QVQLVQ | QPVLTQ | QVQLVQ | QPVLTQ | FTFS | WVS | ARDE | NIGS | LVIY | QVW | prol- |
| | SGGGLV | PLSVSVA | SGGGLV | PLSVSVA | SGGGLV | PLSVSVA | SYW | AIDT | GGG | KNVH | RDSN | DSST | golimab |
| | QPGGSL | LGQTARI | QPGGSL | LGQTARI | QPGGSL | LGQTARI | MY | GGG | TGW | (26- | RPS | A (88- | |
| | RLSCAA | TCGGNN | RLSCAA | TCGGNN | RLSCAA | TCGGNN | (27- | RTYY | GVLK | 33) | (45- | 95) | |
| | SGFTFS | IGSKNVH | SGFTFS | IGSKNVH | SGFTFS | IGSKNVH | 35) | A (47- | DWP | (SEQ | 55) | (SEQ | |
| | SYWMY | WYQQKP | SYWMY | WYQQKP | SYWMY | WYQQKP | (SEQ | 61) | YGLD | ID | (SEQ | ID | |
| | WVRQVP | GQAPVL | WVRQVP | GQAPVL | WVRQVP | GQAPVL | ID | (SEQ | A (97- | NO: | ID | NO: | |
| | GKGLEW | VIYRDSN | GKGLEW | VIYRDSN | GKGLEW | VIYRDSN | NO: | ID | 118) | 316) | NO: | 373) | |
| | VSAIDTG | RPSGIPE | VSAIDTG | RPSGIPE | VSAIDTG | RPSGIPE | 232) | NO: | (SEQ | | 346) | | |
| | GGRTYY | RFSGSN | GGRTYY | RFSGSN | GGRTYY | RFSGSN | | 258) | ID | | | | |
| | ADSVKG | SGNTAT | ADSVKG | SGNTAT | ADSVKG | SGNTAT | | | NO: | | | | |
| | RFAISRV | LTISRAQ | RFAISRV | LTISRAQ | RFAISRV | LTISRAQ | | | 286) | | | | |
| | NAKNTM | AGDEAD | NAKNTM | AGDEAD | NAKNTM | AGDEAD | | | | | | | |
| | YLQMNS | YYCQVW | YLQMNS | YYCQVW | YLQMNS | YYCQVW | | | | | | | |
| | LRAEDT | DSSTAV | LRAEDT | DSSTAV | LRAEDT | DSSTAV | | | | | | | |
| | AVYYCA | FGTGTK | AVYYCA | FGTGTK | AVYYCA | FGTGTK | | | | | | | |
| | RDEGGG | LTVLQRT | RDEGGG | LTVLQ | RDEGGG | LTVLQG | | | | | | | |
| | TGWGVL | VAAPSV | TGWGVL | (SEQ ID | TGWGVL | GGGSGG | | | | | | | |
| | KDWPYG | FIFPPSD | KDWPYG | NO: 142) | KDWPYG | GGSGGG | | | | | | | |
| | LDAWGQ | EQLKSG | LDAWGQ | | LDAWGQ | GSQVQL | | | | | | | |
| | GTLVTVS | TASVVCL | GTLVTVS | | GTLVTVS | VQSGGG | | | | | | | |
| | SASTKG | LNNFYP | S (SEQ | | SGGGGS | LVQPGG | | | | | | | |
| | PSVFPLA | REAKVQ | ID NO: | | GGGGSG | SLRLSCA | | | | | | | |
| | PSSKST | WKVDNA | 112) | | GGGSQP | ASGFTF | | | | | | | |
| | SGGTAA | LQSGNS | | | VLTQPLS | SSYWMY | | | | | | | |
| | LGCLVK | QESVTE | | | VSVALG | WVRQVP | | | | | | | |
| | DYFPEP | QDSKDS | | | QTARITC | GKGLEW | | | | | | | |
| | VTVSWN | TYSLSST | | | GGNNIG | VSAIDTG | | | | | | | |
| | SGALTS | LTLSKAD | | | SKNVHW | GGRTYY | | | | | | | |
| | GVHTFP | YEKHKV | | | YQQKPG | ADSVKG | | | | | | | |
| | AVLQSS | YACEVT | | | QAPVLVI | RFAISRV | | | | | | | |
| | GLYSLSS | HQGLSS | | | YRDSNR | NAKNTM | | | | | | | |
| | VVTVPS | PVTKSF | | | PSGIPER | YLQMNS | | | | | | | |
| | SSLGTQ | NRGEC | | | FSGSNS | LRAEDT | | | | | | | |
| | TYICNVN | (SEQ ID | | | GNTATLT | AVYYCA | | | | | | | |
| | HKPSNT | NO: 82) | | | ISRAQAG | RDEGGG | | | | | | | |
| | KVDKRV | | | | DEADYY | TGWGVL | | | | | | | |
| | EPKSCD | | | | CQVWDS | KDWPYG | | | | | | | |
| | KTHTCP | | | | STAVFG | LDAWGQ | | | | | | | |
| | PCPAPE | | | | TGTKLTV | GTLVTVS | | | | | | | |
| | AAGGPS | | | | LQ (SEQ | S (SEQ | | | | | | | |
| | VFLFPPK | | | | ID NO: | ID NO: | | | | | | | |
| | PKDTLMI | | | | 172) | 202) | | | | | | | |
| | SRTPEV | | | | | | | | | | | | |
| | TCVVVD | | | | | | | | | | | | |
| | VSHEDP | | | | | | | | | | | | |
| | EVKFNW | | | | | | | | | | | | |
| | YVDGVE | | | | | | | | | | | | |
| | VHNAKT | | | | | | | | | | | | |
| | KPREEQ | | | | | | | | | | | | |
| | YNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | ALPAPIE | | | | | | | | | | | | |
| | KTISKAK | | | | | | | | | | | | |
| | GQPREP | | | | | | | | | | | | |
| | QVYTLP | | | | | | | | | | | | |
| | PSREEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSKL | | | | | | | | | | | | |
| | TVDKSR | | | | | | | | | | | | |
| | WQQGN | | | | | | | | | | | | |
| | VFSCSV | | | | | | | | | | | | |
| | MHEALH | | | | | | | | | | | | |
| | NHYTQK | | | | | | | | | | | | |
| | SLSLSPG | | | | | | | | | | | | |
| | K (SEQ | | | | | | | | | | | | |
| | ID NO: | | | | | | | | | | | | |
| | 52) | | | | | | | | | | | | |
| serplulimab | QVQLVE | DIQMTQ | QVQLVE | DIQMTQ | QVQLVE | DIQMTQ | FTFS | WVS | VSYY | QDVT | LLIY | QQH | ser- |
| | SGGGLV | SPSSLSA | SGGGLV | SPSSLSA | SGGGLV | SPSSLSA | NYG | TISG | YGID | TAVA | WAS | YTIP | plulimab |
| | KPGGSL | SVGDRV | KPGGSL | SVGDRV | KPGGSL | SVGDRV | MS | GGS | F (97- | (27- | TRHT | W | |
| | RLSCAA | TITCKAS | RLSCAA | TITCKAS | RLSCAA | TITCKAS | (27- | NIYY | 105) | 34) | (46- | (89- | |
| | SGFTFS | QDVTTA | SGFTFS | QDVTTA | SGFTFS | QDVTTA | 35) | (47- | (SEQ | (SEQ | 56) | 96) | |
| | NYGMS | VAWYQQ | NYGMS | VAWYQQ | NYGMS | VAWYQQ | (SEQ | 60) | ID | ID | (SEQ | (SEQ | |
| | WIRQAP | KPGKAP | WIRQAP | KPGKAP | WIRQAP | KPGKAP | ID | (SEQ | NO: | NO: | ID | ID | |
| | GKGLEW | KLLIYWA | GKGLEW | KLLIYWA | GKGLEW | KLLIYWA | NO: | ID | 287) | 317) | NO: | NO: | |
| | VSTISGG | STRHTG | VSTISGG | STRHTG | VSTISGG | STRHTG | 233) | NO: | | | 347) | 374) | |
| | GSNIYYA | VPSRFS | GSNIYYA | VPSRFS | GSNIYYA | VPSRFS | | 259) | | | | | |
| | DSVKGR | GSGSGT | DSVKGR | GSGSGT | DSVKGR | GSGSGT | | | | | | | |
| | FTISRDN | DFTLTIS | FTISRDN | DFTLTIS | FTISRDN | DFTLTIS | | | | | | | |
| | AKNSLYL | SLQPED | AKNSLYL | SLQPED | AKNSLYL | SLQPED | | | | | | | |
| | QMNSLR | FATYYC | QMNSLR | FATYYC | QMNSLR | FATYYC | | | | | | | |
| | AEDTAV | QQHYTIP | AEDTAV | QQHYTIP | AEDTAV | QQHYTIP | | | | | | | |
| | YYCVSY | WTFGGG | YYCVSY | WTFGGG | YYCVSY | WTFGGG | | | | | | | |
| | YYGIDF | TKLEIKR | YYGIDF | TKLEIK | YYGIDF | TKLEIKG | | | | | | | |
| | WGQGTS | TVAAPS | WGQGTS | (SEQ ID | WGQGTS | GGGSGG | | | | | | | |
| | VTVSSA | VFIFPPS | VTVSS | NO: 143) | VTVSSG | GGSGGG | | | | | | | |
| | STKGPS | DEQLKS | (SEQ ID | | GGGSGG | GSQVQL | | | | | | | |
| | VFPLAPC | GTASVV | NO: 113) | | GGSGGG | VESGGG | | | | | | | |
| | SRSTSE | CLLNNFY | | | GSDIQM | LVKPGG | | | | | | | |
| | STAALG | PREAKV | | | TQSPSS | SLRLSCA | | | | | | | |
| | CLVKDY | QWKVDN | | | LSASVG | ASGFTF | | | | | | | |
| | FPEPVTV | ALQSGN | | | DRVTITC | SNYGMS | | | | | | | |
| | SWNSGA | SQESVT | | | KASQDV | WIRQAP | | | | | | | |
| | LTSGVH | EQDSKD | | | TTAVAW | GKGLEW | | | | | | | |
| | TFPAVLQ | STYSLSS | | | YQQKPG | VSTISGG | | | | | | | |
| | SSGLYSL | TLTLSKA | | | KAPKLLI | GSNIYYA | | | | | | | |
| | SSVVTV | DYEKHK | | | YWASTR | DSVKGR | | | | | | | |
| | PSSSLG | VYACEV | | | HTGVPS | FTISRDN | | | | | | | |
| | TKTYTC | THQGLS | | | RFSGSG | AKNSLYL | | | | | | | |
| | NVDHKP | SPVTKSF | | | SGTDFTL | QMNSLR | | | | | | | |
| | SNTKVD | NRGEC | | | TISSLQP | AEDTAV | | | | | | | |
| | KRVESK | (SEQ ID | | | EDFATY | YYCVSY | | | | | | | |
| | YGPPCP | NO: 83) | | | YCQQHY | YYGIDF | | | | | | | |
| | PCPAPE | | | | TIPWTFG | WGQGTS | | | | | | | |
| | FLGGPS | | | | GGTKLEI | VTVSS | | | | | | | |
| | VFLFPPK | | | | K (SEQ | (SEQ ID | | | | | | | |
| | PKDTLMI | | | | ID NO: | NO: 203) | | | | | | | |
| | SRTPEV | | | | 173) | | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 53) | | | | | | | | | | | | |
| sintilimab | QVQLVQ | DIQMTQ | QVQLVQ | DIQMTQ | QVQLVQ | DIQMTQ | GTFS | WMG | RAEH | QGIS | LLISA | QQA | sin- |
| | SGAEVK | SPSSVS | SGAEVK | SPSSVS | SGAEVK | SPSSVS | SYAI | LIIPM | SSTG | SWLA | ASSL | NHLP | tilimab |
| | KPGSSV | ASVGDR | KPGSSV | ASVGDR | KPGSSV | ASVGDR | S (27- | FDTA | TFDY | (27- | QS | F (89- | |
| | KVSCKA | VTITCRA | KVSCKA | VTITCRA | KVSCKA | VTITCRA | 35) | GY | (98- | 34) | (46- | 96) | |
| | SGGTFS | SQGISS | SGGTFS | SQGISS | SGGTFS | SQGISS | (SEQ | (47- | 109) | (SEQ | 56) | (SEQ | |
| | SYAISWV | WLAWYQ | SYAISWV | WLAWYQ | SYAISWV | WLAWYQ | ID | 60) | (SEQ | ID | (SEQ | ID | |
| | RQAPGQ | QKPGKA | RQAPGQ | QKPGKA | RQAPGQ | QKPGKA | NO: | (SEQ | ID | NO: | ID | NO: | |
| | GLEWMG | PKLLISA | GLEWMG | PKLLISA | GLEWMG | PKLLISA | 234) | ID | NO: | 318) | NO: | 375) | |
| | LIIPMFDT | ASSLQS | LIIPMFDT | ASSLQS | LIIPMFDT | ASSLQS | | NO: | 288) | | 348) | | |
| | AGYAQK | GVPSRF | AGYAQK | GVPSRF | AGYAQK | GVPSRF | | 260) | | | | | |
| | FQGRVAI | SGSGSG | FQGRVAI | SGSGSG | FQGRVAI | SGSGSG | | | | | | | |
| | TVDEST | TDFTLTI | TVDEST | TDFTLTI | TVDEST | TDFTLTI | | | | | | | |
| | STAYME | SSLQPE | STAYME | SSLQPE | STAYME | SSLQPE | | | | | | | |
| | LSSLRSE | DFATYY | LSSLRSE | DFATYY | LSSLRSE | DFATYY | | | | | | | |
| | DTAVYY | CQQANH | DTAVYY | CQQANH | DTAVYY | CQQANH | | | | | | | |
| | CARAEH | LPFTFG | CARAEH | LPFTFG | CARAEH | LPFTFG | | | | | | | |
| | SSTGTF | GGTKVEI | SSTGTF | GGTKVEI | SSTGTF | GGTKVEI | | | | | | | |
| | DYWGQ | KRTVAA | DYWGQ | K (SEQ | DYWGQ | KGGGGS | | | | | | | |
| | GTLVTVS | PSVFIFP | GTLVTVS | ID NO: | GTLVTVS | GGGGSG | | | | | | | |
| | SASTKG | PSDEQL | S (SEQ | 144) | SGGGGS | GGGSQV | | | | | | | |
| | PSVFPLA | KSGTAS | ID NO: | | GGGGSG | QLVQSG | | | | | | | |
| | PCSRST | VVCLLN | 114) | | GGGSDI | AEVKKP | | | | | | | |
| | SESTAAL | NFYPRE | | | QMTQSP | GSSVKV | | | | | | | |
| | GCLVKD | AKVQWK | | | SSVSAS | SCKASG | | | | | | | |
| | YFPEPVT | VDNALQ | | | VGDRVTI | GTFSSY | | | | | | | |
| | VSWNSG | SGNSQE | | | TCRASQ | AISWVR | | | | | | | |
| | ALTSGV | SVTEQD | | | GISSWLA | QAPGQG | | | | | | | |
| | HTFPAVL | SKDSTY | | | WYQQKP | LEWMGL | | | | | | | |
| | QSSGLY | SLSSTLT | | | GKAPKLL | IIPMFDT | | | | | | | |
| | SLSSVVT | LSKADY | | | ISAASSL | AGYAQK | | | | | | | |
| | VPSSSL | EKHKVY | | | QSGVPS | FQGRVAI | | | | | | | |
| | GTKTYT | ACEVTH | | | RFSGSG | TVDEST | | | | | | | |
| | CNVDHK | QGLSSP | | | SGTDFTL | STAYME | | | | | | | |
| | PSNTKV | VTKSFN | | | TISSLQP | LSSLRSE | | | | | | | |
| | DKRVES | RGEC | | | EDFATY | DTAVYY | | | | | | | |
| | KYGPPC | (SEQ ID | | | YCQQAN | CARAEH | | | | | | | |
| | PPCPAP | NO: 84) | | | HLPFTFG | SSTGTF | | | | | | | |
| | EFLGGP | | | | GGTKVEI | DYWGQ | | | | | | | |
| | SVFLFPP | | | | K (SEQ | GTLVTVS | | | | | | | |
| | KPKDTL | | | | ID NO: | S (SEQ | | | | | | | |
| | MISRTPE | | | | 174) | ID NO: | | | | | | | |
| | VTCVVV | | | | | 204) | | | | | | | |
| | DVSQED | | | | | | | | | | | | |
| | PEVQFN | | | | | | | | | | | | |
| | WYVDGV | | | | | | | | | | | | |
| | EVHNAK | | | | | | | | | | | | |
| | TKPREE | | | | | | | | | | | | |
| | QFNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KGLPSSI | | | | | | | | | | | | |
| | EKTISKA | | | | | | | | | | | | |
| | KGQPRE | | | | | | | | | | | | |
| | PQVYTL | | | | | | | | | | | | |
| | PPSQEE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | RLTVDK | | | | | | | | | | | | |
| | SRWQEG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSL | | | | | | | | | | | | |
| | GK (SEQ | | | | | | | | | | | | |
| | ID NO: | | | | | | | | | | | | |
| | 54) | | | | | | | | | | | | |
| tislelizumab | QVQLQE | DIVMTQS | QVQLQE | DIVMTQS | QVQLQE | DIVMTQS | FSLT | WIGV | RAYG | ESVS | LLINY | HQAY | tis- |
| | SGPGLV | PDSLAV | SGPGLV | PDSLAV | SGPGLV | PDSLAV | SYGV | IYAD | NYW | NDVA | AFHR | SSPY | lelizumab |
| | KPSETLS | SLGERA | KPSETLS | SLGERA | KPSETLS | SLGERA | H (27- | GSTN | YIDV | (27- | FT | (89- | |
| | LTCTVS | TINCKSS | LTCTVS | TINCKSS | LTCTVS | TINCKSS | 35) | Y (47- | (97- | 34) | (46- | 96) | |
| | GFSLTSY | ESVSND | GFSLTSY | ESVSND | GFSLTSY | ESVSND | (SEQ | 59) | 107) | (SEQ | 56) | (SEQ | |
| | GVHWIR | VAWYQQ | GVHWIR | VAWYQQ | GVHWIR | VAWYQQ | ID | (SEQ | (SEQ | ID | (SEQ | ID | |
| | QPPGKG | KPGQPP | QPPGKG | KPGQPP | QPPGKG | KPGQPP | NO: | ID | ID | NO: | ID | NO: | |
| | LEWIGVI | KLLINYA | LEWIGVI | KLLINYA | LEWIGVI | KLLINYA | 235) | NO: | NO: | 319) | NO: | 376) | |
| | YADGST | FHRFTG | YADGST | FHRFTG | YADGST | FHRFTG | | 261) | 289) | | 349) | | |
| | NYNPSL | VPDRFS | NYNPSL | VPDRFS | NYNPSL | VPDRFS | | | | | | | |
| | KSRVTIS | GSGYGT | KSRVTIS | GSGYGT | KSRVTIS | GSGYGT | | | | | | | |
| | KDTSKN | DFTLTIS | KDTSKN | DFTLTIS | KDTSKN | DFTLTIS | | | | | | | |
| | QVSLKLS | SLQAED | QVSLKLS | SLQAED | QVSLKLS | SLQAED | | | | | | | |
| | SVTAAD | VAVYYC | SVTAAD | VAVYYC | SVTAAD | VAVYYC | | | | | | | |
| | TAVYYC | HQAYSS | TAVYYC | HQAYSS | TAVYYC | HQAYSS | | | | | | | |
| | ARAYGN | PYTFGQ | ARAYGN | PYTFGQ | ARAYGN | PYTFGQ | | | | | | | |
| | YWYIDV | GTKLEIK | YWYIDV | GTKLEIK | YWYIDV | GTKLEIK | | | | | | | |
| | WGQGTT | RTVAAP | WGQGTT | (SEQ ID | WGQGTT | GGGGSG | | | | | | | |
| | VTVSSA | SVFIFPP | VTVSS | NO: 145) | VTVSSG | GGGSGG | | | | | | | |
| | STKGPS | SDEQLK | (SEQ ID | | GGGSGG | GGSQVQ | | | | | | | |
| | VFPLAPC | SGTASV | NO: 115) | | GGSGGG | LQESGP | | | | | | | |
| | SRSTSE | VCLLNNF | | | GSDIVMT | GLVKPS | | | | | | | |
| | STAALG | YPREAK | | | QSPDSL | ETLSLTC | | | | | | | |
| | CLVKDY | VQWKVD | | | AVSLGE | TVSGFSL | | | | | | | |
| | FPEPVTV | NALQSG | | | RATINCK | TSYGVH | | | | | | | |
| | SWNSGA | NSQESV | | | SSESVS | WIRQPP | | | | | | | |
| | LTSGVH | TEQDSK | | | NDVAWY | GKGLEW | | | | | | | |
| | TFPAVLQ | DSTYSLS | | | QQKPGQ | IGVIYAD | | | | | | | |
| | SSGLYSL | STLTLSK | | | PPKLLIN | GSTNYN | | | | | | | |
| | SSVVTV | ADYEKH | | | YAFHRF | PSLKSR | | | | | | | |
| | PSSSLG | KVYACE | | | TGVPDR | VTISKDT | | | | | | | |
| | TKTYTC | VTHQGL | | | FSGSGY | SKNQVS | | | | | | | |
| | NVDHKP | SSPVTK | | | GTDFTLT | LKLSSVT | | | | | | | |
| | SNTKVD | SFNRGE | | | ISSLQAE | AADTAV | | | | | | | |
| | KRVESK | C (SEQ | | | DVAVYY | YYCARA | | | | | | | |
| | YGPPCP | ID NO: | | | CHQAYS | YGNYWY | | | | | | | |
| | PCPAPP | 85) | | | SPYTFG | IDVWGQ | | | | | | | |
| | VAGGPS | | | | QGTKLEI | GTTVTV | | | | | | | |
| | VFLFPPK | | | | K (SEQ | SS (SEQ | | | | | | | |
| | PKDTLMI | | | | ID NO: | ID NO: | | | | | | | |
| | SRTPEV | | | | 175) | 205) | | | | | | | |
| | TCVVVA | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | VHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSKL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 55) | | | | | | | | | | | | |
| toripalimab | QGQLVQ | DVVMTQ | QGQLVQ | DVVMTQ | QGQLVQ | DVVMTQ | YTFT | WIGV | AREG | QSIV | LLIYK | FQGS | tori- |
| | SGAEVK | SPLSLPV | SGAEVK | SPLSLPV | SGAEVK | SPLSLPV | DYE | IESE | ITTVA | HSN | VSNR | HVPL | palimab |
| | KPGASV | TLGQPA | KPGASV | TLGQPA | KPGASV | TLGQPA | MH | TGGT | TTYY | GNTY | FS | (94- | |
| | KVSCKA | SISCRSS | KVSCKA | SISCRSS | KVSCKA | SISCRSS | (27- | AY | WYF | LE | (51- | 101) | |
| | SGYTFT | QSIVHSN | SGYTFT | QSIVHSN | SGYTFT | QSIVHSN | 35) | (47- | DV | (27- | 61) | (SEQ | |
| | DYEMHW | GNTYLE | DYEMHW | GNTYLE | DYEMHW | GNTYLE | (SEQ | 60) | (97- | 39) | (SEQ | ID | |
| | VRQAPIH | WYLQKP | VRQAPIH | WYLQKP | VRQAPIH | WYLQKP | ID | (SEQ | 114) | (SEQ | ID | NO: | |
| | GLEWIG | GQSPQL | GLEWIG | GQSPQL | GLEWIG | GQSPQL | NO: | ID | (SEQ | ID | NO: | 377) | |
| | VIESETG | LIYKVSN | VIESETG | LIYKVSN | VIESETG | LIYKVSN | 236) | NO: | ID | NO: | 350) | | |
| | GTAYNQ | RFSGVP | GTAYNQ | RFSGVP | GTAYNQ | RFSGVP | | 262) | NO: | 320) | | | |
| | KFKGRV | DRFSGS | KFKGRV | DRFSGS | KFKGRV | DRFSGS | | | 290) | | | | |
| | TITADKS | GSGTDF | TITADKS | GSGTDF | TITADKS | GSGTDF | | | | | | | |
| | TSTAYM | TLKISRV | TSTAYM | TLKISRV | TSTAYM | TLKISRV | | | | | | | |
| | ELSSLRS | EAEDVG | ELSSLRS | EAEDVG | ELSSLRS | EAEDVG | | | | | | | |
| | EDTAVY | VYYCFQ | EDTAVY | VYYCFQ | EDTAVY | VYYCFQ | | | | | | | |
| | YCAREGI | GSHVPL | YCAREGI | GSHVPL | YCAREGI | GSHVPL | | | | | | | |
| | TTVATTY | TFGQGT | TTVATTY | TFGQGT | TTVATTY | TFGQGT | | | | | | | |
| | YWYFDV | KLEIKRT | YWYFDV | KLEIK | YWYFDV | KLEIKGG | | | | | | | |
| | WGQGTT | VAAPSV | WGQGTT | (SEQ ID | WGQGTT | GGSGGG | | | | | | | |
| | VTVSSA | FIFPPSD | VTVSS | NO: 146) | VTVSSG | GSGGGG | | | | | | | |
| | STKGPS | EQLKSG | (SEQ ID | | GGGSGG | SQGQLV | | | | | | | |
| | VFPLAPC | TASVVCL | NO: 116) | | GGSGGG | QSGAEV | | | | | | | |
| | SRSTSE | LNNFYP | | | GSDVVM | KKPGAS | | | | | | | |
| | STAALG | REAKVQ | | | TQSPLSL | VKVSCK | | | | | | | |
| | CLVKDY | WKVDNA | | | PVTLGQ | ASGYTF | | | | | | | |
| | FPEPVTV | LQSGNS | | | PASISCR | TDYEMH | | | | | | | |
| | SWNSGA | QESVTE | | | SSQSIVH | WVRQAP | | | | | | | |
| | LTSGVH | QDSKDS | | | SNGNTY | IHGLEWI | | | | | | | |
| | TFPAVLQ | TYSLSST | | | LEWYLQ | GVIESET | | | | | | | |
| | SSGLYSL | LTLSKAD | | | KPGQSP | GGTAYN | | | | | | | |
| | SSVVTV | YEKHKV | | | QLLIYKV | QKFKGR | | | | | | | |
| | PSSSLG | YACEVT | | | SNRFSG | VTITADK | | | | | | | |
| | TKTYTC | HQGLSS | | | VPDRFS | STSTAY | | | | | | | |
| | NVDHKP | PVTKSF | | | GSGSGT | MELSSL | | | | | | | |
| | SNTKVD | NRGEC | | | DFTLKIS | RSEDTA | | | | | | | |
| | KRVESK | (SEQ ID | | | RVEAED | VYYCAR | | | | | | | |
| | YGPPCP | NO: 86) | | | VGVYYC | EGITTVA | | | | | | | |
| | PCPAPE | | | | FQGSHV | TTYYWY | | | | | | | |
| | FLGGPS | | | | PLTFGQ | FDVWGQ | | | | | | | |
| | VFLFPPK | | | | GTKLEIK | GTTVTV | | | | | | | |
| | PKDTLMI | | | | (SEQ ID | SS (SEQ | | | | | | | |
| | SRTPEV | | | | NO: 176) | ID NO: | | | | | | | |
| | TCVVVD | | | | | 206) | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 56) | | | | | | | | | | | | |
| zimberelimab | QLQLQE | QSALTQ | QLQLQE | QSALTQ | QLQLQE | QSALTQ | ADSI | WIGS | RHLG | SSDV | LMIY | SSYT | zim- |
| | SGPGLV | PASVSG | SGPGLV | PASVSG | SGPGLV | PASVSG | SSTT | ISYS | YNG | GFYN | DVSN | SIST | bere- |
| | KPSETLT | SPGQSIT | KPSETLT | SPGQSIT | KPSETLT | SPGQSIT | YYW | GSTY | RYLP | YVS | RPS | W | limab |
| | LTCTVSA | ISCTGTS | LTCTVSA | ISCTGTS | LTCTVSA | ISCTGTS | V (26- | Y (49- | FDY | (26- | (48- | (91- | |
| | DSISSTT | SDVGFY | DSISSTT | SDVGFY | DSISSTT | SDVGFY | 37) | 61) | (99- | 36) | 58) | 99) | |
| | YYWVWI | NYVSWY | YYWVWI | NYVSWY | YYWVWI | NYVSWY | (SEQ | (SEQ | 112) | (SEQ | (SEQ | (SEQ | |
| | RQPPGK | QQHPGK | RQPPGK | QQHPGK | RQPPGK | QQHPGK | ID | ID | (SEQ | ID | ID | ID | |
| | GLEWIG | APELMIY | GLEWIG | APELMIY | GLEWIG | APELMIY | NO: | NO: | ID | NO: | NO | NO: | |
| | SISYSGS | DVSNRP | SISYSGS | DVSNRP | SISYSGS | DVSNRP | 237) | 263) | NO: | 321) | 351) | 378) | |
| | TYYNPSL | SGVSDR | TYYNPSL | SGVSDR | TYYNPSL | SGVSDR | | | 291) | | | | |
| | KSRVTV | FSGSKS | KSRVTV | FSGSKS | KSRVTV | FSGSKS | | | | | | | |
| | SVDTSK | GNTASL | SVDTSK | GNTASL | SVDTSK | GNTASL | | | | | | | |
| | NQFSLKL | TISGLQA | NQFSLKL | TISGLQA | NQFSLKL | TISGLQA | | | | | | | |
| | NSVAAT | EDEADY | NSVAAT | EDEADY | NSVAAT | EDEADY | | | | | | | |
| | DTALYY | YCSSYT | DTALYY | YCSSYT | DTALYY | YCSSYT | | | | | | | |
| | CARHLG | SISTWVF | CARHLG | SISTWVF | CARHLG | SISTWVF | | | | | | | |
| | YNGRYL | GGGTKL | YNGRYL | GGGTKL | YNGRYL | GGGTKL | | | | | | | |
| | PFDYWG | TVLGQP | PFDYWG | TVL | PFDYWG | TVLGGG | | | | | | | |
| | QGTLVT | KAAPSV | QGTLVT | (SEQ | QGTLVT | GSGGGG | | | | | | | |
| | VSSAST | TLFPPSS | VSS | ID NO: | VSSGGG | SGGGGS | | | | | | | |
| | KGPSVF | EELQAN | (SEQ ID | 147) | GSGGGG | QLQLQE | | | | | | | |
| | PLAPCS | KATLVCL | NO: 117) | | SGGGGS | SGPGLV | | | | | | | |
| | RSTSES | ISDFYPG | | | QSALTQ | KPSETLT | | | | | | | |
| | TAALGCL | AVTVAW | | | PASVSG | LTCTVSA | | | | | | | |
| | VKDYFP | KADSSP | | | SPGQSIT | DSISSTT | | | | | | | |
| | EPVTVS | VKAGVE | | | ISCTGTS | YYWVWI | | | | | | | |
| | WNSGAL | TTTPSK | | | SDVGFY | RQPPGK | | | | | | | |
| | TSGVHT | QSNNKY | | | NYVSWY | GLEWIG | | | | | | | |
| | FPAVLQ | AASSYLS | | | QQHPGK | SISYSGS | | | | | | | |
| | SSGLYSL | LTPEQW | | | APELMIY | TYYNPSL | | | | | | | |
| | SSVVTV | KSHRSY | | | DVSNRP | KSRVTV | | | | | | | |
| | PSSSLG | SCQVTH | | | SGVSDR | SVDTSK | | | | | | | |
| | TKTYTC | EGSTVE | | | FSGSKS | NQFSLKL | | | | | | | |
| | NVDHKP | KTVAPTE | | | GNTASL | NSVAAT | | | | | | | |
| | SNTKVD | CS (SEQ | | | TISGLQA | DTALYY | | | | | | | |
| | KRVESK | ID NO: | | | EDEADY | CARHLG | | | | | | | |
| | YGPPCP | 87) | | | YCSSYT | YNGRYL | | | | | | | |
| | PCPAPE | | | | SISTWVF | PFDYWG | | | | | | | |
| | FLGGPS | | | | GGGTKL | QGTLVT | | | | | | | |
| | VFLFPPK | | | | TVL (SEQ | VSS | | | | | | | |
| | PKDTLMI | | | | ID NO: | (SEQ ID | | | | | | | |
| | SRTPEV | | | | 177) | NO: 207) | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 57) | | | | | | | | | | | | |
| acrixolimab | QVQLVE | DIVMTQT | QVQLVE | DIVMTQT | QVQLVE | DIVMTQT | FTFL | WVA | TTTT | QSLL | LLIYT | MQR | acri- |
| | SGGGVV | PLSLPVT | SGGGVV | PLSLPVT | SGGGVV | PLSLPVT | RYA | VISY | FDS | DSED | LSHR | RDFP | xolimab |
| | QPGRSL | PGEAASI | QPGRSL | PGEAASI | QPGRSL | PGEAASI | MH | DGR | (97- | GNTY | AS | F (95- | |
| | RLSCAA | SCRSSQ | RLSCAA | SCRSSQ | RLSCAA | SCRSSQ | (27- | YKYY | 103) | LD | (52- | 102) | |
| | SGFTFLR | SLLDSED | SGFTFLR | SLLDSED | SGFTFLR | SLLDSED | 35) | (47- | (SEQ | (27- | 62) | (SEQ | |
| | YAMHWV | GNTYLD | YAMHWV | GNTYLD | YAMHWV | GNTYLD | (SEQ | 60) | ID | 40) | (SEQ | ID | |
| | RQAPGK | WYLQKP | RQAPGK | WYLQKP | RQAPGK | WYLQKP | ID | (SEQ | NO: | (SEQ | ID | NO: | |
| | GLEWVA | GQSPQL | GLEWVA | GQSPQL | GLEWVA | GQSPQL | NO: | ID | 292) | ID | NO: | 379) | |
| | VISYDGR | LIYTLSH | VISYDGR | LIYTLSH | VISYDGR | LIYTLSH | 238) | NO: | | NO: | 352) | | |
| | YKYYAD | RASGVP | YKYYAD | RASGVP | YKYYAD | RASGVP | | 264) | | 322) | | | |
| | SVKGRF | QRFSGS | SVKGRF | DRFSGS | SVKGRF | DRFSGS | | | | | | | |
| | TISRDNS | GSGTDF | TISRDNS | GSGTDF | TISRDNS | GSGTDF | | | | | | | |
| | KNTLYLQ | TLEISRV | KNTLYLQ | TLEISRV | KNTLYLQ | TLEISRV | | | | | | | |
| | MNSLRA | EAEDVG | MNSLRA | EAEDVG | MNSLRA | EAEDVG | | | | | | | |
| | EDTAVY | VYYCMQ | EDTAVY | VYYCMQ | EDTAVY | VYYCMQ | | | | | | | |
| | YCTTTTF | RRDFPF | YCTTTTF | RRDFPF | YCTTTTF | RRDFPF | | | | | | | |
| | DSWGQ | TFGQGT | DSWGQ | TFGQGT | DSWGQ | TFGQGT | | | | | | | |
| | GTLVTVS | KVDIKRT | GTLVTVS | KVDIK | GTLVTVS | KVDIKGG | | | | | | | |
| | SASTKG | VAAPSV | S (SEQ | (SEQ ID | SGGGGS | GGSGGG | | | | | | | |
| | PSVFPLA | FIFPPSD | ID NO: | NO: 148) | GGGGSG | GSGGGG | | | | | | | |
| | PCSRST | EQLKSG | 118) | | GGGSDI | SQVQLV | | | | | | | |
| | SESTAAL | TASVVCL | | | VMTQTP | ESGGGV | | | | | | | |
| | GCLVKD | LNNFYP | | | LSLPVTP | VQPGRS | | | | | | | |
| | YFPEPVT | REAKVQ | | | GEAASIS | LRLSCAA | | | | | | | |
| | VSWNSG | WKVDNA | | | CRSSQS | SGFTFLR | | | | | | | |
| | ALTSGV | LQSGNS | | | LLDSED | YAMHWV | | | | | | | |
| | HTFPAVL | QESVTE | | | GNTYLD | RQAPGK | | | | | | | |
| | QSSGLY | QDSKDS | | | WYLQKP | GLEWVA | | | | | | | |
| | SLSSVVT | TYSLSST | | | GQSPQL | VISYDGR | | | | | | | |
| | VPSSSL | LTLSKAD | | | LIYTLSH | YKYYAD | | | | | | | |
| | GTKTYT | YEKHKV | | | RASGVP | SVKGRF | | | | | | | |
| | CNVDHK | YACEVT | | | DRFSGS | TISRDNS | | | | | | | |
| | PSNTKV | HQGLSS | | | GSGTDF | KNTLYLQ | | | | | | | |
| | DKRVES | PVTKSF | | | TLEISRV | MNSLRA | | | | | | | |
| | KYGPPC | NRGEC | | | EAEDVG | EDTAVY | | | | | | | |
| | PPCPAP | | | | VYYCMQ | YCTTTTF | | | | | | | |
| | EFLGGP | (SEQ ID | | | RRDFPF | DSWGQ | | | | | | | |
| | SVFLFPP | NO: 88) | | | TFGQGT | GTLVTVS | | | | | | | |
| | KPKDTL | | | | KVDIK | S (SEQ | | | | | | | |
| | MISRTPE | | | | (SEQ ID | ID NO: | | | | | | | |
| | VTCVVV | | | | NO: 178) | 208) | | | | | | | |
| | DVSQED | | | | | | | | | | | | |
| | PEVQFN | | | | | | | | | | | | |
| | WYVDGV | | | | | | | | | | | | |
| | EVHNAK | | | | | | | | | | | | |
| | TKPREE | | | | | | | | | | | | |
| | QFNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KGLPSSI | | | | | | | | | | | | |
| | EKTISKA | | | | | | | | | | | | |
| | KGQPRE | | | | | | | | | | | | |
| | PQVYTL | | | | | | | | | | | | |
| | PPSQEE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | RLTVDK | | | | | | | | | | | | |
| | SRWQEG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSL | | | | | | | | | | | | |
| | GK (SEQ | | | | | | | | | | | | |
| | ID NO: | | | | | | | | | | | | |
| | 58) | | | | | | | | | | | | |
| zeluvalimab | EVQLLES | DIQMTQ | EVQLLES | DIQMTQ | EVQLLES | DIQMTQ | FTFS | WVSL | ASPS | QGIS | LLIFA | QQA | zeluv |
| | GGGLVQ | SPSSVS | GGGLVQ | SPSSVS | GGGLVQ | SPSSVS | SYD | ISGG | GHYF | NWL | ASSL | ESFP | alimab |
| | PGGSLR | ASVGDR | PGGSLR | ASVGDR | PGGSLR | ASVGDR | MS | GSQT | YAM | A (27- | QS | H (89- | |
| | LSCAAS | VTITCRA | LSCAAS | VTITCRA | LSCAAS | VTITCRA | (27- | YYA | DV | 34) | (46- | 96) | |
| | GFTFSS | SQGISN | GFTFSS | SQGISN | GFTFSS | SQGISN | 35) | (47- | (97- | (SEQ | 56) | (SEQ | |
| | YDMSWV | WLAWYQ | YDMSWV | WLAWYQ | YDMSWV | WLAWYQ | (SEQ | 61) | 109) | ID | (SEQ | ID | |
| | RQAPGK | QKPGKA | RQAPGK | QKPGKA | RQAPGK | QKPGKA | ID | (SEQ | (SEQ | NO: | ID | NO | |
| | GLEWVS | PKLLIFA | GLEWVS | PKLLIFA | GLEWVS | PKLLIFA | NO: | ID | ID | 323) | NO: | 380) | |
| | LISGGGS | ASSLQS | LISGGGS | ASSLQS | LISGGGS | ASSLQS | 229) | NO: | NO: | | 353) | | |
| | QTYYAE | GVPSRF | QTYYAE | GVPSRF | QTYYAE | GVPSRF | | 265) | 293) | | | | |
| | SVKGRF | SGSGSG | SVKGRF | SGSGSG | SVKGRF | SGSGSG | | | | | | | |
| | TISRDNS | TDFTLTI | TISRDNS | TDFTLTI | TISRDNS | TDFTLTI | | | | | | | |
| | KNTLYLQ | SSLQPE | KNTLYLQ | SSLQPE | KNTLYLQ | SSLQPE | | | | | | | |
| | MNSLRA | DFATYY | MNSLRA | DFATYY | MNSLRA | DFATYY | | | | | | | |
| | EDTAVY | CQQAES | EDTAVY | CQQAES | EDTAVY | CQQAES | | | | | | | |
| | FCASPS | FPHTFG | FCASPS | FPHTFG | FCASPS | FPHTFG | | | | | | | |
| | GHYFYA | GGTKVEI | GHYFYA | GGTKVEI | GHYFYA | GGTKVEI | | | | | | | |
| | MDVWG | KRTVAA | MDVWG | K (SEQ | MDVWG | KGGGGS | | | | | | | |
| | QGTTVT | PSVFIFP | QGTTVT | ID NO: | QGTTVT | GGGGSG | | | | | | | |
| | VSSAST | PSDEQL | VSS | 149) | VSSGGG | GGGSEV | | | | | | | |
| | KGPSVF | KSGTAS | (SEQ ID | | GSGGGG | QLLESG | | | | | | | |
| | PLAPSSK | VVCLLN | NO: 119) | | SGGGGS | GGLVQP | | | | | | | |
| | STSGGT | NFYPRE | | | DIQMTQ | GGSLRL | | | | | | | |
| | AALGCL | AKVQWK | | | SPSSVS | SCAASG | | | | | | | |
| | VKDYFP | VDNALQ | | | ASVGDR | FTFSSYD | | | | | | | |
| | EPVTVS | SGNSQE | | | VTITCRA | MSWVR | | | | | | | |
| | WNSGAL | SVTEQD | | | SQGISN | QAPGKG | | | | | | | |
| | TSGVHT | SKDSTY | | | WLAWYQ | LEWVSLI | | | | | | | |
| | FPAVLQ | SLSSTLT | | | QKPGKA | SGGGSQ | | | | | | | |
| | SSGLYSL | LSKADY | | | PKLLIFA | TYYAES | | | | | | | |
| | SSVVTV | EKHKVY | | | ASSLQS | VKGRFTI | | | | | | | |
| | PSSSLG | ACEVTH | | | GVPSRF | SRDNSK | | | | | | | |
| | TQTYICN | QGLSSP | | | SGSGSG | NTLYLQ | | | | | | | |
| | VNHKPS | VTKSFN | | | TDFTLTI | MNSLRA | | | | | | | |
| | NTKVDK | RGEC | | | SSLQPE | EDTAVY | | | | | | | |
| | KVEPKS | (SEQ ID | | | DFATYY | FCASPS | | | | | | | |
| | CDKTHT | NO: 89) | | | CQQAES | GHYFYA | | | | | | | |
| | CPPCPA | | | | FPHTFG | MDVWG | | | | | | | |
| | PELLGG | | | | GGTKVEI | QGTTVT | | | | | | | |
| | PSVFLFP | | | | K (SEQ | VSS | | | | | | | |
| | PKPKDTL | | | | ID NO: | (SEQ ID | | | | | | | |
| | MISRTPE | | | | 179) | NO: 209) | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPCEE | | | | | | | | | | | | |
| | QYGSTY | | | | | | | | | | | | |
| | RCVSVL | | | | | | | | | | | | |
| | TVLHQD | | | | | | | | | | | | |
| | WLNGKE | | | | | | | | | | | | |
| | YKCKVS | | | | | | | | | | | | |
| | NKALPA | | | | | | | | | | | | |
| | PIEKTISK | | | | | | | | | | | | |
| | AKGQPR | | | | | | | | | | | | |
| | EPQVYT | | | | | | | | | | | | |
| | LPPSRE | | | | | | | | | | | | |
| | EMTKNQ | | | | | | | | | | | | |
| | VSLTCLV | | | | | | | | | | | | |
| | KGFYPS | | | | | | | | | | | | |
| | DIAVEW | | | | | | | | | | | | |
| | ESNGQP | | | | | | | | | | | | |
| | ENNYKT | | | | | | | | | | | | |
| | TPPVLDS | | | | | | | | | | | | |
| | DGSFFL | | | | | | | | | | | | |
| | YSKLTVD | | | | | | | | | | | | |
| | KSRWQQ | | | | | | | | | | | | |
| | GNVFSC | | | | | | | | | | | | |
| | SVMHEA | | | | | | | | | | | | |
| | LHNHYT | | | | | | | | | | | | |
| | QKSLSLS | | | | | | | | | | | | |
| | PGK | | | | | | | | | | | | |
| | (SEQ ID | | | | | | | | | | | | |
| | NO: 59) | | | | | | | | | | | | |
| budigalimab | EIQLVQS | DVVMTQ | EIQLVQS | DVVMTQ | EIQLVQS | DVVMTQ | YTFT | WVG | REGE | QSIV | LLIYK | FQGS | budi- |
| | GAEVKK | SPLSLPV | GAEVKK | SPLSLPV | GAEVKK | SPLSLPV | HYG | WVN | GLGF | HSH | VSNR | HIPV | galimab |
| | PGSSVK | TPGEPA | PGSSVK | TPGEPA | PGSSVK | TPGEPA | MNW | TYTG | GD | GDTY | FS | (94- | |
| | VSCKAS | SISCRSS | VSCKAS | SISCRSS | VSCKAS | SISCRSS | (27- | EPTY | (98- | LE | (51- | 101) | |
| | GYTFTH | QSIVHSH | GYTFTH | QSIVHSH | GYTFTH | QSIVHSH | 36) | (47- | 107) | (27- | 61) | (SEQ | |
| | YGMNW | GDTYLE | YGMNW | GDTYLE | YGMNW | GDTYLE | (SEQ | 60) | (SEQ | 39) | (SEQ | ID | |
| | VRQAPG | WYLQKP | VRQAPG | WYLQKP | VRQAPG | WYLQKP | ID | (SEQ | ID | (SEQ | ID | NO: | |
| | QGLEWV | GQSPQL | QGLEWV | GQSPQL | QGLEWV | GQSPQL | NO: | ID | NO: | ID | NO: | 381) | |
| | GWVNTY | LIYKVSN | GWVNTY | LIYKVSN | GWVNTY | LIYKVSN | 239) | NO: | 294) | NO: | 350) | | |
| | TGEPTY | RFSGVP | TGEPTY | RFSGVP | TGEPTY | RFSGVP | | 266) | | 324) | | | |
| | ADDFKG | DRFSGS | ADDFKG | DRFSGS | ADDFKG | DRFSGS | | | | | | | |
| | RLTFTLD | GSGTDF | RLTFTLD | GSGTDF | RLTFTLD | GSGTDF | | | | | | | |
| | TSTSTAY | TLKISRV | TSTSTAY | TLKISRV | TSTSTAY | TLKISRV | | | | | | | |
| | MELSSL | EAEDVG | MELSSL | EAEDVG | MELSSL | EAEDVG | | | | | | | |
| | RSEDTA | VYYCFQ | RSEDTA | VYYCFQ | RSEDTA | VYYCFQ | | | | | | | |
| | VYYCTR | GSHIPVT | VYYCTR | GSHIPVT | VYYCTR | GSHIPVT | | | | | | | |
| | EGEGLG | FGQGTK | EGEGLG | FGQGTK | EGEGLG | FGQGTK | | | | | | | |
| | FGDWG | LEIKRTV | FGDWG | LEIK | FGDWG | LEIKGGG | | | | | | | |
| | QGTTVT | AAPSVFI | QGTTVT | (SEQ ID | QGTTVT | GSGGGG | | | | | | | |
| | VSSAST | FPPSDE | VSS | NO: 150) | VSSGGG | SGGGGS | | | | | | | |
| | KGPSVF | QLKSGT | (SEQ ID | | GSGGGG | EIQLVQS | | | | | | | |
| | PLAPSSK | ASVVCLL | NO: 120) | | SGGGGS | GAEVKK | | | | | | | |
| | STSGGT | NNFYPR | | | DVVMTQ | PGSSVK | | | | | | | |
| | AALGCL | EAKVQW | | | SPLSLPV | VSCKAS | | | | | | | |
| | VKDYFP | KVDNAL | | | TPGEPA | GYTFTH | | | | | | | |
| | EPVTVS | QSGNSQ | | | SISCRSS | YGMNW | | | | | | | |
| | WNSGAL | ESVTEQ | | | QSIVHSH | VRQAPG | | | | | | | |
| | TSGVHT | DSKDST | | | GDTYLE | QGLEWV | | | | | | | |
| | FPAVLQ | YSLSSTL | | | WYLQKP | GWVNTY | | | | | | | |
| | SSGLYSL | TLSKADY | | | GQSPQL | TGEPTY | | | | | | | |
| | SSVVTV | EKHKVY | | | LIYKVSN | ADDFKG | | | | | | | |
| | PSSSLG | ACEVTH | | | RFSGVP | RLTFTLD | | | | | | | |
| | TQTYICN | QGLSSP | | | DRFSGS | TSTSTAY | | | | | | | |
| | VNHKPS | VTKSFN | | | GSGTDF | MELSSL | | | | | | | |
| | NTKVDK | RGEC | | | TLKISRV | RSEDTA | | | | | | | |
| | KVEPKS | (SEQ ID | | | EAEDVG | VYYCTR | | | | | | | |
| | CDKTHT | NO: 90) | | | VYYCFQ | EGEGLG | | | | | | | |
| | CPPCPA | | | | GSHIPVT | FGDWG | | | | | | | |
| | PEAAGG | | | | FGQGTK | QGTTVT | | | | | | | |
| | PSVFLFP | | | | LEIK | VSS | | | | | | | |
| | PKPKDTL | | | | (SEQ ID | (SEQ ID | | | | | | | |
| | MISRTPE | | | | NO: 180) | NO: 210) | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPREE | | | | | | | | | | | | |
| | QYNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KALPAPI | | | | | | | | | | | | |
| | EKTISKA | | | | | | | | | | | | |
| | KGQPRE | | | | | | | | | | | | |
| | PQVYTL | | | | | | | | | | | | |
| | PPSREE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | KLTVDKS | | | | | | | | | | | | |
| | RWQQG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSP | | | | | | | | | | | | |
| | GK (SEQ | | | | | | | | | | | | |
| | ID NO: | | | | | | | | | | | | |
| | 60) | | | | | | | | | | | | |
| ezabenlimab | EVMLVE | EIVLTQS | EVMLVE | EIVLTQS | EVMLVE | EIVLTQS | FTFS | WVA | RHSN | ENID | LLIYV | QQS | eza- |
| | SGGGLV | PATLSLS | SGGGLV | PATLSLS | SGGGLV | PATLSLS | KSA | YISG | VNYY | VSGI | ASNQ | KEVP | benlimab |
| | QPGGSL | PGERAT | QPGGSL | PGERAT | QPGGSL | PGERAT | MS | GGG | AMD | SFMN | GS | W | |
| | RLSCTA | MSCRAS | RLSCTA | MSCRAS | RLSCTA | MSCRAS | (27- | DTYY | Y (98- | (27- | (50- | (93- | |
| | SGFTFS | ENIDVSG | SGFTFS | ENIDVSG | SGFTFS | ENIDVSG | 35) | (47- | 109) | 38) | 60) | 100) | |
| | KSAMSW | ISFMNW | KSAMSW | ISFMNW | KSAMSW | ISFMNW | (SEQ | 60) | (SEQ | (SEQ | (SEQ | (SEQ | |
| | VRQAPG | YQQKPG | VRQAPG | YQQKPG | VRQAPG | YQQKPG | ID | (SEQ | ID | ID | ID | ID | |
| | KGLEWV | QAPKLLI | KGLEWV | QAPKLLI | KGLEWV | QAPKLLI | NO: | ID | NO: | NO: | NO: | NO: | |
| | AYISGG | YVASNQ | AYISGG | YVASNQ | AYISGG | YVASNQ | 240) | NO: | 295) | 325) | 354) | 382) | |
| | GGDTYY | GSGIPAR | GGDTYY | GSGIPAR | GGDTYY | GSGIPAR | | 267) | | | | | |
| | SSSVKG | FSGSGS | SSSVKG | FSGSGS | SSSVKG | FSGSGS | | | | | | | |
| | RFTISRD | GTDFTLT | RFTISRD | GTDFTLT | RFTISRD | GTDFTLT | | | | | | | |
| | NAKNSL | ISRLEPE | NAKNSL | ISRLEPE | NAKNSL | ISRLEPE | | | | | | | |
| | YLQMNS | DFAVYY | YLQMNS | DFAVYY | YLQMNS | DFAVYY | | | | | | | |
| | LRAEDT | CQQSKE | LRAEDT | CQQSKE | LRAEDT | CQQSKE | | | | | | | |
| | AVYYCA | VPWTFG | AVYYCA | VPWTFG | AVYYCA | VPWTFG | | | | | | | |
| | RHSNVN | QGTKLEI | RHSNVN | QGTKLEI | RHSNVN | QGTKLEI | | | | | | | |
| | YYAMDY | KRTVAA | YYAMDY | K (SEQ | YYAMDY | KGGGGS | | | | | | | |
| | WGQGTL | PSVFIFP | WGQGTL | ID NO: | WGQGTL | GGGGSG | | | | | | | |
| | VTVSSA | PSDEQL | VTVSS | 151) | VTVSSG | GGGSEV | | | | | | | |
| | STKGPS | KSGTAS | (SEQ ID | | GGGSGG | MLVESG | | | | | | | |
| | VFPLAPC | VVCLLN | NO: 121) | | GGSGGG | GGLVQP | | | | | | | |
| | SRSTSE | NFYPRE | | | GSEIVLT | GGSLRL | | | | | | | |
| | STAALG | AKVQWK | | | QSPATL | SCTASG | | | | | | | |
| | CLVKDY | VDNALQ | | | SLSPGE | FTFSKSA | | | | | | | |
| | FPEPVTV | SGNSQE | | | RATMSC | MSWVR | | | | | | | |
| | SWNSGA | SVTEQD | | | RASENID | QAPGKG | | | | | | | |
| | LTSGVH | SKDSTY | | | VSGISFM | LEWVAYI | | | | | | | |
| | TFPAVLQ | SLSSTLT | | | NWYQQK | SGGGGD | | | | | | | |
| | SSGLYSL | LSKADY | | | PGQAPK | TYYSSS | | | | | | | |
| | SSVVTV | EKHKVY | | | LLIYVAS | VKGRFTI | | | | | | | |
| | PSSSLG | ACEVTH | | | NQGSGI | SRDNAK | | | | | | | |
| | TKTYTC | QGLSSP | | | PARFSG | NSLYLQ | | | | | | | |
| | NVDHKP | VTKSFN | | | SGSGTD | MNSLRA | | | | | | | |
| | SNTKVD | RGEC | | | FTLTISR | EDTAVY | | | | | | | |
| | KRVESK | (SEQ ID | | | LEPEDFA | YCARHS | | | | | | | |
| | YGPPCP | NO: 91) | | | VYYCQQ | NVNYYA | | | | | | | |
| | PCPAPE | | | | SKEVPW | MDYWG | | | | | | | |
| | FLGGPS | | | | TFGQGT | QGTLVT | | | | | | | |
| | VFLFPPK | | | | KLEIK | VSS | | | | | | | |
| | PKDTLMI | | | | (SEQ ID | (SEQ ID | | | | | | | |
| | SRTPEV | | | | NO: 181) | NO: 211) | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLG | | | | | | | | | | | | |
| | (SEQ ID | | | | | | | | | | | | |
| | NO: 61) | | | | | | | | | | | | |
| iparomlimab | QVQLVQ | DIQMTQ | QVQLVQ | DIQMTQ | QVQLVQ | DIQMTQ | YTFT | WMG | RPGF | QSLF | LLIYG | QND | ipa- |
| | SGAEVK | SPSSLSA | SGAEVK | SPSSLSA | SGAEVK | SPSSLSA | NYWI | EIDP | TYGG | NSG | ASTR | HYYP | romlimab |
| | KPGASV | SVGDRV | KPGASV | SVGDRV | KPGASV | SVGDRV | H (27- | YDSY | MDF | NQK | DS | Y (95- | |
| | KVSCKA | TITCKSS | KVSCKA | TITCKSS | KVSCKA | TITCKSS | 35) | TNY | (98- | NYLA | (52- | 102) | |
| | SGYTFT | QSLFNS | SGYTFT | QSLFNS | SGYTFT | QSLFNS | (SEQ | (47- | 108) | (27- | 62) | (SEQ | |
| | NYWIHW | GNQKNY | NYWIHW | GNQKNY | NYWIHW | GNQKNY | ID | 60) | (SEQ | 40) | (SEQ | ID | |
| | VRQAPG | LAWYQQ | VRQAPG | LAWYQQ | VRQAPG | LAWYQQ | NO: | (SEQ | ID | (SEQ | ID | NO: | |
| | QGLEWM | KPGKVP | QGLEWM | KPGKVP | QGLEWM | KPGKVP | 241) | ID | NO: | ID | NO: | 383) | |
| | GEIDPYD | KLLIYGA | GEIDPYD | KLLIYGA | GEIDPYD | KLLIYGA | | NO: | 296) | NO: | 355) | | |
| | SYTNYN | STRDSG | SYTNYN | STRDSG | SYTNYN | STRDSG | | 268) | | 326) | | | |
| | QKFKGR | VPYRFS | QKFKGR | VPYRFS | QKFKGR | VPYRFS | | | | | | | |
| | VTMTVD | GSGSGT | VTMTVD | GSGSGT | VTMTVD | GSGSGT | | | | | | | |
| | KSTSTVY | DFTLTIS | KSTSTVY | DFTLTIS | KSTSTVY | DFTLTIS | | | | | | | |
| | MELSSL | SLQPED | MELSSL | SLQPED | MELSSL | SLQPED | | | | | | | |
| | RSEDTA | VATYYC | RSEDTA | VATYYC | RSEDTA | VATYYC | | | | | | | |
| | VYYCAR | QNDHYY | VYYCAR | QNDHYY | VYYCAR | QNDHYY | | | | | | | |
| | PGFTYG | PYTFGG | PGFTYG | PYTFGG | PGFTYG | PYTFGG | | | | | | | |
| | GMDFW | GTKVEIK | GMDFW | GTKVEIK | GMDFW | GTKVEIK | | | | | | | |
| | GQGTLV | RTVAAP | GQGTLV | (SEQ ID | GQGTLV | GGGGSG | | | | | | | |
| | TVSSAST | SVFIFPP | TVSS | NO: 152) | TVSSGG | GGGSGG | | | | | | | |
| | KGPSVF | SDEQLK | (SEQ ID | | GGSGGG | GGSQVQ | | | | | | | |
| | PLAPCS | SGTASV | NO: 122) | | GSGGGG | LVQSGA | | | | | | | |
| | RSTSES | VCLLNNF | | | SDIQMT | EVKKPG | | | | | | | |
| | TAALGCL | YPREAK | | | QSPSSL | ASVKVS | | | | | | | |
| | VKDYFP | VQWKVD | | | SASVGD | CKASGY | | | | | | | |
| | EPVTVS | NALQSG | | | RVTITCK | TFTNYWI | | | | | | | |
| | WNSGAL | NSQESV | | | SSQSLF | HWVRQA | | | | | | | |
| | TSGVHT | TEQDSK | | | NSGNQK | PGQGLE | | | | | | | |
| | FPAVLQ | DSTYSLS | | | NYLAWY | WMGEID | | | | | | | |
| | SSGLYSL | STLTLSK | | | QQKPGK | PYDSYT | | | | | | | |
| | SSVVTV | ADYEKH | | | VPKLLIY | NYNQKF | | | | | | | |
| | PSSSLG | KVYACE | | | GASTRD | KGRVTM | | | | | | | |
| | TKTYTQ | VTHQGL | | | SGVPYR | TVDKST | | | | | | | |
| | NVDHKP | SSPVTK | | | FSGSGS | STVYME | | | | | | | |
| | SNTKVD | SFNRGE | | | GTDFTLT | LSSLRSE | | | | | | | |
| | KRVESK | C (SEQ | | | ISSLQPE | DTAVYY | | | | | | | |
| | YGPPCP | ID NO: | | | DVATYY | CARPGF | | | | | | | |
| | PCPAPE | 92) | | | CQNDHY | TYGGMD | | | | | | | |
| | FLGGPS | | | | YPYTFG | FWGQGT | | | | | | | |
| | VFLFPPK | | | | GGTKVEI | LVTVSS | | | | | | | |
| | PKDTLMI | | | | K (SEQ | (SEQ ID | | | | | | | |
| | SRTPEV | | | | ID NO: | NO: 212) | | | | | | | |
| | TCVVVD | | | | 182) | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 62) | | | | | | | | | | | | |
| peresolimab | QVQLVQ | DIQMTQ | QVQLVQ | DIQMTQ | QVQLVQ | DIQMTQ | YSLS | WMGI | TGNP | QSPN | LLIYG | QNN | peres |
| | SGAEVK | SPSSLSA | SGAEVK | SPSSLSA | SGAEVK | SPSSLSA | KYD | IYTS | YYTN | NLLA | ASDL | YYVG | olimab |
| | KPGASV | SVGDRV | KPGASV | SVGDRV | KPGASV | SVGDRV | MS | GYTD | GFNS | (27- | PS | PVSY | |
| | KVSCKV | TITCQAS | KVSCKV | TITCQAS | KVSCKV | TITCQAS | (27- | Y (47- | (97- | 34) | (46- | A (89- | |
| | SGYSLS | QSPNNL | SGYSLS | QSPNNL | SGYSLS | QSPNNL | 35) | 59) | 108) | (SEQ | 56) | 100) | |
| | KYDMSW | LAWYQQ | KYDMSW | LAWYQQ | KYDMSW | LAWYQQ | (SEQ | (SEQ | (SEQ | ID | (SEQ | (SEQ | |
| | VRQAPG | KPGKAP | VRQAPG | KPGKAP | VRQAPG | KPGKAP | ID | ID | ID | NO: | ID | ID | |
| | KGLEWM | KLLIYGA | KGLEWM | KLLIYGA | KGLEWM | KLLIYGA | NO: | NO: | NO: | 327) | NO: | NO: | |
| | GIIYTSG | SDLPSG | GIIYTSG | SDLPSG | GIIYTSG | SDLPSG | 242) | 269) | 297) | | 356) | 384) | |
| | YTDYAQ | VPSRFS | YTDYAQ | VPSRFS | YTDYAQ | VPSRFS | | | | | | | |
| | KFQGRV | GSGSGT | KFQGRV | GSGSGT | KFQGRV | GSGSGT | | | | | | | |
| | TMTEDT | DFTLTIS | TMTEDT | DFTLTIS | TMTEDT | DFTLTIS | | | | | | | |
| | STDTAY | SLQPED | STDTAY | SLQPED | STDTAY | SLQPED | | | | | | | |
| | MELSSL | FATYYC | MELSSL | FATYYC | MELSSL | FATYYC | | | | | | | |
| | RSEDTA | QNNYYV | RSEDTA | QNNYYV | RSEDTA | QNNYYV | | | | | | | |
| | VYYCAT | GPVSYA | VYYCAT | GPVSYA | VYYCAT | GPVSYA | | | | | | | |
| | GNPYYT | FGGGTK | GNPYYT | FGGGTK | GNPYYT | FGGGTK | | | | | | | |
| | NGFNSW | VEIKRTV | NGFNSW | VEIK | NGFNSW | VEIKGG | | | | | | | |
| | GQGTLV | AAPSVFI | GQGTLV | (SEQ ID | GQGTLV | GGSGGG | | | | | | | |
| | TVSSAST | FPPSDE | TVSS | NO: 153) | TVSSGG | GSGGGG | | | | | | | |
| | KGPSVF | QLKSGT | (SEQ ID | | GGSGGG | SQVQLV | | | | | | | |
| | PLAPSSK | ASVVCLL | NO: 123) | | GSGGGG | QSGAEV | | | | | | | |
| | STSGGT | NNFYPR | | | SDIQMT | KKPGAS | | | | | | | |
| | AALGCL | EAKVQW | | | QSPSSL | VKVSCK | | | | | | | |
| | VKDYFP | KVDNAL | | | SASVGD | VSGYSL | | | | | | | |
| | EPVTVS | QSGNSQ | | | RVTITCQ | SKYDMS | | | | | | | |
| | WNSGAL | ESVTEQ | | | ASQSPN | WVRQAP | | | | | | | |
| | TSGVHT | DSKDST | | | NLLAWY | GKGLEW | | | | | | | |
| | FPAVLQ | YSLSSTL | | | QQKPGK | MGIIYTS | | | | | | | |
| | SSGLYSL | TLSKADY | | | APKLLIY | GYTDYA | | | | | | | |
| | SSVVTV | EKHKVY | | | GASDLP | QKFQGR | | | | | | | |
| | PSSSLG | ACEVTH | | | SGVPSR | VTMTED | | | | | | | |
| | TQTYICN | QGLSSP | | | FSGSGS | TSTDTAY | | | | | | | |
| | VNHKPS | VTKSFN | | | GTDFTLT | MELSSL | | | | | | | |
| | NTKVDK | RGEC | | | ISSLQPE | RSEDTA | | | | | | | |
| | KVEPKS | (SEQ ID | | | DFATYY | VYYCAT | | | | | | | |
| | CDKTHT | NO: 93) | | | CQNNYY | GNPYYT | | | | | | | |
| | CPPCPA | | | | VGPVSY | NGFNSW | | | | | | | |
| | PELLGG | | | | AFGGGT | GQGTLV | | | | | | | |
| | PSVFLFP | | | | KVEIK | TVSS | | | | | | | |
| | PKPKDTL | | | | (SEQ ID | (SEQ ID | | | | | | | |
| | MISRTPE | | | | NO: 183) | NO: 213) | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPREE | | | | | | | | | | | | |
| | QYNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KALPAPI | | | | | | | | | | | | |
| | EKTISKA | | | | | | | | | | | | |
| | KGQPRE | | | | | | | | | | | | |
| | PQVYTL | | | | | | | | | | | | |
| | PPSRDE | | | | | | | | | | | | |
| | LTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | KLTVDKS | | | | | | | | | | | | |
| | RWQQG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSP | | | | | | | | | | | | |
| | GK (SEQ | | | | | | | | | | | | |
| | ID NO: | | | | | | | | | | | | |
| | 63) | | | | | | | | | | | | |
| pimivalimab | QVQLVQ | DIQMTQ | QVQLVQ | DIQMTQ | QVQLVQ | DIQMTQ | YTFP | WMGI | RGG | QSIS | LLIYE | QQY | pimiv |
| | SGAEVK | SPSTLSA | SGAEVK | SPSTLSA | SGAEVK | SPSTLSA | SYY | INPE | TYYD | SWLA | ASSL | NSFP | alimab |
| | KPGASV | SVGDRV | KPGASV | SVGDRV | KPGASV | SVGDRV | MH | GGST | YTY | (27- | ES | P (89- | |
| | KVSCKA | TITCRAS | KVSCKA | TITCRAS | KVSCKA | TITCRAS | (27- | AY | (98- | 34) | (46- | 96) | |
| | SGYTFP | QSISSWL | SGYTFP | QSISSWL | SGYTFP | QSISSWL | 35) | (47- | 107) | (SEQ | 56) | (SEQ | |
| | SYYMHW | AWYQQK | SYYMHW | AWYQQK | SYYMHW | AWYQQK | (SEQ | 60) | (SEQ | ID | (SEQ | ID | |
| | VRQAPG | PGKAPK | VRQAPG | PGKAPK | VRQAPG | PGKAPK | ID | (SEQ | ID | NO: | ID | NO: | |
| | QGLEWM | LLIYEAS | QGLEWM | LLIYEAS | QGLEWM | LLIYEAS | NO: | ID | NO: | 328) | NO: | 385) | |
| | GIINPEG | SLESGV | GIINPEG | SLESGV | GIINPEG | SLESGV | 243) | NO: | 298) | | 357) | | |
| | GSTAYA | PSRFSG | GSTAYA | PSRFSG | GSTAYA | PSRFSG | | 270) | | | | | |
| | QKFQGR | SGSGTE | QKFQGR | SGSGTE | QKFQGR | SGSGTE | | | | | | | |
| | VTMTRD | FTLTISSL | VTMTRD | FTLTISSL | VTMTRD | FTLTISSL | | | | | | | |
| | TSTSTVY | QPDDFA | TSTSTVY | QPDDFA | TSTSTVY | QPDDFA | | | | | | | |
| | MELSSL | TYYCQQ | MELSSL | TYYCQQ | MELSSL | TYYCQQ | | | | | | | |
| | RSEDTA | YNSFPP | RSEDTA | YNSFPP | RSEDTA | YNSFPP | | | | | | | |
| | VYYCAR | TFGGGT | VYYCAR | TFGGGT | VYYCAR | TFGGGT | | | | | | | |
| | GGTYYD | KVEIKRT | GGTYYD | KVEIK | GGTYYD | KVEIKGG | | | | | | | |
| | YTYWGQ | VAAPSV | YTYWGQ | (SEQ ID | YTYWGQ | GGSGGG | | | | | | | |
| | GTLVTVS | FIFPPSD | GTLVTVS | NO: 154) | GTLVTVS | GSGGGG | | | | | | | |
| | SASTKG | EQLKSG | S (SEQ | | SGGGGS | SQVQLV | | | | | | | |
| | PSVFPLA | TASVVCL | ID NO: | | GGGGSG | QSGAEV | | | | | | | |
| | PCSRST | LNNFYP | 124) | | GGGSDI | KKPGAS | | | | | | | |
| | SESTAAL | REAKVQ | | | QMTQSP | VKVSCK | | | | | | | |
| | GCLVKD | WKVDNA | | | STLSASV | ASGYTF | | | | | | | |
| | YFPEPVT | LQSGNS | | | GDRVTIT | PSYYMH | | | | | | | |
| | VSWNSG | QESVTE | | | CRASQSI | WVRQAP | | | | | | | |
| | ALTSGV | QDSKDS | | | SSWLAW | GQGLEW | | | | | | | |
| | HTFPAVL | TYSLSST | | | YQQKPG | MGIINPE | | | | | | | |
| | QSSGLY | LTLSKAD | | | KAPKLLI | GGSTAY | | | | | | | |
| | SLSSVVT | YEKHKV | | | YEASSLE | AQKFQG | | | | | | | |
| | VPSSSL | YACEVT | | | SGVPSR | RVTMTR | | | | | | | |
| | GTKTYT | HQGLSS | | | FSGSGS | DTSTSTV | | | | | | | |
| | CNVDHK | PVTKSF | | | GTEFTLT | YMELSS | | | | | | | |
| | PSNTKV | NRGEC | | | ISSLQPD | LRSEDT | | | | | | | |
| | DKRVES | (SEQ ID | | | DFATYY | AVYYCA | | | | | | | |
| | KYGPPC | NO: 94) | | | CQQYNS | RGGTYY | | | | | | | |
| | PPCPAP | | | | FPPTFG | DYTYWG | | | | | | | |
| | EFLGGP | | | | GGTKVEI | QGTLVT | | | | | | | |
| | SVFLFPP | | | | K (SEQ | VSS | | | | | | | |
| | KPKDTL | | | | ID NO: | (SEQ ID | | | | | | | |
| | MISRTPE | | | | 184) | NO: 214) | | | | | | | |
| | VTCVVV | | | | | | | | | | | | |
| | DVSQED | | | | | | | | | | | | |
| | PEVQFN | | | | | | | | | | | | |
| | WYVDGV | | | | | | | | | | | | |
| | EVHNAK | | | | | | | | | | | | |
| | TKPREE | | | | | | | | | | | | |
| | QFNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KGLPSSI | | | | | | | | | | | | |
| | EKTISKA | | | | | | | | | | | | |
| | KGQPRE | | | | | | | | | | | | |
| | PQVYTL | | | | | | | | | | | | |
| | PPSQEE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | RLTVDK | | | | | | | | | | | | |
| | SRWQEG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSL | | | | | | | | | | | | |
| | GK (SEQ | | | | | | | | | | | | |
| | ID NO: | | | | | | | | | | | | |
| | 64) | | | | | | | | | | | | |
| rosnilimab | QVQLVQ | EIVLTQS | QVQLVQ | EIVLTQS | QVQLVQ | EIVLTQS | NYTF | WMG | RDYY | SSVS | LLIYS | HQY | ros- |
| | SGSELK | PATLSLS | SGSELK | PATLSLS | SGSELK | PATLSLS | TDYS | WINI | GRFY | SSYF | TSNL | HRSP | nilimab |
| | KPGASV | PGERAT | KPGASV | PGERAT | KPGASV | PGERAT | MH | ETYY | YAM | H (27- | AS | L (90- | |
| | KVSCKA | LSCTASS | KVSCKA | LSCTASS | KVSCKA | LSCTASS | (26- | PTY | DY | 35) | (47- | 97) | |
| | SNYTFT | SVSSSY | SNYTFT | SVSSSY | SNYTFT | SVSSSY | 35) | (47- | (98- | (SEQ | 57) | (SEQ | |
| | DYSMHW | FHWYQQ | DYSMHW | FHWYQQ | DYSMHW | FHWYQQ | (SEQ | 60) | 110) | ID | (SEQ | ID | |
| | VRQAPG | KPGQAP | VRQAPG | KPGQAP | VRQAPG | KPGQAP | ID | (SEQ | (SEQ | NO: | ID | NO: | |
| | QGLEWM | RLLIYST | QGLEWM | RLLIYST | QGLEWM | RLLIYST | NO: | ID | ID | 329) | NO: | 386) | |
| | GWINIET | SNLASGI | GWINIET | SNLASGI | GWINIET | SNLASGI | 244) | NO: | NO: | | 358) | | |
| | YYPTYA | PARFSG | YYPTYA | PARFSG | YYPTYA | PARFSG | | 271) | 299) | | | | |
| | DQFKGR | SGSGTD | DQFKGR | SGSGTD | DQFKGR | SGSGTD | | | | | | | |
| | FAFSLDT | FTLTISR | FAFSLDT | FTLTISR | FAFSLDT | FTLTISR | | | | | | | |
| | SVSTAYL | LEPEDFA | SVSTAYL | LEPEDFA | SVSTAYL | LEPEDFA | | | | | | | |
| | QISSLKA | VYYCHQ | QISSLKA | VYYCHQ | QISSLKA | VYYCHQ | | | | | | | |
| | EDTAVY | YHRSPL | EDTAVY | YHRSPL | EDTAVY | YHRSPL | | | | | | | |
| | YCARDY | TFGGGT | YCARDY | TFGGGT | YCARDY | TFGGGT | | | | | | | |
| | YGRFYY | KVEIKRT | YGRFYY | KVEIK | YGRFYY | KVEIKGG | | | | | | | |
| | AMDYW | VAAPSV | AMDYW | (SEQ ID | AMDYW | GGSGGG | | | | | | | |
| | GQGTTV | FIFPPSD | GQGTTV | NO: 155) | GQGTTV | GSGGGG | | | | | | | |
| | TVSSAST | EQLKSG | TVSS | | TVSSGG | SQVQLV | | | | | | | |
| | KGPSVF | TASVVCL | (SEQ ID | | GGSGGG | QSGSEL | | | | | | | |
| | PLAPSSK | LNNFYP | NO: 125) | | GSGGGG | KKPGAS | | | | | | | |
| | STSGGT | REAKVQ | | | SEIVLTQ | VKVSCK | | | | | | | |
| | AALGCL | WKVDNA | | | SPATLSL | ASNYTFT | | | | | | | |
| | VKDYFP | LQSGNS | | | SPGERA | DYSMHW | | | | | | | |
| | EPVTVS | QESVTE | | | TLSCTAS | VRQAPG | | | | | | | |
| | WNSGAL | QDSKDS | | | SSVSSS | QGLEWM | | | | | | | |
| | TSGVHT | TYSLSST | | | YFHWYQ | GWINIET | | | | | | | |
| | FPAVLQ | LTLSKAD | | | QKPGQA | YYPTYA | | | | | | | |
| | SSGLYSL | YEKHKV | | | PRLLIYS | DQFKGR | | | | | | | |
| | SSVVTV | YACEVT | | | TSNLAS | FAFSLDT | | | | | | | |
| | PSSSLG | HQGLSS | | | GIPARFS | SVSTAYL | | | | | | | |
| | TQTYICN | PVTKSF | | | GSGSGT | QISSLKA | | | | | | | |
| | VNHKPS | NRGEC | | | DFTLTIS | EDTAVY | | | | | | | |
| | NTKVDK | (SEQ ID | | | RLEPED | YCARDY | | | | | | | |
| | KVEPKS | NO: 95) | | | FAVYYC | YGRFYY | | | | | | | |
| | CDKTHT | | | | HQYHRS | AMDYW | | | | | | | |
| | CPPCPA | | | | PLTFGG | GQGTTV | | | | | | | |
| | PELLGG | | | | GTKVEIK | TVSS | | | | | | | |
| | PSVFLFP | | | | (SEQ ID | (SEQ ID | | | | | | | |
| | PKPKDTL | | | | NO: 185) | NO: 215) | | | | | | | |
| | MISRTPE | | | | | | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPREE | | | | | | | | | | | | |
| | QYNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KALPAPI | | | | | | | | | | | | |
| | EKTISKA | | | | | | | | | | | | |
| | KGQPRE | | | | | | | | | | | | |
| | PQVYTL | | | | | | | | | | | | |
| | PPSRDE | | | | | | | | | | | | |
| | LTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | KLTVDKS | | | | | | | | | | | | |
| | RWQQG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSP | | | | | | | | | | | | |
| | GK (SEQ | | | | | | | | | | | | |
| | ID NO: | | | | | | | | | | | | |
| | 65) | | | | | | | | | | | | |
| rulonilimab | EVQLVE | EIVLTQS | EVQLVE | EIVLTQS | EVQLVE | EIVLTQS | FTFS | WVA | RQK | ESVD | LLIYV | QQS | rulo- |
| | SGGGLV | PATLAVS | SGGGLV | PATLAVS | SGGGLV | PATLAVS | SYG | TISG | DTS | DYGI | ASNQ | KEVP | nilimab |
| | KPGGSL | PGERATI | KPGGSL | PGERATI | KPGGSL | PGERATI | MS | GGR | WFV | SFMN | GS | W | |
| | RLSCAA | SCRASE | RLSCAA | SCRASE | RLSCAA | SCRASE | (27- | DTYY | H (98- | (27- | (50- | (93- | |
| | SGFTFS | SVDDYGI | SGFTFS | SVDDYGI | SGFTFS | SVDDYGI | 35) | (47- | 107) | 38) | 60) | 100) | |
| | SYGMSW | SFMNWF | SYGMSW | SFMNWF | SYGMSW | SFMNWF | (SEQ | 60) | (SEQ | (SEQ | (SEQ | (SEQ | |
| | VRQTPE | QQKPGQ | VRQTPE | QQKPGQ | VRQTPE | QQKPGQ | ID | (SEQ | ID | ID | ID | ID | |
| | KRLEWV | PPKLLIY | KRLEWV | PPKLLIY | KRLEWV | PPKLLIY | NO: | ID | NO: | NO: | NO: | NO: | |
| | ATISGGG | VASNQG | ATISGGG | VASNQG | ATISGGG | VASNQG | 245) | NO: | 300) | 330) | 354) | 382) | |
| | RDTYYP | SGVPAR | RDTYYP | SGVPAR | RDTYYP | SGVPAR | | 272) | | | | | |
| | DSVKGR | FSGSGS | DSVKGR | FSGSGS | DSVKGR | FSGSGS | | | | | | | |
| | FTISRDN | GTDFTL | FTISRDN | GTDFTL | FTISRDN | GTDFTL | | | | | | | |
| | AKNNLYL | NIHPMEE | AKNNLYL | NIHPMEE | AKNNLYL | NIHPMEE | | | | | | | |
| | QMSSLR | DDTAMY | QMSSLR | DDTAMY | QMSSLR | DDTAMY | | | | | | | |
| | SEDTALY | FCQQSK | SEDTALY | FCQQSK | SEDTALY | FCQQSK | | | | | | | |
| | YCARQK | EVPWTF | YCARQK | EVPWTF | YCARQK | EVPWTF | | | | | | | |
| | DTSWFV | GGGTKL | DTSWFV | GGGTKL | DTSWFV | GGGTKL | | | | | | | |
| | HWGQG | EIKRTVA | HWGQG | EIK | HWGQG | EIKGGG | | | | | | | |
| | TLVTVSS | APSVFIF | TLVTVSS | (SEQ | TLVTVSS | GSGGGG | | | | | | | |
| | ASTKGP | PPSDEQ | (SEQ ID | ID NO: | GGGGSG | SGGGGS | | | | | | | |
| | SVFPLAP | LKSGTA | NO: 126) | 156) | GGGSGG | EVQLVE | | | | | | | |
| | SSKSTS | SVVCLLN | | | GGSEIVL | SGGGLV | | | | | | | |
| | GGTAAL | NFYPRE | | | TQSPATL | KPGGSL | | | | | | | |
| | GCLVKD | AKVQWK | | | AVSPGE | RLSCAA | | | | | | | |
| | YFPEPVT | VDNALQ | | | RATISCR | SGFTFS | | | | | | | |
| | VSWNSG | SGNSQE | | | ASESVD | SYGMSW | | | | | | | |
| | ALTSGV | SVTEQD | | | DYGISFM | VRQTPE | | | | | | | |
| | HTFPAVL | SKDSTY | | | NWFQQK | KRLEWV | | | | | | | |
| | QSSGLY | SLSSTLT | | | PGQPPK | ATISGGG | | | | | | | |
| | SLSSVVT | LSKADY | | | LLIYVAS | RDTYYP | | | | | | | |
| | VPSSSL | EKHKVY | | | NQGSGV | DSVKGR | | | | | | | |
| | GTQTYIC | ACEVTH | | | PARFSG | FTISRDN | | | | | | | |
| | NVNHKP | QGLSSP | | | SGSGTD | AKNNLYL | | | | | | | |
| | SNTKVD | VTKSFN | | | FTLNIHP | QMSSLR | | | | | | | |
| | KKVEPK | RGEC | | | MEEDDT | SEDTALY | | | | | | | |
| | SCDKTH | (SEQ ID | | | AMYFCQ | YCARQK | | | | | | | |
| | TCPPCP | NO: 96) | | | QSKEVP | DTSWFV | | | | | | | |
| | APELLG | | | | WTFGGG | HWGQG | | | | | | | |
| | GPSVFLF | | | | TKLEIK | TLVTVSS | | | | | | | |
| | PPKPKD | | | | (SEQ ID | (SEQ ID | | | | | | | |
| | QLMISRT | | | | NO: 186) | NO: 216) | | | | | | | |
| | PEVTCV | | | | | | | | | | | | |
| | VVDVSH | | | | | | | | | | | | |
| | EDPEVK | | | | | | | | | | | | |
| | FNWYVD | | | | | | | | | | | | |
| | GVEVHN | | | | | | | | | | | | |
| | AKTKPR | | | | | | | | | | | | |
| | EEQYAS | | | | | | | | | | | | |
| | TYRVVS | | | | | | | | | | | | |
| | VLTVLHQ | | | | | | | | | | | | |
| | DWLNGK | | | | | | | | | | | | |
| | EYKCKV | | | | | | | | | | | | |
| | SNKALP | | | | | | | | | | | | |
| | APIEKTIS | | | | | | | | | | | | |
| | KAKGQP | | | | | | | | | | | | |
| | REPQVY | | | | | | | | | | | | |
| | TLPPSRE | | | | | | | | | | | | |
| | EMTKNQ | | | | | | | | | | | | |
| | VSLTCLV | | | | | | | | | | | | |
| | KGFYPS | | | | | | | | | | | | |
| | DIAVEW | | | | | | | | | | | | |
| | ESNGQP | | | | | | | | | | | | |
| | ENNYKT | | | | | | | | | | | | |
| | TPPVLDS | | | | | | | | | | | | |
| | DGSFFL | | | | | | | | | | | | |
| | YSKLTVD | | | | | | | | | | | | |
| | KSRWQQ | | | | | | | | | | | | |
| | GNVFSC | | | | | | | | | | | | |
| | SVLHEAL | | | | | | | | | | | | |
| | HNHYTQ | | | | | | | | | | | | |
| | KSLSLSP | | | | | | | | | | | | |
| | GK (SEQ | | | | | | | | | | | | |
| | ID NO: | | | | | | | | | | | | |
| | 66) | | | | | | | | | | | | |
| spartalizumab | EVQLVQ | EIVLTQS | EVQLVQ | EIVLTQS | EVQLVQ | EIVLTQS | YTFT | WMG | TRW | QSLL | LLIY | QND | spar- |
| | SGAEVK | PATLSLS | SGAEVK | PATLSLS | SGAEVK | PATLSLS | TYW | NIYP | TTGT | DSG | WAS | YSYP | tali- |
| | KPGESL | PGERAT | KPGESL | PGERAT | KPGESL | PGERAT | MH | GTG | GAY | NQK | TRES | Y (95- | zumab |
| | RISCKGS | LSCKSS | RISCKGS | LSCKSS | RISCKGS | LSCKSS | (27- | GSNF | (97- | NFLT | (52- | 102) | |
| | GYTFTTY | QSLLDS | GYTFTTY | QSLLDS | GYTFTTY | QSLLDS | 35) | D (47- | 106) | (27- | 62) | (SEQ | |
| | WMHWV | GNQKNF | WMHWV | GNQKNF | WMHWV | GNQKNF | (SEQ | 61) | (SEQ | 40) | (SEQ | ID | |
| | RQATGQ | LTWYQQ | RQATGQ | LTWYQQ | RQATGQ | LTWYQQ | ID | (SEQ | ID | (SEQ | ID | NO: | |
| | GLEWMG | KPGQAP | GLEWMG | KPGQAP | GLEWMG | KPGQAP | NO: | ID | NO: | ID | NO: | 387) | |
| | NIYPGTG | RLLIYWA | NIYPGTG | RLLIYWA | NIYPGTG | RLLIYWA | 246) | NO: | 301) | NO: | 359) | | |
| | GSNFDE | STRESG | GSNFDE | STRESG | GSNFDE | STRESG | | 273) | | 331) | | | |
| | KFKNRV | VPSRFS | KFKNRV | VPSRFS | KFKNRV | VPSRFS | | | | | | | |
| | TITADKS | GSGSGT | TITADKS | GSGSGT | TITADKS | GSGSGT | | | | | | | |
| | TSTAYM | DFTFTIS | TSTAYM | DFTFTIS | TSTAYM | DFTFTIS | | | | | | | |
| | ELSSLRS | SLEAED | ELSSLRS | SLEAED | ELSSLRS | SLEAED | | | | | | | |
| | EDTAVY | AATYYC | EDTAVY | AATYYC | EDTAVY | AATYYC | | | | | | | |
| | YCTRWT | QNDYSY | YCTRWT | QNDYSY | YCTRWT | QNDYSY | | | | | | | |
| | TGTGAY | PYTFGQ | TGTGAY | PYTFGQ | TGTGAY | PYTFGQ | | | | | | | |
| | WGQGTT | GTKVEIK | WGQGTT | GTKVEIK | WGQGTT | GTKVEIK | | | | | | | |
| | VTVSSA | RTVAAP | VTVSS | (SEQ ID | VTVSSG | GGGGSG | | | | | | | |
| | STKGPS | SVFIFPP | (SEQ ID | NO: 157) | GGGSGG | GGGSGG | | | | | | | |
| | VFPLAPC | SDEQLK | NO: 127) | | GGSGGG | GGSEVQ | | | | | | | |
| | SRSTSE | SGTASV | | | GSEIVLT | LVQSGA | | | | | | | |
| | STAALG | VCLLNNF | | | QSPATL | EVKKPG | | | | | | | |
| | CLVKDY | YPREAK | | | SLSPGE | ESLRISC | | | | | | | |
| | FPEPVTV | VQWKVD | | | RATLSC | KGSGYT | | | | | | | |
| | SWNSGA | NALQSG | | | KSSQSLL | FTTYWM | | | | | | | |
| | LTSGVH | NSQESV | | | DSGNQK | HWVRQA | | | | | | | |
| | TFPAVLQ | TEQDSK | | | NFLTWY | TGQGLE | | | | | | | |
| | SSGLYSL | DSTYSLS | | | QQKPGQ | WMGNIY | | | | | | | |
| | SSVVTV | STLTLSK | | | APRLLIY | PGTGGS | | | | | | | |
| | PSSSLG | ADYEKH | | | WASTRE | NFDEKF | | | | | | | |
| | TKTYTC | KVYACE | | | SGVPSR | KNRVTIT | | | | | | | |
| | NVDHKP | VTHQGL | | | FSGSGS | ADKSTS | | | | | | | |
| | SNTKVD | SSPVTK | | | GTDFTFT | TAYMEL | | | | | | | |
| | KRVESK | SFNRGE | | | ISSLEAE | SSLRSE | | | | | | | |
| | YGPPCP | C (SEQ | | | DAATYY | DTAVYY | | | | | | | |
| | PCPAPE | ID NO: | | | CQNDYS | CTRWTT | | | | | | | |
| | FLGGPS | 97) | | | YPYTFG | GTGAYW | | | | | | | |
| | VFLFPPK | | | | QGTKVEI | GQGTTV | | | | | | | |
| | PKDTLMI | | | | K (SEQ | TVSS | | | | | | | |
| | SRTPEV | | | | ID NO: | (SEQ ID | | | | | | | |
| | TCVVVD | | | | 187) | NO: 217) | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLG | | | | | | | | | | | | |
| | (SEQ ID | | | | | | | | | | | | |
| | NO: 67) | | | | | | | | | | | | |
| sudubrilimab | EVQLVE | DIQMTQ | EVQLVE | DIQMTQ | EVQLVE | DIQMTQ | FTFS | WVA | RRH | QDVS | LLIYS | QQFL | sudu- |
| | SGGGLV | SPSSLSA | SGGGLV | SPSSLSA | SGGGLV | SPSSLSA | ETWL | WVS | WPG | TAVA | ASFL | YHPA | brilimab |
| | QPGGSL | SVGDRV | QPGGSL | SVGDRV | QPGGSL | SVGDRV | H (27- | PFGG | GFDY | (27- | YS | (89- | |
| | RLSCAA | TITCRAS | RLSCAA | TITCRAS | RLSCAA | TITCRAS | 35) | STYY | (98- | 34) | (46- | 96) | |
| | SGFTFS | QDVSTA | SGFTFS | QDVSTA | SGFTFS | QDVSTA | (SEQ | (47- | 107) | (SEQ | 56) | (SEQ | |
| | ETWLHW | VAWYQQ | ETWLHW | VAWYQQ | ETWLHW | VAWYQQ | ID | 60) | (SEQ | ID | (SEQ | ID | |
| | VRQAPG | KPGKAP | VRQAPG | KPGKAP | VRQAPG | KPGKAP | NO: | (SEQ | ID | NO: | ID | NO: | |
| | KGLEWV | KLLIYSA | KGLEWV | KLLIYSA | KGLEWV | KLLIYSA | 247) | ID | NO: | 332) | NO: | 388) | |
| | AWVSPF | SFLYSG | AWVSPF | SFLYSG | AWVSPF | SFLYSG | | NO: | 302) | | 360) | | |
| | GGSTYY | VPSRFS | GGSTYY | VPSRFS | GGSTYY | VPSRFS | | 274) | | | | | |
| | ADSVKG | GSGSGT | ADSVKG | GSGSGT | ADSVKG | GSGSGT | | | | | | | |
| | RFTISAD | DFTLTIS | RFTISAD | DFTLTIS | RFTISAD | DFTLTIS | | | | | | | |
| | TSKNTA | SLQPED | TSKNTA | SLQPED | TSKNTA | SLQPED | | | | | | | |
| | YLQMNS | FATYYC | YLQMNS | FATYYC | YLQMNS | FATYYC | | | | | | | |
| | LRAEDT | QQFLYH | LRAEDT | QQFLYH | LRAEDT | QQFLYH | | | | | | | |
| | AVYYCA | PATFGQ | AVYYCA | PATFGQ | AVYYCA | PATFGQ | | | | | | | |
| | RRHWPG | GTKVEIK | RRHWPG | GTKVEIK | RRHWPG | GTKVEIK | | | | | | | |
| | GFDYWG | RTVAAP | GFDYWG | (SEQ ID | GFDYWG | GGGGSG | | | | | | | |
| | QGTLVT | SVFIFPP | QGTLVT | NO: 158) | QGTLVT | GGGSGG | | | | | | | |
| | VSSAST | SDEQLK | VSS | | VSSGGG | GGSEVQ | | | | | | | |
| | KGPSVF | SGTASV | (SEQ ID | | GSGGGG | LVESGG | | | | | | | |
| | PLAPSSK | VCLLNNF | NO: 128) | | SGGGGS | GLVQPG | | | | | | | |
| | STSGGT | YPREAK | | | DIQMTQ | GSLRLS | | | | | | | |
| | AALGCL | VQWKVD | | | SPSSLSA | CAASGF | | | | | | | |
| | VKDYFP | NALQSG | | | SVGDRV | TFSETW | | | | | | | |
| | EPVTVS | NSQESV | | | TITCRAS | LHWVRQ | | | | | | | |
| | WNSGAL | TEQDSK | | | QDVSTA | APGKGL | | | | | | | |
| | TSGVHT | DSTYSLS | | | VAWYQQ | EWVAWV | | | | | | | |
| | FPAVLQ | STLTLSK | | | KPGKAP | SPFGGS | | | | | | | |
| | SSGLYSL | ADYEKH | | | KLLIYSA | TYYADS | | | | | | | |
| | SSVVTV | KVYACE | | | SFLYSG | VKGRFTI | | | | | | | |
| | PSSSLG | VTHQGL | | | VPSRFS | SADTSK | | | | | | | |
| | TQTYICN | SSPVTK | | | GSGSGT | NTAYLQ | | | | | | | |
| | VNHKPS | SFNRGE | | | DFTLTIS | MNSLRA | | | | | | | |
| | NTKVDK | C (SEQ | | | SLQPED | EDTAVY | | | | | | | |
| | KVEPKS | ID NO: | | | FATYYC | YCARRH | | | | | | | |
| | CDKTHT | 98) | | | QQFLYH | WPGGFD | | | | | | | |
| | CPPCPA | | | | PATFGQ | YWGQGT | | | | | | | |
| | PELLGG | | | | GTKVEIK | LVTVSS | | | | | | | |
| | PSVFLFP | | | | (SEQ ID | (SEQ ID | | | | | | | |
| | PKPKDTL | | | | NO: 188) | NO: 218) | | | | | | | |
| | MISRTPE | | | | | | | | | | | | |
| | VTCVVV | | | | | | | | | | | | |
| | DVSHED | | | | | | | | | | | | |
| | PEVKFN | | | | | | | | | | | | |
| | WYVDGV | | | | | | | | | | | | |
| | EVHNAK | | | | | | | | | | | | |
| | TKPREE | | | | | | | | | | | | |
| | QYASTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KALPAPI | | | | | | | | | | | | |
| | EKTISKA | | | | | | | | | | | | |
| | KGQPRE | | | | | | | | | | | | |
| | PQVYTL | | | | | | | | | | | | |
| | PPSREE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | KLTVDKS | | | | | | | | | | | | |
| | RWQQG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSP | | | | | | | | | | | | |
| | G (SEQ | | | | | | | | | | | | |
| | ID NO: | | | | | | | | | | | | |
| | 68) | | | | | | | | | | | | |
| finotonlimab | EVQLVE | EIVLTQS | EVQLVE | EIVLTQS | EVQLVE | EIVLTQS | FTFS | WVA | RQY | ESVD | LLIYA | QQS | fino- |
| | SGGGLV | PATLSLS | SGGGLV | PATLSLS | SGGGLV | PATLSLS | SYG | TISG | GTV | SYGN | ASNQ | KEVP | tonlimab |
| | KPGGSL | PGERAT | KPGGSL | PGERAT | KPGGSL | PGERAT | MS | GGR | WFF | SFMH | GS | W | |
| | RLSCAA | LSCRAS | RLSCAA | LSCRAS | RLSCAA | LSCRAS | (27- | DTYY | N (98- | (27- | (50- | (93- | |
| | SGFTFS | ESVDSY | SGFTFS | ESVDSY | SGFTFS | ESVDSY | 35) | S (47- | 107) | 38) | 60) | 100) | |
| | SYGMSW | GNSFMH | SYGMSW | GNSFMH | SYGMSW | GNSFMH | (SEQ | 61) | (SEQ | (SEQ | (SEQ | (SEQ | |
| | VRQAPG | WYQQKP | VRQAPG | WYQQKP | VRQAPG | WYQQKP | ID | (SEQ | ID | ID | ID | ID | |
| | KRLEWV | GQPPRL | KRLEWV | GQPPRL | KRLEWV | GQPPRL | NO: | ID | VO: | NO: | NO: | NO: | |
| | ATISGGG | LIYAASN | ATISGGG | LIYAASN | ATISGGG | LIYAASN | 245) | NO: | 303) | 333) | 361) | 382) | |
| | RDTYYS | QGSGVP | RDTYYS | QGSGVP | RDTYYS | QGSGVP | | 272) | | | | | |
| | DSVKGR | ARFSGS | DSVKGR | ARFSGS | DSVKGR | ARFSGS | | | | | | | |
| | FTISRDN | GSGTDF | FTISRDN | GSGTDF | FTISRDN | GSGTDF | | | | | | | |
| | AKNNLYL | TLTISSL | AKNNLYL | TLTISSL | AKNNLYL | TLTISSL | | | | | | | |
| | QMNSLR | EPEDFA | QMNSLR | EPEDFA | QMNSLR | EPEDFA | | | | | | | |
| | AEDTAV | MYFCQQ | AEDTAV | MYFCQQ | AEDTAV | MYFCQQ | | | | | | | |
| | YYCSRQ | SKEVPW | YYCSRQ | SKEVPW | YYCSRQ | SKEVPW | | | | | | | |
| | YGTVWF | TFGQGT | YGTVWF | TFGQGT | YGTVWF | TFGQGT | | | | | | | |
| | FNWGQ | KVEIKRT | FNWGQ | KVEIK | FNWGQ | KVEIKGG | | | | | | | |
| | GTLVTVS | VAAPSV | GTLVTVS | (SEQ ID | GTLVTVS | GGSGGG | | | | | | | |
| | SASTKG | FIFPPSD | S (SEQ | NO: 159) | SGGGGS | GSGGGG | | | | | | | |
| | PSVFPLA | EQLKSG | ID NO: | | GGGGSG | SEVQLV | | | | | | | |
| | PCSRST | TASVVCL | 129) | | GGGSEI | ESGGGL | | | | | | | |
| | SESTAAL | LNNFYP | | | VLTQSP | VKPGGS | | | | | | | |
| | GCLVKD | REAKVQ | | | ATLSLSP | LRLSCAA | | | | | | | |
| | YFPEPVT | WKVDNA | | | GERATL | SGFTFS | | | | | | | |
| | VSWNSG | LQSGNS | | | SCRASE | SYGMSW | | | | | | | |
| | ALTSGV | QESVTE | | | SVDSYG | VRQAPG | | | | | | | |
| | HTFPAVL | QDSKDS | | | NSFMHW | KRLEWV | | | | | | | |
| | QSSGLY | TYSLSST | | | YQQKPG | ATISGGG | | | | | | | |
| | SLSSVVT | LTLSKAD | | | QPPRLLI | RDTYYS | | | | | | | |
| | VPSSSL | YEKHKV | | | YAASNQ | DSVKGR | | | | | | | |
| | GTKTYT | YACEVT | | | GSGVPA | FTISRDN | | | | | | | |
| | CNVDHK | HQGLSS | | | RFSGSG | AKNNLYL | | | | | | | |
| | PSNTKV | PVTKSF | | | SGTDFTL | QMNSLR | | | | | | | |
| | DKRVES | NRGEC | | | TISSLEP | AEDTAV | | | | | | | |
| | KYGPPC | (SEQ ID | | | EDFAMY | YYCSRQ | | | | | | | |
| | PPCPAP | NO: 99) | | | FCQQSK | YGTVWF | | | | | | | |
| | EFLGGP | | | | EVPWTF | FNWGQ | | | | | | | |
| | SVFLFPP | | | | GQGTKV | GTLVTVS | | | | | | | |
| | KPKDTL | | | | EIK (SEQ | S (SEQ | | | | | | | |
| | MISRTPE | | | | ID NO: | ID NO: | | | | | | | |
| | VTCVVV | | | | 189) | 219) | | | | | | | |
| | DVSQED | | | | | | | | | | | | |
| | PEVQFN | | | | | | | | | | | | |
| | WYVDGV | | | | | | | | | | | | |
| | EVHNAK | | | | | | | | | | | | |
| | TKPREE | | | | | | | | | | | | |
| | QFNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KGLPSSI | | | | | | | | | | | | |
| | EKTISKA | | | | | | | | | | | | |
| | KGQPRE | | | | | | | | | | | | |
| | PQVYTL | | | | | | | | | | | | |
| | PPSQEE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | RLTVDK | | | | | | | | | | | | |
| | SRWQEG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSL | | | | | | | | | | | | |
| | GK (SEQ | | | | | | | | | | | | |
| | ID NO: | | | | | | | | | | | | |
| | 69) | | | | | | | | | | | | |
| cetrelimab | QVQLVQ | EIVLTQS | QVQLVQ | EIVLTQS | QVQLVQ | EIVLTQS | GTFS | WMG | RPGL | QSVR | LLIYD | QQR | ce- |
| | SGAEVK | PATLSLS | SGAEVK | PATLSLS | SGAEVK | PATLSLS | SYAI | GIIPI | AAAY | SYLA | ASNR | NYW | trelimab |
| | KPGSSV | PGERAT | KPGSSV | PGERAT | KPGSSV | PGERAT | S (27- | FDTA | DTGS | (27- | AT | PL | |
| | KVSCKA | LSCRAS | KVSCKA | LSCRAS | KVSCKA | LSCRAS | 35) | NY | LDY | 34) | (46- | (89- | |
| | SGGTFS | QSVRSY | SGGTFS | QSVRSY | SGGTFS | QSVRSY | (SEQ | (47- | (98- | (SEQ | 56) | 96) | |
| | SYAISWV | LAWYQQ | SYAISWV | LAWYQQ | SYAISWV | LAWYQQ | ID | 60) | 112) | ID | (SEQ | (SEQ | |
| | RQAPGQ | KPGQAP | RQAPGQ | KPGQAP | RQAPGQ | KPGQAP | NO: | (SEQ | (SEQ | NO: | ID | ID | |
| | GLEWMG | RLLIYDA | GLEWMG | RLLIYDA | GLEWMG | RLLIYDA | 234) | ID | ID | 334) | NO: | NO: | |
| | GIIPIFDT | SNRATGI | GIIPIFDT | SNRATGI | GIIPIFDT | SNRATGI | | NO: | NO: | | 344) | 389) | |
| | ANYAQK | PARFSG | ANYAQK | PARFSG | ANYAQK | PARFSG | | 275) | 304) | | | | |
| | FQGRVTI | SGSGTD | FQGRVTI | SGSGTD | FQGRVTI | SGSGTD | | | | | | | |
| | TADEST | FTLTISSL | TADEST | FTLTISSL | TADEST | FTLTISSL | | | | | | | |
| | STAYME | EPEDFA | STAYME | EPEDFA | STAYME | EPEDFA | | | | | | | |
| | LSSLRSE | VYYCQQ | LSSLRSE | VYYCQQ | LSSLRSE | VYYCQQ | | | | | | | |
| | DTAVYY | RNYWPL | DTAVYY | RNYWPL | DTAVYY | RNYWPL | | | | | | | |
| | CARPGL | TFGQGT | CARPGL | TFGQGT | CARPGL | TFGQGT | | | | | | | |
| | AAAYDT | KVEIKRT | AAAYDT | KVEIK | AAAYDT | KVEIKGG | | | | | | | |
| | GSLDYW | VAAPSV | GSLDYW | (SEQ ID | GSLDYW | GGSGGG | | | | | | | |
| | GQGTLV | FIFPPSD | GQGTLV | NO: 160) | GQGTLV | GSGGGG | | | | | | | |
| | TVSSAST | EQLKSG | TVSS | | TVSSGG | SQVQLV | | | | | | | |
| | KGPSVF | TASVVCL | (SEQ ID | | GGSGGG | QSGAEV | | | | | | | |
| | PLAPCS | LNNFYP | NO: 130) | | GSGGGG | KKPGSS | | | | | | | |
| | RSTSES | REAKVQ | | | SEIVLTQ | VKVSCK | | | | | | | |
| | TAALGCL | WKVDNA | | | SPATLSL | ASGGTF | | | | | | | |
| | VKDYFP | LQSGNS | | | SPGERA | SSYAISW | | | | | | | |
| | EPVTVS | QESVTE | | | TLSCRA | VRQAPG | | | | | | | |
| | WNSGAL | QDSKDS | | | SQSVRS | QGLEWM | | | | | | | |
| | TSGVHT | TYSLSST | | | YLAWYQ | GGIIPIF | | | | | | | |
| | FPAVLQ | LTLSKAD | | | QKPGQA | DTANYAQ | | | | | | | |
| | SSGLYSL | YEKHKV | | | PRLLIYD | KFQGRV | | | | | | | |
| | SSVVTV | YACEVT | | | ASNRAT | TITADES | | | | | | | |
| | PSSSLG | HQGLSS | | | GIPARFS | TSTAYM | | | | | | | |
| | TKTYTC | PVTKSF | | | GSGSGT | ELSSLRS | | | | | | | |
| | NVDHKP | NRGEC | | | DFTLTIS | EDTAVY | | | | | | | |
| | SNTKVD | (SEQ ID | | | SLEPEDF | YCARPG | | | | | | | |
| | KRVESK | NO: 100) | | | AVYYCQ | LAAAYDT | | | | | | | |
| | YGPPCP | | | | QRNYWP | GSLDYW | | | | | | | |
| | PCPAPE | | | | LTFGQG | GQGTLV | | | | | | | |
| | FLGGPS | | | | TKVEIK | TVSS | | | | | | | |
| | VFLFPPK | | | | (SEQ ID | (SEQ ID | | | | | | | |
| | PKDTLMI | | | | NO: 190) | NO: 220) | | | | | | | |
| | SRTPEV | | | | | | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 70) | | | | | | | | | | | | |
| nofazinlimab | QVQLVQ | DVVMTQ | QVQLVQ | DVVMTQ | QVQLVQ | DVVMTQ | FTFT | YLGY | IIGYF | QSLL | RLIYL | MQLT | no- |
| | SGAEVK | SPLSLPV | SGAEVK | SPLSLPV | SGAEVK | SPLSLPV | TYYI | INMG | DY | DSD | VSTL | HWP | fazinlimab |
| | KPGSSV | TLGQPA | KPGSSV | TLGQPA | KPGSSV | TLGQPA | S (27- | SGGT | (98- | GGTY | GS | Y (94- | |
| | KVSCKA | SISCRSS | KVSCKA | SISCRSS | KVSCKA | SISCRSS | 35) | NYN | 104) | LY | (51- | 101) | |
| | SGFTFTT | QSLLDS | SGFTFTT | QSLLDS | SGFTFTT | QSLLDS | (SEQ | (47- | (SEQ | (27- | 61) | (SEQ | |
| | YYISWV | DGGTYL | YYISWV | DGGTYL | YYISWV | DGGTYL | ID | 61) | ID | 39) | (SEQ | ID | |
| | RQAPGQ | YWFQQR | RQAPGQ | YWFQQR | RQAPGQ | YWFQQR | NO: | (SEQ | NO: | (SEQ | ID | NO: | |
| | GLEYLG | PGQSPR | GLEYLG | PGQSPR | GLEYLG | PGQSPR | 248) | ID | 305) | ID | NO: | 390) | |
| | YINMGS | RLIYLVS | YINMGS | RLIYLVS | YINMGS | RLIYLVS | | NO: | | NO: | 362) | | |
| | GGTNYN | TLGSGV | GGTNYN | TLGSGV | GGTNYN | TLGSGV | | 276) | | 335) | | | |
| | EKFKGR | PDRFSG | EKFKGR | PDRFSG | EKFKGR | PDRFSG | | | | | | | |
| | VTITADK | SGSGTD | VTITADK | SGSGTD | VTITADK | SGSGTD | | | | | | | |
| | STSTAY | FTLKISR | STSTAY | FTLKISR | STSTAY | FTLKISR | | | | | | | |
| | MELSSL | VEAEDV | MELSSL | VEAEDV | MELSSL | VEAEDV | | | | | | | |
| | RSEDTA | GVYYCM | RSEDTA | GVYYCM | RSEDTA | GVYYCM | | | | | | | |
| | VYYCAII | QLTHWP | VYYCAII | QLTHWP | VYYCAII | QLTHWP | | | | | | | |
| | GYFDYW | YTFGQG | GYFDYW | YTFGQG | GYFDYW | YTFGQG | | | | | | | |
| | GQGTMV | TKLEIKR | GQGTMV | TKLEIK | GQGTMV | TKLEIKG | | | | | | | |
| | TVSSAST | TVAAPS | TVSS | (SEQ ID | TVSSGG | GGGSGG | | | | | | | |
| | KGPSVF | VFIFPPS | (SEQ ID | NO: 161) | GGSGGG | GGSGGG | | | | | | | |
| | PLAPCS | DEQLKS | NO: 131) | | GSGGGG | GSQVQL | | | | | | | |
| | RSTSES | GTASVV | | | SDVVMT | VQSGAE | | | | | | | |
| | TAALGCL | CLLNNFY | | | QSPLSLP | VKKPGS | | | | | | | |
| | VKDYFP | PREAKV | | | VTLGQP | SVKVSC | | | | | | | |
| | EPVTVS | QWKVDN | | | ASISCRS | KASGFT | | | | | | | |
| | WNSGAL | ALQSGN | | | SQSLLD | FTTYYIS | | | | | | | |
| | TSGVHT | SQESVT | | | SDGGTY | WVRQAP | | | | | | | |
| | FPAVLQ | EQDSKD | | | LYWFQQ | GQGLEY | | | | | | | |
| | SSGLYSL | STYSLSS | | | RPGQSP | LGYINM | | | | | | | |
| | SSVVTV | TLTLSKA | | | RRLIYLV | GSGGTN | | | | | | | |
| | PSSSLG | DYEKHK | | | STLGSG | YNEKFK | | | | | | | |
| | TKTYTC | VYACEV | | | VPDRFS | GRVTITA | | | | | | | |
| | NVDHKP | THQGLS | | | GSGSGT | DKSTST | | | | | | | |
| | SNTKVD | SPVTKSF | | | DFTLKIS | AYMELS | | | | | | | |
| | KRVESK | NRGEC | | | RVEAED | SLRSED | | | | | | | |
| | YGPPCP | (SEQ ID | | | VGVYYC | TAVYYC | | | | | | | |
| | PCPAPE | NO: 101) | | | MQLTHW | AIIGYFD | | | | | | | |
| | FLGGPS | | | | PYTFGQ | YWGQGT | | | | | | | |
| | VFLFPPK | | | | GTKLEIK | MVTVSS | | | | | | | |
| | PKDTLMI | | | | (SEQ ID | (SEQ ID | | | | | | | |
| | SRTPEV | | | | NO: 191) | NO: 221) | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 71) | | | | | | | | | | | | |
| pucotenlimab | EVQLVQ | DIVLTQS | EVQLVQ | DIVLTQS | EVQLVQ | DIVLTQS | FTFS | WVA | ARQK | ESVD | LLIYA | QQS | puco- |
| | SGGGLV | PASLAVS | SGGGLV | PASLAVS | SGGGLV | PASLAVS | SYG | TISG | GEA | NYGI | ASNK | KEVP | tenlimab |
| | QPGGSL | PGQRATI | QPGGSL | PGQRATI | QPGGSL | PGQRATI | MS | GGR | WFA | SFMN | GT | W | |
| | KLSCAA | TCRASE | KLSCAA | TCRASE | KLSCAA | TCRASE | (27- | DTYY | Y (97- | (27- | (50- | (93- | |
| | SGFTFS | SVDNYGI | SGFTFS | SVDNYGI | SGFTFS | SVDNYGI | 35) | P (47- | 107) | 38) | 60) | 100) | |
| | SYGMSW | SFMNWF | SYGMSW | SFMNWF | SYGMSW | SFMNWF | (SEQ | 61) | (SEQ | (SEQ | (SEQ | (SEQ | |
| | VRQAPG | QQKPGQ | VRQAPG | QQKPGQ | VRQAPG | QQKPGQ | ID | (SEQ | ID | ID | ID | ID | |
| | KGLDWV | PPKLLIY | KGLDWV | PPKLLIY | KGLDWV | PPKLLIY | NO: | ID | NO: | NO: | VO: | NO: | |
| | ATISGGG | AASNKG | ATISGGG | AASNKG | ATISGGG | AASNKG | 245) | NO: | 306) | 336) | 363) | 382) | |
| | RDTYYP | TGVPAR | RDTYYP | TGVPAR | RDTYYP | TGVPAR | | 272) | | | | | |
| | DSVKGR | FSGSGS | DSVKGR | FSGSGS | DSVKGR | FSGSGS | | | | | | | |
| | FTISRDN | GTDFTL | FTISRDN | GTDFTL | FTISRDN | GTDFTL | | | | | | | |
| | SKNNLYL | NINPMEE | SKNNLYL | NINPMEE | SKNNLYL | NINPMEE | | | | | | | |
| | QMNSLR | NDTAMY | QMNSLR | NDTAMY | QMNSLR | NDTAMY | | | | | | | |
| | AEDTALY | FCQQSK | AEDTALY | FCQQSK | AEDTALY | FCQQSK | | | | | | | |
| | YCARQK | EVPWTF | YCARQK | EVPWTF | YCARQK | EVPWTF | | | | | | | |
| | GEAWFA | GGGTKL | GEAWFA | GGGTKL | GEAWFA | GGGTKL | | | | | | | |
| | YWGQGT | EIKRTVA | YWGQGT | EIK | YWGQGT | EIKGGG | | | | | | | |
| | LVTVSSA | APSVFIF | LVTVSS | (SEQ | LVTVSS | GSGGGG | | | | | | | |
| | STKGPS | PPSDEQ | (SEQ ID | ID NO: | GGGGSG | SGGGGS | | | | | | | |
| | VFPLAPC | LKSGTA | NO: 132) | 162) | GGGSGG | EVQLVQ | | | | | | | |
| | SRSTSE | SVVCLLN | | | GGSDIVL | SGGGLV | | | | | | | |
| | STAALG | NFYPRE | | | TQSPAS | QPGGSL | | | | | | | |
| | CLVKDY | AKVQWK | | | LAVSPG | KLSCAA | | | | | | | |
| | FPEPVTV | VDNALQ | | | QRATITC | SGFTFS | | | | | | | |
| | SWNSGA | SGNSQE | | | RASESV | SYGMSW | | | | | | | |
| | LTSGVH | SVTEQD | | | DNYGISF | VRQAPG | | | | | | | |
| | TFPAVLQ | SKDSTY | | | MNWFQ | KGLDWV | | | | | | | |
| | SSGLYSL | SLSSTLT | | | QKPGQP | ATISGGG | | | | | | | |
| | SSVVTV | LSKADY | | | PKLLIYA | RDTYYP | | | | | | | |
| | PSSSLG | EKHKVY | | | ASNKGT | DSVKGR | | | | | | | |
| | TKTYTC | ACEVTH | | | GVPARF | FTISRDN | | | | | | | |
| | NVDHKP | QGLSSP | | | SGSGSG | SKNNLYL | | | | | | | |
| | SNTKVD | VTKSFN | | | TDFTLNI | QMNSLR | | | | | | | |
| | KRVESK | RGEC | | | NPMEEN | AEDTALY | | | | | | | |
| | YGPPCP | (SEQ ID | | | DTAMYF | YCARQK | | | | | | | |
| | PCPAPE | NO: 102) | | | CQQSKE | GEAWFA | | | | | | | |
| | FLGGPS | | | | VPWTFG | YWGQGT | | | | | | | |
| | VFLFPPK | | | | GGTKLEI | LVTVSS | | | | | | | |
| | PKDTLMI | | | | K (SEQ | (SEQ ID | | | | | | | |
| | TRTPEVT | | | | ID NO: | NO: 222) | | | | | | | |
| | CVVVDV | | | | 192) | | | | | | | | |
| | SQEDPE | | | | | | | | | | | | |
| | VQFNWY | | | | | | | | | | | | |
| | VDGVEV | | | | | | | | | | | | |
| | HNAKTK | | | | | | | | | | | | |
| | PREEQF | | | | | | | | | | | | |
| | NSTYRV | | | | | | | | | | | | |
| | VSVLTPL | | | | | | | | | | | | |
| | HQDWLN | | | | | | | | | | | | |
| | GKEYKC | | | | | | | | | | | | |
| | KVSNKG | | | | | | | | | | | | |
| | LPSSIEK | | | | | | | | | | | | |
| | TISKAKG | | | | | | | | | | | | |
| | QPREPQ | | | | | | | | | | | | |
| | VYTLPPS | | | | | | | | | | | | |
| | QEEMTK | | | | | | | | | | | | |
| | NQVSLT | | | | | | | | | | | | |
| | CLVKGF | | | | | | | | | | | | |
| | YPSDIAV | | | | | | | | | | | | |
| | EWESNG | | | | | | | | | | | | |
| | QPENNY | | | | | | | | | | | | |
| | KTTPPVL | | | | | | | | | | | | |
| | DSDGSF | | | | | | | | | | | | |
| | FLYSRLT | | | | | | | | | | | | |
| | VDKSRW | | | | | | | | | | | | |
| | QEGNVF | | | | | | | | | | | | |
| | SCSVMH | | | | | | | | | | | | |
| | EALHAH | | | | | | | | | | | | |
| | YTQKSL | | | | | | | | | | | | |
| | SLSLGK | | | | | | | | | | | | |
| | (SEQ ID | | | | | | | | | | | | |
| | NO: 72) | | | | | | | | | | | | |
| sasanlimab | QVQLVQ | DIVMTQS | QVQLVQ | DIVMTQS | QVQLVQ | DIVMTQS | YTFT | WMG | RLST | QSL | LLIY | QND | sasan- |
| | SGAEVK | PDSLAV | SGAEVK | PDSLAV | SGAEVK | PDSLAV | SYWI | NIYP | GTFA | WDS | WTS | YFYP | limab |
| | KPGASV | SLGERA | KPGASV | SLGERA | KPGASV | SLGERA | N (27- | GSSL | Y (98- | GNQ | YRES | H (95- | |
| | KVSCKA | TINCKSS | KVSCKA | TINCKSS | KVSCKA | TINCKSS | 35) | TNYN | 106) | KNFL | (52- | 102) | |
| | SGYTFT | QSLWDS | SGYTFT | QSLWDS | SGYTFT | QSLWDS | (SEQ | (47- | (SEQ | T (27- | 62) | (SEQ | |
| | SYWINW | GNQKNF | SYWINW | GNQKNF | SYWINW | GNQKNF | ID | 61) | ID | 40) | (SEQ | ID | |
| | VRQAPG | LTWYQQ | VRQAPG | LTWYQQ | VRQAPG | LTWYQQ | NO: | (SEQ | NO: | (SEQ | ID | NO: | |
| | QGLEWM | KPGQPP | QGLEWM | KPGQPP | QGLEWM | KPGQPP | 249) | ID | 307) | ID | NO: | 391) | |
| | GNIYPGS | KLLIYWT | GNIYPGS | KLLIYWT | GNIYPGS | KLLIYWT | | NO: | | NO: | 364) | | |
| | SLTNYN | SYRESG | SLTNYN | SYRESG | SLTNYN | SYRESG | | 277) | | 337) | | | |
| | EKFKNR | VPDRFS | EKFKNR | VPDRFS | EKFKNR | VPDRFS | | | | | | | |
| | VTMTRD | GSGSGT | VTMTRD | GSGSGT | VTMTRD | GSGSGT | | | | | | | |
| | TSTSTVY | DFTLTIS | TSTSTVY | DFTLTIS | TSTSTVY | DFTLTIS | | | | | | | |
| | MELSSL | SLQAED | MELSSL | SLQAED | MELSSL | SLQAED | | | | | | | |
| | RSEDTA | VAVYYC | RSEDTA | VAVYYC | RSEDTA | VAVYYC | | | | | | | |
| | VYYCAR | QNDYFY | VYYCAR | QNDYFY | VYYCAR | QNDYFY | | | | | | | |
| | LSTGTFA | PHTFGG | LSTGTFA | PHTFGG | LSTGTFA | PHTFGG | | | | | | | |
| | YWGQGT | GTKVEIK | YWGQGT | GTKVEIK | YWGQGT | GTKVEIK | | | | | | | |
| | LVTVSSA | RTVAAP | LVTVSS | (SEQ ID | LVTVSS | GGGGSG | | | | | | | |
| | STKGPS | SVFIFPP | (SEQ ID | NO: 163) | GGGGSG | GGGSGG | | | | | | | |
| | VFPLAPC | SDEQLK | NO: 133) | | GGGSGG | GGSQVQ | | | | | | | |
| | SRSTSE | SGTASV | | | GGSDIV | LVQSGA | | | | | | | |
| | STAALG | VCLLNNF | | | MTQSPD | EVKKPG | | | | | | | |
| | CLVKDY | YPREAK | | | SLAVSLG | ASVKVS | | | | | | | |
| | FPEPVTV | VQWKVD | | | ERATINC | CKASGY | | | | | | | |
| | SWNSGA | NALQSG | | | KSSQSL | TFTSYWI | | | | | | | |
| | LTSGVH | NSQESV | | | WDSGN | NWVRQA | | | | | | | |
| | TFPAVLQ | TEQDSK | | | QKNFLT | PGQGLE | | | | | | | |
| | SSGLYSL | DSTYSLS | | | WYQQKP | WMGNIY | | | | | | | |
| | SSVVTV | STLTLSK | | | GQPPKL | PGSSLT | | | | | | | |
| | PSSSLG | ADYEKH | | | LIYWTSY | NYNEKF | | | | | | | |
| | TKTYTC | KVYACE | | | RESGVP | KNRVTM | | | | | | | |
| | NVDHKP | VTHQGL | | | DRFSGS | TRDTST | | | | | | | |
| | SNTKVD | SSPVTK | | | GSGTDF | STVYME | | | | | | | |
| | KRVESK | SFNRGE | | | TLTISSL | LSSLRSE | | | | | | | |
| | YGPPCP | C (SEQ | | | QAEDVA | DTAVYY | | | | | | | |
| | PCPAPE | ID NO: | | | VYYCQN | CARLST | | | | | | | |
| | FLGGPS | 103) | | | DYFYPH | GTFAYW | | | | | | | |
| | VFLFPPK | | | | TFGGGT | GQGTLV | | | | | | | |
| | PKDTLMI | | | | KVEIK | TVSS | | | | | | | |
| | SRTPEV | | | | (SEQ ID | (SEQ ID | | | | | | | |
| | TCVVVD | | | | NO: 193) | NO: 223) | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 73) | | | | | | | | | | | | |
| balstilimab | QVQLVE | EIVMTQS | QVQLVE | EIVMTQS | QVQLVE | EIVMTQS | FTFS | WVA | SNG | QSVS | LLIYG | QQY | bals- |
| | SGGGVV | PATLSVS | SGGGVV | PATLSVS | SGGGVV | PATLSVS | SYG | VIWY | DH | SNLA | ASTR | NNW | tilimab |
| | QPGRSL | PGERAT | QPGRSL | PGERAT | QPGRSL | PGERAT | MH | DGS | (98- | (27- | AT | PR | |
| | RLSCAA | LSCRAS | RLSCAA | LSCRAS | RLSCAA | LSCRAS | (27- | NKYY | 102) | 34) | (46- | (89- | |
| | SGFTFS | QSVSSN | SGFTFS | QSVSSN | SGFTFS | QSVSSN | 35) | (47- | (SEQ | (SEQ | 56) | 96) | |
| | SYGMH | LAWYQQ | SYGMH | LAWYQQ | SYGMH | LAWYQQ | (SEQ | 60) | ID | ID | (SEQ | (SEQ | |
| | WVRQAP | KPGQAP | WVRQAP | KPGQAP | WVRQAP | KPGQAP | ID | (SEQ | NO: | NO: | ID | ID | |
| | GKGLEW | RLLIYGA | GKGLEW | RLLIYGA | GKGLEW | RLLIYGA | NO: | ID | 308) | 338) | NO: | NO: | |
| | VAVIWY | STRATGI | VAVIWY | STRATGI | VAVIWY | STRATGI | 250) | NO: | | | 365) | 392) | |
| | DGSNKY | PARFSG | DGSNKY | PARFSG | DGSNKY | PARFSG | | 278) | | | | | |
| | YADSVK | SGSGTE | YADSVK | SGSGTE | YADSVK | SGSGTE | | | | | | | |
| | GRFTISR | FTLTISSL | GRFTISR | FTLTISSL | GRFTISR | FTLTISSL | | | | | | | |
| | DNSKNT | QSEDFA | DNSKNT | QSEDFA | DNSKNT | QSEDFA | | | | | | | |
| | LYLQMN | VYYCQQ | LYLQMN | VYYCQQ | LYLQMN | VYYCQQ | | | | | | | |
| | SLRAED | YNNWPR | SLRAED | YNNWPR | SLRAED | YNNWPR | | | | | | | |
| | TAVYYC | TFGQGT | TAVYYC | TFGQGT | TAVYYC | TFGQGT | | | | | | | |
| | ASNGDH | KVEIKRT | ASNGDH | KVEIK | ASNGDH | KVEIKGG | | | | | | | |
| | WGQGTL | VAAPSV | WGQGTL | (SEQ ID | WGQGTL | GGSGGG | | | | | | | |
| | VTVSSA | FIFPPSD | VTVSS | NO: 164) | VTVSSG | GSGGGG | | | | | | | |
| | STKGPS | EQLKSG | (SEQ ID | | GGGSGG | SQVQLV | | | | | | | |
| | VFPLAPC | TASVVCL | NO: 134) | | GGSGGG | ESGGGV | | | | | | | |
| | SRSTSE | LNNFYP | | | GSEIVMT | VQPGRS | | | | | | | |
| | STAALG | REAKVQ | | | QSPATL | LRLSCAA | | | | | | | |
| | CLVKDY | WKVDNA | | | SVSPGE | SGFTFS | | | | | | | |
| | FPEPVTV | LQSGNS | | | RATLSC | SYGMH | | | | | | | |
| | SWNSGA | QESVTE | | | RASQSV | WVRQAP | | | | | | | |
| | LTSGVH | QDSKDS | | | SSNLAW | GKGLEW | | | | | | | |
| | TFPAVLQ | TYSLSST | | | YQQKPG | VAVIWY | | | | | | | |
| | SSGLYSL | LTLSKAD | | | QAPRLLI | DGSNKY | | | | | | | |
| | SSVVTV | YEKHKV | | | YGASTR | YADSVK | | | | | | | |
| | PSSSLG | YACEVT | | | ATGIPAR | GRFTISR | | | | | | | |
| | TKTYTC | HQGLSS | | | FSGSGS | DNSKNT | | | | | | | |
| | NVDHKP | PVTKSF | | | GTEFTLT | LYLQMN | | | | | | | |
| | SNTKVD | NRGEC | | | ISSLQSE | SLRAED | | | | | | | |
| | KRVESK | (SEQ ID | | | DFAVYY | TAVYYC | | | | | | | |
| | YGPPCP | NO: 104) | | | CQQYNN | ASNGDH | | | | | | | |
| | PCPAPE | | | | WPRTFG | WGQGTL | | | | | | | |
| | FLGGPS | | | | QGTKVEI | VTVSS | | | | | | | |
| | VFLFPPK | | | | K (SEQ | (SEQ ID | | | | | | | |
| | PKDTLMI | | | | ID NO: | NO: 224) | | | | | | | |
| | SRTPEV | | | | 194) | | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLGK | | | | | | | | | | | | |
| | (SEQ ID | | | | | | | | | | | | |
| | NO: 74) | | | | | | | | | | | | |
| geptanolimab | QIQLVQS | DIVLTQS | QIQLVQS | DIVLTQS | QIQLVQS | DIVLTQS | YTFT | WMG | CARD | ESVD | LLIYR | QQS | gep- |
| | GSELKK | PASLAVS | GSELKK | PASLAVS | GSELKK | PASLAVS | NFG | WISG | VFDY | NYGY | ASNL | NADP | tanolimab |
| | PGASVK | PGQRATI | PGASVK | PGQRATI | PGASVK | PGQRATI | MNW | YTRE | (96- | SFMN | ES | T (93- | |
| | VSCKAS | TCRASE | VSCKAS | TCRASE | VSCKAS | TCRASE | (27- | PTY | 103) | (27- | (50- | 100) | |
| | GYTFTN | SVDNYG | GYTFTN | SVDNYG | GYTFTN | SVDNYG | 36) | (47- | (SEQ | 38) | 60) | (SEQ | |
| | FGMNWV | YSFMNW | FGMNWV | YSFMNW | FGMNWV | YSFMNW | (SEQ | 60) | ID | (SEQ | (SEQ | ID | |
| | RQAPGQ | FQQKPG | RQAPGQ | FQQKPG | RQAPGQ | FQQKPG | ID | (SEQ | NO: | ID | ID | NO: | |
| | GLKWMG | QPPKLLI | GLKWMG | QPPKLLI | GLKWMG | QPPKLLI | NO: | ID | 309) | NO: | NO: | 393) | |
| | WISGYT | YRASNL | WISGYT | YRASNL | WISGYT | YRASNL | 251) | NO: | | 339) | 366) | | |
| | REPTYA | ESGVPA | REPTYA | ESGVPA | REPTYA | ESGVPA | | 279) | | | | | |
| | ADFKGR | RFSGSG | ADFKGR | RFSGSG | ADFKGR | RFSGSG | | | | | | | |
| | FVISLDT | SRTDFTL | FVISLDT | SRTDFTL | FVISLDT | SRTDFTL | | | | | | | |
| | SVSTAYL | TINPVEA | SVSTAYL | TINPVEA | SVSTAYL | TINPVEA | | | | | | | |
| | QISSLKA | DDTANY | QISSLKA | DDTANY | QISSLKA | DDTANY | | | | | | | |
| | EDTAVY | YCQQSN | EDTAVY | YCQQSN | EDTAVY | YCQQSN | | | | | | | |
| | YCARDV | ADPTFG | YCARDV | ADPTFG | YCARDV | ADPTFG | | | | | | | |
| | FDYWGQ | QGTKLEI | FDYWGQ | QGTKLEI | FDYWGQ | QGTKLEI | | | | | | | |
| | GTLVTVS | KRTVAA | GTLVTVS | K (SEQ | GTLVTVS | KGGGGS | | | | | | | |
| | SASTKG | PSVFIFP | S (SEQ | ID NO: | SGGGGS | GGGGSG | | | | | | | |
| | PSVFPLA | PSDEQL | ID NO: | 165) | GGGGSG | GGGSQI | | | | | | | |
| | PCSRST | KSGTAS | 135) | | GGGSDI | QLVQSG | | | | | | | |
| | SESTAAL | VVCLLN | | | VLTQSP | SELKKP | | | | | | | |
| | GCLVKD | NFYPRE | | | ASLAVSP | GASVKV | | | | | | | |
| | YFPEPVT | AKVQWK | | | GQRATIT | SCKASG | | | | | | | |
| | VSWNSG | VDNALQ | | | CRASES | YTFTNF | | | | | | | |
| | ALTSGV | SGNSQE | | | VDNYGY | GMNWV | | | | | | | |
| | HTFPAVL | SVTEQD | | | SFMNWF | RQAPGQ | | | | | | | |
| | QSSGLY | SKDSTY | | | QQKPGQ | GLKWMG | | | | | | | |
| | SLSSVVT | SLSSTLT | | | PPKLLIY | WISGYT | | | | | | | |
| | VPSSSL | LSKADY | | | RASNLE | REPTYA | | | | | | | |
| | GTKTYT | EKHKVY | | | SGVPAR | ADFKGR | | | | | | | |
| | CNVDHK | ACEVTH | | | FSGSGS | FVISLDT | | | | | | | |
| | PSNTKV | QGLSSP | | | RTDFTLT | SVSTAYL | | | | | | | |
| | DKRVES | VTKSFN | | | INPVEAD | QISSLKA | | | | | | | |
| | KYGPPC | RGEC | | | DTANYY | EDTAVY | | | | | | | |
| | PPCPAP | (SEQ ID | | | CQQSNA | YCARDV | | | | | | | |
| | EFLGGP | NO: 105) | | | DPTFGQ | FDYWGQ | | | | | | | |
| | SVFLFPP | | | | GTKLEIK | GTLVTVS | | | | | | | |
| | KPKDTL | | | | (SEQ ID | S (SEQ | | | | | | | |
| | MISRTPE | | | | NO: 195) | ID NO: | | | | | | | |
| | VTCVVV | | | | | 225) | | | | | | | |
| | DVSQED | | | | | | | | | | | | |
| | PEVQFN | | | | | | | | | | | | |
| | WYVDGV | | | | | | | | | | | | |
| | EVHNAK | | | | | | | | | | | | |
| | TKPREE | | | | | | | | | | | | |
| | QFNSTY | | | | | | | | | | | | |
| | RVVSVLT | | | | | | | | | | | | |
| | VLHQDW | | | | | | | | | | | | |
| | LNGKEY | | | | | | | | | | | | |
| | KCKVSN | | | | | | | | | | | | |
| | KGLPSSI | | | | | | | | | | | | |
| | EKTISKA | | | | | | | | | | | | |
| | KGQPRE | | | | | | | | | | | | |
| | PQVYTL | | | | | | | | | | | | |
| | PPSQEE | | | | | | | | | | | | |
| | MTKNQV | | | | | | | | | | | | |
| | SLTCLVK | | | | | | | | | | | | |
| | GFYPSDI | | | | | | | | | | | | |
| | AVEWES | | | | | | | | | | | | |
| | NGQPEN | | | | | | | | | | | | |
| | NYKTTP | | | | | | | | | | | | |
| | PVLDSD | | | | | | | | | | | | |
| | GSFFLYS | | | | | | | | | | | | |
| | RLTVDK | | | | | | | | | | | | |
| | SRWQEG | | | | | | | | | | | | |
| | NVFSCS | | | | | | | | | | | | |
| | VMHEAL | | | | | | | | | | | | |
| | HNHYTQ | | | | | | | | | | | | |
| | KSLSLSL | | | | | | | | | | | | |
| | G (SEQ | | | | | | | | | | | | |
| | ID NO: | | | | | | | | | | | | |
| | 75) | | | | | | | | | | | | |
| retifanlimab | QVQLVQ | EIVLTQS | QVQLVQ | EIVLTQS | QVQLVQ | EIVLTQS | YSFT | WIGV | REHY | ESVD | LLIHA | QQS | re- |
| | SGAEVK | PATLSLS | SGAEVK | PATLSLS | SGAEVK | PATLSLS | SYW | IHPS | GTSP | NYG | ASNQ | KEVP | tifanlimab |
| | KPGASV | PGERAT | KPGASV | PGERAT | KPGASV | PGERAT | MN | DSET | FAY | MSF | GS | Y (93- | |
| | KVSCKA | LSCRAS | KVSCKA | LSCRAS | KVSCKA | LSCRAS | (27- | WLD | (98- | MN | (50- | 100) | |
| | SGYSFT | ESVDNY | SGYSFT | ESVDNY | SGYSFT | ESVDNY | 35) | (47- | 108) | (27- | 60) | SEQ | |
| | SYWMN | GMSFMN | SYWMN | GMSFMN | SYWMN | GMSFMN | (SEQ | 61) | (SEQ | 38) | (SEQ | ID | |
| | WVRQAP | WFQQKP | WVRQAP | WFQQKP | WVRQAP | WFQQKP | ID | (SEQ | ID | (SEQ | ID | NO: | |
| | GQGLEW | GQPPKL | GQGLEW | GQPPKL | GQGLEW | GQPPKL | NO: | ID | NO: | ID | NO: | 394) | |
| | IGVIHPS | LIHAASN | IGVIHPS | LIHAASN | IGVIHPS | LIHAASN | 252) | NO: | 310) | NO: | 367) | | |
| | DSETWL | QGSGVP | DSETWL | QGSGVP | DSETWL | QGSGVP | | 280) | | 340) | | | |
| | DQKFKD | SRFSGS | DQKFKD | SRFSGS | DQKFKD | SRFSGS | | | | | | | |
| | RVTITVD | GSGTDF | RVTITVD | GSGTDF | RVTITVD | GSGTDF | | | | | | | |
| | KSTSTAY | TLTISSL | KSTSTAY | TLTISSL | KSTSTAY | TLTISSL | | | | | | | |
| | MELSSL | EPEDFA | MELSSL | EPEDFA | MELSSL | EPEDFA | | | | | | | |
| | RSEDTA | VYFCQQ | RSEDTA | VYFCQQ | RSEDTA | VYFCQQ | | | | | | | |
| | VYYCAR | SKEVPY | VYYCAR | SKEVPY | VYYCAR | SKEVPY | | | | | | | |
| | EHYGTS | TFGGGT | EHYGTS | TFGGGT | EHYGTS | TFGGGT | | | | | | | |
| | PFAYWG | KVEIKRT | PFAYWG | KVEIK | PFAYWG | KVEIKGG | | | | | | | |
| | QGTLVT | VAAPSV | QGTLVT | (SEQ ID | QGTLVT | GGSGGG | | | | | | | |
| | VSSAST | FIFPPSD | VSS | NO: 166) | VSSGGG | GSGGGG | | | | | | | |
| | KGPSVF | EQLKSG | (SEQ ID | | GSGGGG | SQVQLV | | | | | | | |
| | PLAPCS | TASVVCL | NO: 136) | | SGGGGS | QSGAEV | | | | | | | |
| | RSTSES | LNNFYP | | | EIVLTQS | KKPGAS | | | | | | | |
| | TAALGCL | REAKVQ | | | PATLSLS | VKVSCK | | | | | | | |
| | VKDYFP | WKVDNA | | | PGERAT | ASGYSF | | | | | | | |
| | EPVTVS | LQSGNS | | | LSCRAS | TSYWMN | | | | | | | |
| | WNSGAL | QESVTE | | | ESVDNY | WVRQAP | | | | | | | |
| | TSGVHT | QDSKDS | | | GMSFMN | GQGLEW | | | | | | | |
| | FPAVLQ | TYSLSST | | | WFQQKP | IGVIHPS | | | | | | | |
| | SSGLYSL | LTLSKAD | | | GQPPKL | DSETWL | | | | | | | |
| | SSVVTV | YEKHKV | | | LIHAASN | DQKFKD | | | | | | | |
| | PSSSLG | YACEVT | | | QGSGVP | RVTITVD | | | | | | | |
| | TKTYTC | HQGLSS | | | SRFSGS | KSTSTAY | | | | | | | |
| | NVDHKP | PVTKSF | | | GSGTDF | MELSSL | | | | | | | |
| | SNTKVD | NRGEC | | | TLTISSL | RSEDTA | | | | | | | |
| | KRVESK | (SEQ ID | | | EPEDFA | VYYCAR | | | | | | | |
| | YGPPCP | NO: 106) | | | VYFCQQ | EHYGTS | | | | | | | |
| | PCPAPE | | | | SKEVPY | PFAYWG | | | | | | | |
| | FLGGPS | | | | TFGGGT | QGTLVT | | | | | | | |
| | VFLFPPK | | | | KVEIK | VSS | | | | | | | |
| | PKDTLMI | | | | (SEQ ID | (SEQ ID | | | | | | | |
| | SRTPEV | | | | NO: 196) | NO: 226) | | | | | | | |
| | TCVVVD | | | | | | | | | | | | |
| | VSQEDP | | | | | | | | | | | | |
| | EVQFNW | | | | | | | | | | | | |
| | YVDGVE | | | | | | | | | | | | |
| | VHNAKT | | | | | | | | | | | | |
| | KPREEQ | | | | | | | | | | | | |
| | FNSTYR | | | | | | | | | | | | |
| | VVSVLTV | | | | | | | | | | | | |
| | LHQDWL | | | | | | | | | | | | |
| | NGKEYK | | | | | | | | | | | | |
| | CKVSNK | | | | | | | | | | | | |
| | GLPSSIE | | | | | | | | | | | | |
| | KTISKAK | | | | | | | | | | | | |
| | GQPREP | | | | | | | | | | | | |
| | QVYTLP | | | | | | | | | | | | |
| | PSQEEM | | | | | | | | | | | | |
| | TKNQVS | | | | | | | | | | | | |
| | LTCLVKG | | | | | | | | | | | | |
| | FYPSDIA | | | | | | | | | | | | |
| | VEWESN | | | | | | | | | | | | |
| | GQPENN | | | | | | | | | | | | |
| | YKTTPPV | | | | | | | | | | | | |
| | LDSDGS | | | | | | | | | | | | |
| | FFLYSRL | | | | | | | | | | | | |
| | TVDKSR | | | | | | | | | | | | |
| | WQEGNV | | | | | | | | | | | | |
| | FSCSVM | | | | | | | | | | | | |
| | HEALHN | | | | | | | | | | | | |
| | HYTQKS | | | | | | | | | | | | |
| | LSLSLG | | | | | | | | | | | | |
| | (SEQ ID | | | | | | | | | | | | |
| | NO: 76) | | | | | | | | | | | | |

In some embodiments, the extracellular binding domain of the anti-PD-1 CAR comprises an scFv derived from any of the anti-PD-1 antibodies shown in Table 8. scFvs derived from the anti-PD-1 antibodies shown in Table 8. also comprise the heavy chain variable region (V_{H}) and the light chain variable region (V_{L}) of the anti-PD-1 antibody connected by a linker. Exemplary amino acid sequences of the anti-PD-1 antibody-derived scFv are shown in Table 8. In some embodiments, the extracellular binding domain specific to PD-1 comprises an scFv derived from a portion of an anti-PD-1 antibody set forth in Table 8.

In some embodiments, the anti-PD-1 scFv comprises V_{H} a comprising or consisting of an amino acid sequence set forth in SEQ ID NO: 5, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 5.

In some embodiments, the anti-PD-1 scFv comprises a V_{H} comprising or consisting of an amino acid sequence set forth in SEQ ID NO: 5, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 5.

In some embodiments, the anti-PD-1 scFv comprises a V_{H} comprising or consisting of an amino acid sequence set forth in SEQ ID NO: 5, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 5.

In some embodiments, the anti-PD-1 scFv comprises a V_{H} comprising or consisting of an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to any of the amino acid sequences set forth in Table 8.

In some embodiments, the anti-PD-1 scFv comprises a V_{H} comprising or consisting of an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to any of the amino acid sequences set forth in Table 8.

In some embodiments, the anti-PD-1 scFv comprises a V_{H} comprising or consisting of an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to any of the amino acid sequences set forth in Table 8.

In some embodiments, the anti-PD-1 scFv comprises a V_{L} comprising or consisting of an amino acid sequence set forth in SEQ ID NO: 10, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 10.

In some embodiments, the anti-PD-1 scFv comprises a V_{L} comprising or consisting of an amino acid sequence set forth in SEQ ID NO: 10, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 10.

In some embodiments, the anti-PD-1 scFv comprises a V_{L} comprising or consisting of an amino acid sequence set forth in SEQ ID NO: 10, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 10.

In some embodiments, the anti-PD-1 scFv comprises a V_{L} comprising or consisting of an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to any of the amino acid sequences set forth in Table 8.

In some embodiments, the anti-PD-1 scFv comprises a V_{L} comprising or consisting of an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or about 100% identical) to any of the amino acid sequences set forth in Table 8.

In some embodiments, the anti-PD-1 scFv comprises a V_{L} comprising or consisting of an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to any of the amino acid sequences set forth in Table 8.

In some embodiments, the V_{H} and V_{L} are connected in either orientation by a linker (i.e., V_{H}-linker-V_{L} or V_{L}-linker-V_{H}), for example, a 3×GGGGS linker (SEQ ID NO: 9). In some embodiments, the anti-PD-1 scFv comprises or consists of an amino acid sequence set forth in SEQ ID NO: 4 or SEQ ID NO: 14.

In some embodiments, the anti-PD-1 scFv comprises or consists of an amino acid sequence an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 4 or SEQ ID NO: 14.

In some embodiments, the anti-PD-1 scFv comprises or consists of an amino acid sequence an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 4 or SEQ ID NO: 14.

In some embodiments, the anti-PD-1 scFv comprises or consists of an amino acid sequence an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 4 or SEQ ID NO: 14.

In some embodiments, a different linker, for example, a Whitlow linker (SEQ ID NO: 15), may be used.

In some embodiments, the anti-PD-1 scFv comprises one or more (e.g., two, three, four, five, or six) CDRs having amino acid sequences set forth in SEQ ID NOs: 6-8 and 11-13. In some embodiments, the anti-PD-1 scFv comprises one or more (e.g., two, three, four, five, or six) CDRs having amino acid sequences set forth in Table 8. In some embodiments, the anti-PD-1 scFv may comprise a heavy chain with one or more CDRs having amino acid sequences set forth in SEQ ID NOs: 6-8. In some embodiments, the anti-PD-1 scFv may comprise a heavy chain with one or more CDRs having amino acid sequences set forth in Table 8. In some embodiments, the anti-PD-1 scFv may comprise a light chain with one or more CDRs having amino acid sequences set forth in SEQ ID NOS: 11-13. In some embodiments, the anti-PD-1 scFv may comprise a light chain with one or more CDRs having amino acid sequences set forth in Table 8.

In some embodiments, the anti-PD-1 scFv may comprise one or more CDRs comprising one or more amino acid substitutions or comprising a sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to any of the sequences identified.

In some embodiments, the anti-PD-1 scFv may comprise one or more CDRs comprising one or more amino acid substitutions or comprising a sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to any of the sequences identified.

In some embodiments, the anti-PD-1 scFv may comprise one or more CDRs comprising one or more amino acid substitutions or comprising a sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to any of the sequences identified.

In some embodiments, the anti-PD-1 scFv comprises a V_{H}, wherein the V_{H} comprises (1) a CDR1 having an amino acid sequence set forth in SEQ ID NO: 6, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 6; (2) a CDR2 having an amino acid sequence set forth in SEQ ID NO: 7, or an amino acid sequence that is about 50% (e.g., about 50%, about 55%, about 60%, about 65%, about 70%, about 75%, about 80%, about 85%, about 90%, about 95%, about 96%, about 97%, about 98%, about 99%, or about 100% identical) identical to the amino acid sequence set forth in SEQ ID NO: 7; and/or (3) a CDR3 having an amino acid sequence set forth in SEQ ID NO: 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 8.

In some embodiments, the anti-PD-1 scFv comprises a V_{H}, wherein the V_{H} comprises (1) a CDR1 having an amino acid sequence set forth in SEQ ID NO: 6, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 6; (2) a CDR2 having an amino acid sequence set forth in SEQ ID NO: 7, or an amino acid sequence that is at least 50% (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) identical to the amino acid sequence set forth in SEQ ID NO: 7; and/or (3) a CDR3 having an amino acid sequence set forth in SEQ ID NO: 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 8.

In some embodiments, the anti-PD-1 scFv comprises a V_{H}, wherein the V_{H} comprises (1) a CDR1 having an amino acid sequence set forth in SEQ ID NO: 6, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 6; (2) a CDR2 having an amino acid sequence set forth in SEQ ID NO: 7, or an amino acid sequence that is at least about 50% (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) identical to the amino acid sequence set forth in SEQ ID NO: 7; and/or (3) a CDR3 having an amino acid sequence set forth in SEQ ID NO: 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 8.

In some embodiments, the anti-PD-1 scFv comprises a V_{H}, wherein the V_{H} comprises (1) a CDR1 having an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in Table 8; (2) a CDR2 having an amino acid sequence set forth in Table 8, or an amino acid sequence that is about 50% (e.g., about 50%, about 55%, about 60%, about 65%, about 70%, about 75%, about 80%, about 85%, about 90%, about 95%, about 96%, about 97%, about 98%, about 99%, or about 100% identical) identical to the amino acid sequence set forth in Table 8; and/or (3) a CDR3 having an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in Table 8.

In some embodiments, the anti-PD-1 scFv comprises a V_{H}, wherein the V_{H} comprises (1) a CDR1 having an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in Table 8; (2) a CDR2 having an amino acid sequence set forth in Table 8, or an amino acid sequence that is at least 50% (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) identical to the amino acid sequence set forth in Table 8; and/or (3) a CDR3 having an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in Table 8.

In some embodiments, the anti-PD-1 scFv comprises a V_{H}, wherein the V_{H} comprises (1) a CDR1 having an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in Table 8; (2) a CDR2 having an amino acid sequence set forth in Table 8, or an amino acid sequence that is at least about 50% (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) identical to the amino acid sequence set forth in Table 8; and/or (3) a CDR3 having an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in Table 8.

In some embodiments, the anti-PD-1 scFv comprises a V_{L}, wherein the V_{L} comprises (1) a CDR1 having an amino acid sequence set forth in SEQ ID NO: 11, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 11; (2) a CDR2 having an amino acid sequence set forth in SEQ ID NO: 12, or an amino acid sequence that is about 50% (e.g., about 50%, about 55%, about 60%, about 65%, about 70%, about 75%, about 80%, about 85%, about 90%, about 95%, about 96%, about 97%, about 98%, about 99%, or about 100% identical) identical to the amino acid sequence set forth in SEQ ID NO: 12; and/or (3) a CDR3 having an amino acid sequence set forth in SEQ ID NO: 13, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in SEQ ID NO: 13.

In some embodiments, the anti-PD-1 scFv comprises a V_{L}, wherein the V_{L} comprises (1) a CDR1 having an amino acid sequence set forth in SEQ ID NO: 11, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 11; (2) a CDR2 having an amino acid sequence set forth in SEQ ID NO: 12, or an amino acid sequence that is at least 50% (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) identical to the amino acid sequence set forth in SEQ ID NO: 12; and/or (3) a CDR3 having an amino acid sequence set forth in SEQ ID NO: 13, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% identical) to the amino acid sequence set forth in SEQ ID NO: 13.

In some embodiments, the anti-PD-1 scFv comprises a V_{L}, wherein the V_{L} comprises (1) a CDR1 having an amino acid sequence set forth in SEQ ID NO: 11, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 11; (2) a CDR2 having an amino acid sequence set forth in SEQ ID NO: 12, or an amino acid sequence that is at least about 50% (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) identical to the amino acid sequence set forth in SEQ ID NO: 12; and/or (3) a CDR3 having an amino acid sequence set forth in SEQ ID NO: 13, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in SEQ ID NO: 13.

In some embodiments, the anti-PD-1 scFv comprises a V_{L}, wherein the V_{L} comprises (1) a CDR1 having an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in Table 8; (2) a CDR2 having an amino acid sequence set forth in SEQ ID NO: 12, or an amino acid sequence that is about 50% (e.g., about 50%, about 55%, about 60%, about 65%, about 70%, about 75%, about 80%, about 85%, about 90%, about 95%, about 96%, about 97%, about 98%, about 99%, or about 100% identical) identical to the amino acid sequence set forth in Table 8; and/or (3) a CDR3 having an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or about 100% identical) to the amino acid sequence set forth in Table 8.

In some embodiments, the anti-PD-1 scFv comprises a V_{L}, wherein the V_{L} comprises (1) a CDR1 having an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or about 100% identical) to the amino acid sequence set forth in Table 8; (2) a CDR2 having an amino acid sequence set forth in SEQ ID NO: 12, or an amino acid sequence that is at least 50% (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or about 100% identical) identical to the amino acid sequence set forth in Table 8; and/or (3) a CDR3 having an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or about 100% identical) to the amino acid sequence set forth in Table 8.

In some embodiments, the anti-PD-1 scFv comprises a V_{L}, wherein the V_{L} comprises (1) a CDR1 having an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in Table 8; (2) a CDR2 having an amino acid sequence set forth in SEQ ID NO: 12, or an amino acid sequence that is at least about 50% (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) identical to the amino acid sequence set forth in Table 8; and/or (3) a CDR3 having an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical) to the amino acid sequence set forth in Table 8.

TABLE 2
Exemplary sequences of anti-PD-1scFv and components

| SEQ ID NO: | Sequence | Description |
|---|---|---|
| 4 | QVQLVQSGVEVKKPGASVKVSCKA | Pembrolizumab-derived |
| | SGYTFTNYYMYWVRQAPGQGLEW | anti-PD-1 scFv (V_{H}- |
| | MGGINPSNGGTNFNEKFKNRVTLT | linker-V_{L}) amino acid |
| | TDSSTTTAYMELKSLQFDDTAVYYC | sequence |
| | ARRDYRFDMGFDYWGQGTTVTVS | |
| | SASTKGGGGGSGGGGSGGGGSEI | |
| | VLTQSPATLSLSPGERATLSCRASK | |
| | GVSTSGYSYLHWYQQKPGQAPRLL | |
| | IYLASYLESGVPARFSGSGSGTDFT | |
| | LTISSLEPEDFAVYYCQHSRDLPLTF | |
| | GGGTKVEIKRTVAAPSVFIFPPSDE | |
| | QLKSGTA | |
| 5 | QVQLVQSGVEVKKPGASVKVSCKA | Pembrolizumab-derived |
| | SGYTFTNYYMYWVRQAPGQGLEW | anti-PD-1 scFv heavy |
| | MGGINPSNGGTNFNEKFKNRVTLT | chain variable region |
| | TDSSTTTAYMELKSLQFDDTAVYYC | (V_{H}) amino acid |
| | ARRDYRFDMGFDYWGQGTTVTVS | sequence |
| | SASTKG | |
| 6 | YTFTNYYMY | Pembrolizumab-derived |
| | | anti-PD-1 scFv V_{H} |
| | | CDR1 amino acid |
| | | sequence |
| 7 | WMGGINPSNGGTNF | Pembrolizumab-derived |
| | | anti-PD-1 scFv V_{H} |
| | | CDR2 amino acid |
| | | sequence |
| 8 | RRDYRFDMGFDY | Pembrolizumab-derived |
| | | anti-PD-1 scFv V_{H} |
| | | CDR3 amino acid |
| | | sequence |
| 9 | GGGGSGGGGSGGGGS | 3xGGGGS linker |
| 10 | EIVLTQSPATLSLSPGERATLSCRA | Pembrolizumab-derived |
| | SKGVSTSGYSYLHWYQQKPGQAP | anti-PD-1 scFv light |
| | RLLIYLASYLESGVPARFSGSGSGT | chain variable region |
| | DFTLTISSLEPEDFAVYYCQHSRDL | (V_{L}) amino acid |
| | PLTFGGGTKVEIKRTVAAPSVFIFPP | sequence |
| | SDEQLKSGTA | |
| 11 | KGVSTSGYSYLH | Pembrolizumab-derived |
| | | anti-PD-1 scFv V_{L} |
| | | CDR1 amino acid |
| | | sequence |
| 12 | LLIYLASYLES | Pembrolizumab-derived |
| | | anti-PD-1 scFv V_{L} |
| | | CDR2 amino acid |
| | | sequence |
| 13 | QHSRDLPL | Pembrolizumab-derived |
| | | anti-PD-1 scFv V_{L} |
| | | CDR3 amino acid |
| | | sequence |
| 14 | EIVLTQSPATLSLSPGERATLSCRA | Pembrolizumab-derived |
| | SKGVSTSGYSYLHWYQQKPGQAP | anti-PD-1 scFv (V_{L}- |
| | RLLIYLASYLESGVPARFSGSGSGT | linker-V_{H}) amino acid |
| | DFTLTISSLEPEDFAVYYCQHSRDL | sequence |
| | PLTFGGGTKVEIKRTVAAPSVFIFPP | |
| | SDEQLKSGTAGGGGSGGGGSGGG | |
| | GSQVQLVQSGVEVKKPGASVKVSC | |
| | KASGYTFTNYYMYWVRQAPGQGL | |
| | EWMGGINPSNGGTNFNEKFKNRVT | |
| | LTTDSSTTTAYMELKSLQFDDTAVY | |
| | YCARRDYRFDMGFDYWGQGTTVT | |
| | VSSASTKG | |
| 15 | GSTSGSGKPGSGEGSTKG | Whitlow linker |

In some embodiments, the CAR may comprise a hinge domain, also referred to as a spacer, in between the extracellular binding domain and the transmembrane domain. The terms “hinge” and “spacer” may be used interchangeably in the present technology. A hinge domain may be of various lengths depending on the special requirement of the CAR design. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below. The hinge domain may be a short sequence (about 12 amino acids), a medium sequence (about 119 amino acids), or a long sequence (about 223 amino acids). Table 3 also shows exemplary anti-PD-1 CARs (e.g., derived from pembrolizumab) with short, medium, and long hinge domains. These short, medium, and long hinge domains may also be included with an anti-PD-1 CAR comprising one or more amino acid sequences set forth in Table 8.

TABLE 3
Exemplary sequences of hinge domains

| SEQ ID NO: | Sequence | Description |
|---|---|---|
| 16 | TTTPAPRPPTPAPTIASQPLSLRPEACRPA | CD8a hinge domain |
| | AGGAVHTRGLDFACD | |
| 17 | IEVMYPPPYLDNEKSNGTIIHVKGKHLCPS | CD28 hinge domain |
| | PLFPGPSKP | |
| 18 | ESKYGPPCPPCP | IgG4 hinge domain |
| | | (short) |
| 19 | ESKYGPPCPPCPGQPREPQVYTLPPSQE | IgG4 hinge domain |
| | EMTKNQVSLTCLVKGFYPSDIAVEWESNG | (medium) |
| | QPENNYKTTPPVLDSDGSFFLYSRLTVDK | |
| | SRWQEGNVFSCSVMHEALHNHYTQKSLS | |
| | LSLGK | |
| 20 | ESKYGPPCPPCPGPSVFLFPPKPKDTLMI | IgG4 hinge domain |
| | SRTPEVTCVVVDVSQEDPEVQFNWYVDG | (long) |
| | VEVHNAKTKPREEQFQSTYRVVSVLTVLH | |
| | QDWLNGKEYKCKVSNKGLPSSIEKTISKA | |
| | KGQPREPQVYTLPPSQEEMTKNQVSLTC | |
| | LVKGFYPSDIAVEWESNGQPENNYKTTPP | |
| | VLDSDGSFFLYSRLTVDKSRWQEGNVFS | |
| | CSVMHEALHNHYTQKSLSLSLGK | |

In some embodiments, the CAR may comprise a transmembrane domain. In other embodiments, the transmembrane domain may comprise a transmembrane region of CD3ζ, CD3ε, CD3γ, CD3δ, CD4, CD5, CD8α, CD8β, CD9, CD16, CD22, CD28, CD32, CD33, CD34, CD37, CD40, CD45, CD64, CD80, CD86, OX40/CD134, 4-1BB/CD137, CD40L/CD154, FAS, FcεRIγ, FGFR2B, TCRα, TCRβ, or VEGFR2, or a functional variant thereof, including the human versions of each of these sequences. Table 4 provides the amino acid sequences of exemplary transmembrane domains.

TABLE 4
Exemplary sequences of transmembrane domains

| SEQ | | |
|---|---|---|
| ID | | |
| NO: | Sequence | Description |
| 21 | IYIWAPLAGTCG | CD8α transmembrane domain |
| | VLLLSLVITLYC | |
| 22 | (M)FWVLVVVGG | CD28 transmembrane domain |
| | VLACYSLLVTVA | (including an optional |
| | FIIFWV | methionine (M) at the N- |
| | | terminal end) |

In some embodiments, the CAR may comprise an intracellular costimulatory domain and/or intracellular signaling domain. In some embodiments, the intracellular costimulatory domain and/or intracellular signaling domain may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function-associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain comprises one or more signaling domains selected from a CD3 zeta (ζ) domain, an ITAM, a CD28 domain, 4-1BB domain, or a functional variant thereof. Table 5 provides the amino acid sequences of a few exemplary intracellular signaling domains. 4-1BB, also known as CD137, transmits a potent costimulatory signal to T cells, promoting differentiation and enhancing long-term survival of T lymphocytes. CD28 is another costimulatory molecule on T cells. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain (SEQ ID NO: 25) may have a mutation, e.g., a glutamine (Q) to lysine (K) mutation, at amino acid position 14 (SEQ ID NO: 26).

TABLE 5
Exemplary sequences of intracellular signaling domains

| SEQ ID NO: | Sequence | Description |
|---|---|---|
| 23 | KRGRKKLLYIFKQPFMRPVQTTQEE | 4-1BB costimulatory domain |
| | DGCSCRFPEEEEGGCEL | |
| 24 | RSKRSRLLHSDYMNMTPRRPGPTR | CD28 costimulatory domain |
| | KHYQPYAPPRDFAAYRS | |
| 25 | RVKFSRSADAPAYQQGQNQLYNEL | CD3ζ signaling domain |
| | NLGRREEYDVLDKRRGRDPEMGGK | |
| | PRRKNPQEGLYNELQKDKMAEAYS | |
| | EIGMKGERRRGKGHDGLYQGLSTA | |
| | TKDTYDALHMQALPPR | |
| 26 | RVKFSRSADAPAYKQGQNQLYNEL | CD32 signaling domain |
| | NLGRREEYDVLDKRRGRDPEMGGK | (with Q to K mutation at |
| | PRRKNPQEGLYNELQKDKMAEAYS | position 14) |
| | EIGMKGERRRGKGHDGLYQGLSTA | |
| | TKDTYDALHMQALPPR | |

In some embodiments, the anti-PD-1 CAR comprises a GM-CSF signal peptide having an amino acid sequence set forth in SEQ ID NO: 3, an anti-PD-1 scFv having an amino acid sequence set forth in SEQ ID NO: 4 or SEQ ID NO: 14, an IgG4 hinge domain having an amino acid sequence set forth in any one of SEQ ID NOs: 18-20, a CD28 transmembrane domain having an amino acid sequence set forth in SEQ ID NO: 22, a 4-1BB costimulatory domain having an amino acid sequence set forth in SEQ ID NO: 23, and/or a CD3ζ signaling domain having an amino acid sequence set forth in SEQ ID NO: 25 or SEQ ID NO: 26, or variants (i.e., having a sequence that is at least 50%, at least 55%, at least 60%, at least 65%, at least 70%, at least 75%, at least 80% identical, for example, at least 80%, at least 85%, at least 90%, at least 95%, at least 96%, at least 97%, at least 98%, or at least 99 identical to the sequences of the present technology) thereof. Amino acid sequences of exemplary anti-PD-1 CARs according to various embodiments of the present technology are provided in Table 6 below:

TABLE 6
Exemplary sequences of anti-PD-1 CARs

| SEQ ID NO: | Sequence | Description |
|---|---|---|
| 27 | MLLLVTSLLLCELPHPAFLLIPQVQLVQSG | Anti-PD-1 CAR with |
| | VEVKKPGASVKVSCKASGYTFTNYYMYW | pembrolizumab-derived |
| | VRQAPGQGLEWMGGINPSNGGTNFNEKF | scFv (V_{H}-linker-V_{L}), |
| | KNRVTLTTDSSTTTAYMELKSLQFDDTAV | short IgG4 hinge |
| | YYCARRDYRFDMGFDYWGQGTTVTVSSA | domain, CD28 |
| | STKGGGGGSGGGGSGGGGSEIVLTQSPA | transmembrane domain, |
| | TLSLSPGERATLSCRASKGVSTSGYSYLH | 4-1BB costimulatory |
| | WYQQKPGQAPRLLIYLASYLESGVPARFS | domain, and CD3ζ |
| | GSGSGTDFTLTISSLEPEDFAVYYCQHSR | signaling domain |
| | DLPLTFGGGTKVEIKRTVAAPSVFIFPPSD | |
| | EQLKSGTAESKYGPPCPPCPMFWVLVVV | |
| | GGVLACYSLLVTVAFIIFWVKRGRKKLLYIF | |
| | KQPFMRPVQTTQEEDGCSCRFPEEEEGG | |
| | CELRVKFSRSADAPAYQQGQNQLYNELN | |
| | LGRREEYDVLDKRRGRDPEMGGKPRRKN | |
| | PQEGLYNELQKDKMAEAYSEIGMKGERR | |
| | RGKGHDGLYQGLSTATKDTYDALHMQAL | |
| | PPR | |
| 28 | MLLLVTSLLLCELPHPAFLLIPQVQLVQSG | Anti-PD-1 CAR with |
| | VEVKKPGASVKVSCKASGYTFTNYYMYW | pembrolizumab-derived |
| | VRQAPGQGLEWMGGINPSNGGTNFNEKF | scFv (V_{H}-linker-V_{L}), |
| | KNRVTLTTDSSTTTAYMELKSLQFDDTAV | medium IgG4 hinge |
| | YYCARRDYRFDMGFDYWGQGTTVTVSSA | domain, CD28 |
| | STKGGGGGSGGGGSGGGGSEIVLTQSPA | transmembrane domain, |
| | TLSLSPGERATLSCRASKGVSTSGYSYLH | 4-1BB costimulatory |
| | WYQQKPGQAPRLLIYLASYLESGVPARFS | domain, and CD34 |
| | GSGSGTDFTLTISSLEPEDFAVYYCQHSR | signaling domain |
| | DLPLTFGGGTKVEIKRTVAAPSVFIFPPSD | |
| | EQLKSGTAESKYGPPCPPCPGQPREPQV | |
| | YTLPPSQEEMTKNQVSLTCLVKGFYPSDI | |
| | AVEWESNGQPENNYKTTPPVLDSDGSFF | |
| | LYSRLTVDKSRWQEGNVFSCSVMHEALH | |
| | NHYTQKSLSLSLGKMFWVLVVVGGVLAC | |
| | YSLLVTVAFIIFWVKRGRKKLLYIFKQPFMR | |
| | PVQTTQEEDGCSCRFPEEEEGGCELRVK | |
| | FSRSADAPAYQQGQNQLYNELNLGRREE | |
| | YDVLDKRRGRDPEMGGKPRRKNPQEGLY | |
| | NELQKDKMAEAYSEIGMKGERRRGKGHD | |
| | GLYQGLSTATKDTYDALHMQALPPR | |
| 29 | MLLLVTSLLLCELPHPAFLLIPQVQLVQSG | Anti-PD-1 CAR with |
| | VEVKKPGASVKVSCKASGYTFTNYYMYW | pembrolizumab-derived |
| | VRQAPGQGLEWMGGINPSNGGTNFNEKF | scFv (V_{H}-linker-V_{L}), long |
| | KNRVTLTTDSSTTTAYMELKSLQFDDTAV | IgG4 hinge domain, |
| | YYCARRDYRFDMGFDYWGQGTTVTVSSA | CD28 transmembrane |
| | STKGGGGGSGGGGSGGGGSEIVLTQSPA | domain, 4-1BB |
| | TLSLSPGERATLSCRASKGVSTSGYSYLH | costimulatory domain, |
| | WYQQKPGQAPRLLIYLASYLESGVPARFS | and CD3ζ signaling |
| | GSGSGTDFTLTISSLEPEDFAVYYCQHSR | domain |
| | DLPLTFGGGTKVEIKRTVAAPSVFIFPPSD | |
| | EQLKSGTAESKYGPPCPPCPGPSVFLFPP | |
| | KPKDTLMISRTPEVTCVVVDVSQEDPEVQ | |
| | FNWYVDGVEVHNAKTKPREEQFQSTYRV | |
| | VSVLTVLHQDWLNGKEYKCKVSNKGLPS | |
| | SIEKTISKAKGQPREPQVYTLPPSQEEMTK | |
| | NQVSLTCLVKGFYPSDIAVEWESNGQPEN | |
| | NYKTTPPVLDSDGSFFLYSRLTVDKSRWQ | |
| | EGNVFSCSVMHEALHNHYTQKSLSLSLGK | |
| | MFWVLVVVGGVLACYSLLVTVAFIIFWVKR | |
| | GRKKLLYIFKQPFMRPVQTTQEEDGCSCR | |
| | FPEEEEGGCELRVKFSRSADAPAYQQGQ | |
| | NQLYNELNLGRREEYDVLDKRRGRDPEM | |
| | GGKPRRKNPQEGLYNELQKDKMAEAYSE | |
| | IGMKGERRRGKGHDGLYQGLSTATKDTY | |
| | DALHMQALPPR | |
| 30 | MLLLVTSLLLCELPHPAFLLIPEIVLTQSPA | Anti-PD-1 CAR with |
| | TLSLSPGERATLSCRASKGVSTSGYSYLHW | pembrolizumab-derived |
| | YQQKPGQAPRLLIYLASYLESGVPARFSG | scFv (V_{L}-linker-V_{H}), |
| | SGSGTDFTLTISSLEPEDFAVYYCQHSRDL | short IgG4 hinge |
| | PLTFGGGTKVEIKRTVAAPSVFIFPPSDEQ | domain, CD28 |
| | LKSGTAGGGGSGGGGSGGGGSQVQLVQ | transmembrane domain, |
| | SGVEVKKPGASVKVSCKASGYTFTNYYM | 4-1BB costimulatory |
| | YWVRQAPGQGLEWMGGINPSNGGTNFN | domain, and CD32 |
| | EKFKNRVTLTTDSSTTTAYMELKSLQFDD | signaling domain |
| | TAVYYCARRDYRFDMGFDYWGQGTTVTV | |
| | SSASTKGESKYGPPCPPCPMFWVLVVVGG | |
| | VLACYSLLVTVAFIIFWVKRGRKKLLYIFK | |
| | QPFMRPVQTTQEEDGCSCRFPEEEEGGC | |
| | ELRVKFSRSADAPAYQQGQNQLYNELNL | |
| | GRREEYDVLDKRRGRDPEMGGKPRRKN | |
| | PQEGLYNELQKDKMAEAYSEIGMKGERR | |
| | RGKGHDGLYQGLSTATKDTYDALHMQAL | |
| | PPR | |
| 31 | MLLLVTSLLLCELPHPAFLLIPEIVLTQSPAT | Anti-PD-1 CAR with |
| | LSLSPGERATLSCRASKGVSTSGYSYLHW | pembrolizumab-derived |
| | YQQKPGQAPRLLIYLASYLESGVPARFSG | scFv (V_{L}-linker-V_{H}), |
| | SGSGTDFTLTISSLEPEDFAVYYCQHSRDL | medium IgG4 hinge |
| | PLTFGGGTKVEIKRTVAAPSVFIFPPSDEQ | domain, CD28 |
| | LKSGTAGGGGSGGGGSGGGGSQVQLVQ | transmembrane domain, |
| | SGVEVKKPGASVKVSCKASGYTFTNYYM | 4-1BB costimulatory |
| | YWVRQAPGQGLEWMGGINPSNGGTNFN | domain, and CD3ζ |
| | EKFKNRVTLTTDSSTTTAYMELKSLQFDD | signaling domain |
| | TAVYYCARRDYRFDMGFDYWGQGTTVTV | |
| | SSASTKGESKYGPPCPPCPGQPREPQVY | |
| | TLPPSQEEMTKNQVSLTCLVKGFYPSDIA | |
| | VEWESNGQPENNYKTTPPVLDSDGSFFL | |
| | YSRLTVDKSRWQEGNVFSCSVMHEALHN | |
| | HYTQKSLSLSLGKMFWVLVVVGGVLACYS | |
| | LLVTVAFIIFWVKRGRKKLLYIFKQPFMRPV | |
| | QTTQEEDGCSCRFPEEEEGGCELRVKFS | |
| | RSADAPAYQQGQNQLYNELNLGRREEYD | |
| | VLDKRRGRDPEMGGKPRRKNPQEGLYNE | |
| | LQKDKMAEAYSEIGMKGERRRGKGHDGL | |
| | YQGLSTATKDTYDALHMQALPPR | |
| 32 | MLLLVTSLLLCELPHPAFLLIPEIVLTQSPAT | Anti-PD-1 CAR with |
| | LSLSPGERATLSCRASKGVSTSGYSYLHW | pembrolizumab-derived |
| | YQQKPGQAPRLLIYLASYLESGVPARFSG | scFv (V_{L}-linker-V_{H}), long |
| | SGSGTDFTLTISSLEPEDFAVYYCQHSRDL | IgG4 hinge domain, |
| | PLTFGGGTKVEIKRTVAAPSVFIFPPSDEQ | CD28 transmembrane |
| | LKSGTAGGGGSGGGGSGGGGSQVQLVQ | domain, 4-1BB |
| | SGVEVKKPGASVKVSCKASGYTFTNYYM | costimulatory domain, |
| | YWVRQAPGQGLEWMGGINPSNGGTNFN | and CD3ζ signaling |
| | EKFKNRVTLTTDSSTTTAYMELKSLQFDD | domain |
| | TAVYYCARRDYRFDMGFDYWGQGTTVTV | |
| | SSASTKGESKYGPPCPPCPGPSVFLFPPK | |
| | PKDTLMISRTPEVTCVVVDVSQEDPEVQF | |
| | NWYVDGVEVHNAKTKPREEQFQSTYRVV | |
| | SVLTVLHQDWLNGKEYKCKVSNKGLPSSI | |
| | EKTISKAKGQPREPQVYTLPPSQEEMTKN | |
| | QVSLTCLVKGFYPSDIAVEWESNGQPENN | |
| | YKTTPPVLDSDGSFFLYSRLTVDKSRWQE | |
| | GNVFSCSVMHEALHNHYTQKSLSLSLGK | |
| | MFWVLVVVGGVLACYSLLVTVAFIIFWVKR | |
| | GRKKLLYIFKQPFMRPVQTTQEEDGCSCR | |
| | FPEEEEGGCELRVKFSRSADAPAYQQGQ | |
| | NQLYNELNLGRREEYDVLDKRRGRDPEM | |
| | GGKPRRKNPQEGLYNELQKDKMAEAYSE | |
| | IGMKGERRRGKGHDGLYQGLSTATKDTY | |
| | DALHMQALPPR | |
| 33 | MLLLVTSLLLCELPHPAFLLIPQVQLVQSG | Anti-PD-1 CAR with |
| | VEVKKPGASVKVSCKASGYTFTNYYMYW | pembrolizumab-derived |
| | VRQAPGQGLEWMGGINPSNGGTNFNEKF | scFv (V_{H}-linker-V_{L}), |
| | KNRVTLTTDSSTTTAYMELKSLQFDDTAV | short IgG4 hinge |
| | YYCARRDYRFDMGFDYWGQGTTVTVSSA | domain, CD28 |
| | STKGGGGGSGGGGGGGGSEIVLTQSPA | transmembrane domain, |
| | TLSLSPGERATLSCRASKGVSTSGYSYLH | 4-1BB costimulatory |
| | WYQQKPGQAPRLLIYLASYLESGVPARFS | domain, and CD3ζ |
| | GSGSGTDFTLTISSLEPEDFAVYYCQHSR | signaling domain |
| | DLPLTFGGGTKVEIKRTVAAPSVFIFPPSD | T2A |
| | EQLKSGTAESKYGPPCPPCPMFWVLVVV | truncated EGFR |
| | GGVLACYSLLVTVAFIIFWVKRGRKKLLYIF | (tEGFR) |
| | KQPFMRPVQTTQEEDGCSCRFPEEEEGG | T2A |
| | CELRVKFSRSADAPAYQQGQNQLYNELN | C46 fusion inhibitor |
| | LGRREEYDVLDKRRGRDPEMGGKPRRKN | |
| | PQEGLYNELQKDKMAEAYSEIGMKGERR | |
| | RGKGHDGLYQGLSTATKDTYDALHMQAL | |
| | PPRLEGGGEGRGSLLTCGDVEENPGPRML | |
| | LLVTSLLLCELPHPAFLLIPRKVQNGIGIGE | |
| | FKDSLSINATNIKHFKNCTSISGDLHILPVA | |
| | FRGDSFTHTPPLDPQELDILKTVKEITGFLL | |
| | IQAWPENRTDLHAFENLEIIRGRTKQHGQF | |
| | SLAVVSLNITSLGLRSLKEISDGDVIISGNK | |
| | NLCYANTINWKKLFGTSGQKTKIISNRGEN | |
| | SCKATGQVCHALCSPEGCWGPEPRDCVS | |
| | CRNVSRGRECVDKCNLLEGEPREFVENS | |
| | ECIQCHPECLPQAMNITCTGRGPDNCIQC | |
| | AHYIDGPHCVKTCPAGVMGENNTLVWKY | |
| | ADAGHVCHLCHPNCTYGCTGPGLEGCPT | |
| | NGPKIPSIATGMVGALLLLLVVALGIGLFM | |
| | LEGGGEGRGSLLTCGDVEENPGPRMGAG | |
| | ATGRAMDGPRLLLLLLLGVSLGGARSWM | |
| | EWDREINNYTSLIHSLIEESQNQQEKNEQE | |
| | LLELDKWASLWNWFRSERKCCVECPPCP | |
| | APPVAGPLIALVTSGALLAVLGITGYFLMN | |
| | RRSWSPTGERLELEP | |
| 34 | RKVCNGIGIGEFKDSLSINATNIKHFKNCTS | tEGFR amino acid |
| | ISGDLHILPVAFRGDSFTHTPPLDPQELDIL | sequence |
| | KTVKEITGFLLIQAWPENRTDLHAFENLEII | |
| | RGRTKQHGQFSLAVVSLNITSLGLRSLKEI | |
| | SDGDVIISGNKNLCYANTINWKKLFGTSGQ | |
| | KTKIISNRGENSCKATGQVCHALCSPEGC | |
| | WGPEPRDCVSCRNVSRGRECVDKCNLLE | |
| | GEPREFVENSECIQCHPECLPQAMNITCT | |
| | GRGPDNCIQCAHYIDGPHCVKTCPAGVM | |
| | GENNTLVWKYADAGHVCHLCHPNCTYGC | |
| | TGPGLEGCPTNGPKIPSIATGMVGALLLLL | |
| | VVALGIGLFM | |
| 35 | MGAGATGRAMDGPRLLLLLLLGVSLGGA | c46 fusion inhibitor |
| | RSWMEWDREINNYTSLIHSLIEESQNQQE | amino acid sequence |
| | KNEQELLELDKWASLWNWFRSERKCCVE | |
| | CPPCPAPPVAGPLIALVTSGALLAVLGITG | |
| | YFLMNRRSWSPTGERLELEP | |

In some embodiments, the anti-PD-1 CAR comprises or consists of an amino acid sequence set forth in any one of SEQ ID NOs: 27-32, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in any one of SEQ ID NOs: 27-32. In some embodiments, the anti-PD-1 CAR comprises or consists of an amino acid sequence set forth in SEQ ID NO: 27, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 27. In some embodiments, the anti-PD-1 CAR comprises or consists of an amino acid sequence set forth in SEQ ID NO: 28, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 28. In some embodiments, the anti-PD-1 CAR comprises or consists of an amino acid sequence set forth in SEQ ID NO: 29, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 29. In some embodiments, the anti-PD-1 CAR comprises or consists of an amino acid sequence set forth in SEQ ID NO: 30, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 30. In some embodiments, the anti-PD-1 CAR comprises or consists of an amino acid sequence set forth in SEQ ID NO: 31, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 31. In some embodiments, the anti-PD-1 CAR comprises or consists of an amino acid sequence set forth in SEQ ID NO: 32, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 32. In some embodiments, the anti-PD-1 CAR comprises or consists of an amino acid sequence set forth in Table 8, or an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in Table 8. In any of these embodiments, the anti-PD-1 CAR may comprise one or more amino acid substitutions, including, for example, conservative substitutions, insertions, and/or deletions from the referenced amino acid sequences.

In some embodiments, the anti-PD-1 CAR comprises or consists of an amino acid sequence set forth in any one of SEQ ID NOs: 27-32, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in any one of SEQ ID NOs: 27-32. In some embodiments, the anti-PD-1 CAR comprises or consists of an amino acid sequence set forth in SEQ ID NO: 27, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 27. In some embodiments, the anti-PD-1 CAR comprises or consists of an amino acid sequence set forth in SEQ ID NO: 28, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 28. In some embodiments, the anti-PD-1 CAR comprises or consists of an amino acid sequence set forth in SEQ ID NO: 29, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 29. In some embodiments, the anti-PD-1 CAR comprises or consists of an amino acid sequence set forth in SEQ ID NO: 30, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 30. In some embodiments, the anti-PD-1 CAR comprises or consists of an amino acid sequence set forth in SEQ ID NO: 31, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 31. In some embodiments, the anti-PD-1 CAR comprises or consists of an amino acid sequence set forth in SEQ ID NO: 32, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 32. In some embodiments, the anti-PD-1 CAR comprises or consists of an amino acid sequence set forth in Table 8, or an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in Table 8. In any of these embodiments, the anti-PD-1 CAR may comprise one or more amino acid substitutions, including, for example, conservative substitutions, insertions, and/or deletions from the referenced amino acid sequences.

In some embodiments, the anti-PD-1 CAR comprises or consists of an amino acid sequence set forth in any one of SEQ ID NOs: 27-32, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in any one of SEQ ID NOs: 27-32. In some embodiments, the anti-PD-1 CAR comprises or consists of an amino acid sequence set forth in SEQ ID NO: 27, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 27. In some embodiments, the anti-PD-1 CAR comprises or consists of an amino acid sequence set forth in SEQ ID NO: 28, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 28. In some embodiments, the anti-PD-1 CAR comprises or consists of an amino acid sequence set forth in SEQ ID NO: 29, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 29. In some embodiments, the anti-PD-1 CAR comprises or consists of an amino acid sequence set forth in SEQ ID NO: 30, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 30. In some embodiments, the anti-PD-1 CAR comprises or consists of an amino acid sequence set forth in SEQ ID NO: 31, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 31. In some embodiments, the anti-PD-1 CAR comprises or consists of an amino acid sequence set forth in SEQ ID NO: 32, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 32. In some embodiments, the anti-PD-1 CAR comprises or consists of an amino acid sequence set forth in Table 8, or an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in Table 8. In any of these embodiments, the anti-PD-1 CAR may comprise one or more amino acid substitutions, including, for example, conservative substitutions, insertions, and/or deletions from the referenced amino acid sequences.

In some embodiments, the present technology comprises nucleic acids comprising a nucleotide sequence encoding an anti-PD-1 CAR according to various embodiments of the present technology. The nucleic acids may be used (for example, in the form of a vector) to transfect or transduce a host cell (e.g., a T cell) so that the host cell would express the encoded anti-PD-1 CAR. In certain of these embodiments, the nucleic acids comprise a nucleotide sequence that is codon-optimized for a host cell (for example, a human cell) according to techniques known to one of ordinary skill in the art. Codon-optimized sequences include sequences that are partially or fully codon-optimized.

In some embodiments, the nucleic acids comprising a nucleotide sequence encoding an anti-PD-1 CAR according to various embodiments of the present technology may be present in the form of a vector (e.g., a plasmid or a viral vector) or packaged into a virus for introduction into a host cell (e.g., a T cell). The vector may be any type of vector suitable for introduction of nucleotide sequences into a host cell, including, for example, plasmids, adenoviral vectors, adeno-associated viral (AAV) vectors, retroviral vectors, lentiviral vectors, phages, and homology-directed repair (HDR)-based donor vectors. The virus may be any type of virus suitable for transducing a host cell and introducing nucleotide sequences into the host cell, including, for example, adenoviruses, AAVs, retroviruses, lentiviruses, and phages. In some embodiments, the nucleotide sequence is in a vector (e.g., a viral vector) or a virus which facilitates integration of the nucleotide sequence into a host cell's genome upon introduction into the host cell and thereby replication along with the host genome. In some embodiments, such nucleotide sequence may be present inside a host cell, for example, integrated into the genome of the host cell, for production of the anti-PD-1 CAR in the host cell.

In some embodiments, the nucleic acids according to various embodiments of the present technology may be delivered to a host cell via one or more non-viral delivery methods and/or using one or more non-viral vectors, including, but not limited to, physical/mechanical methods, inorganic particles, and synthetic or natural biodegradable particles. Non-limiting examples of physical/mechanical methods include needle injection, ballistic injection, gene gun, electroporation, sonoporation, photoporation, optoporation, magnetofection, and hydroporation. Non-limiting examples of inorganic particles include calcium phosphate, silica, gold, and magnetic particles. Non-limiting examples of synthetic or natural biodegradable particles include polymeric-based non-viral vectors (e.g., poly lactic-co-glycolic acid, poly lactic acid, polyethylene imine, chitosan, dendrimers, polymethacrylates), cationic lipid-based non-viral vectors (e.g., cationic liposomes, cationic emulsions, solid lipid nanoparticles), and peptide-based non-viral vectors (e.g., poly-L-lysine).

In some embodiments, the nucleic acids according to various embodiments of the present technology may be operatively linked to certain regulatory elements of the vector. As known to a skilled artisan, expression vectors are typically engineered to contain polynucleotide sequences that are needed to effect the expression and processing of coding sequences to which they are operatively linked. Expression control sequences may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In some embodiments, the vector may comprise a promoter that drives constitutive gene expression in mammalian cells. Those frequently used promoters include, for example, murine stem cell virus (MSCV) promotor, elongation factor 1 alpha (EF1α) promoter, cytomegalovirus (CMV) immediate-early promoter, simian vacuolating virus 40 (SV40) early promoter, spleen focus-forming virus (SFFV) promoter, phosphoglycerate kinase (PGK) promoter, human beta actin promoter, polyubiquitin C gene (UBC) promoter, and CAG promoter.

In some embodiments, the vector may comprise an inducible promoter that allows controlled expression of a transgene (e.g., anti-PD-1 CAR). Unlike constitutive promoters, inducible promoters may switch between an on and an off state in response to certain stimuli (e.g., extracellular signals, ligands, chemical agents, temperature, light) and may be regulated in tissue- or cell-specific manners. Non-limiting examples of frequently used inducible promoters include the tetracycline On (Tet-On) system, the tetracycline Off (Tet-Off) system, AlcA, LexA, and Cre.

In some embodiments, the vector may comprise a functional domain that allows inducible expression of a transgene (e.g., anti-PD-1 CAR). For a non-limiting example, the transgene (e.g., anti-PD-1 CAR) may comprise an inducible hepatitis C-derived NS3 protease domain, which allows control of the transgene expression by administration or withholding of a protease inhibitor, e.g., grazoprevir. See Li et al., Cancer Cell (2022) 40(11):1294-1305.e4. The NS3 protease domain may be placed within the anti-PD-1 CAR between different domains, for example, between the transmembrane domain and the intracellular signaling domain. The presence of the protease inhibitor (e.g., grazoprevir) switches the system on to allow expression of the anti-PD-1 CAR, and expression is switch offed by withholding the protease inhibitor.

In some embodiments, the vector may comprise a Kozak consensus sequence, usually upstream of the coding sequence. A Kozak consensus sequence is a nucleic acid motif that functions as the protein translation initiation site in most eukaryotic mRNA transcripts and mediates ribosome assembly and translation initiation. In some embodiments, the Kozak consensus sequence comprises or consists of the sequence of (gcc) gccrccatgg (SEQ ID NO: 36), wherein r is a purine (i.e., a or g). The use of “( )” around a sequence in the present technology means that the enclosed sequence is optional.

In some embodiments, the vector may comprise a Woodchuck Hepatitis Virus (WHV) Posttranscriptional Regulatory Element (WPRE). A WPRE is a DNA sequence that, when transcribed, creates a tertiary structure enhancing expression. The WPRE sequence is commonly used to increase expression of genes delivered by viral vectors. In some embodiments, the WPRE sequence comprises or consists of the sequence of

(SEQ ID NO: 37)
aatcaacctctggattacaaaatttgtgaaagattgactggtattc
ttaactatgttgctccttttacgctatgtggatacgctgctttaa
tgcctttgtatcatgctattgcttcccgtatggctttcattttct
cctccttgtataaatcctggttgctgtctctttatgaggagttgt
ggcccgttgtcaggcaacgtggcgtggtgtgcactgtgtttgctg
acgcaacccccactggttggggcattgccaccacctgtcagctcc
tttccgggactttcgctttccccctccctattgccacggeggaac
tcategccgcctgccttgcccgctgctggacaggggctcggctgt
tgggcactgacaattccgtggtgttgtcggggaaatcatcgtcct
ttccttggctgctcgcctgtgttgccacctggattctgcgcggga
cgtccttctgctacgtcccttcggccctcaatccageggaccttc
cttcccgcggcctgctgccggctctgcggcctcttccgcgtcttc
gccttcgccctcagacgagtcggatctccctttgggccgcctccc
cgc.

In some embodiments, the vector may additionally comprise a selection marker that allows identification, detection, selection, enrichment, sorting, and/or elimination of the transduced cells. In some embodiments, the selection marker comprises a truncated epidermal growth factor receptor (tEGFR), an RQR polypeptide, a truncated low-affinity nerve growth factor (INGFR), a truncated CD19 (tCD19), a truncated CD34 (tCD34), or any combination thereof. Transcripts of the selection marker and the anti-PD-1 CAR may be located in separate vectors, or, alternatively, in the same vector and connected by an internal ribosomal entry site (IRES) or a self-cleaving site (e.g., a 2A site) to achieve co-expression of multiple genes.

2A peptides are a class of 18-22 amino acid-long peptides first discovered in picornaviruses and may induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 7. A glycine-serine-glycine (GSG) linker is optionally added to the N-terminal of a 2A peptide to increase cleavage efficiency.

TABLE 7
Sequences of 2A peptides

| SEQ | | |
|---|---|---|
| ID | | |
| NO: | Sequence | Description |
| 38 | (GSG) EGRGSLLTCGDVEENPGP | T2A |
| 39 | (GSG) ATNFSLLKQAGDVEENPGP | P2A |
| 40 | (GSG) QCTNYALLKLAGDVESNPGP | E2A |
| 41 | (GSG) VKQTLNFDLLKLAGDVESNPGP | F2A |

In some embodiments, the selection marker is tEGFR having an amino acid sequence set forth in SEQ ID NO: 34.

In some embodiments, the selection marker is tEGFR having an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 34.

In some embodiments, the selection marker is tEGFR having an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 34.

In some embodiments, the selection marker is tEGFR having an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 34.

In some embodiments, the vector may additionally comprise an antiviral peptide that affords protection against infection of a virus, for example, the HIV or SIV virus. As described herein, when introduced to a host cell (e.g., a T cell), expression of the antiviral peptide from the vector may allow the modified host cell to be immune against HIV or SIV infection, and the resulting host cells (e.g., T cells) may be used for the treatment of HIV/AIDS, whose main target is T cells. Thus, in some embodiments, the vector may additionally comprise an antiviral peptide that renders a host cell resistant to HIV infection after introduction of the host cell with the vector. Transcripts of the antiviral peptide and the anti-PD-1 CAR may be located in separate vectors, or, alternatively, in the same vector and connected by an IRES or a self-cleaving site (e.g., a 2A site) to achieve co-expression of multiple genes.

In some embodiments, the antiviral peptide is c46 fusion inhibitor peptide having an amino acid sequence set forth in SEQ ID NO: 35, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 35.

In some embodiments, the vector allows co-transcription/expression of an anti-PD-1 CAR, a selection marker, and/or an antiviral peptide as described, which may be connected by an IRES or a self-cleaving site (e.g., a 2A site). In some embodiments, the vector comprises a nucleotide sequence encoding an amino acid sequence set forth in SEQ ID NO: 33.

In some embodiments, the vector comprises a nucleotide sequence encoding an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 33.

In some embodiments, the vector comprises a nucleotide sequence encoding an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 33.

In some embodiments, the vector comprises a nucleotide sequence encoding an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 33.

In some embodiments, the vector or virus comprising a nucleotide sequence encoding an anti-PD-1 CAR according to various embodiments of the present technology may be present in a composition. In some embodiments, the composition may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells of the present technology further comprise a suitable infusion media.

In some embodiments, the composition containing the vector or virus comprising a nucleotide sequence encoding an anti-PD-1 CAR according to various embodiments of the present technology may further comprise a gene editing system or a site-directed nuclease as described for integrating the nucleotide sequence into the genome of the host cell. In some embodiments, the composition containing the vector or virus may be administered in combination with a gene editing system or a site-directed nuclease as described for host cell integration.

Introduction of Nucleic Acids and/or Vectors Thereof into Host Cells

In some embodiments, the nucleic acids encoding an anti-PD-1 CAR according to various embodiments of the present technology may be introduced into a host cell (e.g., a T cell), so that the host cell will express the encoded anti-PD-1 CAR for use in adoptive cell therapy.

### Transfection or Transduction of Host Cells

Host cells may be transformed to incorporate the nucleic acids (e.g., in the form of a vector) by any known method in the field, including, for example, viral transduction, calcium phosphate transfection, lipid-mediated transfection, DEAE-dextran, electroporation, microinjection, nucleoporation, liposomes, nanoparticles, or other methods. In some embodiments, the nucleic acids comprising a nucleotide sequence encoding an anti-PD-1 CAR according to various embodiments of the present technology may be in the form of a viral vector or packaged into a virus for introduction into a population of host cells. The virus may be any type of virus suitable for transducing a host cell and introducing nucleotide sequences into the host cell, including, for example, adenoviruses, AAVs, retroviruses, lentiviruses, and phages. The transformed host cells may be collected and/or screened using known techniques, and the various subpopulations or combinations thereof may be enriched or depleted by known techniques, such as by affinity binding to antibodies, flow cytometry, fluorescence activated cell sorting (FACS), or immunomagnetic selection. After introduction into the host cell, the nucleic acids may be integrated into the genome of the host cell either through random insertion or through site-directed insertion (knock-in) as described.

In some embodiments, after being introduced into a host cell, the nucleic acids comprising a nucleotide sequence encoding an anti-PD-1 CAR according to various embodiments of the present technology may be integrated in the genome of the host cell.

### Random Insertion

In some embodiments, the nucleic acids encoding an anti-PD-1 CAR according to various embodiments of the present technology are inserted into a random genomic locus of a host cell. As known to a person skilled in the art, viral vectors, including, for example, retroviral vectors, lentiviral vectors, adenoviral vectors, and AAV vectors, are commonly used to deliver genetic material into host cells and randomly insert the foreign or exogenous gene into the host cell genome to facilitate stable expression and replication of the gene.

### Site-Directed Insertion (Knock-In)

In some embodiments, the nucleic acids encoding an anti-PD-1 CAR according to various embodiments of the present technology are inserted into a specific genomic locus of the host cell. A number of gene editing methods may be used for inserting a transgene into a specific genomic locus of choice. Gene editing is a type of genetic engineering in which a nucleotide sequence may be inserted, deleted, modified, or replaced in the genome of a living organism. A number of gene editing systems may be used for inserting the nucleotide sequence into a specific genomic locus of the host cell, and some of these systems generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and may occur during any phase of the cell cycle, but it is more error prone. In gene editing, NHEJ is generally used to produce insertion/deletion mutations (indels), which may produce targeted loss of function in a target gene by shifting the open reading frame (ORF) and producing alterations in the coding region or an associated regulatory region. HDR, on the other hand, is a preferred pathway for producing targeted knock-ins, knock-outs, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. In some embodiments, the gene editing systems of the present technology for site-specific insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains may thus potentially bind to an extended nucleotide sequence that is unique within a cell's genome.

Various zinc fingers of known specificity may be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers may be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR may then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### TALENS

TALENs are another example of an artificial nuclease which may be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease may be produced specific to any desired DNA sequence. Similar to ZFNs, TALENs may be introduced into a cell to generate DSBs at a desired target site in the genome, and so may be used to knock out genes or knock-in mutations in similar, HDR-mediated pathways. See Boch, Nature Biotech. (2011) 29:135-136; Boch et al., Science (2009) 326:1509-1512; Moscou et al., Science (2009) 326:3501.

### Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods, including mutagenesis and high throughput screening, have been used to create meganuclease variants that recognize unique sequences. Strategies for engineering a meganuclease with altered DNA binding specificity, for example, to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases may create DSBs in the genomic DNA, which may create a frameshift mutation if improperly repaired, for example, via NHEJ, leading to a decrease in the expression of a target gene in a cell. Alternatively, foreign DNA may be introduced into the cell along with the meganuclease. Depending on the sequences of the foreign DNA and chromosomal sequence, this process may be used to modify the target gene. See Silva et al., Current Gene Therapy (2011) 11:11-27.

### Transposases

Transposases are enzymes that bind to the end of a transposon and catalyze its movement to another part of the genome by a cut and paste mechanism or a replicative transposition mechanism. By linking transposases to other systems, such as the CRISPR/Cas system, new gene editing tools may be developed to enable site-specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems, which use a complex of multiple Cas proteins to degrade nucleic acids; and class 2 systems, which use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096): 816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 may be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells, including human cells. In its use in gene editing applications, artificially designed, synthetic gRNAs have replaced the original crRNA: tracrRNA complexes, including in some embodiments via a single gRNA. For example, the gRNAs may be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One may change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′ where “N” may be any nucleotide. Other Cas nuclease variants with alternative PAMs have also been characterized and successfully used for genome editing.

### Additional Modifications

In some embodiments, in addition to introducing the nucleic acids comprising a nucleotide sequence encoding an anti-PD-1 CAR according to various embodiments of the present technology into a host cell and integrating the nucleic acids into the genome of the host cell, the host cell may be further modified for better use in adoptive therapy as described.

In some embodiments, the host cells may be further modified to express one or more safety switches. A safety switch may be used to induce death or apoptosis of the transduced host cells, for example if the cells grow and divide in an undesired manner or cause excessive undesired effects to the host. Alternatively, a safety switch may be used to “shut down” expression of the transgene (e.g., anti-PD-1 CAR) in the host cells so that they cease functioning in the designed manner. Thus, the use of safety switches enables one to conditionally eliminate or stop the host cells in vivo and may be a critical step for the application of cell therapies in the clinic. In some embodiments, the safety switch may cause cell death in a controlled manner, for example, in the presence of a drug or prodrug or upon activation by a selective exogenous compound. In some embodiments, the safety switch comprises a “suicide gene” or “suicide switch”. The suicide gene may cause the death of the cells should they grow and divide in an undesired manner. The suicide gene may encode a protein that results in cell killing only when activated by a specific compound, for example, an enzyme that selectively converts a nontoxic compound into highly toxic metabolites. In some embodiments, the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, tEGFR, GD2, HER1, HER2, MUC1, PSMA, and RQR8.

In some embodiments, the safety switch is an inducible hepatitis C-derived NS3 protease domain as described. In some embodiments, the safety switch is tEGFR having an amino acid sequence set forth in SEQ ID NO: 34.

In some embodiments, the safety switch is tEGFR having an amino acid sequence that is about 50% identical (e.g., about 50%, about 55%, about 60%, about 65%, about 70%, about 75%, about 80%, about 85%, about 90%, about 95%, about 96%, about 97%, about 98%, about 99%, or 100% identical) to the amino acid sequence set forth in SEQ ID NO: 34.

In some embodiments, the safety switch is tEGFR having an amino acid sequence that is at least 50% identical (e.g., at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical) to the amino acid sequence set forth in SEQ ID NO: 34.

In some embodiments, the safety switch is tEGFR having an amino acid sequence that is at least about 50% identical (e.g., at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical) to the amino acid sequence set forth in SEQ ID NO: 34.

In some embodiments, the host cells may be further modified to reduce or eliminate expression of the endogenous TCR, so that the host cell would only express the anti-PD-1 CAR encoded by the transgene. Disruption of expression of the endogenous TCR may be accomplished by knocking out, knocking down, or otherwise modifying one or more endogenous TCR genes (e.g., TRAC, TRBC1, and/or TRBC2) using a gene editing system as described. As used herein, “knock out” includes deleting all or a portion of the target nucleotide sequence in a way that interferes with the function of the target gene. For example, a knockout may be achieved by altering a target nucleotide sequence by inducing an indel in a functional domain of the target nucleotide sequence (e.g., a DNA binding domain) or where base editing and prime editing may be used to change single nucleic acid bases to an alternate base in order to alter the genome sequence. “Knock down” refers to genetic modifications that result in reduced expression of the edited gene. As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A gene editing system, for example, the CRISPR/Cas system, of the present technology may be used to induce an indel of any length in a target polynucleotide sequence. In some embodiments, a transgene (i.e., a nucleic acid encoding an anti-PD-1 CAR according to various embodiments of the present technology) may be inserted into an endogenous TCR gene locus (knocking in) to disrupt expression of that gene using a gene editing system as described, for example, the CRISPR/Cas system.

In some embodiments, the one or more endogenous TCR genes to be knocked out, knocked down, or otherwise modified include, but are not limited to, TRAC, TRBC1, and/or TRBC2. TCRs recognize foreign antigens processed as small peptides and bound to MHC molecules at the surface of APCs. Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes. The TRAC gene encodes the TCR alpha chain constant region. The human TRAC gene resides on chromosome 14 at 22,547,506-22,552, 156, forward strand. The TRAC genomic sequence is set forth in Ensembl ID ENSG00000277734. The TRBC gene encodes the TCR beta chain constant region. TRBC1 and TRBC2 are analogs of the same gene, and T cells mutually exclusively express either TRBC1 or TRBC2. The human TRBC1 gene resides on chromosome 7 at 142,791,694-142,793,368, forward strand, and its genomic sequence is set forth in Ensembl ID ENSG00000211751. The human TRBC2 gene resides on chromosome 7 at 142,801,041-142,802,748, forward strand, and its genomic sequence is set forth in Ensembl ID ENSG00000211772. In some embodiments, the knocking out, knocking down, or otherwise modifying one or more endogenous TCR genes (e.g., TRAC, TRBC1, and/or TRBC2) occurs in one or both alleles of the genomic locus.

In some embodiments, the host cells may be further modified to reduce the immunogenicity of host cells, in order to generate hypoimmunogenic host cells and thereby reduce potential graft-versus-host risks after infusion into a recipient or risks of being eliminated by the recipient's innate immune system. In cell therapy, when the host cells are allogeneic (i.e., derived from a person other than the recipient), additional modifications are needed to reduce potential graft-versus-host risks after infusion into the recipient or risks of being eliminated by the recipient's innate immune system. In some embodiments, the additional modifications comprise reducing or eliminating the expression of MHC class I and/or II molecules (or HLA class I and/or II molecules in humans) in the host cells. In some embodiments, the allogeneic host cells may be modified by knocking out, knocking down, or otherwise modifying one or more HLA loci, such as HLA-A, HLA-B, HLA-C, HLA-DR, HLA-DQ, HLA-DP, HLA-DM, and/or HLA-DO, or one or more genes encoding proteins which otherwise regulate or alter expression of one or more HLA genes, for example, by using the CRISPR/Cas system as described. By modulating (e.g., reducing or deleting) expression of any of the HLA genes, the cells may be rendered hypoimmunogenic and have a reduced ability to induce an immune response in a recipient subject. In some embodiments, the host cells may be further modified to protect them from natural killer (NK) cell-mediated killing after infusion into a recipient, for example, by additionally expressing one or more NK inhibitory ligands, and/or by expressing nonclassical HLA-E and/or HLA-G. See Biernacki et al., Cancer J. (2019) 25 (3): 179-190; Torikai et al., Blood (2013) 122 (8): 1341-1349, the entire contents of each of which are incorporated by reference herein.

### Host Cells and Compositions Thereof

In some embodiments, the present technology comprises host cells, such as T cells, that contain the nucleic acids comprising a nucleotide sequence encoding an anti-PD-1 CAR, and/or express the anti-PD-1 CAR, according to various embodiments of the present technology.

In some embodiments, the host cell is a T cell. In some embodiments, the T cell is a CD4+ T cell, a CD8+ T cell, a CD4− CD8− double negative T cell, a naive T cell (CD62L+, CCR-7+, CD45RA+, CD25−, CD45RO−), a central memory T cell (CD62L+, CCR-7+, CD45RA−, CD45RO+, CD25+, CD127+), an effector memory T cell (CD62L−, CCR-7−, CD45RA−, CD45RO+, CD25−, CD127+), a stem cell memory T cell (CD62L+, CCR-7+, CD45RA+, CD45RO+), or any combination thereof. In some embodiments, the T cell is a cytotoxic T cell, or a CD8+ T cell. T cells are a type of lymphocyte, which develops in the thymus gland and plays a central role in the immune response. More specifically, the T cells may be naive T cells (not exposed to antigen; increased expression of CD62L, CCR7, CD28, CD3, CD127, and CD45RA, and decreased expression of CD45RO as compared to TCM), memory T cells (antigen-experienced and long-lived), or effector cells (antigen-experienced, cytotoxic). Memory T cells may be further divided into subsets of TCM (increased expression of CD62L, CCR7, CD28, CD127, CD45RO, and CD95, and decreased expression of CD54RA as compared to naive T cells) and TEM (decreased expression of CD62L, CCR7, CD28, CD45RA, and increased expression of CD127 as compared to naive T cells or TCM). Effector T cells refer to antigen-experienced CD8+ or CD4+ cytotoxic T cells that have decreased expression of CD62L, CCR7, CD28, and are positive for granzyme, perforin, and/or TNFR ligands, as compared to TCM. Helper T cells may be CD4+ cells that influence the activity of other immune cells by releasing cytokines.

In some embodiments, the T cells are autologous (i.e., obtained from the subject who will receive the T cells after modification). In some embodiments, the T cells are allogeneic (i.e., obtained from someone other than the subject who will receive the T cells after modification). In either of these embodiments, the T cells may be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, for example, in the case of allogeneic T cells, the T cells may be derived or differentiated from pluripotent stem cells (PSCs), such as embryonic stem cells (ESCs) or induced pluripotent cells (IPSCs).

In some embodiments, the host cell is a natural killer (NK) cell. NK cells (also defined as large granular lymphocytes) represent a cell lineage differentiated from the common lymphoid progenitor (which also gives rise to B lymphocytes and T lymphocytes). Unlike T cells, NK cells do not naturally express CD3 at the plasma membrane. Importantly, NK cells do not express TCRs and typically also lack other antigen-specific cell surface receptors. NK cells' cytotoxic activity does not require sensitization but is enhanced by activation with a variety of cytokines including IL-2. The NK cells may be autologous or allogeneic, and may be primary NK cells or be derived or differentiated from ESCs or iPSCs.

In some embodiments, the host cell is a natural killer T (NKT) cell. NKT cells are a heterogeneous group of T cells that share properties of both T cells and NK cells. Many of these cells recognize the non-polymorphic CD1d molecule, an antigen-presenting molecule that binds self and foreign lipids and glycolipids. They constitute only approximately 1% of all peripheral blood T cells. The NKT cells may be autologous or allogeneic, and may be primary NK cells or be derived or differentiated from ESCs or iPSCs.

In some embodiments, the present technology comprises pharmaceutical compositions comprising a host cell according to various embodiments of the present technology.

In some embodiments, the host cell (e.g., a T cell expressing an anti-PD-1 CAR of the present technology) may be present in the pharmaceutical composition in an amount greater than about 10²/ml, for example, up to about 10³/ml, up to about 10⁴/ml, up to about 10⁵/ml, up to about 10⁶/ml, up to about 10⁷/ml, up to about 10⁸/ml, up to about 10⁹/ml, or about 10¹⁰/ml or more.

In some embodiments, the pharmaceutical composition may have various formulations, for example, injectable formulations, lyophilized formulations, liquid formulations, oral formulations, depending on the suitable routes of administration. In some embodiments, the pharmaceutical compositions may have various formulations for injection and/or infusion. Non-limiting examples of formulations for injection and/or infusion include intravenous injection, intraperitoneal injection, intertumoral injection, bone marrow injection, lymph node injection, subcutaneous injection, and cerebrospinal fluid injection.

In some embodiments, the pharmaceutical compositions may be co-formulated in the same dosage unit or may be individually formulated in separate dosage units. The terms “dose unit” and “dosage unit” herein refer to a portion of a pharmaceutical composition that contains an amount of a therapeutic agent, such as a host cell, suitable for a single administration to provide a therapeutic effect. Such dosage units may be administered one to a plurality (i.e., 1 to about 10, 1 to 8, 1 to 6, 1 to 4, or 1 to 2) of times per day, or as many times as needed to elicit a therapeutic response.

In some embodiments, the pharmaceutical compositions may further comprise one or more cytokines, growth factors, and other factors that may be used to manipulate the recipient's immune response toward anticancer activity and/or support engraftment of the host cell into the recipient. Cytokines useful for promoting immune anticancer or antitumor response include, for example, IFN-α, IL-2, IL-3, IL-4, IL-7, IL-9, IL-10, IL-12, IL-13, IL-15, IL-16, IL-17, IL-18, IL-21, IL-24, and GM-CSF.

In some embodiments, the pharmaceutical compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like, and combinations thereof. In some embodiments, compositions comprising host cells of the present technology further comprise a suitable infusion media.

### Methods of Treatment

In some embodiments, the present technology comprises methods for treating and/or preventing HIV infection, symptoms associated with HIV infection, and/or AIDS in a subject in need thereof, the method comprising administering to the subject a therapeutically effective amount of a host cell (e.g., a T cell) containing and/or expressing an anti-PD-1 CAR, or a pharmaceutical composition containing the same, according to various embodiments of the present technology.

In some embodiments, the present technology comprises methods for treating a PD-1-positive hematologic cancer in a subject in need thereof, the method comprising administering to the subject a therapeutically effective amount of a host cell (e.g., a T cell) containing and/or expressing an anti-PD-1 CAR, or a pharmaceutical composition containing the same, according to various embodiments of the present technology. Non-limiting examples of hematologic malignancies include myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma. In some embodiments, the T-cell lymphoma may comprise angioimmunoblastic T-cell lymphoma, peripheral T-cell lymphoma with a follicular helper T cell (T_{FH}) phenotype, and/or T follicular helper lymphoma.

In some embodiments, the present technology comprises methods for treating an autoimmune disease in a subject in need thereof, the method comprising administering to the subject a therapeutically effective amount of a host cell (e.g., a T cell) containing and/or expressing an anti-PD-1 CAR, or a pharmaceutical composition containing the same, according to various embodiments of the present technology. Non-limiting examples of autoimmune diseases include type 1 diabetes, lupus, systemic lupus erythematosus (SLE), rheumatoid arthritis, psoriasis, psoriatic arthritis, multiple sclerosis, inflammatory bowel disease, Crohn's disease, ulcerative colitis, Addison's disease, Graves' disease, Sjogren's syndrome, Hashimoto's thyroiditis, myasthenia gravis, autoimmune vasculitis, pernicious anemia, and celiac disease. As demonstrated in the working examples, the major target of anti-PD-1 CAR-modified T cells include T_{FH} cells, which play a role in several autoimmune diseases. See, e.g., Wei & Niu, J. Autoimmunity (2023) 134:102976; Jin et al., Front. Immunol. (2022) 2; 13:928359; Rao et al., Nature (2017) 542:110-114; Liarski et al., Sci. Transl. Med. (2014) 6(230):230ra46; Choi et al., Arthritis Rheumatol. (2015) 67(4):988-999; Zhang et al., Lupus (2015) 24(9):909-917.

In some embodiments, the host cell (e.g., a T cell) or pharmaceutical composition containing the same, may be administered in a manner appropriate to the disease, condition, or disorder to be treated as determined by persons skilled in the medical art. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the host cell (e.g., a T cell) or pharmaceutical composition containing the same may be administered systemically or locally. In some embodiments, the host cell or pharmaceutical composition containing the same is administered by extracorporeal administration, intravenous administration, subcutaneous administration, intralesional administration, intralymphatic administration, intranodal administration, or intraperitoneal administration. In some embodiments, the host cell or pharmaceutical composition containing the same is delivered preferentially to a tumor or other diseased tissue, for example, by local injection or intralesional injection.

In some embodiments, the host cell (e.g., a T cell) or pharmaceutical composition containing the same is administered in at least one dose. In some embodiments, the host cell or pharmaceutical composition containing the same is administered in multiple doses (e.g., two doses, three doses, four doses, five doses, or more than five doses).

In some embodiments, the host cell (e.g., a T cell) or pharmaceutical composition containing the same is typically administered to the subject in an amount of greater than about 10² cells, for example, up to about 10³ cells, up to about 10⁴ cell, up to about 10⁵ cells, up to about 10⁶ cells, up to about 10⁷ cells, up to about 10⁸ cells, up to about 10⁹ cells, about 10¹⁰ cells, or more per dose.

In some embodiments, the method comprises administering to the subject the host cell or pharmaceutical composition containing the same once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the host cell or pharmaceutical composition containing the same may be administered every day, every other day, every third day, weekly, biweekly (i.e., every other week), every third week, monthly, every other month, every third month, every fourth month, every fifth month, every sixth month, every ninth month, every year, every 18 months, or every 2 years. In some embodiments, the host cell or pharmaceutical composition containing the same may be administered continuously or intermittently, for example, in one or more cycles. In those embodiments, within each cycle, the host cell or pharmaceutical composition containing the same may be administered at various lengths and/or frequencies as described above. For example, the dose regimens listed above could be repeated after a period of about 1 week, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, or more than about 5 years. In some embodiments, the administration schedule is a hybrid of these periods. In some embodiments, treatment is continued until the disease is eliminated, until no further improvement is achieved, or as long as the disease does not progress.

In some embodiments, the host cell (e.g., a T cell) or pharmaceutical composition containing the same may be administered over a pre-determined time period. Alternatively, the host cell or the pharmaceutical composition containing the same may be administered until a particular therapeutic benchmark is reached. In some embodiments, the methods of the present technology include a step of evaluating one or more therapeutic benchmarks in a biological sample, such as, but not limited to, viral loads, levels of a cancer biomarker, to determine whether to continue administration of the host cell or pharmaceutical composition containing the same.

In some embodiments, the method further entails, for treatment of HIV/AIDS, administering one or more antiretroviral therapies (ARTs); or for treatment of hematologic cancers, one or more other cancer therapies, such as surgery, immunotherapy, radiotherapy, chemotherapy, and/or transplantation, to the subject sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an antiretroviral agent, an immunotherapy agent, a chemotherapy agent, and a biologic agent. In some embodiments, the subject was administered the one or more additional therapeutic agents before administration of the host cell or pharmaceutical composition containing the same. In some embodiments, the subject is co-administered the one or more additional therapeutic agents and the host cell or pharmaceutical composition containing the same. In some embodiments, the subject was administered the one or more additional therapeutic agents after administration of the host cell or pharmaceutical composition containing the same.

As one of ordinary skill in the art would understand, the one or more additional therapeutic agents, and the host cell or pharmaceutical composition containing the same, may be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

### Example 1: Anti-PD-1 Chimeric Antigen Receptor (CAR) T Cells Efficiently Target SIV-Infected CD4+ T Cells in Germinal Centers of Rhesus Macaques

Programmed cell death protein 1 (PD-1) is an immune checkpoint marker commonly expressed on memory T cells and enriched in latently infected CD4+ T cells containing replication-competent human immune deficiency virus 1 (HIV) provirus in people with HIV on antiretroviral therapy (ART). CAR T cells that may efficiently kill PD-1 expressing cells in vitro and in vivo were engineered to assess the impact of PD-1 depletion on viral reservoirs and rebound dynamics in simian immunodeficiency virus (SIV) mac239-infected rhesus macaques (RMs). Adoptive transfer experiments of anti-PD-1 CAR T cells were done in 2 SIV-naive and 4 SIV-infected RMs on ART. In 3 of 6 RMs (one SIV-naive and 2 SIV+ RMs), anti-PD-1 CAR T cells expanded efficiently and persisted for up to 100 days concomitant with the depletion of PD-1+ memory T cells in blood and tissues, including CD4+ follicular helper T cells (T_{FH}). This depletion of T_{FH} in lymph nodes was also associated with depletion of detectable SIV RNA from the germinal center (GC). However, following ART interruption, there was a marked increase in SIV replication in extrafollicular portions of lymph nodes, a concomitant 2-log higher viral load set points and accelerated disease progression, associated with the acute depletion of CD8+ memory T cells after CAR T infusion in SIV+ RMs on ART. These data indicate anti-PD-1 CAR T cells may target and deplete PD-1+ T cells in vivo including GC T_{FH} cells and eradicate SIV from this immunological sanctuary.

Here, PD-1 was targeted as a cellular marker for HIV persistence and replication. It was hypothesized that there would be a selective increase in PD-1+ cells in SIV-infected RMs. Moreover, it was tested whether CAR T cells could enter the germinal center (GC) and eliminate PD-1-expressing CD4+ T_{FH} cells where latent SIV/HIV infection often occurs. An anti-PD-1 CAR with low tonic signaling that efficiently kills PD-1-expressing cells and may inhibit SIVmac239-infection in vitro was developed. In vivo, in both SIV-naive and SIVmac239-infected RMs on ART, anti-PD-1 CAR T cells expanded in blood and tissues, gained access to immune privileged sites such as B cell follicles, and depleted PD-1+ CD4+ T cells as well as strongly reduced numbers of SIV RNA+ cells in GCs. Anti-PD-1 CAR T cells persisted without signs of exhaustion as they did not express detectable cell surface PD-1, likely through an interaction in cis with the anti-PD-1 CAR. However, the concomitant destruction of PD-1+ memory CD8+ T cells exacerbated plasma viral load levels and SIV replication was apparently shifted to PD-1− cells in the T cell zone in lymphoid tissues. Persistence of the anti-PD-1 CAR T cells coincided with extended lymphopenia and accelerated SIV disease progression, the latter likely due to profound, persistent depletion of both the CD4+ and CD8+ memory T cell compartment. These data indicate that depletion of SIV cellular reservoirs is possible through destruction of anti-PD-1 CAR T cells.

### Materials and Methods

### Infusion of CAR T Cells in SIV-Naive and SIV-Infected RMs

Infusion of CAR T cells in SIV-naive RMs was done in two male RMs (Macaca mulatta), of Indian genetic background. Each recipient received a single dose of cyclophosphamide (Baxter, 30 mg/kg) for lymphodepletion on day −4 and −3 prior to CAR T cell infusion. In addition, anti-IL-6 antibody Tocilizumab was administered at 8 mg/kg once per day for 3 days, starting with the day of CAR T cell infusion in each animal (animal A and animal B).

Infusion of CAR T cells in SIV-infected RMs used four RMs (Macaca mulatta), one male and three females, one Trim5 Q/Q and three Trim5 Q/CypA of Indian genetic background. These RMs were specific pathogen-free as defined by being free of cercopithecine herpesvirus 1, D-type simian retrovirus, simian T-lymphotropic virus type 1. The RMs were i.v. inoculated with 500 TZM-bl assay focus-forming units of RM PBMC-expanded SIVmac239M and placed on ART starting at 12 days post-infection and maintained on ART. ART consisted of a subcutaneous injection of 5.1 mg kg⁻¹ d⁻¹ tenofovir disoproxil, 40 mg kg⁻¹ d⁻¹ emtricitabine (FTC), and 2.5 mg kg⁻¹ d⁻¹ dolutegravir in a solution containing 15% (v/v) kleptose at pH 4.2, as previously described. Each recipient received a single dose of cyclophosphamide (Baxter, 30 mg/kg) for lymphodepletion on day −5 prior to CAR T cell infusion. In addition, anti-IL-6 antibody Tocilizumab was administered at 8 mg/kg once per day for 3 days, starting with the day of CAR T cell infusion. Anti-PD-1 CAR T cells were i.v. infused at doses of 6×10⁶ or 20.8×10⁶ EGFR+ T cells/kg. ART was stopped on day 14 post-CAR T cell infusion. Whole blood, peripheral lymph nodes (Peri.LN), mesenteric lymph nodes (Mes.LN), spleen, bronchoalveolar lavage (BAL), and bone marrow aspirates (BM), liver, duodenum, and colon were collected longitudinally in all SIV-infected recipient RMs as previously described. Recipient RMs were followed for a minimum of 80 days post-ART cessation for the onset of plasma viremia.

### Virus Detection Assays

Plasma SIV RNA levels were determined using a gag-targeted quantitative real-time/digital RT-PCR format assay, as previously described, with 6 replicate reactions analyzed per extracted sample for assay thresholds of 15 SIV RNA copies/mL.

### Cell Culture

K562 cells (ATCC), Molt-4 cells, clone 8 (NIH HIV Reagent Program), Jurkat, and Jurkat Nur77 t2a NeonGreen cells were cultured in RPMI1640 (Gibco), 10% fetal bovine serum and antibiotics. Lenti-X 293T cells (Takara) were cultured in DMEM (Gibco), 10% fetal bovine serum and antibiotics.

### Anti-PD-1 CAR Design and Cloning

A pcl20 SIV lentivirus transfer vector containing a MSCV promoter and a GM-CSF-signal peptide CAR-(GM-CSF-signal peptide-binder-extracellular linker-CD28TM-41BB-CD3ζ)-EGFRt-c46 wPRE expression cassette was amplified by PCR with primers (Primer For 5′-tggaatcagcagaaag-3′ (SEQ ID NO: 42) and rev 5′-atgttctgggtgctc-3′ (SEQ ID NO: 43)) placed in the GM-CSF peptide and the CD28TM sequence and used as a source to generate a lentiviral backbone to generate the various anti-PD-1 CAR vectors.

The amino acid sequence of heavy and light chain of the anti-PD-1 antibody pembrolizumab was retrieved from the PDB database (PDB: 5DK3) and the paratope regions of the variable heavy (V_{H}) and light (V_{L}) chain were identified with the paratome algorithm. The first 125 and 134 amino acids of the V_{H} and V_{L}, respectively, were used to design 2 scFv with a V_{H} V_{L} and V_{L} V_{H} orientation interconnected with 4×GGGGS and 15 bp overlaps with the GM-CSF and extracellular linker sequence in silico and reverse translated it in silico. The composition of the extracellular spacer may affect CAR T-cell recognition, thus, the spacer lengths of the CAR were varied by different portions of the IgG4 Fc of various length. Short, medium and long extracellular linker sequences were retrieved from the NCBI database and in silico designed with a 15 base pair overlap with anti-PD-1 scFv and the CD28TM sequence. Putative splice sites in the DNA sequences were identified and removed with the Splice Site Prediction by Neural Network online tool https://www.fruitfly.org/seq_tools/splice.html. Gene blocks for the scFv and extracellular linker were ordered from IDT and assembled with the pCL20 lentiviral backbone with a NEBuilder HiFi DNA Assembly Cloning Kit (New England Biolabs) according to the manufacturer's protocol to generate a total of 6 anti-PD-1 CAR with 2 different scFv orientations with each 3 different extracellular linkers.

To co-express CXCR5 for B cell follicle targeting in the same expression cassette as the anti-PD-1 CAR, the CXCR5 cassette was transferred from a pCL20 plasmid containing a CAP256-VRC26.25 CAR EGFRt-c46-CXCR5 wPRE expression cassette by restriction digestion with BspE1 and Not1 and ligation into the pCL20 V_{H} V_{L} S and V_{H} V_{L} M anti-PD-1 CAR EGFRt.

A binding-deficient, a signaling-deficient and a binding- and signaling-deficient version of the anti-PD-1 CAR in the V_{H} V_{L} S orientation was built based on a pCL20 V_{H} V_{L} S anti-PD-1 CAR EGFRt backbone. The plasmid was sequentially digested with AgeI and BspEI and the original scFv as well as the partial EGFRt domain with or without the CD3ζ domain were amplified by PCR. The binding-deficient V_{H} V_{L} S scFv containing the transmembrane as well as the 41BB domain was ordered from IDT and amino acids implicated in the interaction with PD-1 were changed to either Alanine or Phenylalanine; these are T52A, Y55F, N74A, S76A, N77A, S80A, N81A, R121A, Y123F, R124A, S194A, Y196F, Y215F, Y219F, S257A, and D259A.

V_{H} V_{L} S anti-PD-1 CAR (SEQ ID NO: 27):
MLLLVTSLLLCELPHPAFLLIPQVQLVQSGVEVKKPGASVKVSCK
ASGYTFTNYYMYWVRQAPGQGLEWMGGINPSNGGTNFNEKFKNRV
TLTTDSSTTTAYMELKSLQFDDTAVYYCARRDYRFDMGFDYWGQG
TTVTVSSASTKGGGGGSGGGGSGGGGSEIVLTQSPATLSLSPGER
ATLSCRASKGVSTSGYSYLHWYQQKPGQAPRLLIYLASYLESGVP
ARFSGSGSGTDFTLTISSLEPEDFAVYYCQHSRDLPLTFGGGTKV
EIKRTVAAPSVFIFPPSDEQLKSGTAESKYGPPCPPCPMFWVLVV
VGGVLACYSLLVTVAFIIFWVKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR
V_{H} V_{L} M anti-PD-1 CAR (SEQ ID NO: 28):
MLLLVTSLLLCELPHPAFLLIPQVQLVQSGVEVKKPGASVKVSCK
ASGYTFTNYYMYWVRQAPGQGLEWMGGINPSNGGTNFNEKFKNRV
TLTTDSSTTTAYMELKSLQFDDTAVYYCARRDYRFDMGFDYWGQG
TTVTVSSASTKGGGGGSGGGGSGGGGSEIVLTQSPATLSLSPGER
ATLSCRASKGVSTSGYSYLHWYQQKPGQAPRLLIYLASYLESGVP
ARFSGSGSGTDFTLTISSLEPEDFAVYYCQHSRDLPLTFGGGTKV
EIKRTVAAPSVFIFPPSDEQLKSGTAESKYGPPCPPCPGQPREPQ
VYTLPPSQEEMTKNQVSLTCLVKGFYPSDIAVEWESNGQPENNYK
TTPPVLDSDGSFFLYSRLTVDKSRWQEGNVFSCSVMHEALHNHYT
QKSLSLSLGKMFWVLVVVGGVLACYSLLVTVAFIIFWVKRGRKKL
LYIFKQPFMRPVQTTQEEDGCSCRFPEEEEGGCELRVKFSRSADA
PAYQQGQNQLYNELNLGRREEYDVLDKRRGRDPEMGGKPRRKNPQ
EGLYNELQKDKMAEAYSEIGMKGERRRGKGHDGLYQGLSTATKDT
YDALHMQALPPR
V_{H} V_{L} L anti-PD-1 CAR (SEQ ID NO: 29):
MLLLVTSLLLCELPHPAFLLIPQVQLVQSGVEVKKPGASVKVSCK
ASGYTFTNYYMYWVRQAPGQGLEWMGGINPSNGGTNFNEKFKNRV
TLTTDSSTTTAYMELKSLQFDDTAVYYCARRDYRFDMGFDYWGQG
TTVTVSSASTKGGGGGSGGGGSGGGGSEIVLTQSPATLSLSPGER
ATLSCRASKGVSTSGYSYLHWYQQKPGQAPRLLIYLASYLESGVP
ARFSGSGSGTDFTLTISSLEPEDFAVYYCQHSRDLPLTFGGGTKV
EIKRTVAAPSVFIFPPSDEQLKSGTAESKYGPPCPPCPGPSVFLF
PPKPKDTLMISRTPEVTCVVVDVSQEDPEVQFNWYVDGVEVHNAK
TKPREEQFQSTYRVVSVLTVLHQDWLNGKEYKCKVSNKGLPSSIE
KTISKAKGQPREPQVYTLPPSQEEMTKNQVSLTCLVKGFYPSDIA
VEWESNGQPENNYKTTPPVLDSDGSFFLYSRLTVDKSRWQEGNVF
SCSVMHEALHNHYTQKSLSLSLGKMFWVLVVVGGVLACYSLLVTV
AFIIFWVKRGRKKLLYIFKQPFMRPVQTTQEEDGCSCRFPEEEEG
GCELRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
V_{L} V_{H} S anti-PD-1 CAR (SEQ ID NO: 30):
MLLLVTSLLLCELPHPAFLLIPEIVLTQSPATLSLSPGERATLSC
RASKGVSTSGYSYLHWYQQKPGQAPRLLIYLASYLESGVPARFSG
SGSGTDFTLTISSLEPEDFAVYYCQHSRDLPLTFGGGTKVEIKRT
VAAPSVFIFPPSDEQLKSGTAGGGGSGGGGSGGGGSQVQLVQSGV
EVKKPGASVKVSCKASGYTFTNYYMYWVRQAPGQGLEWMGGINPS
NGGTNFNEKFKNRVTLTTDSSTTTAYMELKSLQFDDTAVYYCARR
DYRFDMGFDYWGQGTTVTVSSASTKGESKYGPPCPPCPMFWVLVV
VGGVLACYSLLVTVAFIIFWVKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR
V_{L} V_{H} M anti-PD-1 CAR (SEQ ID NO: 31):
MLLLVTSLLLCELPHPAFLLIPEIVLTQSPATLSLSPGERATLSC
RASKGVSTSGYSYLHWYQQKPGQAPRLLIYLASYLESGVPARFSG
SGSGTDFTLTISSLEPEDFAVYYCQHSRDLPLTFGGGTKVEIKRT
VAAPSVFIFPPSDEQLKSGTAGGGGSGGGGSGGGGSQVQLVQSGV
EVKKPGASVKVSCKASGYTFTNYYMYWVRQAPGQGLEWMGGINPS
NGGTNFNEKFKNRVTLTTDSSTTTAYMELKSLQFDDTAVYYCARR
DYRFDMGFDYWGQGTTVTVSSASTKGESKYGPPCPPCPGQPREPQ
VYTLPPSQEEMTKNQVSLTCLVKGFYPSDIAVEWESNGQPENNYK
TTPPVLDSDGSFFLYSRLTVDKSRWQEGNVFSCSVMHEALHNHYT
QKSLSLSLGKMFWVLVVVGGVLACYSLLVTVAFIIFWVKRGRKKL
LYIFKQPFMRPVQTTQEEDGCSCRFPEEEEGGCELRVKFSRSADA
PAYQQGQNQLYNELNLGRREEYDVLDKRRGRDPEMGGKPRRKNPQ
EGLYNELQKDKMAEAYSEIGMKGERRRGKGHDGLYQGLSTATKDT
YDALHMQALPPR
V_{L} V_{H} L anti-PD-1 CAR (SEQ ID NO: 32):
MLLLVTSLLLCELPHPAFLLIPEIVLTQSPATLSLSPGERATLSC
RASKGVSTSGYSYLHWYQQKPGQAPRLLIYLASYLESGVPARFSG
SGSGTDFTLTISSLEPEDFAVYYCQHSRDLPLTFGGGTKVEIKRT
VAAPSVFIFPPSDEQLKSGTAGGGGSGGGGSGGGGSQVQLVQSGV
EVKKPGASVKVSCKASGYTFTNYYMYWVRQAPGQGLEWMGGINPS
NGGTNFNEKFKNRVTLTTDSSTTTAYMELKSLQFDDTAVYYCARR
DYRFDMGFDYWGQGTTVTVSSASTKGESKYGPPCPPCPGPSVFLF
PPKPKDTLMISRTPEVTCVVVDVSQEDPEVQFNWYVDGVEVHNAK
TKPREEQFQSTYRVVSVLTVLHQDWLNGKEYKCKVSNKGLPSSIE
KTISKAKGQPREPQVYTLPPSQEEMTKNQVSLTCLVKGFYPSDIA
VEWESNGQPENNYKTTPPVLDSDGSFFLYSRLTVDKSRWQEGNVF
SCSVMHEALHNHYTQKSLSLSLGKMFWVLVVVGGVLACYSLLVTV
AFIIFWVKRGRKKLLYIFKQPFMRPVQTTQEEDGCSCRFPEEEEG
GCELRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
V_{H} V_{L} S anti-PD-1 CAR BD (binding-defective)
(SEQ ID NO: 44):
MLLLVTSLLLCELPHPAFLLIPQVQLVQSGVEVKKPGASVKVSCK
ASGYTFANYFMYWVRQAPGQGLEWMGGIAPAAGGAAFNEKFKNRV
TLTTDSSTTTAYMELKSLQFDDTAVYYCARADFAFDMGFDYWGQG
TTVTVSSASTKGGGGGSGGGGSGGGGSEIVLTQSPATLSLSPGER
ATLSCRASKGVSTAGFSYLHWYQQKPGQAPRLLIFLASFLESGVP
ARFSGSGSGTDFTLTISSLEPEDFAVYYCQHARALPLTFGGGTKV
EIKRTVAAPSVFIFPPSDEQLKSGTAESKYGPPCPPCPMFWVLVV
VGGVLACYSLLVTVAFIIFWVKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR
V_{H} V_{L} S anti-PD-1 CAR ACD3z (SEQ ID NO: 45):
MLLLVTSLLLCELPHPAFLLIPQVQLVQSGVEVKKPGASVKVSCK
ASGYTFTNYYMYWVRQAPGQGLEWMGGINPSNGGTNFNEKFKNRV
TLTTDSSTTTAYMELKSLQFDDTAVYYCARRDYRFDMGFDYWGQG
TTVTVSSASTKGGGGGSGGGGSGGGGSEIVLTQSPATLSLSPGER
ATLSCRASKGVSTSGYSYLHWYQQKPGQAPRLLIYLASYLESGVP
ARFSGSGSGTDFTLTISSLEPEDFAVYYCQHSRDLPLTFGGGTKV
EIKRTVAAPSVFIFPPSDEQLKSGTAESKYGPPCPPCPMFWVLVV
VGGVLACYSLLVTVAFIIFWVKRGRKKLLYIFKQPFMRPVQTTQE
EDGCSCRFPEEEEGGCEL
V_{H} V_{L} S anti-PD-1 CAR BD ACD3z (SEQ ID NO: 46):
MLLLVTSLLLCELPHPAFLLIPQVQLVQSGVEVKKPGASVKVSCK
ASGYTFANYFMYWVRQAPGQGLEWMGGIAPAAGGAAFNEKFKNRV
TLTTDSSTTTAYMELKSLQFDDTAVYYCARADFAFDMGFDYWGQG
TTVTVSSASTKGGGGGSGGGGSGGGGSEIVLTQSPATLSLSPGER
ATLSCRASKGVSTAGFSYLHWYQQKPGQAPRLLIFLASFLESGVP
ARFSGSGSGTDFTLTISSLEPEDFAVYYCQHARALPLTFGGGTKV
EIKRTVAAPSVFIFPPSDEQLKSGTAESKYGPPCPPCPMFWVLVV
VGGVLACYSLLVTVAFIIFWVKRGRKKLLYIFKQPFMRPVQTTQE
EDGCSCRFPEEEEGGCEL

### Generation of K562 Cells and KG PD-1 Cells

HIV-based lentivirus transfer vector carrying a PD-1 GFPSpark expression cassette under the control of a CMV promoter was purchased from Sino Biological (HG10377-ACGLN). GFPSpark was removed by PCR of the PD-1 nucleotide sequence, which was then reinserted into the parental vector between the NheI and XhoI site using the NEBuilder HiFi DNA Assembly kit (New England Biolabs), and the plasmid was renamed pLV PD-1. Lentivirus was produced using the psPax2 and pMD2.G plasmid on 293T cells. K562 cells were transduced to express PD-1-GFP and sorted to obtain three cell lines (K562 PD-1-GFP high, K562 PD-1-GFP medium, K562 PD-1-GFP low) that express differential levels of PD-1 and GFP. K562 GFP cells and K562 expressing GFP and PD-1 (KG PD-1 cells) from two separate expression cassettes were made by sequential transduction with CL20 MSCV GFP lentivirus, fluorescence assisted cell sorting for GFP expression and a second transduction with LV PD-1 and fluorescence assisted cell sorting for PD-1 expression.

### Absolute Quantification of PD-1 Cell Surface Protein Expression

CD279 PD-1 surface molecule number was calculated with the Quantibrite kit (Becton Dickinson Biosciences) and a PE-conjugated anti-PD-1 antibody (clone EH12.2H7, biolegend) according to the manufacturer's protocol. In brief, 150,000 cells (K562 PD-1-GFP high, K562 PD-1-GFP medium, K562 PD-1-GFP low, K562 GFP control cells) were stained with a serial dilution of the anti-PD-1 antibody and PE fluorescence intensity was analyzed on a flow cytometer. Cell surface expression was calculated at fluorescence saturation.

### Lentivirus Production

Small scale batches of SIV-based lentiviruses were produced in Lenti-X 293T cells (Takara Bio). Cells were cultured with Dulbecco's modified Eagle's medium containing 10% fetal bovine serum (FBS) and 100 U/mL Pen/Strep were transfected using polyethylimine (linear, MW 25000, Polysciences) with four plasmids, 10 μg of the CAR transfer vector, 6 μg of the pCAG-SIVgprre plasmid carrying the gag/pol and rev responsive element, 2 μg of the rev/tat expression plasmid pCAG4-RTR-SIV, and 2 μg of the pMD2.CocalG containing the glycoprotein G of the cocal virus (a generous gift from Dr. Kiem, Fred Hutchinson Cancer Research Center, Seattle, WA). Sixteen hours after transfection, the medium was replaced. The lentivirus-containing media was harvested 30 hours later and cleared by centrifugation at 500×g for 5 minutes followed by filtration on a 0.45 μm filter (Millipore-Sigma). The lentivirus preparation was then layered on top of a 10% sucrose, 0.5 mM ethylene diamine tetra acetic acid (EDTA) in PBS (Gibco) and concentrated by centrifugation at 3,000 g overnight at 4° C. The supernatant was discarded, and the lentivirus pellet was resuspended in PBS (100× concentration), and then stored at −80° C.

The lentivirus preparation was titrated by adding various amounts of lentivirus to Jurkat cells on fibronectin-coated plates followed by spinoculation for 2 hours at 1,200×g. Plates were coated with 20 μg/mL fibronectin (Millipore-Sigma) in PBS overnight at 4° C. Two days later, EGFR expression was assessed by flow cytometry.

### T Cell Culture and CAR T Cell Expansion

CD8+ and CD4+ rhesus macaque T cells were isolated from frozen PBMC by sequential positive selection of CD8 T cells with nonhuman primate CD8 microbeads (Miltenyi) followed by enrichment of CD4 T cells with an EasySep Nonhuman primate CD4+ T cell isolation kit (Stemcell Technologies). CD4+ and CD8+ T cells were stimulated with custom-made anti-CD2/CD3/CD28 immunocult activators (Stemcell Technologies) and cultured separately in presence of X-VIVO15, 10% FBS (Gibco), 50 μM β-mercaptoethanol (SigmaAldrich), antibiotics, (cX15) at 1×10⁶ cells/mL. CD4+ and CD8+ T cell cultures were supplemented with 50 IU/mL recombinant human interleukin-2 rhuIL-2 (Peprotech) and 0.5 ng/ml rhuIL-15 (Peprotech) for CD8+ T cell cultures and 5 ng/ml rhuIL-7 and 0.5 ng/ml rhuIL-15 for CD4+ T cell cultures, respectively (Peprotech). 3 days post-activation, T cell cultures were adjusted to 1×10\hat{ }6/mL again with cX15 and cytokines and 0.5×10\hat{ }6 cells were transduced with lentivirus for the various anti-PD-1 CAR constructs in a 24-well plate. The following day, the transduced cells were transferred to a G-Rex 24 multi-well cell culture plate (Wilson Wolf) in a total of 7 mL cX15 with cytokines and cultured for 7 days. Cytokines were replenished every 2 days and 6 mL of cX15 media was replenished once on day 4. On day 7, T cell cultures were collected and EGFRt+ CAR T cells were isolated by EGFRt positive selection prior to functional experiments.

### Production of CAR T Cell Infusion Products

Infusion products were prepared as previously described. In brief, lymphocyte-enriched, leukapheresis-derived peripheral blood mononuclear cells (PBMCs) were isolated serially by bead-based CD4-positive selection, followed by bead-based CD8-negative selection (StemCell Technologies, Vancouver, BC, Canada). T cells were stimulated with an artificial antigen-presenting cell (aAPC) line engineered to express CD64, CD86, and an anti-CD3 single-chain variable fragment. aAPC cultures were irradiated at a dose of 100 Gy, cryopreserved, and thawed and mixed with NHP T cells at a ratio of 1 aAPC:2 T cells. Stimulated CD4 and CD8 T-cell cultures were plated separately at a concentration of 2-3 10⁶/mL and incubated at 37° C., 5% carbon dioxide for 3 days. Subsequently, lentiviral vector transductions were performed by adding vectors to cells at a multiplicity of infection of approximately 10; cells were transduced in culture media plus protamine sulfate at a concentration of 4×10⁶/mL.

Following 4 hours of transduction at 37° C. with rotation, cells were replated and incubated overnight at 37° C., 5% carbon dioxide. The next day, CD4 and CD8 cells were seeded at a 1:1 ratio into either G-Rex10 or G-Rex100 expansion flasks (Wilson Wolf, St. Paul, MN), and then expanded for 4-7 days, replenishing media once on day 4. Before infusion, a small fraction of the CAR T-cell product was reserved for flow cytometry.

### Biotinylation of Cetuximab and EGFRt Positive Selection of CAR T Cells

EGFRt positive selection was adopted from a previously published method. In brief, clinical grade Cetuximab was biotinylated with an EZ-Link™ Sulfo-NHS-LC-biotinylation kit (ThermoFisher) at 50-fold molar excess (50:1) of biotin for 1 hour at room temperature. Biotinlyated Cetuximab was then dialyzed twice with PBS to remove excess biotin followed by one final round of dialysis with PBS/50% glycerol. The antibody was cryopreserved at −20° C.

For EGFRt positive selection, transduced T cells were washed once with PBS, 1% BSA and resuspended at 2.5×10\hat{ }6 cells/mL in PBS, 1% BSA, 0.1 μg/mL Cetuximab-biotin and incubated for 5 minutes and washed twice with PBS, 1% BSA. Subsequently, positive selection was performed with ultrapure anti-biotin microbeads and LS columns according to the manufacturer's manual (Miltenyi).

### CAR Cell Surface Expression and Paratope Accessibility

CAR cell surface expression was assessed with a PE- or biotin-conjugated human PD-1 Fc fusion protein (PD-1-Fc-PE) (Acrobiosystems) on Jurkat or Molt-4 cells that expressed the various anti-PD-1 CAR-EGFRt. The biotin-conjugated PD-1-Fc protein was counterstained with BV421-conjugated Streptavidin (Invitrogen). In brief, 1×10⁵ cells were stained with PD-1-Fc-PE and mouse anti-EGFRt antibody clone 31G7 Alexa647 (Biotum) or PD-1-Fc-biotin Streptavidin BV421 in combination with either PE-conjugated Erbitux or anti-EGFRt antibody clone Emab134 (Becton Dickinson) and cell surface expression was assessed by flow cytometry.

### Nur77 Assay for Tonic Signaling and for Antigen-Specific Signaling

2×10⁵ Jurkat cells that expressed the various CAR-EGFRt expression cassette were plated in cRPMI in a 96 well plate. The cells were stimulated with 2.5 μL anti-CD3/anti-28 immunocult activator (Stemcell) in cRPMI, 2×10\hat{ }5 K562 PD-1-GFP, 2×10\hat{ }5 GFP or vehicle for 4 hours. EGFRt cell surface expression was expressed with mouse anti-EGFRt antibody clone 31G7 Alexa647 (Biotum). Subsequently, the cells were stained with the Transcription Factor Buffer Set (Becton Dickinson) and mouse-Nur77 antibody clone 12.14 PE (eBioscience). Antigen-independent (tonic signaling) and antigen-dependent activation were assessed by flow cytometry.

### CAR T Cell Killing Live Cell Imaging Assay

In a BioCoat 96-Well, Poly-D Lysine-Treated, Flat-Bottom 96-well plate (Corning), 2×10⁴ target cells (K562 PD-1 GFP high, medium, low, KG PD-1 cells or autologous primary SIVmac239 NefIRESGFP-infected CD4 T cells) in 100 μL cx15 were combined with 100 μL cX15 containing 60,000, 20,000 or 6,666 effector cells (anti-PD-1 CAR or control T cells as indicated) to reach effector to target ratios of 3:1; 1:1 or 1:3. E: T ratios were calculated based on the % EGFRt+ cells. GFP expression was monitored, and images were acquired every 3 hours with an Incucyte S3 Live cell imaging system placed in standard cell culture incubator at 37*C, 5% CO₂ for up to 4 days.

### CAR T Cell Detection and Immunophenotyping

To evaluate the CAR T cells ex vivo, whole blood and mononuclear cell preparations from tissue biopsies were stained with antibodies labeled with fluorochromes for cytometric analysis.

All samples from the study in SIV-naive RM were viability stained with live dead aqua (Life Technologies) at room temperature, anti-CCR7 (3D12: BUV395, BD Biosciences) and anti-CXCR5 (MUSUBEE: PE-Cy7, eBioscience) at 37° C., anti-CD28 (CD28.2: BUV496, BD Biosciences), anti-CD137 (4B4-1: APC, BD Biosciences), anti-CD8 (RPA-T8: APC-Cy7, BD Biosciences), anti-TIGIT (MBSAF43: FITC, ThermoFisher) anti-CD95 (DX2: BV421, BD Biosciences), anti-CD20 (2H7: BV570, BioLegend), anti-CD4 (OKT4: BV605, BioLegend), anti-CD3 (Sp34-2: BV650, BD Biosciences), anti-HLA-DR (L243: BV711, BioLegend), anti-PD-1 (EH12.2H7: BV786, BD Biosciences), anti-CD45 (D058-1283: PECF594, BD Biosciences), and anti-EGFR (PE-conjugated Cetuximab) at 4° C. Cells were then washed and permeabilized for ICS with BD Cytofix/Cytoperm fixation/permeabilization solution kit as per kit instructions and stained with anti-Ki67 (B56: AF700, BD Biosciences) in a subset of experiments. Individual FMO controls for EGFR staining were run alongside full stained cells in all experiments. Experiments were acquired on a 5 laser LSRII 18 color analyzer (BD Biosciences) and analyzed using FlowJo v10 (BD).

All samples from the study in SIV-infected RMs (100 μL whole blood or 10⁶ small lymphocytes from tissue samples or cultured CAR T cells) were initially stained with anti-EGFR (Hu1: Biotin, R&D Systems), and Live/Dead Fixable Aqua Dead Cell Stain Kit (Thermo Fisher) for 30 minutes. After washing, the samples were stained with surface antibodies for 30 minutes: anti-CD45 (D058-1283: BUV395, BD Biosciences), anti-CD8a (SK1: BUV737, BD Biosciences), streptavidin (BV421, BD Biosciences), anti-CXCR5 (MUSUBEE: Super Bright 600, eBioscience), anti-CCR7 (150503: BV711, BD Biosciences), anti-CD4 (L200: BV786, BD Biosciences), anti-CD95 (DX2: PE, BioLegend), anti-CD28 (CD28.2: PE-DAZZ, BioLegend), anti-PD-1 (J105, PerCP-eFluor710, eBioscience), anti-CCR5 (3A9: APC, BD Biosciences), anti-CD3 (SP34-2, Alexa Fluor700, BD Biosciences), and anti-CD20 (2H7: APC/Fire750, BioLegend). Intracellular staining with anti-Ki67 (B56: FITC, BD Biosciences) was performed for 45 minutes after lyse/Fix (BD Biosciences) and permeabilizations. Polychromatic (8-14 parameter) flow cytometric analysis was performed on a LSR II BD instrument as previously described. List mode multiparameter data files were analyzed using FlowJo v10 (BD).

### Microscopy

Tissue samples were collected and combined immunofluorescence, and CAR RNA FISH was performed as previously described with some modifications. In brief, formalin-fixed, paraffin-embedded (FFPE) tissue blocks were sectioned to 4 μm thickness on a Leica RM2255 microtome. Sections were heated at 60° C. for 1 hour, dewaxed and processed for RNAscope pretreatment on a Leica Bond RX according to the manufacturer's recommendations with Leica Bond Epitope Retrieval Solution 2 (ACDbio) for 15 minutes at 95° C. Subsequently, the slides were handled manually according to the manufacturer's manual for the RNAscope Multiplex Fluorescent V2 Assay with two modifications. First, lymphoid tissue sections were digested for 15 minutes with protease III diluted 1 to 5 with PBS at 40° C. WPRE-01 probes (ACDbio) directed against the Woodchuck Hepatitis Virus Posttranscriptional Regulatory Element, which is part of the 3′UTR of the CAR expression cassette, were used to detect CAR T cells. In addition probes against mmu CD8α (ACDbio) and SIVmac239 (ACDbio) were used as indicated. All probes were purchased from ACDbio and developed with multiplex RNAscope version 2 kit using Opal540, Opal620 or Opal650 (PerkinElmer). Second, after the last probe development cycle was finished, the tissue sections were washed once with PBS and blocked with 2% donkey serum (Jackson ImmunoResearch), 1× Casein (Vector Laboratories), 2% BSA (Gemini Bio-Products) and 0.2% v/v Triton X-100 (SigmaAldrich) in dH₂O for 30 min.

The tissue sections were incubated overnight at 4° C. with the primary antibodies monoclonal rat-anti CD3e (1:200, clone CD3-12, Abcam), monoclonal mouse anti-CD20 (1:50, clone L26, ebiosciences) polyclonal rabbit anti-PD-1 (1:50, HPA035981, Sigma Aldrich), or polyclonal rabbit anti-Ki67 (1:400, ab15580, abcam) diluted in blocking buffer. Subsequently, the sections were washed 3 times briefly with PBS Tween (0.05% v/v) and incubated with the secondary antibodies donkey anti-rat Alexa488 (1:100, Invitrogen) donkey anti-rabbit AlexaFlour546 (1:100, Invitrogen) and donkey anti-mouse DyLight680 (1:100, Invitrogen) diluted in blocking buffer. The sections were washed with PBS Tween (0.05% v/v), counterstained with DAPI (ACDbio) and mounted with ProLong Gold antifade (Thermofisher).

Images were acquired with a Leica SP8 confocal microscope equipped with laser lines at 405 nm, 440 nm, and an adjustable white light laser using a 20× or 40× objective.

### The Anti-PD-1 CAR Signals Specifically in Presence of PD-1+ Cells

To target PD-1 with a CAR, the anti-PD-1 antibody pembrolizumab, an immune checkpoint inhibitor therapeutic, was engineered into the binding domain of a CAR. Mechanistically, pembrolizumab binds to PD-1 and thus shields PD-1-expressing T cells from the interaction with PD-L1, which is commonly expressed on cancer cells to suppress antitumor T cell responses. By contrast, an anti-PD-1 CAR would work through eliminating PD-1+ cells.

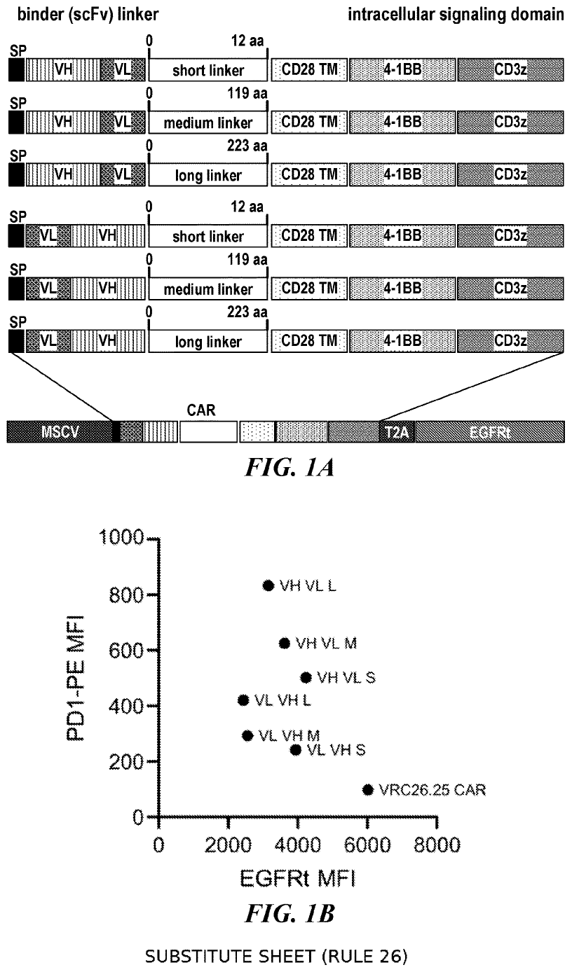

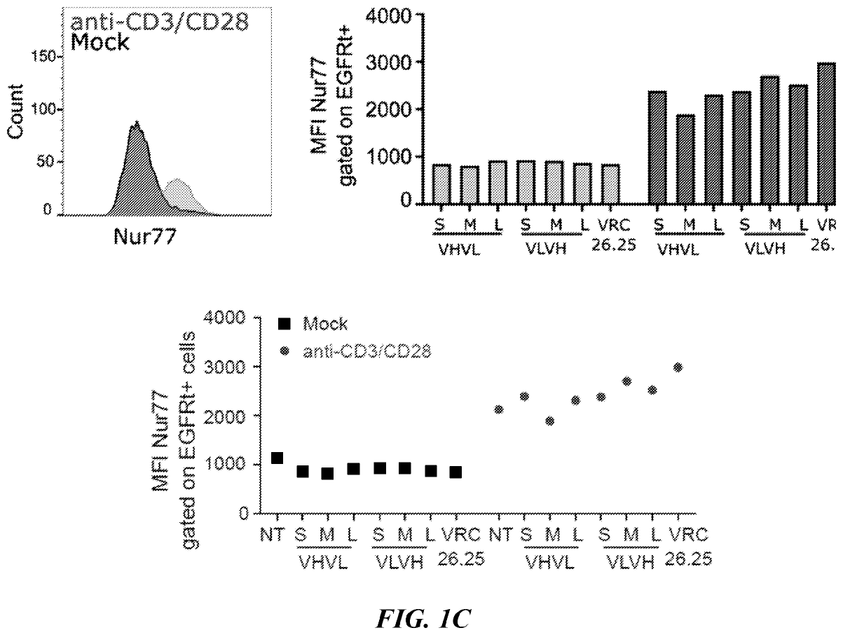

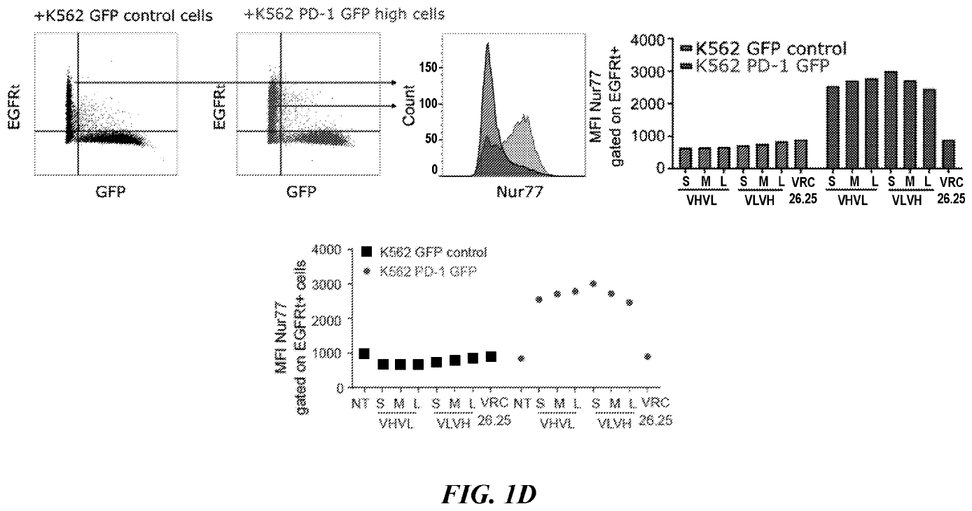

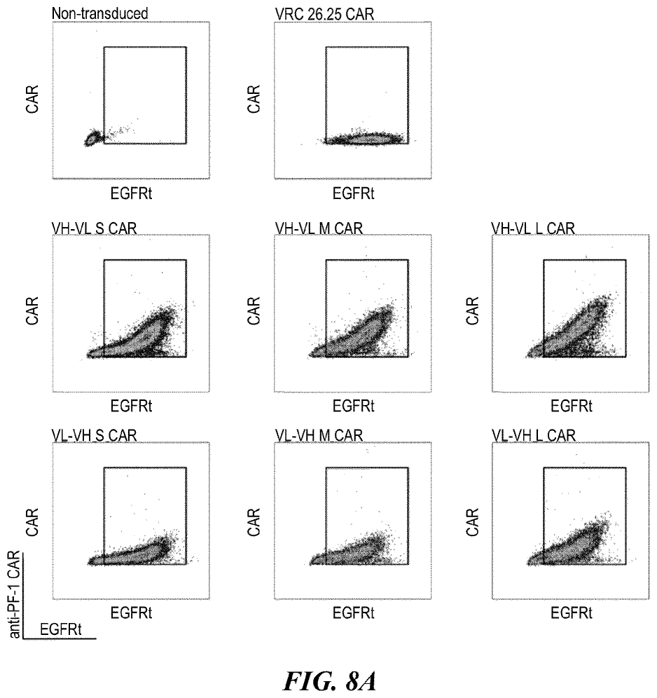

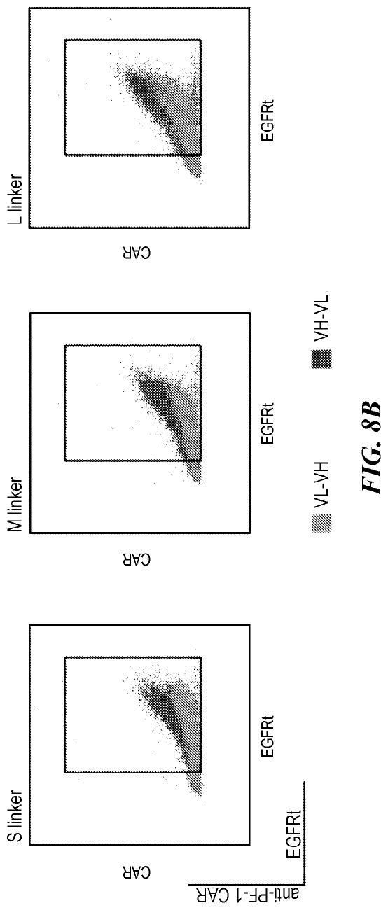

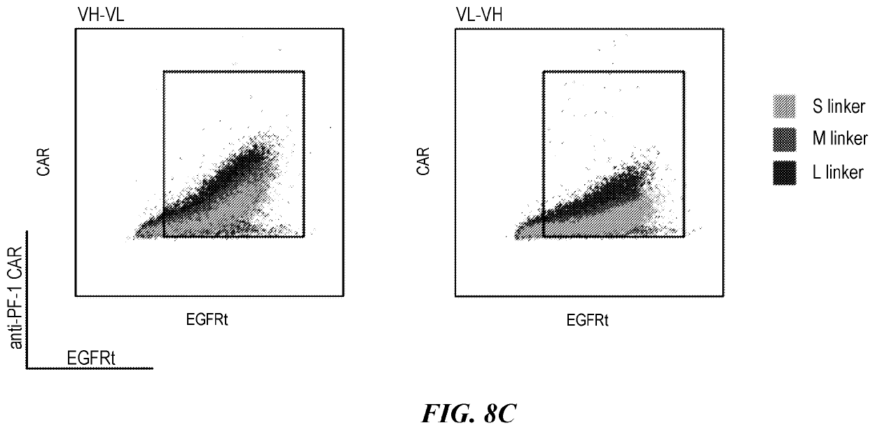

Six pembrolizumab-derived anti-PD-1 CARs were engineered with two different heavy (V_{H}) and light (V_{L}) chain orientations and an IgG4 Fc-derived hinge region of various lengths (FIG. 1A). The six pembrolizumab-derived anti-PD-1 CARs were cloned in an SIV-based lentiviral vector under the control of a murine stem cell virus (MSCV) promoter. The CAR expression cassette also contains a CD28 transmembrane Domain™, 4-1BB intracellular domain and CD3z domain upstream of a truncated EGFRt for marking. These were expressed upstream of a truncated version of EGFR for cell marking and a c46 fusion inhibitor polypeptide that confers gene protection of CD4+ T cells against HIV/SIV infection (FIG. 1A). Transduced Jurkat cells were used to characterize the 6 anti-PD1 CAR constructs. Binding properties and accessibility of the anti-PD-1 CAR paratope were characterized with a PE-conjugated PD-1 Fc and differed by scFv orientation V_{H} V_{L}>V_{L} V_{H} and the linker length (L>M>S) whereas EGFRt expression was similar between constructs (FIGS. 1B and 8A-8C).

Upregulation of the transcription factor Nur77 downstream of endogenous TCR signaling chain has been used to study tonic signaling in absence of antigen, a predictor for poor in vivo performance, and antigen-specific signaling in CAR high throughput screens. Baseline Nur77 expression was low for the 6 anti-PD-1 CAR variants and comparable to another CAR based on the broadly neutralizing antibody CAP256-VRC26.25 (CAPCAR), whereas stimulation through the endogenous TCR induced expression of Nur77 (FIG. 1C). Antigen-specific signaling of the 6 anti-PD-1 CAR or the CAPCAR was tested by coculture of the CAR Jurkat cell lines with K562 PD-1 GFP high or K562-GFP as control cells. Nur77 expression was upregulated in all six anti-PD-1 CAR Jurkat cell lines but not in CAPCAR Jurkat cells in the presence of the coculture of K562 PD-1 GFP high (FIG. 1D). By contrast, Nur77 expression in the anti-PD-1 CAR and CAPCAR remained at the baseline in the presence of K562 GFP control cells (FIG. 1D). The control VRC26.25 CAR did not upregulate Nur77 in the presence of PD-1-expressing cells.

Combined, these data indicate that the six anti-PD-1 CAR constructs do not show tonic signaling and that the scFv orientation and extracellular linker length affect the paratope accessibility of the anti-PD-1 CAR. Antigen-specific signaling appeared not to be affected by the scFv orientation or extracellular linker length.

### Primary Anti-PD-1 CAR T Cells Kill Efficiently PD-1-Expressing Cells and do not Express PD-1

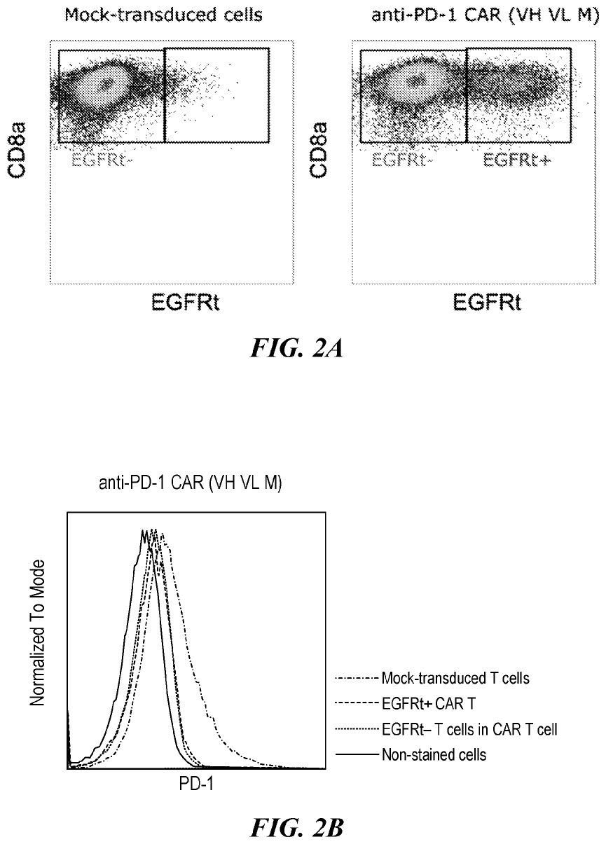

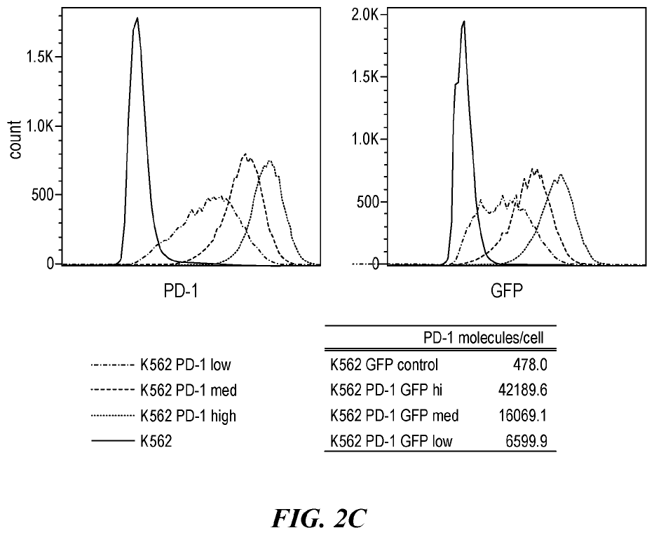

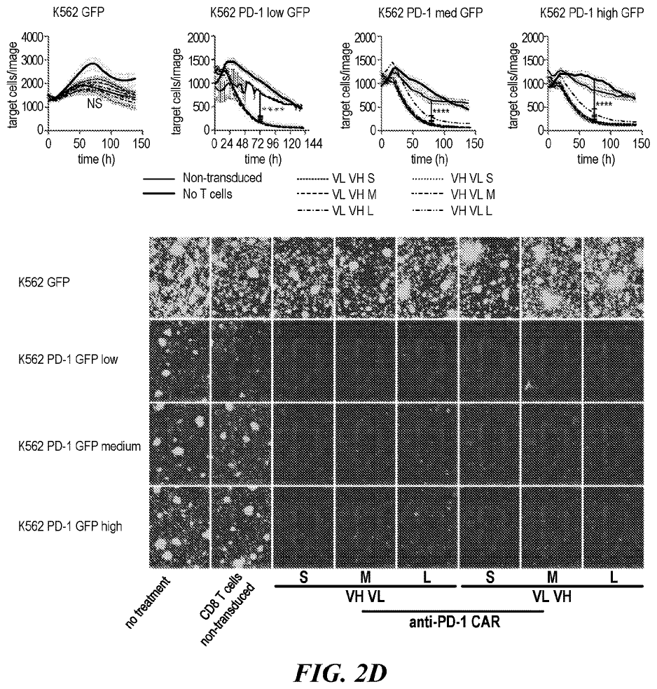

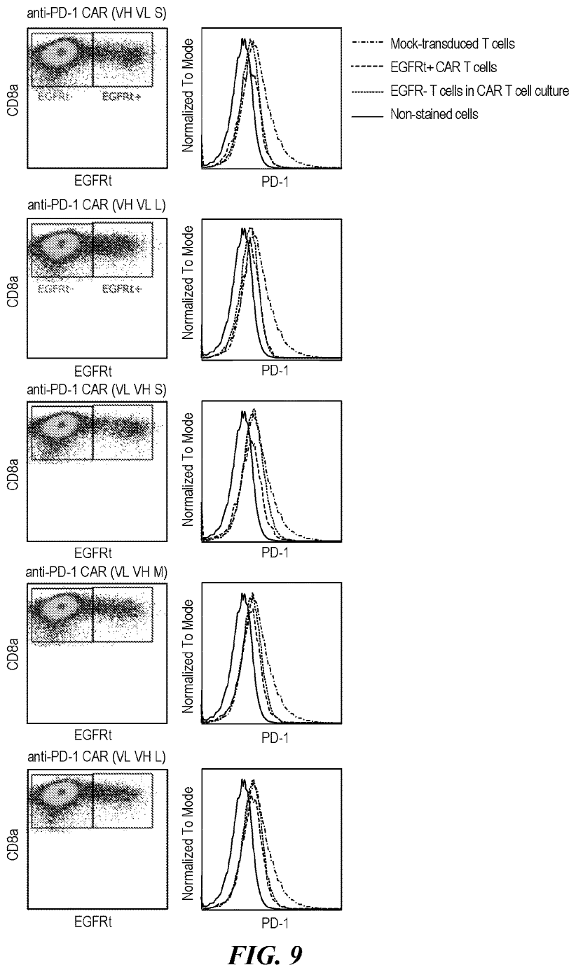

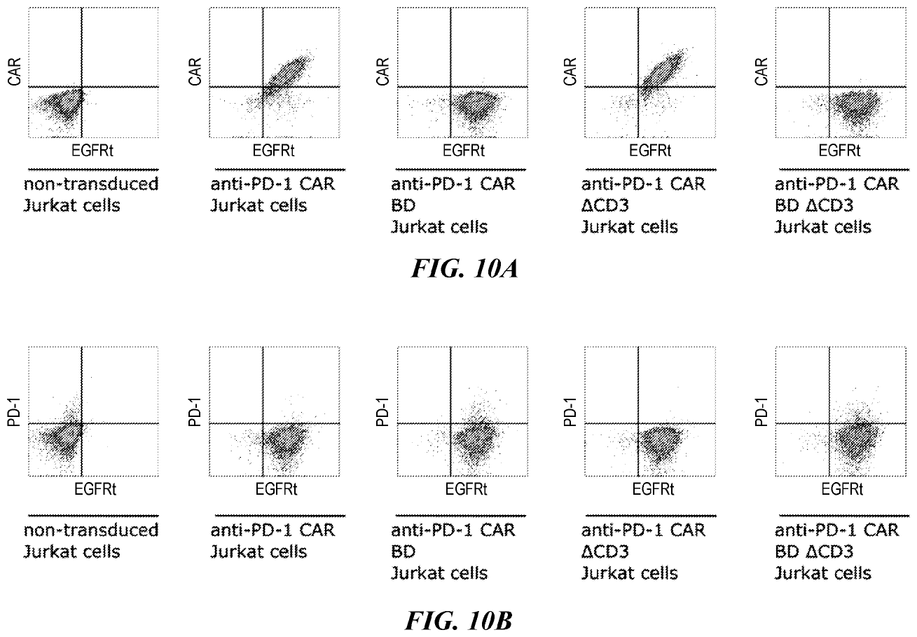

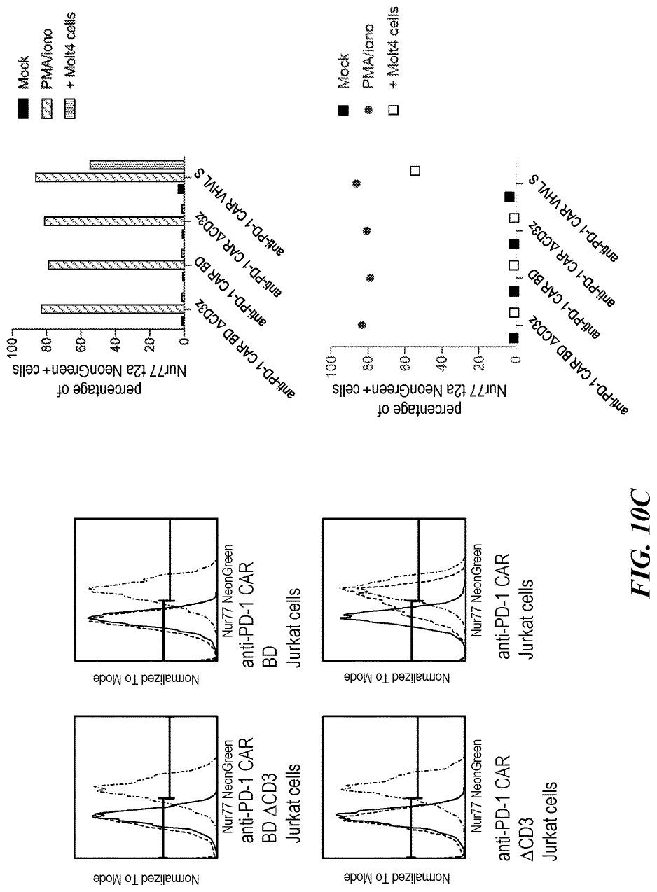

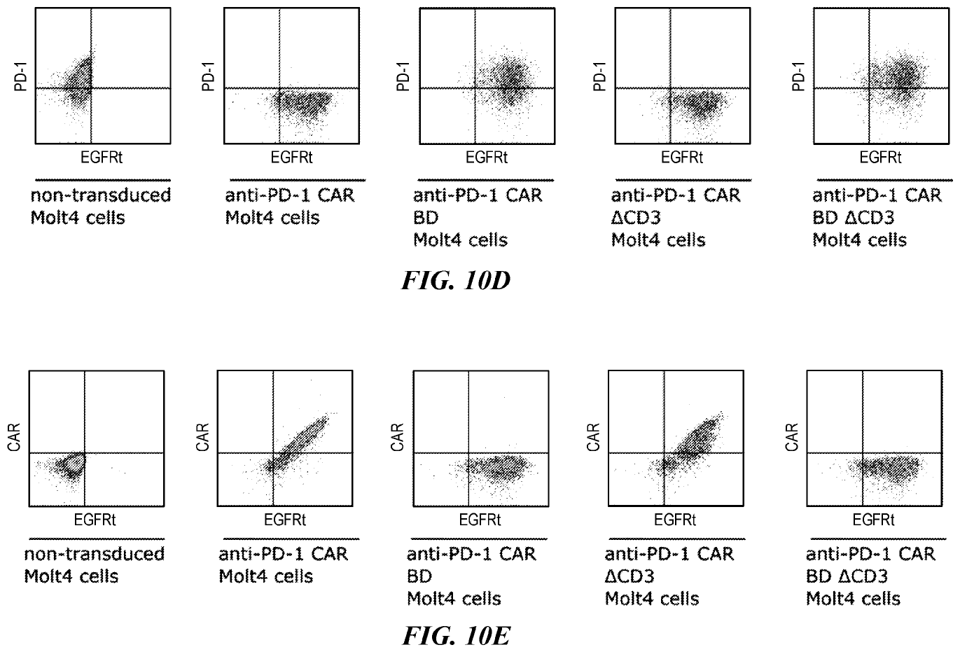

To test the anti-PD-1 CAR in primary T cells, RM CD8+ T cells were transduced with the six anti-PD-1 CAR variants. Gating on EGFRt+ and EGFRt-cells in the culture revealed a loss of PD-1 expression on CAR T and bystander cells in CAR T cell culture in comparison to non-transduced T cells. The EGFRt-tag was detected as a surrogate marker for CAR expression for the six constructs (FIGS. 2A and 9). CARs directed against antigens expressed in T cells may at times lead to fratricide and hamper CAR T cell production. PD-1 expression was decreased for EGFRt+ and EGFRt-T cells in transduced cultures in comparison to Mock-transduced T cells (FIGS. 2B and 9), compatible with ongoing killing of PD-1+ non-transduced T cells through the anti-PD-1 CAR T cells. Pembrolizumab reportedly may shield PD-1 from detection with the anti-PD-1 antibody EH12.2H7 by flow cytometry. To test whether pembrolizumab-based scFv binding to PD-1 on the cell surface in cis may explain low PD-1 levels on anti-PD-1 CAR T cells, Jurkat Nur77-NeonGreen reporter cells were transduced with the V_{H} V_{L} S anti-PD-1 CAR or 3 CAR mutants, which were defective for PD-1 binding (anti-PD-1 CAR BD) by mutation of amino acids implicated in the interaction of pembrolizumab and PD-1, deletion of the CD3z signaling domain (anti-PD-1 CAR ΔCD3ζ) or a combination of both (anti-PD-1 CAR BD ΔCD3ζ). The anti-PD-1 CAR ΔCD3ζ and anti-PD-1 CAR BD ΔCD3ζ constructs were used to rule out a potential feed forward loop of PD-1 interaction with the CAR resulting in activation and upregulation of PD-1. Only the original anti-PD-1 V_{H} V_{L} S CAR and the anti-PD-1 CAR ΔCD3ζ showed binding of PD-1 Fc whereas binding was ablated for anti-PD-1 CAR BD and anti-PD-1 CAR BD ΔCD3ζ variants (FIG. 10A). None of the cell lines expressed PD-1 (FIG. 10B). To validate the loss of PD-1 sensing, Jurkat Nur77-NeonGreen reporter cells expressing the original V_{H} V_{L} S CAR, or the altered constructs were cocultured with the PD-1+ acute lymphoblastic leukemia cell line Molt4 cells. Only the original V_{H} V_{L} S CAR upregulated Nur77 NeonGreen expression as a surrogate marker for CAR signaling (FIG. 10C), indicating that the binding- and signaling-deficient receptors do not signal in the presence of PD-1.

To further study the interaction of the anti-PD-1 CAR with PD-1, Molt-4 that expresses high levels of PD-1 constitutively was used (FIG. 10D). Expression of the anti-PD-1 CAR and the three defective variants significantly decreased detectable cell surface PD-1 in the Molt-4 cell lines that expressed binding-competent anti-PD-1 CAR (anti-PD-1 CAR and the anti-PD-1 CAR ΔCD3ζ) whereas PD-1 expression was unchanged in the Molt-4 cells expressing the binding-deficient receptors (anti-PD-1 CAR BD and anti-PD-1 CAR BD ΔCD3ζ) (FIG. 10D). Interestingly, the CAR paratope was still detectable in presence of the binding-competent anti-PD-1 CAR variants (anti-PD-1 CAR and the anti-PD-1 CAR ΔCD3ζ) indicating that the CAR paratope were not saturated through the cell surface PD-1 and remains functional (FIG. 10E).

To assess killing potential of the six receptor variants in dependence of PD-1 expression, K562 cells were transduced with lentiviral vector to express a PD-1 GFP fusion protein and generated three cell lines coined K562 PD-1 GFP low, medium, and high with differential PD-1 expression. Titration of PD-1 with mouse monoclonal antibody EH12.2H7, which shares the binding site with pembrolizumab and nivolumab, indicated that the three K562 PD-1 GFP lines expressed between approximately 6000 to 40000 PD-1 molecules per cell (FIG. 2C).

To assess killing, RM anti-PD-1 CAR CD8+ T cells after positive selection for EGFRt were cocultured with K562 cells expressing GFP or different levels of PD-1 GFP chimeric protein and GFP expression, used as a surrogate marker for cell killing, was followed over time by life cell imaging (FIG. 2D). Independent of scFv orientation and linker length of the CAR as well as PD-1 expression levels on target cells, the anti-PD-1 CAR T cells efficiently killed and inhibited outgrowth of K562 PD-1 GFP low, medium, and high cells and failed to control K562 GFP cells in the absence ectopic expression of PD-1 (FIG. 2D). Combined, these data indicate that the six anti-PD-1 CAR constructs may interact with PD-1 in cis with little effect on CAR T cell function and that the six anti-PD-1 CAR constructs kill PD-1 expressing cell to a similar degree within the range of the assays. The V_{H} V_{L} S anti-PD-1 CAR construct alone was used in further experimentation.

### Rhesus Macaque Anti-PD-1 CAR T Cells Kill SIV-Infected Cells In Vitro

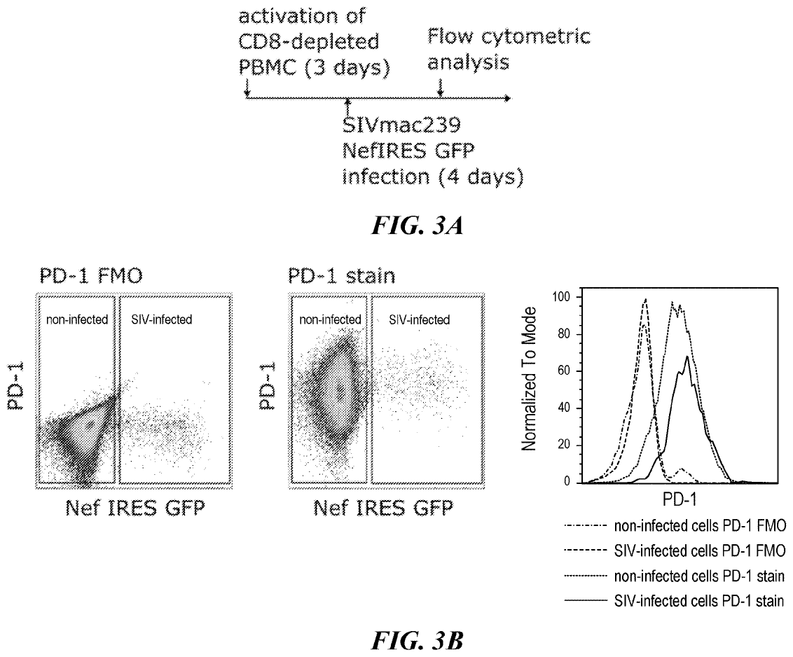

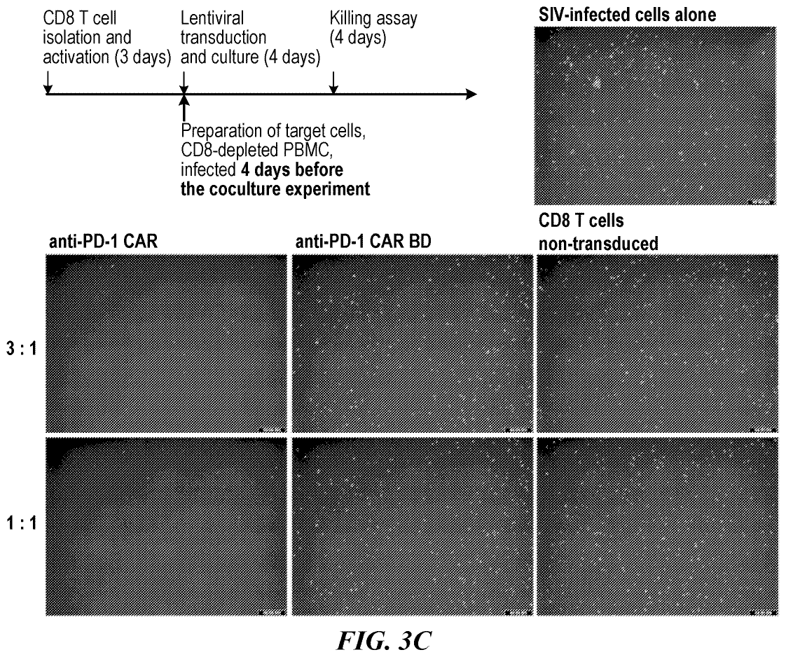

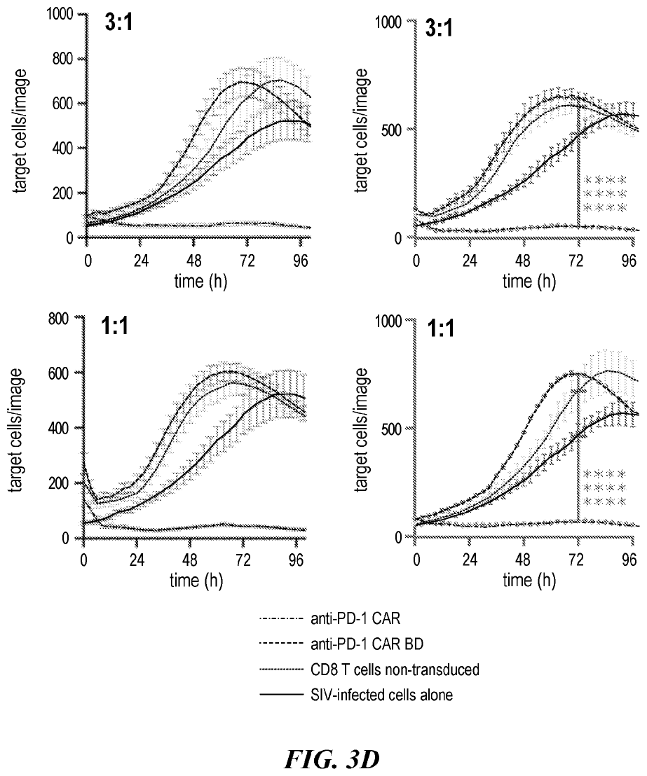

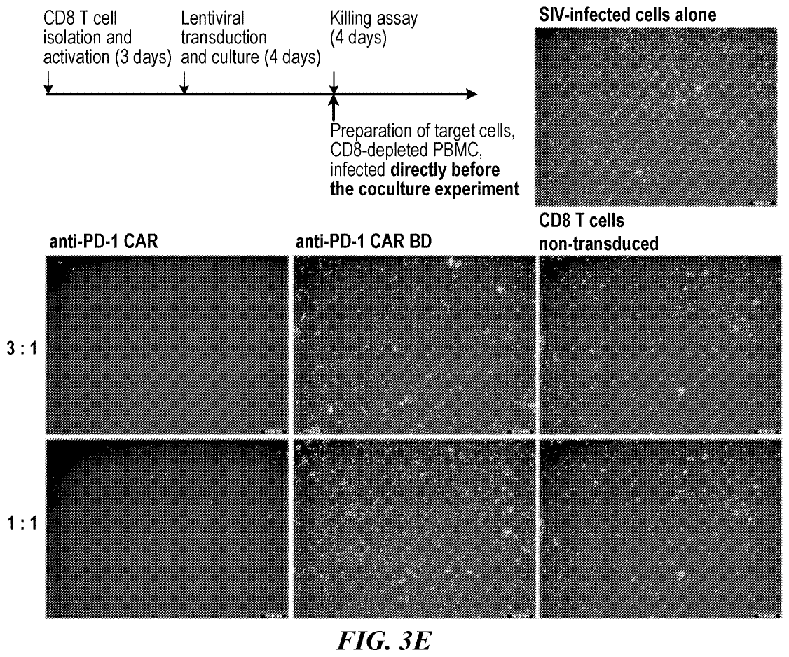

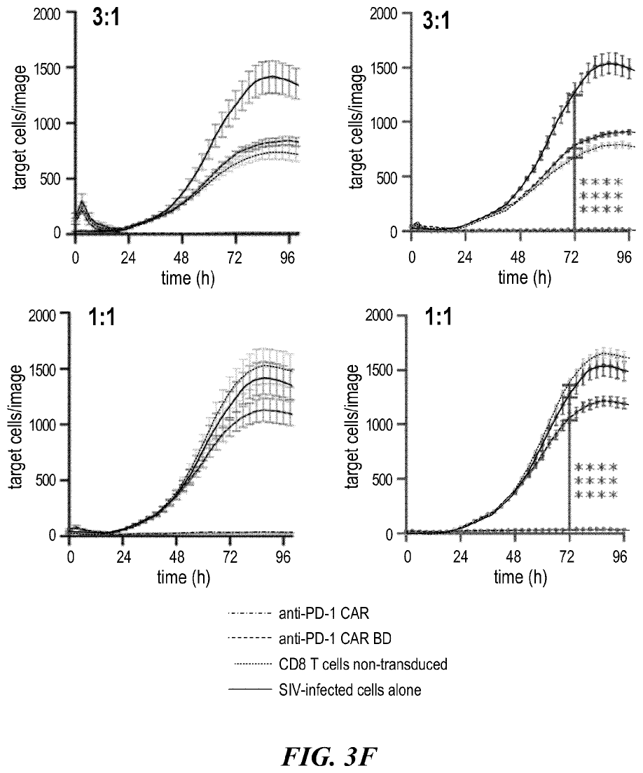

We tested whether anti-PD-1 CAR T cells may attenuate viral outgrowth in vitro as PD-1+ CD4 T cells are a major compartment for HIV and SIV persistence and replication on and off ART. It was initially evaluated whether SIVmac239 preferentially replicates in cells that express PD-1 in an in vitro system. To this aim, activated CD8-depleted PBMC were infected with the SIVmac239 NefIRESGFP virus strain, that express GFP downstream of Nef through an IRES sequences. PD-1 expression on GFP+ cells was assessed after 4 days (FIG. 3A). Infected, GFP+ cells exhibited a higher MFI for PD-1 than non-infected cells. Interestingly, the PD-1 expression levels on infected cells did not differ between infected and non-infected cells (FIG. 3B). Of note, in the in vitro model >95% of CD4 T cells differentiated into an effector memory phenotype based on CD28^{low}CD95^{high} expression by day 4 post-infection and day 7 post-activation, respectively (data not shown). To test whether anti-PD-1 CAR T cells may attenuate SIV replication, a live cell imaging killing assay was used with autologous cells to prepare the CAR T cells from CD8+ T cells and cocultured them with autologous SIVmac239 NefIRESGFP-infected CD8-depleted PBMC as target cells for 96 hours. CD8 T cells expressing the anti-PD-1 CAR, the binding-deficient anti-PD-1 CAR BD or non-transduced CD8+ T cells were used as effector cells. The target cells were infected either directly or 4 days prior to the start of the coculture experiment to test whether the anti-PD-1 CAR may prevent viral outgrowth and ongoing infection, respectively.

When the target cells were infected for 4 days prior to the killing assay, anti-PD-1 CAR T cells efficiently depleted SIV-infected cells at an effector: target (E:T) ratio of 3:1 and 1:1 (FIGS. 3C and 3D). By contrast, non-transduced CD8+ T cells and anti-PD-1 CAR BD cells failed to attenuate viral infection in the culture (FIGS. 3C and 3D) and viral outgrowth kinetics were similar to SIV-infected cells alone (FIG. 3D). Similarly, when the target cells were infected immediately before the killing assay, anti-PD-1 CAR T cells efficiently depleted SIV-infected cells at E:T ratio of 3:1 and 1:1 (FIGS. 3E and 3F). By contrast, non-transduced CD8+ T cells and anti-PD-1 CAR BD cells failed to attenuate viral infection in the culture (FIGS. 3E and 3F) and viral outgrowth kinetics were similar to SIV-infected cells alone (FIG. 3F).

Combined, these data indicate that anti-PD-1 CAR T cells may attenuate SIVmac239 viral outgrowth in vitro.

### PD-1+ Cell Aplasia Following Adoptive Transfer and In Vivo Expansion of Anti-PD-1 CAR T Cells

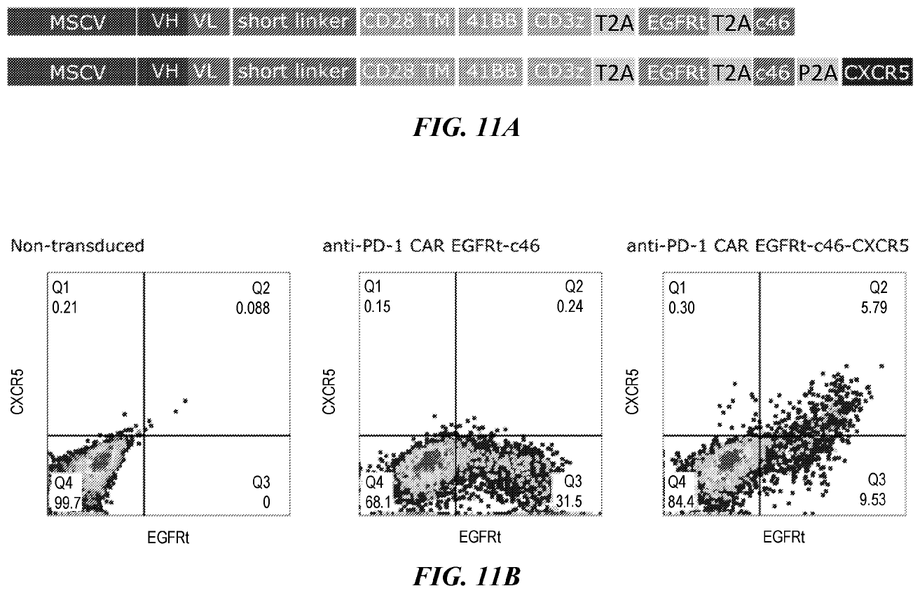

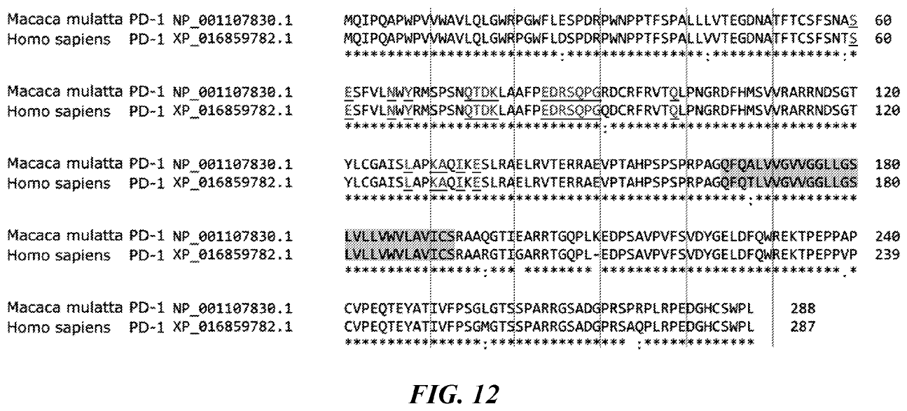

A key feature of HIV infection is its ability to persist in immunologically privileged sites such as lymph node GCs. The NHP model was used to evaluate if the anti-PD-1 CAR T cells could traffic into GC and reduce SIV-infected PD-1+ T cells in vivo. As such, a series of adoptive transfer experiments in both SIV-naive and SIVmac239-infected RMs on ART were performed. 2 pilot studies were performed with first, 2 animals with no SIV infection who received CAR T cells expressing the anti-PD-1 CAR EGFRt c46 construct and second, 4 SIV-infected RMs who were infused with the anti-PD-1 CAR EGFRt c46 construct (n=2) or with an anti-PD-1 CAR EGFRt c46 CXCR5 construct (n=2) (FIG. 11A), which differentially expressed CXCR5 in vitro (FIG. 11B). As previously described CAR T cell production protocol that aims to achieve a 1:1 ratio of CD4 and CD8 CAR T cells was used. Of note, human and RM PD-1 are 96% identical in protein sequences and the amino acids implicated in the interaction with pembrolizumab are 100% conserved between human and RM (alignment shown using CLUSTAL O (1.2.4) multiple sequence alignment tool) (FIG. 12), which may indicate similar anti-PD-1 CAR affinity and signaling strength in response to human and macaque PD-1.

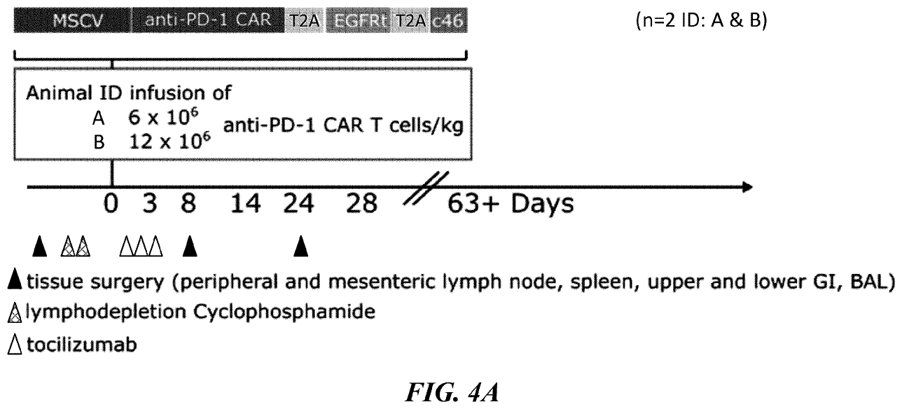

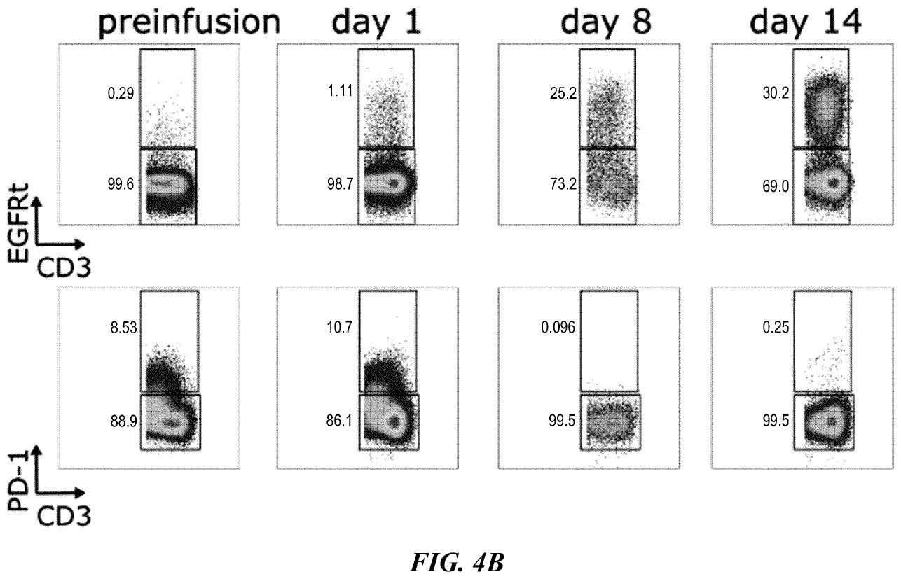

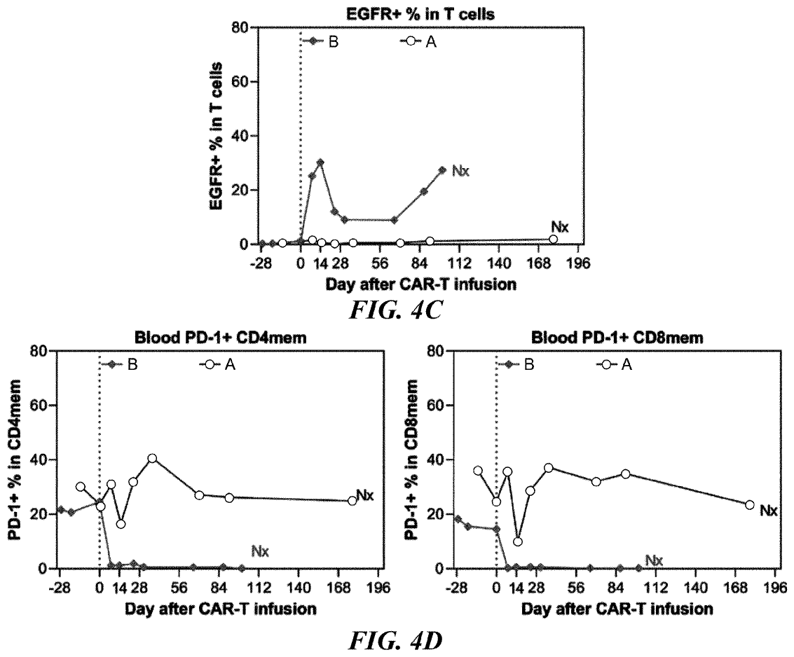

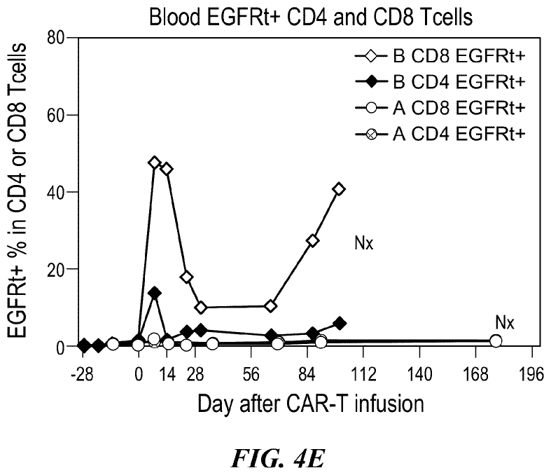

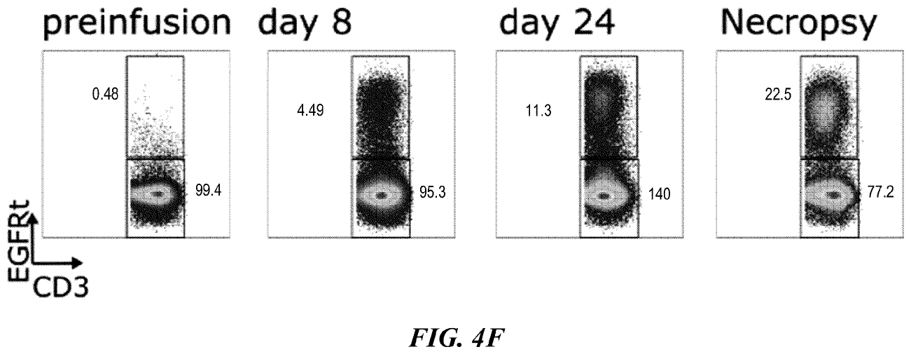

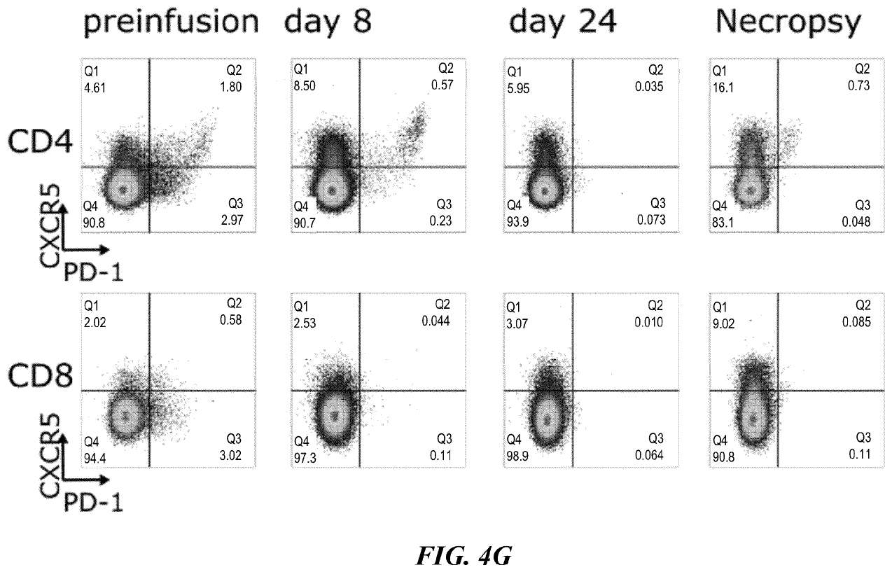

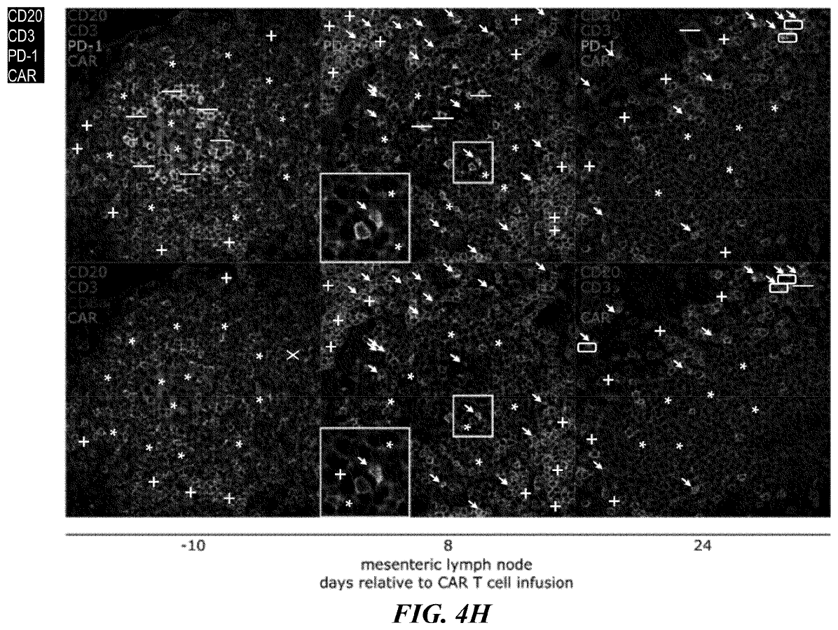

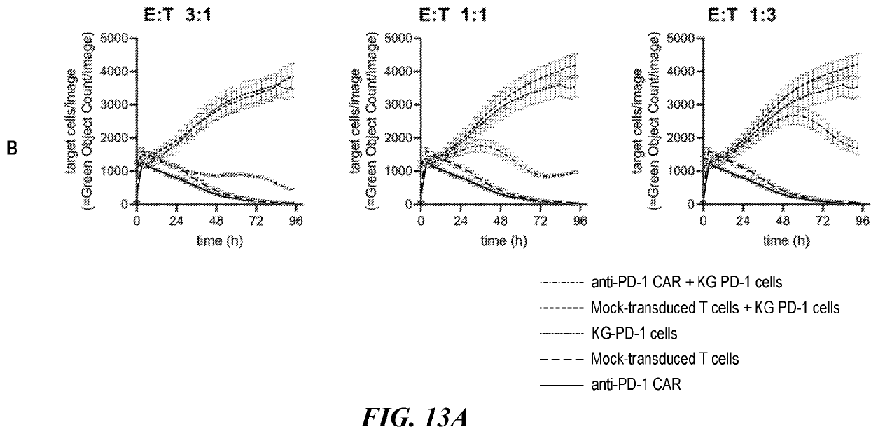

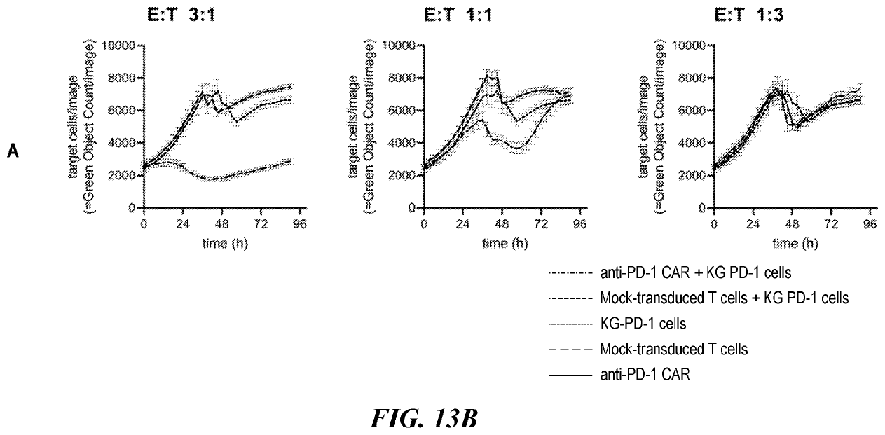

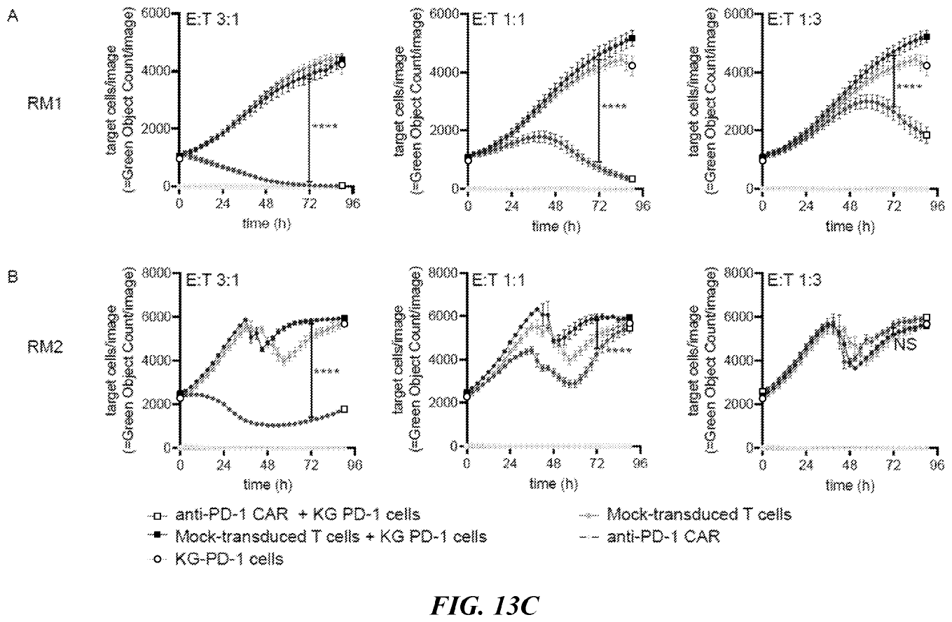

The schema used for adoptive transfer of anti-PD-1 CAR T cells into two SIV-naive RMs is outlined in FIG. 4A. Adoptive transfer of anti-PD-1 CAR T cells into 2 SIV-naive RMs was performed with a dose of 6-12×10⁶ cells/kg T cells expressing the anti-PD-1 CAR-EGFRt-C46 construct (animal ID: animal A and animal B, Table 9). In vitro killing assays to assess functionality of the infusion product showed potent killing in the presence of PD-1+KG cells at a ET ratio of 3:1 for both animal A and animal B (FIGS. 13A-13C), however, efficient killing at a lower ET ratio indicated that the animal B infusion product was more potent than the animal A infusion product (FIG. 13B). E: T ratio were calculated based % EGFRt of CD3+ cells in the infusion product.

Lymphodepletion was performed 3 and 4 days prior to CAR T cell infusion (FIG. 4A) and proliferation post adoptive transfer was successful in the animal receiving the higher dose (ID: animal B) with a bimodal CAR T cell expansion peaking at day 14 with 30.2% EGFRt+ cells of total CD3, an intermittent phase of lower frequency and a second peak at day 100 (Table 9, FIGS. 4B and 4C). For the second RM (ID: animal A), more moderate CAR T cell expansion (up to 6.49% EGFRt+ cells of total CD3) was observed, peaking at day 3 with a lack of CAR T cell persistence and depletion of PD-1+ T cells (FIG. 4C). Depletion of total PD-1+ memory CD4 and CD8 T cells based on CD28+/CD95+ and CD28−/CD95+ expression was maintained for 100 days in animal B whereas animal A showed no sign of depletion (FIG. 4D). The majority of expanding and persisting anti-PD-1 CAR T cells were CD8+ (FIG. 4E). Anti-PD-1 CAR T cell expansion was detectable in lymphoid tissues of animal B on day 8, 24 and 100 (necropsy time point)-post-infusion by flow cytometry (FIG. 4F) and occurred concomitantly with depletion of memory PD-1+ CD8, PD-1+ CD4 as well as T_{FH} cells based on co-expression of PD-1 and CXCR5 (FIG. 4G). Multicolor RNA FISH and immunofluorescence microscopy were used to confirm access of anti-PD-1 CAR CD8 T cells to B cell follicles along with depletion of T_{FH} cells. Intrafollicular PD-1 expression markedly decreased at day 8 and 24 post-infusion and was accompanied with the presence of CD8a+ CAR RNA+ T cells within the perimeter (FIG. 4H). Combined, these data indicate that anti-PD-1 CAR T cells may expand in blood and tissues in SIV-naive RMs and deplete PD-1+ CD8+ and CD4+ memory T cells including T_{FH} cells. The ability to successfully transfer functional T cells and the evidence of trafficking into GC with expansion of CAR T cells post-infusion in the initial pilot led to the experiments with SIV-infected RMs.

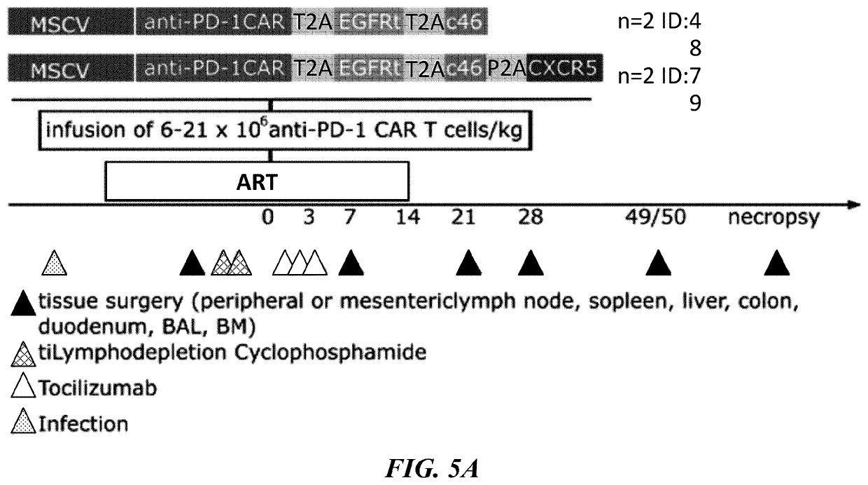

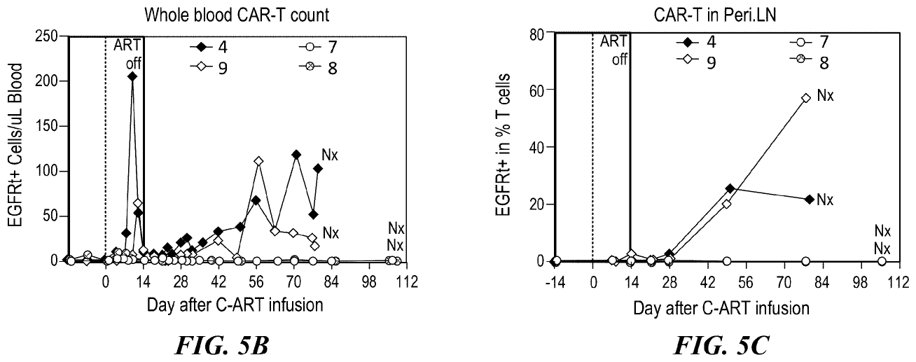

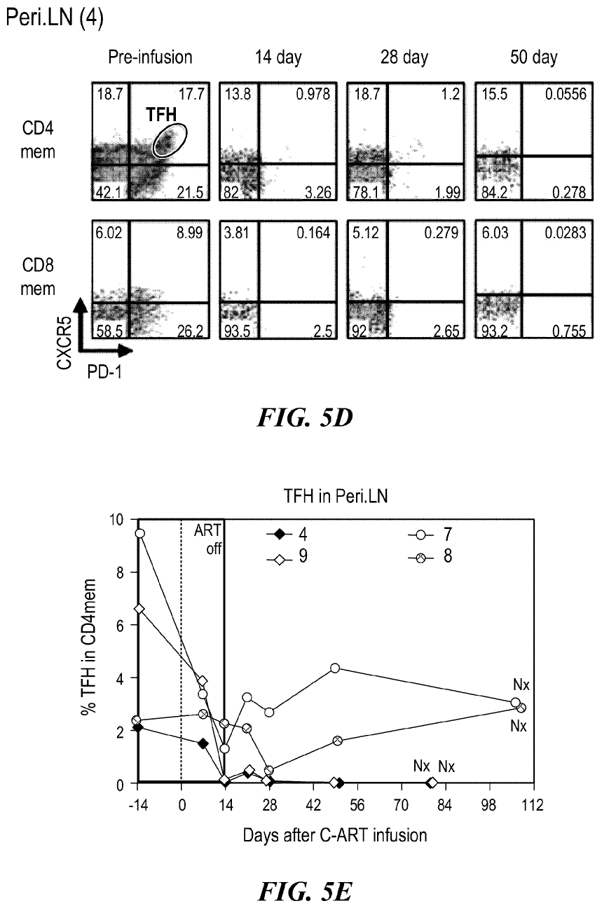

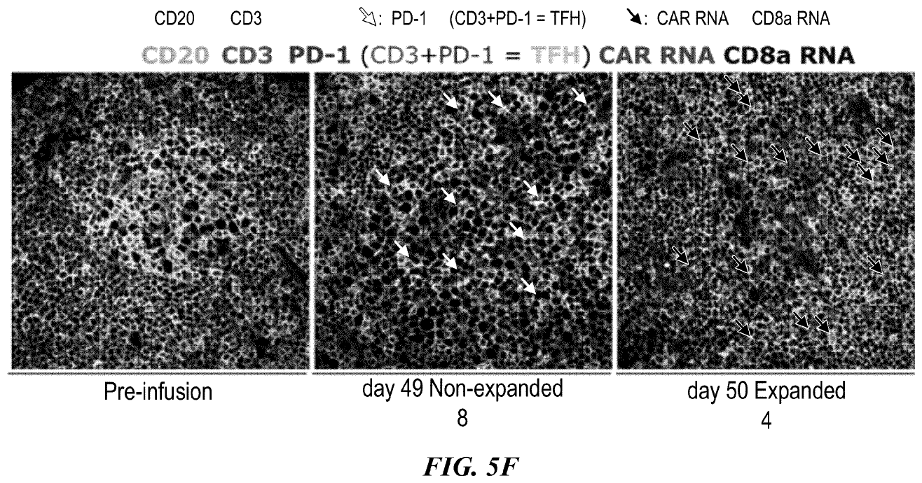

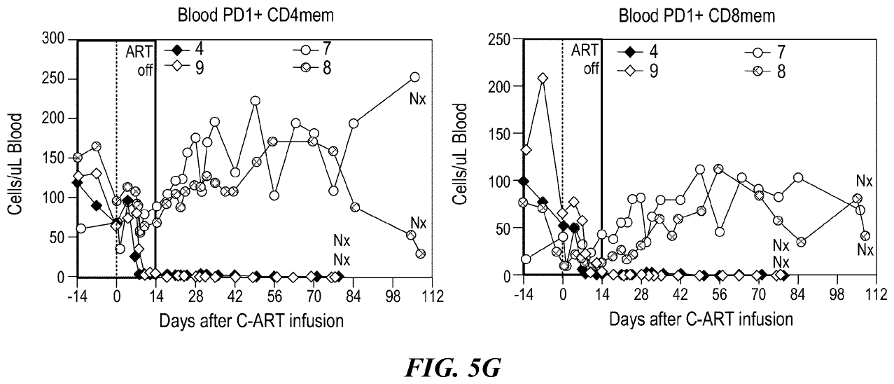

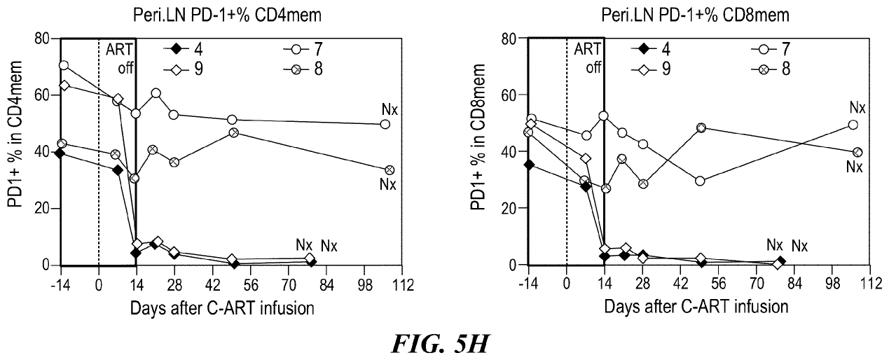

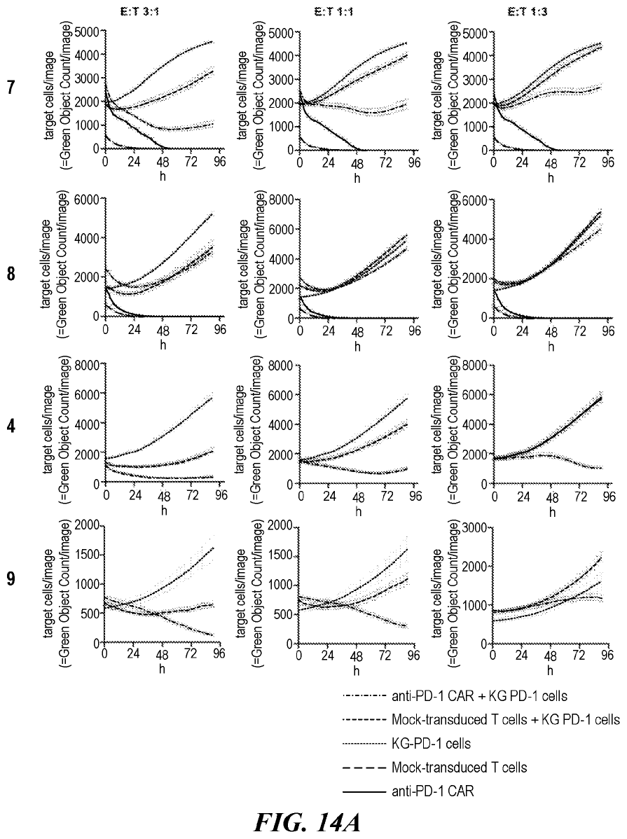

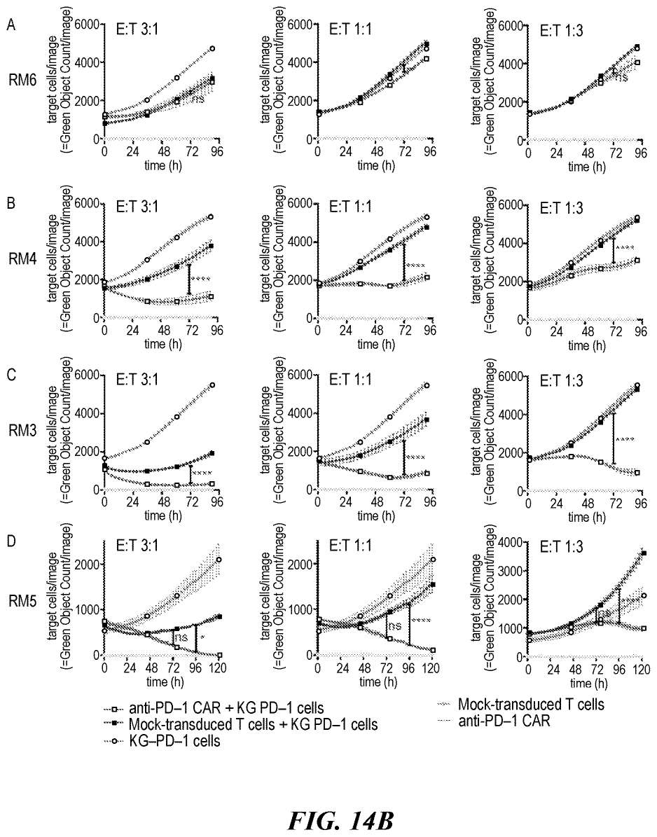

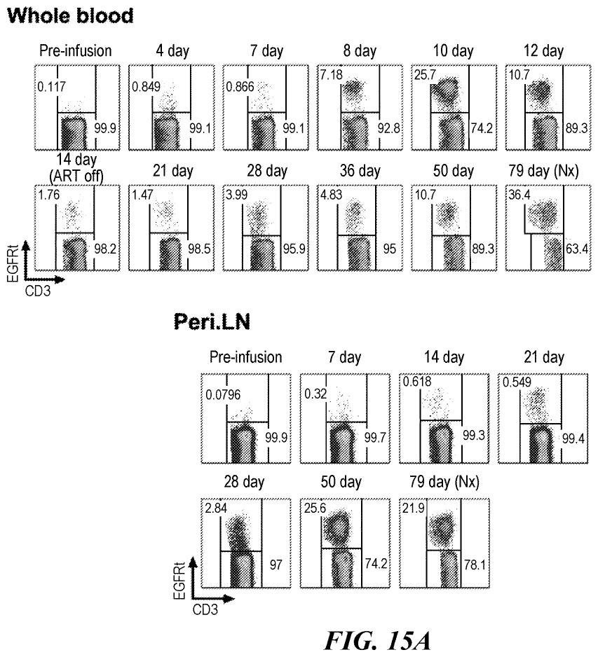

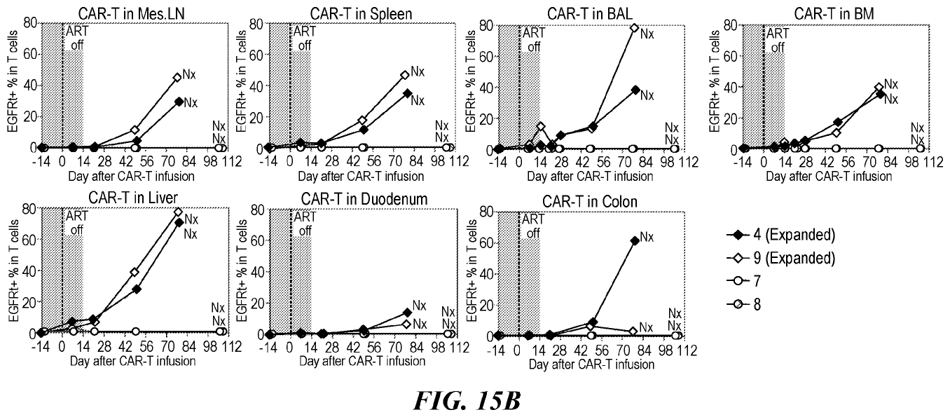

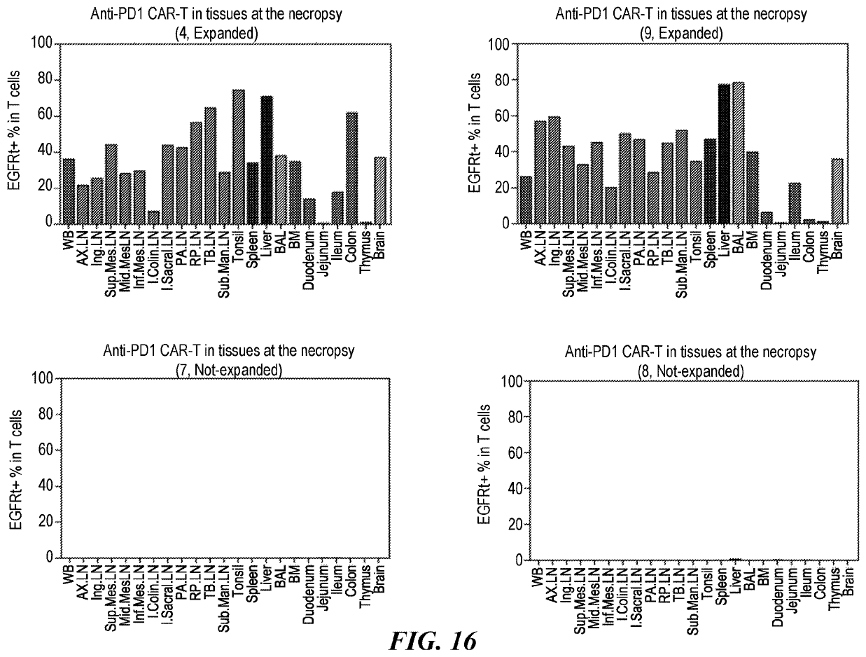

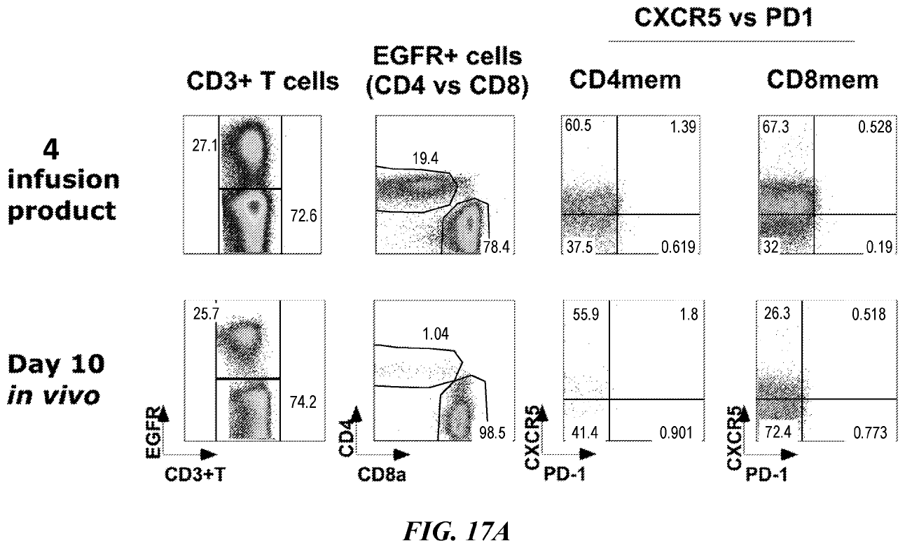

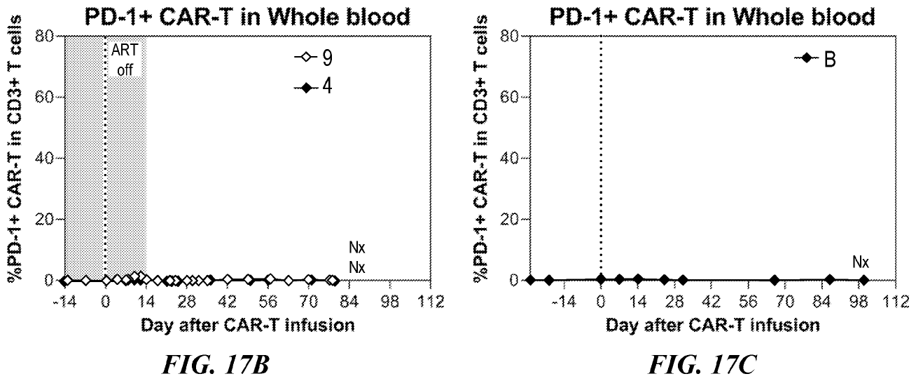

The schema used for adoptive transfer of anti-PD-1 CAR T cells into four SIV-infected ART-treated RMs is outlined in FIG. 5A. The RMs were inoculated with SIVmac239, and ART was initiated 12 days post-inoculation. The animals were infused with T cells engineered to express an anti-PD-1 CAR-EGFRt-c46 (n=2, ID animal 7 and animal 4) or an anti-PD-1 CAR-EGFRt-c46-CXCR5 expression cassette (n=2, ID animal 8 and animal 9) at least 3 months after ART initiation and released from ART 14 days post-CAR T cell infusion (FIG. 5A). The cell dosage ranged between 6 and 20.8×10⁶ CAR T cells/kg and additional characteristics for each infusion product are shown in Table 9. Killing efficacy was assessed for each infusate which indicated that the infusion product for animal 8 had only little potency in vitro whereas the infusates for animal 7, animal 4, and animal 9 were potent at killing KG PD-1 cells across different E: T ratios (FIGS. 14A and 14B). E:T ratio were calculated based % EGFRt of CD3+ cells in the infusion product. All infusions were well tolerated without occurrence of CAR T cell-related adverse events. No clinical neurotoxicity was observed. Anti-PD-1 CAR T cells expanded in peripheral blood of two of the four animals (animal 4 and animal 9) with initial peak expansion of 200 and 60 EGFRt+ cells/μL blood at day 10 and 14, respectively (FIG. 5B, Table 9, FIG. 15A). Peripheral lymph node trafficking of anti-PD-1 CAR T cells occurred in both animals with evidence of expansion in blood (FIGS. 5C and 15A). CAR T cells were detected also in spleen, mesenteric lymph nodes, liver, lung, bone marrow and to a lesser extent in the gastro-intestinal tract (FIG. 15B). At necropsy, CAR T cells were detectable by flow cytometry at >10% in most lymphoid tissues, liver, lung, bone marrow, ileum, and perfused brain of animal 4 and animal 9 but not in the 2 animals with no evidence of post-infusion expansion: animals animal 8 and animal 7 (FIG. 16). Interestingly, CAR T cells were not detectable in jejunum and thymus at necropsy (FIG. 16). In accordance with the in vitro data, anti-PD-1 CAR T cell infusion products and in vivo-expanded CAR T cells lacked PD-1 expression (FIG. 17A). Analysis of longitudinal samples indicated that anti-PD-1 CAR T cells had no measurable PD-1 expression at any point throughout the study time course in either the SIV-infected (FIG. 17B) or SIV-naive RMs (FIG. 17C).

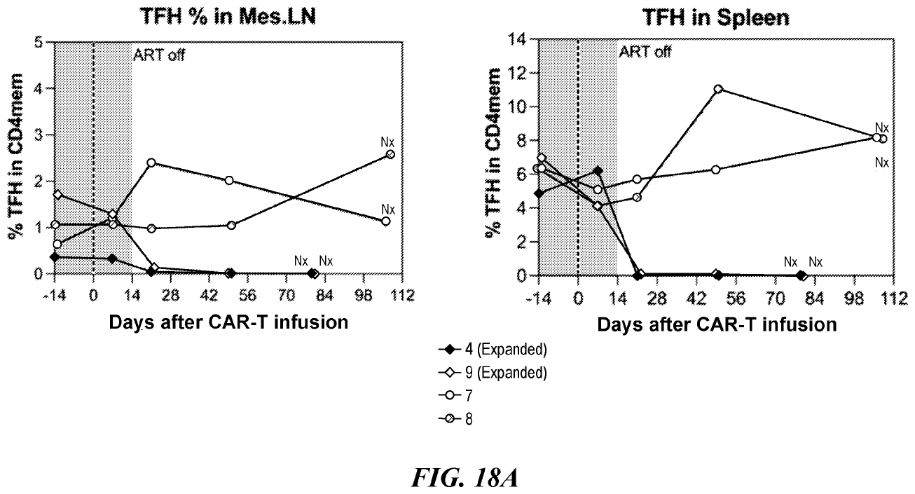

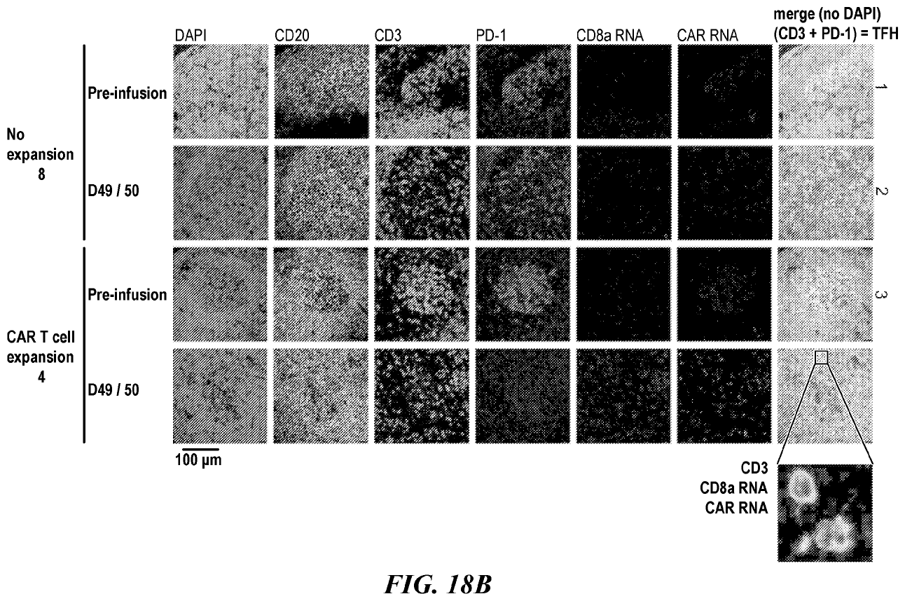

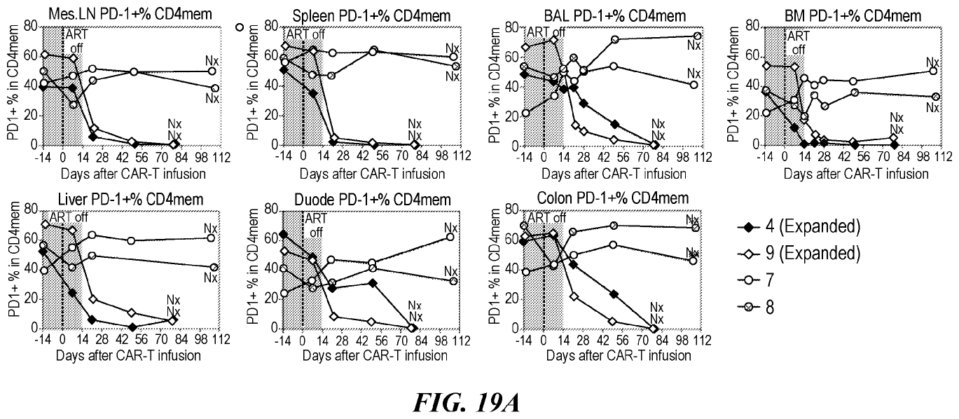

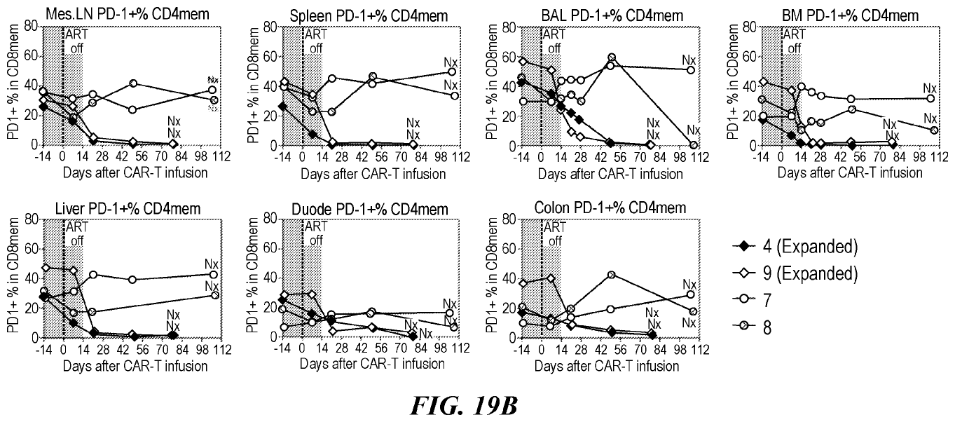

Depletion of T_{FH} cells, characterized by high co-expression of PD-1 and CXCR5, occurred in animals with CAR T cells expansion in peripheral lymph nodes at the time of ART release (FIGS. 5D and 5E) as well as in mesenteric lymph nodes and the spleen (FIG. 18A). To confirm trafficking of anti-PD-1 CAR CD8+ T cells to B cell follicles as well as CAR T cell-mediated depletion of T_{FH} cells, a combined immunofluorescence and RNA FISH assay were used for CAR RNA, CD8α RNA, PD-1, CD20, and CD3. Intrafollicular anti-PD-1 CAR CD8+ T cells were used and depletion of T_{FH} cells in GCs of the B cell follicles in day 49/50 tissue section in the two animals with anti-PD-1 CAR T cell expansion (animal 4, animal 9), but not in the RMs without CAR T cell expansion (animal 8, animal 7) (FIGS. 5F and 18B). Of note, there appeared to be no difference in B cell follicles access between anti-PD-1 CAR T cells that co-expressed CXCR5 (animal 9) or did not (animal 4) (data not shown). In summary, these studies in SIV-naive and SIV-infected RMs indicate that anti PD-1 CAR T cells are efficient in accessing immune privileged sites such as the B cell follicles and may deplete T_{FH} cells as a major site of HIV replication (Table 10). Depletion of PD-1+ memory CD8+ and CD4+ occurred also in other secondary lymphoid tissues, the lung, liver, duodenum, and colon (FIGS. 19A and 19B). Depletion of peripheral blood memory PD-1+ CD4+ and CD8+ T cells occurred concomitantly with CAR T cell expansion in animal 4 and animal 9 but not in animal 8 and animal 7 (FIG. 5G). Depletion of memory PD-1+ CD4+ and CD8+ T cells was long-lasting until necropsy, consistent with persistence of CAR T-cell expansion (FIG. 5G). Similarly, long-lasting depletion of PD-1+ memory CD8+ and CD4+ T cells was also documented in peripheral lymph nodes in animals with CAR T cell expansion (FIG. 5H).

### Viral Rebound Occurs in the Absence of T_{FH} Cells in Extrafollicular T Cells

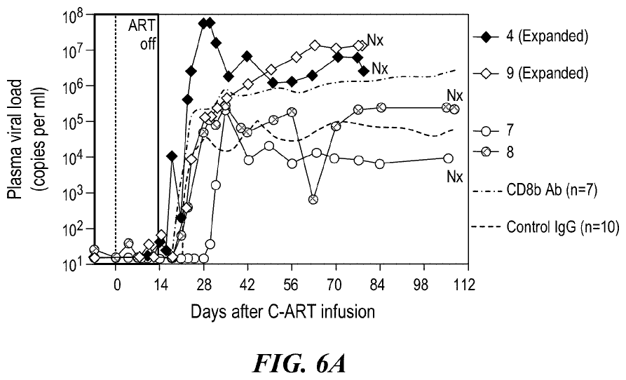

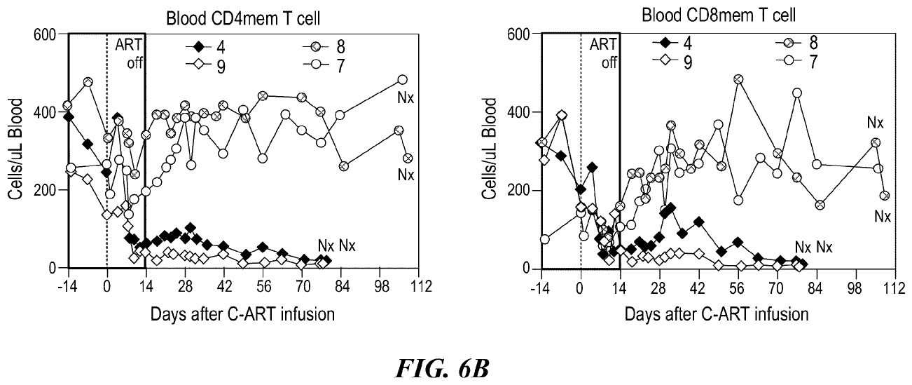

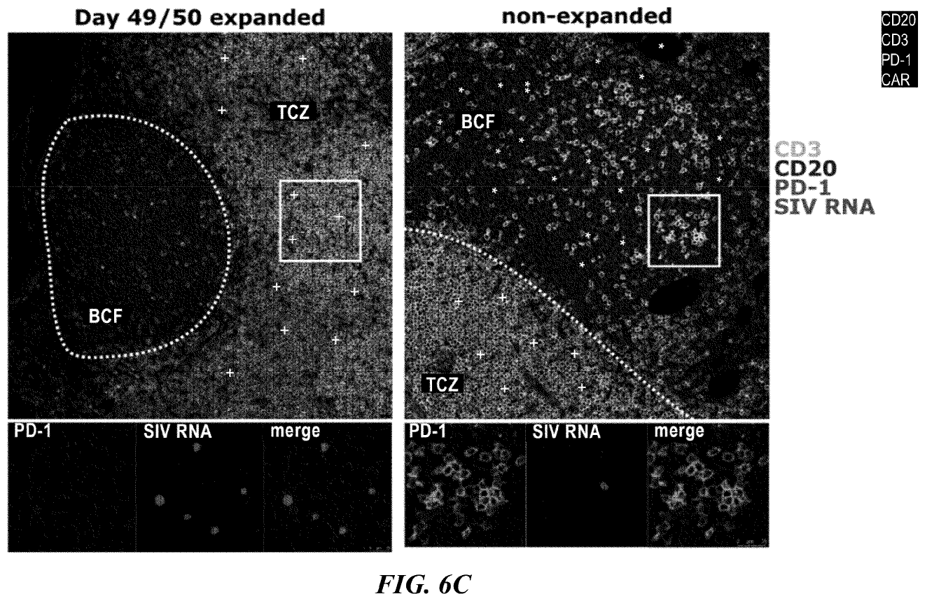

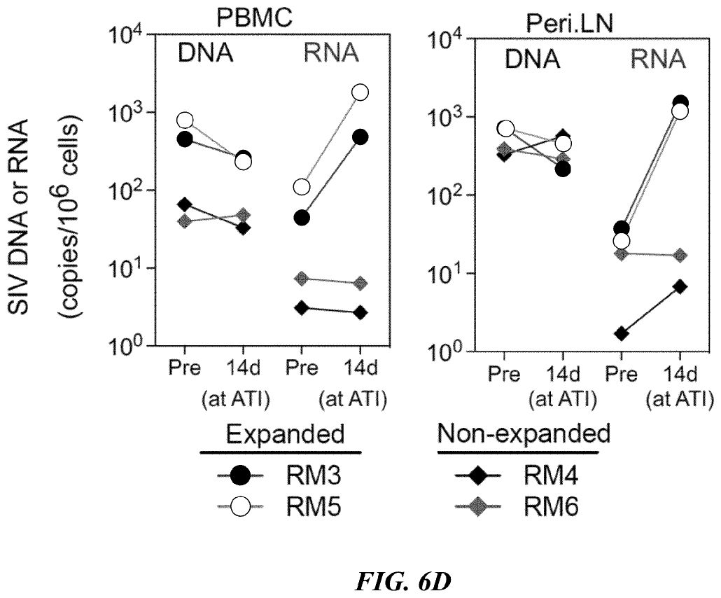

PD-1+ CD4 T cells and T_{FH} cells play a central role in HIV and SIV infection and persistence and were successfully depleted by anti-PD-1 CAR T cells. However, viral rebound after removal of ART occurred in all four animals independent of CAR T cell expansion and depletion of PD-1+T_{FH} cells. In addition, plasma viral load was notably higher (~2 log) in animal 4 and animal 9 (FIG. 6A), the animals with CAR T-cell expansion. This loss of viral control in the RMs with anti-PD-1 CAR T cell expansion was comparable to historic data from a previous study in which CD8+ T cells were depleted with a rhesusized anti-CD8B monoclonal antibody at the time of ART removal. Accordingly, a significant depletion of memory CD4 and CD8 T cells was observed in the RMs with CAR T cell expansion (FIG. 6B) indicating that potentially the loss of SIV-specific memory CD8+ T cells in the extrafollicular areas led to the higher plasma viral load levels. Precluding the increased high viral load in the 2 RM with anti-PD-1 CAR T cell expansion was a 42.8- and 13.2-fold increase in cell-associated viral RNA levels measured in PBMC and LN in the 2 RM with anti-PD-1 CAR T cell expansion (vs. 2.5- and 0.9-fold in the two RM without anti-PD-1 CAR T cell expansion) on day 14 compared to a pre-infusion time point (FIG. 6D). By contrast, cell-associated DNA levels remained stable in the same time frame.

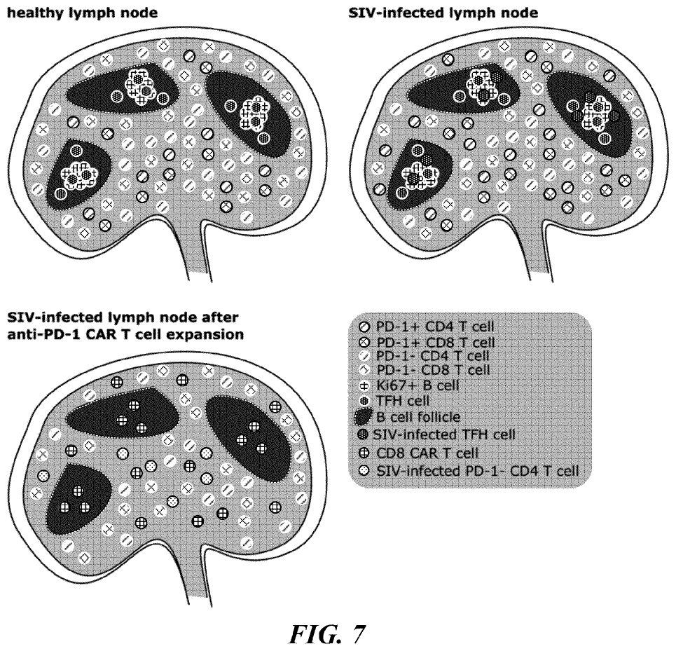

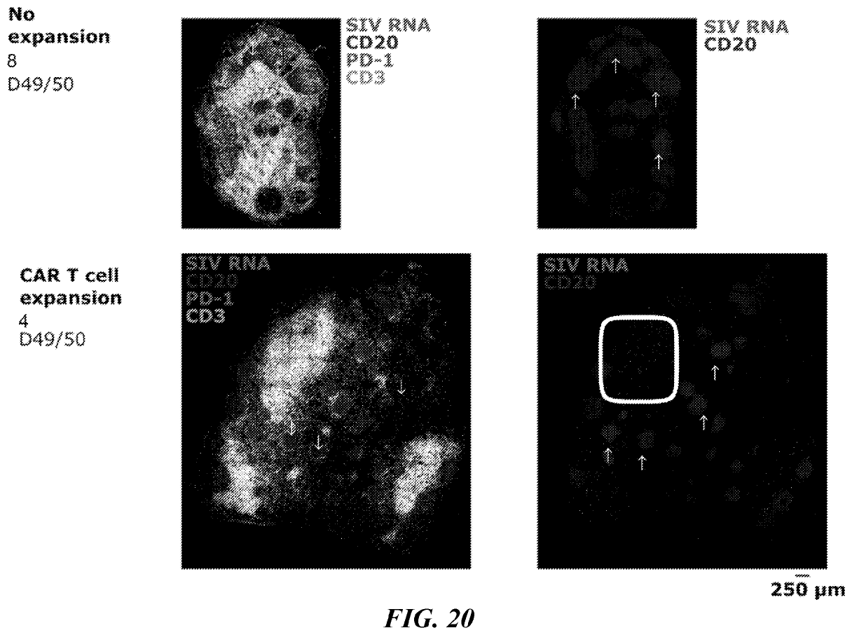

Using combined immunofluorescence and RNA FISH for SIV RNA, PD-1, CD20, and CD3, SIV+ PD-1− T cells were detected in the perifollicular zone in day 49/50 tissue section in the two animals with anti-PD-1 CAR T cell expansion (animal 4, animal 9), but not in the ones which failed to expand (animal 8, animal 7) (FIG. 6C). Of note, SIV RNA+ cells were not detected in lymphoid tissues of animal 7 at this timepoint (data not shown), which is also reflected in the low plasma viral load post-ART removal (FIG. 6A). Intrafollicular SIV RNA+ cells were largely absent in animal 4 and animal 9, 49/50 days relative to CAR T infusion (FIG. 6C). However, single viral particles were present on the follicular dendritic cell network, indicating the specificity of the CAR T cells for eradicating PD-1-expressing SIV+ cells (FIG. 6C). In animal 8, which had no CAR T cell expansion, PD-1+ SIV RNA+ T cells were observed within B cell follicles (FIGS. 6C and 20). These data indicate that anti-PD-1 CAR T cells may successfully expand in SIV-infected RMs and deplete T_{FH} as well as PD-1+ CD4+ and CD8+ T cells (FIG. 7).

TFH cells are localized to the B cell follicles where they may support B cell responses. Extrafollicular PD-1 expressing T cells are part of the memory compartment or have been recently exposed to antigen. During lentiviral infection, there may be an increase of TFH cells, extrafollicular PD-1+ CD4+ and CD8+ T cells and viral replication occurring predominantly in TFH cells. During successful expansion, anti-PD-1 CAR T cells enter lymphoid tissues and kill extrafollicular PD-1+ T cells as well as TFH cells in the follicles. This results in relatively lower viral replication in the follicles due to depletion of TFH cells and increases viral replication in the extrafollicular T cell zone where it occurs in PD-1− CD4+ T cells. SIV-specific T cells are likely part of extrafollicular PD-1+ T cell population and are lost due to depletion of the anti-PD-1 CAR T cells. Concomitantly, GC B cell proliferation may be attenuated due to the loss of TFH cells. Overtime, this leads to an anti-PD-1 CAR T cell-mediated immunodeficiency.

Furthermore, depletion of PD-1+ CD4+ T cells, a major cell compartment that harbors the SIV replication-competent reservoir and an important site for persistent virus replication, at the time of ART removal focused viral replication to the extrafollicular zone, which was associated with plasma viral rebound of SIV. Specific depletion of PD-1+ CD4+ and CD8+ T cells appears to have induced a form of CAR-mediated immunodeficiency with an attenuation of cellular and humoral responses, which likely exacerbated SIV infection.

### Persistence of Anti-PD-1 CAR T Cells in SIV-Naive and SIV-Infected Rhesus Macaques Mediates an Anti-PD-1 CAR T Cell-Induced Immunodeficiency

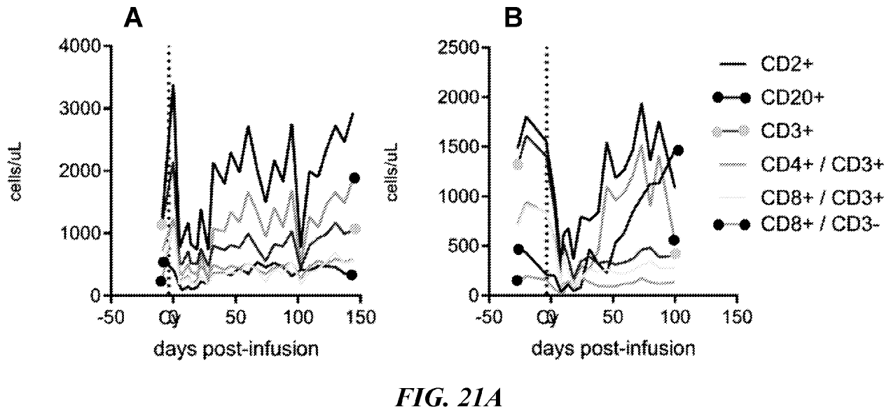

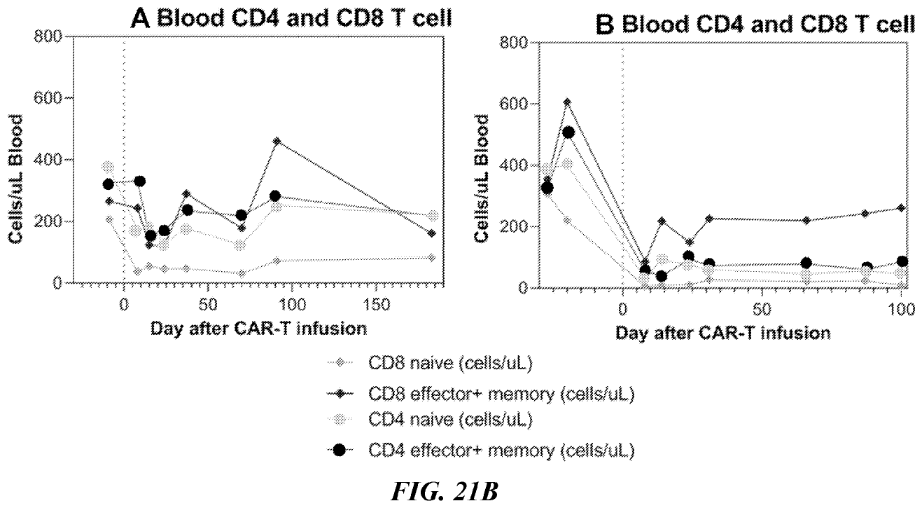

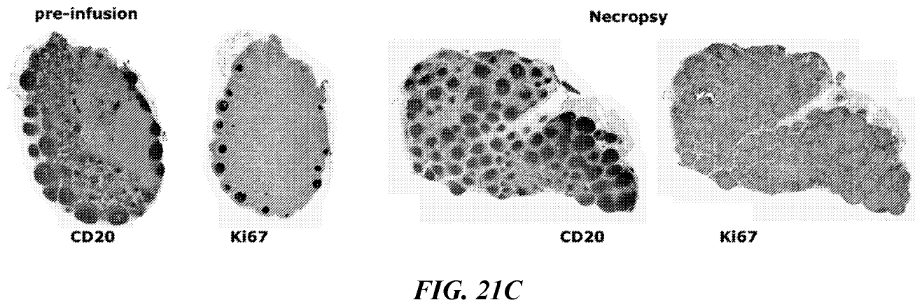

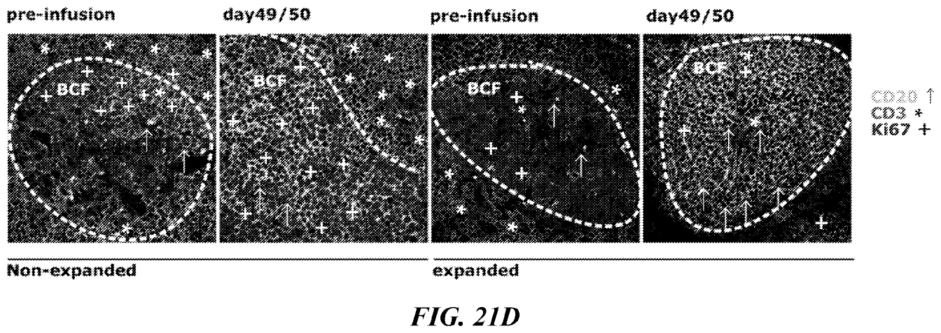

The infusion and expansion of anti-PD-1 CAR T cells concomitant with depletion of PD-1+ T cells occurred without laboratory or clinical evidence of cytokine release syndrome. No evidence of the neurological symptoms reported with anti-CD19 or anti-CD20 CAR T cells were noted. The long-term persistence of the CAR T cells was observed in this study. From 10% to 40% CAR T cells of total T cells in PBMCs were seen at greater than 30 days post-infusion. This anti-PD-1 CAR expansion and persistence was associated with long-lasting depletion of memory CD4 and CD8 T cells in both SIV-infected animals (ID: animal 4 and animal 9) (FIG. 6B). This immunosuppression was associated with monocytosis, hyperalbuminemia, diarrhea, and anemia (Table 10 and Table 12). Recovery of total CD4+ T cells was also hampered in the SIV-naive animal (ID: animal B) (FIG. 21A). Interestingly, absolute memory CD8+ T cells count remained stable at a higher level in the SIV-naive animal with CAR T cell expansion (ID: animal B: 200 cells/μL+/−60 cells/μL blood) post-infusion (FIG. 21B), than observed in SIV-infected animals (ID: animal 4 and animal 9) (FIG. 6B). RMs animal B, animal 4 and animal 9 were necropsied on days 100, 79 and 78 post-infusion, respectively, due to meeting clinical end points. At necropsy, high frequencies of CAR T cells were seen in lymphoid tissue as well as lung, liver, and brain (FIG. 16). Anti-PD-1 CAR T cells were not seen in animals that did not show initial CAR T cell-proliferation post-infusion.

TABLE 9
Characteristics of six types of anti-PD-1 CAR T cells for infusion

| | | | | | | | | | | | CAR T expansion | |
|---|---|---|---|---|---|---|---|---|---|---|---|---|
| | | | | Profile of infused CAR T cells | | | | | | | in vivo | |
| | | | | | | | Total | | | | | Initial |
| | | | | | Total | EGFRt+ | infused | Infused | | | Peak % | peak |
| | | | | Infusion | Infused | CAR T | CAR T | CAR T | CD4+ | CD8+ | EGFRt+ | expan- |
| | | | | day after | T cells | % in | cells | cells | % in | % in | CAR T | sion |
| Animal | | | SIV | lentiviral | (10\hat{ }6 | infused | (10\hat{ }6 | (10\hat{ }6 | EGFR + | EGFR+ | in | (Post- |
| ID | Weight | CAR ID | status | transduction | cells/kg) | cells | cells/kg) | cells/kg) | CAR T | CAR T | PBMC | infusion) |
| animal B | 9.4 kg | PD1 | Negative | 4 day | 24.8 | 48.2% | 112 | 12.0 | 55.8% | 43.2% | 58.40% | 14 day |
| animal 4 | 6.6 kg | PD1 | Positive | 4 day | 77.0 | 27.0% | 137 | 20.8 | 19.4% | 78.4% | 25.70% | 10 day |
| animal 9 | 12.2 kg | PD1- | Positive | 7 day | 41.6 | 12.3% | 82 | 6.7 | 33.0% | 64.1% | 13.90% | 12 day |
| | | CXCR5 | | | | | | | | | | |
| animal A | 8.7 kg | PD1 | Negative | 4 day | 36.7 | 17.0% | 52 | 6.0 | 42.4% | 57.6% | 6.49% | 3 day |
| animal 8 | 7.4 kg | PD1- | Positive | 4 day | 66.7 | 18.0% | 88 | 12.0 | 53.1% | 41.4% | 0.77% | 7 day |
| | | CXCR5 | | | | | | | | | | |
| animal 7 | 7.6 kg | PD1 | Positive | 4 day | 25.7 | 30.1% | 59 | 7.7 | 17.7% | 80.3% | 0.68% | 21 day |

TABLE 10
Comparison of anti-PD-1 observations
in SIV-naive and SIV-infected RMs

| | SIV-infected RM | healthy RM |
|---|---|---|
| CD4+ T cells | depletion of PD-1 + | depletion of PD-1 + |
| | memory CD4+ T cells | memory CD4+ T cells |
| CD8+ T cells | depletion of PD-1 + | depletion of PD-1 + |
| | memory CD8+ T cells | memory CD8+ T cells |
| | | was less pronounced |
| lymphocytopenia | | total CD4+ subset |
| GC B cells | loss of Ki67+ GC | loss of Ki67+ GC |
| | B cells | B cells |
| germinal centers | underdeveloped | underdeveloped |
| Monocytes | Monocytosis | Monocytosis |

TABLE 11
Anti-PD-1 CAR T cell expansion and depletion of
T_{FH} cells does not affect antibody recall responses.

| | Post-infusion | ID50¹ | ID50^{1, 2} |
|---|---|---|---|
| Animal | timepoint (weeks) | SVA-MLV | SIVmac251.6 |
| 3 | 0 | 232 | 11,969 |
| 3 | 0.5 | <200 | 15,747 |
| 3 | 1 | <200 | 11,299 |
| 3 | 2 | <200 | 14,230 |
| 3 | 5 | <200 | 124,828 |
| 3 | NX | <200 | 92,906 |
| 4 | 0 | 411 | 3,530 |
| 4 | 0.5 | <200 | 999 |
| 4 | 1 | <200 | 4,269 |
| 4 | 2 | <200 | 2,553 |
| 4 | 5 | <200 | 43,802 |
| 4 | NX | <200 | 89,206 |
| 6 | 0 | 395 | 8,819 |
| 6 | 0.5 | <200 | 8,534 |
| 6 | 1 | <200 | 10,580 |
| 6 | 2 | <200 | 20,858 |
| 6 | 5 | <200 | 165,034 |
| 6 | NX | <200 | 204,072 |
| 5 | 0 | 381 | 20,557 |
| 5 | 0.5 | <200 | 15,217 |
| 5 | 1 | <200 | 10,037 |
| 5 | 2 | <200 | 21,239 |
| 5 | 5 | <200 | 367,931 |
| 5 | NX | <200 | 52,799 |

¹Values are the purified IgG concentration or flowthrough dilutions at which relative luminescence units (RLUs) were reduced 50% compared to virus control wells (no test sample).
²Values considered positive for neutralizing antibody activity in the sample based on the criterion of >3X background signal against the negative control MLV-pseudotyped virus.

TABLE 12
Summary of adverse event and necropsy findings

| Animal | | | | | | |
|---|---|---|---|---|---|---|
| ID | Animal 8 | Animal 7 | Animal A | Animal 9 | Animal 4 | Animal B |
| SIV | Positive | Positive | Negative | Positive | Positive | Negative |
| status | | | | | | |
| CAR T | No | No | No | Yes | Yes | Yes |
| cell | | | | | | |
| expansion | | | | | | |
| Clinical | no significant | no significant | no significant | Hypoalbuminemia, | Slow healing | Monocytosis, |
| observation | findings | findings | findings | anemia | wounds, | CD4+ |
| | | | | (HCT 27%, | monocytosis, | lymphocytopenia |
| | | | | RBC 4.01) | hypoalbuminemia, | Slight anemia with |
| | | | | w/1.8% | treated with | decreased RBC |
| | | | | reticulocytes, | cefazolin | No weight loss |
| | | | | monocytosis, | | |
| | | | | platelets | | |
| | | | | were | | |
| | | | | decreasing | | |
| | | | | but still in the | | |
| | | | | normal | | |
| | | | | range. | | |
| | | | | diarrhea and | | |
| | | | | low appetite | | |
| | | | | treated with | | |
| | | | | enrofloxacin | | |
| | | | | and | | |
| | | | | cefazolin. | | |
| | | | | Weight | | |
| | | | | hadn't | | |
| | | | | decreased | | |
| | | | | yet. | | |
| Autopsy | | | 1. Mild to moderate, | | | 1. Mild to moderate, |
| report | | | diffuse, eosinophilic, | | | pulmonary, multifocal, |
| Final | | | lymphoplasmacytic | | | arterial and arteriolar, |
| principal | | | and histiocytic gastro- | | | nodular, subintimal to |
| Diagnosis | | | entero-colitis with | | | medial, connective |
| | | | enteric villar blunting | | | tissue and smooth |
| | | | and fusion, and with | | | muscular |
| | | | moderate to | | | proliferations: nodular |
| | | | extensive, near- | | | pulmonary |
| | | | diffuse, large | | | proliferative |
| | | | intestinal | | | arteriopathy |
| | | | spirochetosis | | | 2. Moderate, diffuse, |
| | | | 2. Chronic-fibrous | | | eosinophilic, |
| | | | encapsulated, focal, | | | lymphoplasmacytic |
| | | | deep dermal to | | | and histiocytic gastro- |
| | | | subcutaneous | | | entero-colitis with |
| | | | abscess | | | enteric villar blunting |
| | | | | | | and fusion, and with |
| | | | | | | moderate to |
| | | | | | | extensive, multifocal, |
| | | | | | | large intestinal |
| | | | | | | spirochetosis 3. Low |
| | | | | | | to moderate follicular |
| | | | | | | activity with generally |
| | | | | | | sparsely populated |
| | | | | | | lymphoid follicles: |
| | | | | | | lymph nodes and |
| | | | | | | spleen 4. Moderate, |
| | | | | | | multifocal, adrenal, |
| | | | | | | corticomedullary |
| | | | | | | calcification |
| Histology | Staining for | Staining for | The inflammatory | Atypical | Moderate to | Histology Comments: |
| comments | lymphocrypto- | lymphocrypto- | component of | leukocyte | marked | The nodular |
| | viral RNA, | viral RNA, | diagnosis #1, which | proliferation | atypical | proliferative |
| | carried out on | carried out | can cause diarrhea | was present | leukocyte | pulmonary |
| | select tissues, | on select | and potentially other | in lymphoid | proliferation | arteriopathy was |
| | revealed rare | tissues, | sequelae thereof, | and most | was present | unexpected |
| | positive | revealed | represents typical | major | in most | particularly as this |
| | leukocytes in | rare positive | changes in this | organs, | lymph nodes | lesion is typically |
| | the spleen, | leukocytes | species in this colony, | largely | are well as | associated with |
| | axillary lymph | in the | and they have been | associated | the spleen | certain strains of |
| | node, and | spleen, | previously discussed. | with positive | and thymus, | lentivirus. In this |
| | ileocecal | axillary | Inflammatory | lymphocrypto- | which in | case, consequences |
| | junction. This | lymph node, | changes present are | viral (LCV) | areas | of the experimental |
| | indicates a | inguinal | consistent with food | RNA staining | disrupted the | manipulations is |
| | minimally | lymph node, | allergy/hypersensitivity/ | were | normal | favored as the cause. |
| | active | middle | dietary | assessed. | architecture. | The lesions were |
| | opportunistic | mesenteric | intolerance/IBD. The | The | This was | currently clinically |
| | infection likely | lymph node, | bacterial organisms | pleomorphic | largely | silent, but could have |
| | due to | jejunum, | diagnosed should be | nature, high | associated | progressed. The |
| | immuno- | and ileum. | considered | mitotic rate, | with positive | lymphoid follicular |
| | compromise. | This | commensals in my | presence in | lymphocrypto- | changes also are |
| | | indicates a | opinion. Please | multiple | viral (LCV) | favored as being |
| | | minimal | contact me if you | tissues, and | RNA staining | secondary to |
| | | active | wish to discuss these | vascular | where | experimental |
| | | opportunistic | changes further. | invasion | assessed. | manipulations. |
| | | infection due | As previously | indicate a | LCV staining | The inflammatory |
| | | to the | mentioned, the | neoplastic | was | component of |
| | | experimental | encapsulated | process of | multifocally | diagnosis #2, which |
| | | protocol. | abscess was an | probable | present | can cause diarrhea |
| | | | incidental lesion. | large cell | within the | and potentially other |
| | | | | lymphoma. | tonsil and | sequelae thereof, |
| | | | | LCV is a | bone | represents typical |
| | | | | well- | marrow. LCV | changes in this |
| | | | | documented | is a well- | species in this colony, |
| | | | | inciting cause | documented | and they have been |
| | | | | of atypical | inciting | previously discussed. |
| | | | | lymphocytosis | cause of | Inflammatory |
| | | | | and non- | atypical | changes present are |
| | | | | Hodgkins-like | lymphocytosis | consistent with food |
| | | | | lymphoma | and non- | allergy/ |
| | | | | (NHL) in SIV- | Hodgkins | hypersensitivity/ |
| | | | | infected | lymphoma | dietary |
| | | | | rhesus | (NHL) in | intolerance/IBD. The |
| | | | | macaques. | SIV-infected | bacterial organisms |
| | | | | These | rhesus | diagnosed should be |
| | | | | lymphomas | macaques. | considered |
| | | | | are typically, | As with | commensals in my |
| | | | | B cell-derived | another | opinion. |
| | | | | and hence | animal in this | |
| | | | | CD20+. LCV | cohort, the | |
| | | | | NHLs, | pleomorphic | |
| | | | | however, are | leukocytic | |
| | | | | primarily | proliferation | |
| | | | | extranodal; | was mostly | |
| | | | | an aspect in | present | |
| | | | | which this | within lymph | |
| | | | | case | nodes, which | |
| | | | | deviates. | would be | |
| | | | | | abnormal for | |
| | | | | | LCV NHLS | |
| | | | | | that are | |
| | | | | | generally | |
| | | | | | extranodal. | |

Interestingly, total B cell counts increased over time in peripheral blood in the SIV-naive RMs (FIG. 21A). Proliferating Ki67+ B cells are located predominately in GCs within the follicles in lymphoid tissues and are dependent on the presence of T_{FH} cells to provide cytokines. However, at necropsy, a loss of Ki67+ B cells was observed within the GCs of the SIV-naive RM animal B with CAR T cell expansion (FIG. 21C). For the SIV-infected RMs, multicolor immunofluorescence staining was performed for CD3, CD20, and Ki67 to confirm the loss of Ki67+ B cells in GCs on day 49/50 post-infusion. Anti-PD-1 CAR T cell expansion notably decreased Ki67+ B cells whereas the two SIV-infected RMs in which no anti-PD-1 CAR T-cell expansion occurred retained fully developed B cell follicles with abundant Ki67+ cells in GCs (FIG. 21D). To assess how the depletion of TFH cells affected memory B cell responses and antibody recall responses, longitudinal serum samples pre- and post-anti-PD-1 CAR T cell infusion were assessed for the presence of anti-SIV neutralizing antibodies. We noted substantial increases in neutralization titers was observed between wk2 and wk5 post-infusion, with a 1-2 log increase in titers for the 4 RMs (Table 11 regardless of anti-PD-1 CAR T cell proliferation; suggesting memory B-cell responses are maintained.

Post-mortem analysis of the SIV-infected RMs indicated that increased atypical leukocyte proliferation was present in lymphoid tissue, most major organs, and bone marrow, mostly associated with lymphocryptovirus (LCV) RNA+ staining in animals with CAR T cell expansion (Table 12). LCV is a well-documented etiological agent of atypical non-Hodgkin lymphoma (NHL)-like lymphocytosis in SIV-infected RMs and the earlier onset in the absence of immune effector cells such as CD8+ T cells has been observed previously. These studies indicate that anti-PD-1 CAR T cells may induce T cell-related immunodeficiency in a healthy RM and contributes to total loss of memory T cells, AIDS-related opportunistic infections, and accelerated disease progression in SIV-infected RMs.

This study developed an anti-PD-1 CAR to target PD-1 expressing cells and tested its safety profile, expansion and tissue trafficking in vivo in both SIV-uninfected and ART-treated SIVmac239-infected RMs. Anti-PD-1 CAR T cells expanded successfully in 3 out of 6 RMs; all 3 of these animals, whether SIV-infected or not infected; consistently depleted PD-1+ T cells in both blood and tissues. Rapid depletion of CD4+ T_{FH} was achieved in all animals with CAR T-cell expansion. This CAR T cell expansion was associated with high density infiltration of anti-PD-1 CAR T cells in the GC; subsequent elimination of T_{FH} cells and significant loss of SIV RNA+ cells in the GC in association with absence of T_{FH} cells. This demonstrated successful inhibition of viral replication in this immune privileged viral sanctuary and offers potential for a conceptual advancement in the HIV cure field. This depletion of CD4+ T_{FH} cells was partially achieved as early as day 7 post-infusion; a week prior to ART release. CD4+ T_{FH} cells were completely ablated by day 14 post-infusion; notably the last day of ART; suggesting that release from ART may not be necessary for T_{FH} depletion and elimination of SIV/HIV from this reservoir and that rapid elimination of the anti-PD-1 CAR T cells may be a potential strategy for reducing the effects of their long-term persistence. Interestingly, the anti-PD-1 CAR T cells exhibited prolonged biologically active persistence in vivo. Among the 3 RMs with initial expansion, all exhibited continued expansion of anti-PD-1 CAR T cells in peripheral blood over the 3-month follow-up period, often at high levels. This expansion was associated with widespread depletion of PD-1+ T cells and loss of both memory CD4+ and CD8+ T cells as well as loss of Ki67+ B cells. Loss of the T cell memory compartment by the approach has two implications; first the anti-PD-1 CAR did not discriminate between higher PD-1 expression on T_{FH} cells and relatively lower PD-1 expression on memory CD4+ and CD8+ T cells and second, PD-1 is expressed on all memory CD4+ and CD8+ T cells during their maturation from naive T cells. Elimination of the memory T cell pool was associated with a clinical immunodeficiency.

In SIV-infected RMs that showed CAR T cell expansion, SIVmac239 replication increased, despite near complete depletion of PD-1+ CD4+ T cells at the time of ART removal; post-ART plasma viral load set points were about 2 logs higher relative to RMs without CAR T cell expansion. This expansion of viral RNA in plasma was associated with a remarkable increase in SIV-infected extrafollicular PD-1-CD4+ T cells; a pattern seen in chronic-phase viremic SIVmac239-infected RMs that progress to AIDS rapidly but not in elite controller RMs. This further illustrates the importance of antiviral activity of the T lymphocyte population in these tissue sites. Future studies should focus on more detailed kinetics of this reactivation of lentivirus infection in these anatomic sites, and the role and mechanism that endogenous immune responses play in viral containment both during suppressive therapy and release. Long-term persistence of the anti-PD-1 CAR T cells appeared to accelerate disease progression in the SIV-infected animals, potentially due to diminished SIV-specific effector CD4 and CD8 T cell responses (FIG. 7). Interestingly, memory CD8 T cell depletion was less pronounced in the SIV-uninfected RM, however, loss of memory CD4 and Ki67+GC B cells was demonstrated. Thus, while the primary hypothesis that anti-PD-1 CAR T cells could selectively abrogate CD4 T_{FH} cells containing SIV turned out to be correct and achievable, the associated “on target” immunodeficiency indicates that additional genetic engineering is required to increase anti-PD-1 CAR specificity toward CD4 T cells alone, and to include safety switches to abrogate anti-PD-1 CAR T cells in vivo to minimize the long-term effects of CAR T-cell persistence.

The development of anti-PD-1 CAR-mediated or -accelerated immunodeficiency characterized by loss of memory CD4+ and CD8+ T cells, increased SIV replication, and development of an abnormal nodular lymphoma-like condition associated with LCV RNA+ cells was likely mediated by one or several mechanisms that hampered the antiviral responses of the T and B cell compartment. With respect to the T cell compartment, PD-1 expression on CD4+ and CD8+ T cells is quickly upregulated during T cell activation and exhaustion including HIV-specific T cells, in the presence of pro-inflammatory cytokines, as well as homeostatic T cell proliferation during lymphopenia. Arguably, all of these mechanisms were present due to CAR T cell infusion, lymphodepletion prior to infusion and, for the SIV-infected RMs, rebound of SIV after cessation of ART and may have contributed to the almost complete depletion of the memory T cell compartment during the initial expansion, and created a window of extended lymphopenia. Notably, antibody-mediated depletion of CD8+ T cells in SIVmac239-infected RMs showed a similar pattern of an approximately 2-log increase in post-ART plasma viremia relative to controls after cessation of ART. Stronger depletion of CD8+ memory T cells in the SIV-infected RMs was observed compared to the SIV-naive animal, perhaps due to the ongoing viral replication and T cell-responses in the SIV-infected RMs. The functional outcome of anti-PD-1 CAR T cell expansion on the B cell compartment was not assessed, however, one may speculate that the loss of T_{FH} cells, which secrete IL-21 to maintain B-cell proliferation in the GC to facilitate the affinity maturation of antibodies, impaired de novo antibody response. Whether the loss of GC Ki67+ B cells is a consequence of T_{FH} cell depletion or through killing of B cells by the anti-PD-1 CAR is not known. The main factors that contributed to the effects seen in the T and B cell compartment in RMs with CAR T cell expansion were unable to be resolved considering the limited number of animals; however, the development of an anti-PD-1 CAR-related immunodeficiency remains an observation of this study.

Although expansion of the CAR T cells was not achieved in 3 of the 6 infused animals, the contrast in depletion and SIV infection in the animals in which the cells did not expand was useful in defining many of the unexpected observations from this study. The infusate for animal 8 did not show in vitro killing of KG PD-1 cells in the potency release assay and may explain why it did not expand in vivo. Why the animal 7 and animal A infusates failed to expand remains elusive and may be related to unappreciated differences in product quality, CAR T cell fratricide or in vivo expression dynamics of PD-1.

### Additional Embodiments

Various embodiments of the present technology are set forth below in paragraphs [0311] to [0381]:

A chimeric antigen receptor (CAR) comprising a signal peptide, an extracellular binding domain specific to programmed death protein 1 (PD-1), a hinge domain, a transmembrane domain, an intracellular costimulatory domain, and/or an intracellular signaling domain.

The CAR of embodiment 1, wherein the extracellular binding domain specific to PD-1 comprises an scFv derived from a portion of pembrolizumab.

The CAR of embodiment 2, wherein the scFv comprises one or more complementarity determining regions (CDRs) having amino acid sequences at least about 50%, 55%, 60%, 65%, 70%, 75%, 80%, 85%, 90%, 95%, or 100% identical to SEQ ID NOs: 6-8 and 11-13.

The CAR of embodiment 3, wherein the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and/or a light chain variable region comprising an amino acid sequence that is at least about 50%, 55%, 60%, 65%, 70%, 75%, 80%, 85%, 90%, 95%, or 100% identical to SEQ ID NO: 10.

The CAR of embodiment 4, wherein the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and a light chain variable region comprising an amino acid sequence that is at least about 50%, 55%, 60%, 65%, 70%, 75%, 80%, 85%, 90%, 95%, or 100% identical to SEQ ID NO: 10.

The CAR of embodiment 4, wherein the scFV comprises an amino acid sequence that is at least about 50%, 55%, 60%, 65%, 70%, 75%, 80%, 85%, 90%, 95%, or 100% identical to SEQ ID NO: 4 or SEQ ID NO: 14.

The CAR of embodiment 6, wherein the scFV comprises an amino acid sequence that at least about 50%, 55%, 60%, 65%, 70%, 75%, 80%, 85%, 90%, 95%, or 100% identical to SEQ ID NO: 4.

The CAR of embodiment 6, wherein the scFV comprises an amino acid sequence that is at least about 50%, 55%, 60%, 65%, 70%, 75%, 80%, 85%, 90%, 95%, or 100% identical to SEQ ID NO: 14.

The CAR of any one of embodiments 1-8, wherein the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

The CAR of any one of embodiments 1-9, wherein the hinge domain comprises an IgG4 hinge domain having an amino acid sequence at least about 50%, 55%, 60%, 65%, 70%, 75%, 80%, 85%, 90%, 95%, or 100% identical to SEQ ID NOs: 18-20.

The CAR of any one of embodiments 1-10, wherein the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

The CAR of any one of embodiments 1-11, wherein the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

The CAR of any one of embodiments 1-12, wherein the intracellular signaling domain comprises a CD3ζ signaling domain having an amino acid sequence at least about 50%, 55%, 60%, 65%, 70%, 75%, 80%, 85%, 90%, 95%, or 100% identical to SEQ ID NO: 25.

The CAR of any one of embodiments 1 to 13, wherein the extracellular binding domain specific to PD-1 comprises or consists of an amino acid sequence set forth in any one of SEQ ID NOs: 27-32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in any one of SEQ ID NOs: 27-32.

The CAR of any one of embodiments 1 to 13, wherein the extracellular binding domain specific to PD-1 comprises or consists of an amino acid sequence set forth in SEQ ID NO: 27, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 27.

The CAR of any one of embodiments 1 to 13, wherein the extracellular binding domain specific to PD-1 comprises or consists of an amino acid sequence set forth in SEQ ID NO: 28, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 28.

The CAR of any one of embodiments 1 to 13, wherein the extracellular binding domain specific to PD-1 comprises or consists of an amino acid sequence set forth in SEQ ID NO: 29, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 29.

The CAR of any one of embodiments 1 to 13, wherein the extracellular binding domain specific to PD-1 comprises or consists of an amino acid sequence set forth in SEQ ID NO: 30, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 30.

The CAR of any one of embodiments 1 to 13, wherein the extracellular binding domain specific to PD-1 comprises or consists of an amino acid sequence set forth in SEQ ID NO: 31, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 31.

The CAR of any one of embodiments 1 to 13, wherein the extracellular binding domain specific to PD-1 comprises or consists of an amino acid sequence set forth in SEQ ID NO: 32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 32.

The CAR of any one of embodiments 1 to 13, further comprising an inducible hepatitis C-derived NS3 protease domain.

A nucleic acid comprising a nucleotide sequence encoding the CAR of any one of embodiments 1-21.

The nucleic acid of embodiment 22, wherein the nucleotide sequence is codon-optimized.

The nucleic acid of embodiment 22 or 23, further comprising a nucleotide sequence encoding a truncated epidermal growth factor receptor (tEGFR) having an amino acid sequence at least about 50%, 55%, 60%, 65%, 70%, 75%, 80%, 85%, 90%, 95%, or 100% identical to SEQ ID NO: 34.

The nucleic acid of any one of embodiments 22-24, further comprising a nucleotide sequence encoding a c46 fusion inhibitor peptide having an amino acid sequence at least about 50%, 55%, 60%, 65%, 70%, 75%, 80%, 85%, 90%, 95%, or 100% identical to SEQ ID NO: 35.

The nucleic acid of any one of embodiments 22-25, wherein the nucleic acid comprises a nucleotide sequence encoding an amino acid sequence that is at least about 50%, 55%, 60%, 65%, 70%, 75%, 80%, 85%, 90%, 95%, or 100% identical to SEQ ID NO: 33.

A vector comprising the nucleic acid of any one of embodiments 22-26.

The vector of embodiment 27, wherein the vector is a plasmid, an adenoviral vector, an adeno-associated viral vector, a retroviral vector, or a lentiviral vector.

A virus comprising the nucleic acid of any one of embodiments 22-26, or the vector of embodiment 27 or 28.

The virus of embodiment 29, wherein the virus is an adenovirus, an adeno-associated virus, a retrovirus, a lentivirus, or a phage.

A composition comprising the vector of embodiment 27 or 28, or the virus of embodiment 29 or 30.

A host cell expressing the CAR of any one of embodiments 1-20, comprising the nucleic acid of any one of embodiments 22-26, and/or comprising the vector of embodiment 27 or 28.

The host cell of embodiment 32, wherein the host cell is a T cell.

The host cell of embodiment 33, wherein the T cell is a CD4+ T cell, a CD8+ T cell, or a mixture thereof.

The host cell of any one of embodiments 32-34, wherein the host cell is modified to express a safety switch.

The host cell of embodiment 35, wherein the safety switch is an inducible hepatitis C-derived NS3 protease domain or a tEGFR.

The host cell of any one of embodiments 32-36, wherein the host cell is modified to have reduced or eliminated expression of an endogenous TCR.

A pharmaceutical composition, comprising the host cell of any one of embodiments 32-37.

A method of treating and/or preventing HIV infection, symptoms associated with HIV infection, and/or AIDS in a subject in need thereof, comprising administering to the subject a therapeutically effective amount of the host cell of any one of embodiments 32-37, or the pharmaceutical composition of embodiment 38.

The method of embodiment 39, the method further comprising administering to the subject an antiretroviral therapy (ART).

A method of treating and/or preventing a PD-1-positive hematologic cancer in a subject in need thereof, comprising administering to the subject a therapeutically effective amount of the host cell of any one of embodiments 32-37, or the pharmaceutical composition of embodiment 38.

The method of embodiment 41, wherein the hematologic cancer is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T cell lymphoma, and B-cell lymphoma.

The method of embodiment 42, wherein the T-cell lymphoma is selected from the group consisting of angioimmunoblastic T-cell lymphoma, peripheral T-cell lymphoma with a follicular helper T cell (T_{FH}) phenotype, and T follicular helper lymphoma.

The method of any one of embodiments 39-43, the method further comprising administering to the subject one or more additional therapeutic agents selected from the group consisting of an immunotherapy agent, a chemotherapy agent, and a biologic agent.

A method of treating and/or preventing an autoimmune disease in a subject in need thereof, comprising administering to the subject a therapeutically effective amount of the host cell of any one of embodiments 32-37, or the pharmaceutical composition of embodiment 38.

The method of embodiment 45, wherein the autoimmune disease is selected from the group consisting of type 1 diabetes, lupus, systemic lupus erythematosus (SLE), rheumatoid arthritis, psoriasis, psoriatic arthritis, multiple sclerosis, inflammatory bowel disease, Crohn's disease, ulcerative colitis, Addison's disease, Graves' disease, Sjogren's syndrome, Hashimoto's thyroiditis, myasthenia gravis, autoimmune vasculitis, pernicious anemia, and celiac disease.

The CAR of embodiment 1, wherein the extracellular binding domain specific to PD-1 comprises an scFv derived from a portion of an anti-PD-1 antibody at least about 50%, 55%, 60%, 65%, 70%, 75%, 80%, 85%, 90%, 95%, or 100% identical to an antibody in Table 8.

The CAR of embodiment 47, wherein the scFv comprises one or more complementarity determining regions (CDRs) having amino acid sequences at least about 50%, 55%, 60%, 65%, 70%, 75%, 80%, 85%, 90%, 95%, or 100% identical to a sequence in Table 8.

The CAR of embodiment 48, wherein the scFV comprises a heavy chain variable region comprising an amino acid sequence that is at least about 50%, 55%, 60%, 65%, 70%, 75%, 80%, 85%, 90%, 95%, or 100% identical to any of the heavy chain variable regions set forth in Table 8 and/or a light chain variable region comprising an amino acid sequence that is at least about 80% identical to any of the light chain variable regions set forth in Table 8.

The CAR of any one of embodiments 47-49, wherein the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

The CAR of any one of embodiments 47-50, wherein the hinge domain comprises an IgG4 hinge domain having an amino acid sequence set forth at least about 50%, 55%, 60%, 65%, 70%, 75%, 80%, 85%, 90%, 95%, or 100% identical to one or more of SEQ ID NOs: 18-20.

The CAR of any one of embodiments 47-51, wherein the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

The CAR of any one of embodiments 47-52, wherein the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

The CAR of any one of embodiments 47-53, wherein the intracellular signaling domain comprises a CD3ζ signaling domain having an amino acid sequence at least about 50%, 55%, 60%, 65%, 70%, 75%, 80%, 85%, 90%, 95%, or 100% identical to SEQ ID NO: 25.

The CAR of any one of embodiments 47-54, wherein the anti-PD-1 CAR comprises or consists of an amino acid sequence set forth in Table 8, or an amino acid sequence that is at least 80% identical, at least 85% identical, at least 90% identical, at least 95% identical, at least 96% identical, at least 97% identical, at least 98% identical, at least 99% identical, or 100% identical to any one of the amino acid sequences set forth in Table 8.

A nucleic acid comprising a nucleotide sequence encoding the CAR of any one of embodiments 47-55.

The nucleic acid of embodiment 56, further comprising a nucleotide sequence encoding a truncated epidermal growth factor receptor (tEGFR) having an amino acid sequence set at least about 50%, 55%, 60%, 65%, 70%, 75%, 80%, 85%, 90%, 95%, or 100% identical to SEQ ID NO: 34.

The nucleic acid of embodiment 56 or embodiment 57, further comprising a nucleotide sequence encoding a c46 fusion inhibitor peptide having an amino acid sequence at least about 50%, 55%, 60%, 65%, 70%, 75%, 80%, 85%, 90%, 95%, or 100% identical to SEQ ID NO: 35.

The nucleic acid of any one of embodiments 56-58, wherein the nucleic acid comprises a nucleotide sequence encoding an amino acid sequence that is at least about 80% identical to an amino acid sequence at least about 50%, 55%, 60%, 65%, 70%, 75%, 80%, 85%, 90%, 95%, or 100% identical to a sequence in Table 8.

A vector comprising the nucleic acid of any one of embodiments 56-59.

The vector of embodiment 60, wherein the vector is a plasmid, an adenoviral vector, an adeno-associated viral vector, a retroviral vector, or a lentiviral vector.

A virus comprising the nucleic acid of any one of embodiments 56-59, or the vector of embodiment 60 or 61.

A composition comprising the vector of embodiment 60 or 61, or the virus of embodiment 62.

A host cell expressing the CAR of any one of embodiments 47-55, comprising the nucleic acid of any one of embodiments 56-59, and/or comprising the vector of embodiment 60 or 61.

A pharmaceutical composition, comprising the host cell of embodiment 64.

A method of treating and/or preventing HIV infection, symptoms associated with HIV infection, and/or AIDS in a subject in need thereof, comprising administering to the subject a therapeutically effective amount of the host cell of embodiment 64, or the pharmaceutical composition of embodiment 65.

A method of treating and/or preventing a PD-1-positive hematologic cancer in a subject in need thereof, comprising administering to the subject a therapeutically effective amount of the host cell of embodiment 64, or the pharmaceutical composition of embodiment 65.

A method of treating and/or preventing an autoimmune disease in a subject in need thereof, comprising administering to the subject a therapeutically effective amount of the host cell of embodiment 64, or the pharmaceutical composition of embodiment 65.

Use of the host cell of embodiment 64 or the pharmaceutical composition of embodiment 65 for treating and/or preventing HIV infection, symptoms associated with HIV infection, and/or AIDS in a subject in need thereof.

Use of the host cell of embodiment 64 or the pharmaceutical composition of embodiment 65 for treating and/or preventing a PD-1-positive hematologic cancer in a subject in need thereof.

Use of the host cell of embodiment 64 or the pharmaceutical composition of embodiment 65 for treating and/or preventing an autoimmune disease in a subject in need thereof.

I/We claim:

1. A chimeric antigen receptor (CAR) comprising a signal peptide, an extracellular binding domain specific to programmed death protein 1 (PD-1), a hinge domain, a transmembrane domain, an intracellular costimulatory domain, and/or an intracellular signaling domain.

2. The CAR of claim 1, wherein the extracellular binding domain specific to PD-1 comprises an scFv derived from a portion of pembrolizumab.

3. The CAR of claim 2, wherein the scFv comprises one or more complementarity determining regions (CDRs) having amino acid sequences at least about 50%, 55%, 60%, 65%, 70%, 75%, 80%, 85%, 90%, 95%, or 100% identical to SEQ ID NOs: 6-8 and 11-13.

4. The CAR of claim 3, wherein the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and/or a light chain variable region comprising an amino acid sequence that is at least about 50%, 55%, 60%, 65%, 70%, 75%, 80%, 85%, 90%, 95%, or 100% identical to SEQ ID NO: 10.

5. The CAR of claim 4, wherein the scFV comprises a heavy chain variable region comprising an amino acid sequence that is at least about 50%, 55%, 60%, 65%, 70%, 75%, 80%, 85%, 90%, 95%, or 100% identical to SEQ ID NO: 5; and a light chain variable region comprising an amino acid sequence that is at least about 50%, 55%, 60%, 65%, 70%, 75%, 80%, 85%, 90%, 95%, or 100% identical to SEQ ID NO: 10.

6. The CAR of claim 4, wherein the scFV comprises an amino acid sequence that is at least about 50%, 55%, 60%, 65%, 70%, 75%, 80%, 85%, 90%, 95%, or 100% identical to SEQ ID NO: 4 or SEQ ID NO: 14.

7. The CAR of claim 6, wherein the scFV comprises an amino acid sequence that at least about 50%, 55%, 60%, 65%, 70%, 75%, 80%, 85%, 90%, 95%, or 100% identical to SEQ ID NO: 4.

8. The CAR of claim 6, wherein the scFV comprises an amino acid sequence that is at least about 50%, 55%, 60%, 65%, 70%, 75%, 80%, 85%, 90%, 95%, or 100% identical to SEQ ID NO: 14.

9. The CAR of any one of claims 1-8, wherein the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

10. The CAR of any one of claims 1-9, wherein the hinge domain comprises an IgG4 hinge domain having an amino acid sequence at least about 50%, 55%, 60%, 65%, 70%, 75%, 80%, 85%, 90%, 95%, or 100% identical to SEQ ID NOs: 18-20.

11. The CAR of any one of claims 1-10, wherein the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

12. The CAR of any one of claims 1-11, wherein the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

13. The CAR of any one of claims 1-12, wherein the intracellular signaling domain comprises a CD3ζ signaling domain having an amino acid sequence at least about 50%, 55%, 60%, 65%, 70%, 75%, 80%, 85%, 90%, 95%, or 100% identical to SEQ ID NO: 25.

14. The CAR of any one of claims 1 to 13, wherein the extracellular binding domain specific to PD-1 comprises or consists of an amino acid sequence set forth in any one of SEQ ID NOs: 27-32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in any one of SEQ ID NOs: 27-32.

15. The CAR of any one of claims 1 to 13, wherein the extracellular binding domain specific to PD-1 comprises or consists of an amino acid sequence set forth in SEQ ID NO: 27, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 27.

16. The CAR of any one of claims 1 to 13, wherein the extracellular binding domain specific to PD-1 comprises or consists of an amino acid sequence set forth in SEQ ID NO: 28, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 28.

17. The CAR of any one of claims 1 to 13, wherein the extracellular binding domain specific to PD-1 comprises or consists of an amino acid sequence set forth in SEQ ID NO: 29, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 29.

18. The CAR of any one of claims 1 to 13, wherein the extracellular binding domain specific to PD-1 comprises or consists of an amino acid sequence set forth in SEQ ID NO: 30, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 30.

19. The CAR of any one of claims 1 to 13, wherein the extracellular binding domain specific to PD-1 comprises or consists of an amino acid sequence set forth in SEQ ID NO: 31, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 31.

20. The CAR of any one of claims 1 to 13, wherein the extracellular binding domain specific to PD-1 comprises or consists of an amino acid sequence set forth in SEQ ID NO: 32, or an amino acid sequence that is at least 50% identical, at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, at least 99% identical, or 100% identical to the amino acid sequence set forth in SEQ ID NO: 32.

21. The CAR of any one of claims 1 to 13, further comprising an inducible hepatitis C-derived NS3 protease domain.

22. A nucleic acid comprising a nucleotide sequence encoding the CAR of any one of claims 1-21.

23. The nucleic acid of claim 22, wherein the nucleotide sequence is codon-optimized.

24. The nucleic acid of claim 22 or 23, further comprising a nucleotide sequence encoding a truncated epidermal growth factor receptor (tEGFR) having an amino acid sequence at least about 50%, 55%, 60%, 65%, 70%, 75%, 80%, 85%, 90%, 95%, or 100% identical to SEQ ID NO: 34.

25. The nucleic acid of any one of claims 22-24, further comprising a nucleotide sequence encoding a c46 fusion inhibitor peptide having an amino acid sequence at least about 50%, 55%, 60%, 65%, 70%, 75%, 80%, 85%, 90%, 95%, or 100% identical to SEQ ID NO: 35.

26. The nucleic acid of any one of claims 22-25, wherein the nucleic acid comprises a nucleotide sequence encoding an amino acid sequence that is at least about 50%, 55%, 60%, 65%, 70%, 75%, 80%, 85%, 90%, 95%, or 100% identical to SEQ ID NO: 33.

27. A vector comprising the nucleic acid of any one of claims 22-26.

28. The vector of claim 27, wherein the vector is a plasmid, an adenoviral vector, an adeno-associated viral vector, a retroviral vector, or a lentiviral vector.

29. A virus comprising the nucleic acid of any one of claims 22-26, or the vector of claim 27 or 28.

30. The virus of claim 29, wherein the virus is an adenovirus, an adeno-associated virus, a retrovirus, a lentivirus, or a phage.

31. A composition comprising the vector of claim 27 or 28, or the virus of claim 29 or 30.

32. A host cell expressing the CAR of any one of claims 1-20, comprising the nucleic acid of any one of claims 22-26, and/or comprising the vector of claim 27 or 28.

33. The host cell of claim 32, wherein the host cell is a T cell.

34. The host cell of claim 33, wherein the T cell is a CD4+ T cell, a CD8+ T cell, or a mixture thereof.

35. The host cell of any one of claims 32-34, wherein the host cell is modified to express a safety switch.

36. The host cell of claim 35, wherein the safety switch is an inducible hepatitis C-derived NS3 protease domain or a tEGFR.

37. The host cell of any one of claims 32-36, wherein the host cell is modified to have reduced or eliminated expression of an endogenous TCR.

38. A pharmaceutical composition, comprising the host cell of any one of claims 32-37.

39. A method of treating and/or preventing HIV infection, symptoms associated with HIV infection, and/or AIDS in a subject in need thereof, comprising administering to the subject a therapeutically effective amount of the host cell of any one of claims 32-37, or the pharmaceutical composition of claim 38.

40. The method of claim 39, the method further comprising administering to the subject an antiretroviral therapy (ART).

41. A method of treating and/or preventing a PD-1-positive hematologic cancer in a subject in need thereof, comprising administering to the subject a therapeutically effective amount of the host cell of any one of claims 32-37, or the pharmaceutical composition of claim 38.

42. The method of claim 41, wherein the hematologic cancer is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T cell lymphoma, and B-cell lymphoma.

43. The method of claim 42, wherein the T-cell lymphoma is selected from the group consisting of angioimmunoblastic T-cell lymphoma, peripheral T-cell lymphoma with a follicular helper T cell (T_{FH}) phenotype, and T follicular helper lymphoma.

44. The method of any one of claims 39-43, the method further comprising administering to the subject one or more additional therapeutic agents selected from the group consisting of an immunotherapy agent, a chemotherapy agent, and a biologic agent.

45. A method of treating and/or preventing an autoimmune disease in a subject in need thereof, comprising administering to the subject a therapeutically effective amount of the host cell of any one of claims 32-37, or the pharmaceutical composition of claim 38.

46. The method of claim 45, wherein the autoimmune disease is selected from the group consisting of type 1 diabetes, lupus, systemic lupus erythematosus (SLE), rheumatoid arthritis, psoriasis, psoriatic arthritis, multiple sclerosis, inflammatory bowel disease, Crohn's disease, ulcerative colitis, Addison's disease, Graves' disease, Sjogren's syndrome, Hashimoto's thyroiditis, myasthenia gravis, autoimmune vasculitis, pernicious anemia, and celiac disease.

47. The CAR of claim 1, wherein the extracellular binding domain specific to PD-1 comprises an scFv derived from a portion of an anti-PD-1 antibody at least about 50%, 55%, 60%, 65%, 70%, 75%, 80%, 85%, 90%, 95%, or 100% identical to an antibody in Table 8.

48. The CAR of claim 47, wherein the scFv comprises one or more complementarity determining regions (CDRs) having amino acid sequences at least about 50%, 55%, 60%, 65%, 70%, 75%, 80%, 85%, 90%, 95%, or 100% identical to a sequence in Table 8.

49. The CAR of claim 48, wherein the scFV comprises a heavy chain variable region comprising an amino acid sequence that is at least about 50%, 55%, 60%, 65%, 70%, 75%, 80%, 85%, 90%, 95%, or 100% identical to any of the heavy chain variable regions set forth in Table 8 and/or a light chain variable region comprising an amino acid sequence that is at least about 80% identical to any of the light chain variable regions set forth in Table 8.

50. The CAR of any one of claims 47-49, wherein the signal peptide comprises a GM-CSF signal peptide having an amino acid sequence at least about 50%, 55%, 60%, 65%, 70%, 75%, 80%, 85%, 90%, 95%, or 100% identical to SEQ ID NO: 3.

51. The CAR of any one of claims 47-50, wherein the hinge domain comprises an IgG4 hinge domain having an amino acid sequence set forth at least about 50%, 55%, 60%, 65%, 70%, 75%, 80%, 85%, 90%, 95%, or 100% identical to one or more of SEQ ID NOs: 18-20.

52. The CAR of any one of claims 47-51, wherein the transmembrane domain comprises a CD28 transmembrane domain having an amino acid sequence at least about 50%, 55%, 60%, 65%, 70%, 75%, 80%, 85%, 90%, 95%, or 100% identical to SEQ ID NO: 17.

53. The CAR of any one of claims 47-52, wherein the intracellular costimulatory domain comprises a 4-1BB costimulatory domain having an amino acid sequence at least about 50%, 55%, 60%, 65%, 70%, 75%, 80%, 85%, 90%, 95%, or 100% identical to SEQ ID NO: 23.

54. The CAR of any one of claims 47-53, wherein the intracellular signaling domain comprises a CD3ζ signaling domain having an amino acid sequence at least about 50%, 55%, 60%, 65%, 70%, 75%, 80%, 85%, 90%, 95%, or 100% identical to SEQ ID NO: 25.

55. The CAR of any one of claims 47-54, wherein the anti-PD-1 CAR comprises or consists of an amino acid sequence set forth in Table 8, or an amino acid sequence that is at least 80% identical, at least 85% identical, at least 90% identical, at least 95% identical, at least 96% identical, at least 97% identical, at least 98% identical, at least 99% identical, or 100% identical to any one of the amino acid sequences set forth in Table 8.

56. A nucleic acid comprising a nucleotide sequence encoding the CAR of any one of claims 47-55.

57. The nucleic acid of claim 56, further comprising a nucleotide sequence encoding a truncated epidermal growth factor receptor (tEGFR) having an amino acid sequence set at least about 50%, 55%, 60%, 65%, 70%, 75%, 80%, 85%, 90%, 95%, or 100% identical to SEQ ID NO: 34.

58. The nucleic acid of claim 56 or claim 57, further comprising a nucleotide sequence encoding a c46 fusion inhibitor peptide having an amino acid sequence at least about 50%, 55%, 60%, 65%, 70%, 75%, 80%, 85%, 90%, 95%, or 100% identical to SEQ ID NO: 35.

59. The nucleic acid of any one of claims 56-58, wherein the nucleic acid comprises a nucleotide sequence encoding an amino acid sequence that is at least about 80% identical to an amino acid sequence at least about 50%, 55%, 60%, 65%, 70%, 75%, 80%, 85%, 90%, 95%, or 100% identical to a sequence in Table 8.

60. A vector comprising the nucleic acid of any one of claims 56-59.

61. The vector of claim 60, wherein the vector is a plasmid, an adenoviral vector, an adeno-associated viral vector, a retroviral vector, or a lentiviral vector.

62. A virus comprising the nucleic acid of any one of claims 56-59, or the vector of claim 60 or 61.

63. A composition comprising the vector of claim 60 or 61, or the virus of claim 62.

64. A host cell expressing the CAR of any one of claims 47-55, comprising the nucleic acid of any one of claims 56-59, and/or comprising the vector of claim 60 or 61.

65. A pharmaceutical composition, comprising the host cell of claim 64.

66. A method of treating and/or preventing HIV infection, symptoms associated with HIV infection, and/or AIDS in a subject in need thereof, comprising administering to the subject a therapeutically effective amount of the host cell of claim 64, or the pharmaceutical composition of claim 65.

67. A method of treating and/or preventing a PD-1-positive hematologic cancer in a subject in need thereof, comprising administering to the subject a therapeutically effective amount of the host cell of claim 64, or the pharmaceutical composition of claim 65.

68. A method of treating and/or preventing an autoimmune disease in a subject in need thereof, comprising administering to the subject a therapeutically effective amount of the host cell of claim 64, or the pharmaceutical composition of claim 65.

69. Use of the host cell of claim 64 or the pharmaceutical composition of claim 65 for treating and/or preventing HIV infection, symptoms associated with HIV infection, and/or AIDS in a subject in need thereof.

70. Use of the host cell of claim 64 or the pharmaceutical composition of claim 65 for treating and/or preventing a PD-1-positive hematologic cancer in a subject in need thereof.

71. Use of the host cell of claim 64 or the pharmaceutical composition of claim 65 for treating and/or preventing an autoimmune disease in a subject in need thereof.
